(19) United States
(12) Patent Application Publication
(10) Pub. No.: US 2026/0125370 A1
(43) Pub. Date: May 7, 2026

(54) SULFOXIMINE COMPOUNDS AS HISTONE DEACETYLASE 6 INHIBITOR, AND PHARMACEUTICAL COMPOSITION COMPRISING THE SAME

(71) Applicant: CHONG KUN DANG PHARMACEUTICAL CORP., Seoul (KR)

(72) Inventors: Jung Taek OH, Gyeonggi-do (KR); Hyeseung SONG, Gyeonggi-do (KR); Chang Sik LEE, Gyeonggi-do (KR)

(21) Appl. No.: 19/118,934

(22) PCT Filed: Oct. 12, 2023
(86) PCT No.: PCT/IB2023/060289

§ 371 Date: Apr. 7, 2025

(30) Foreign Application Priority Data
Oct. 14, 2022 (KR) 10-2022-0132710

(51) Int. Cl. C07D 413/04; A61K 31/4245; A61K 31/4995; A61K 31/541; A61K 31/547; A61K 31/675; C07D 295/15; C07D 413/12; C07D 413/14; C07D 417/04; C07D 487/08; C07D 487/10; C07D 495/08; C07D 495/10; C07F 9/6558

(52) U.S. Cl. CPC C07D 413/04; A61K 31/4245; A61K 31/4995; A61K 31/541; A61K 31/547; A61K 31/675; C07D 295/15; C07D 413/12; C07D 413/14; C07D 417/04; C07D 487/08; C07D 487/10; C07D 495/08; C07D 495/10; C07F 9/65583

(57) ABSTRACT

The present invention relates to a sulfoximine compound having a novel structure, stereoisomers thereof or pharmaceutically acceptable salts thereof, and a use thereof for preventing or treating histone deacetylase-mediated diseases The sulfoximine compound having a novel structure according to the present invention may be represented by Formula (I) below.

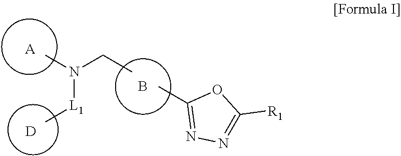

### TECHNICAL FIELD

The present invention relates to a sulfoximine compound having a histone deacetylase 6 (HDAC6) inhibitory activity, stereoisomers thereof or pharmaceutically acceptable salts thereof, and a use thereof.

### BACKGROUND ART

In cells, a post-translational modification such as acetylation serves as a very important regulatory module at the hub of biological processes, and is also strictly controlled by a number of enzymes. As a core protein constituting chromatin, histone functions as an axis, around which DNA winds, and thus helps a DNA condensation. Also, a balance between acetylation and deacetylation of histone plays a very important role in gene expression.

As an enzyme for removing an acetyl group from lysine residue of histone protein, which constitutes chromatin, histone deacetylase (HDAC) is known to be associated with gene silencing and induce a cell cycle arrest, angiogenic inhibition, immunoregulation, apoptosis, etc. (Hassig et al., Curr. Opin. Chem. Biol. 1997, 1, 300-308). Also, it is reported that the inhibition of HDAC enzyme functions induces cancer cells into committing apoptosis for themselves by lowering an activity of cancer cell survival-related factors and activating cancer cell death-related factors in the body (Warrell et al., J. Natl. Cancer Inst. 1998, 90, 1621-1625).

For humans, 18 HDACs are known and classified into four classes according to homology with yeast HDAC. In this case, eleven HDACs using zinc as a cofactor may be divided into three groups: Class I (HDAC1, 2, 3, 8), Class II (IIa: HDAC4, 5, 7, 9; IIb: HDAC6, 10) and Class IV (HDAC11). Further, seven HDACs of Class III (SIRT 1-7) use NAD+ as a cofactor instead of zinc (Bolden et al., Nat. Rev. Drug Discov. 2006, 5(9), 769-784).

Various HDAC inhibitors are now in a preclinical or clinical development stage, but only non-selective HDAC inhibitors have been known as an anti-cancer agent so far. Vorinostat (SAHA) and romidepsin (FK228) have obtained an approval as a therapeutic agent for cutaneous T-cell lymphoma, while panobinostat (LBH-589) has won an approval as a therapeutic agent for multiple myeloma. However, it is known that the non-selective HDAC inhibitors generally bring about side effects such as fatigue, nausea and the like at high doses (Piekarz et al., Pharmaceuticals 2010, 3, 2751-2767). It is reported that the side effects are caused by the inhibition of class I HDACs. Due to the side effects, etc., the non-selective HDAC inhibitors have been subject to restriction on drug development in other fields than an anticancer agent (Witt et al., Cancer Letters 277, (2009), 8-21).

Meanwhile, it is reported that the selective inhibition of class II HDACs would not show toxicity, which have occurred in the inhibition of class I HDACs. In case of developing the selective HDAC inhibitors, it would be likely to solve side effects such as toxicity, etc., caused by the non-selective inhibition of HDACs. Accordingly, there is a chance that the selective HDAC inhibitors may be developed as an effective therapeutic agent for various diseases (Matthias et al., Mol. Cell. Biol. 2008, 28, 1688-1701).

HDAC6, one of the class IIb HDACs, is known to be mainly present in cytoplasma and contain a tubulin protein, thus being involved in the deacetylation of a number of non-histone substrates (HSP90, cortactin, etc.) (Yao et al., Mol. Cell 2005, 18, 601-607). HDAC6 has two catalytic domains, in which a zinc finger domain of C-terminal may bind to an ubiquitinated protein. HDAC6 is known to have a number of non-histone proteins as a substrate, and thus play an important role in various diseases such as cancer, inflammatory diseases, autoimmune diseases, neurological diseases, neurodegenerative disorders and the like (Santo et al., Blood 2012 119: 2579-2589; Vishwakarma et al., International Immunopharmacology 2013, 16, 72-78; Hu et al., J. Neurol. Sci. 2011, 304, 1-8).

A structural feature that various HDAC inhibitors have in common is comprised of a cap group, a linker group and a zinc binding group (ZBG) as shown in a following structure of vorinostat. Many researchers have conducted a study on the inhibitory activity and selectivity with regard to enzymes through a structural modification of the cap group and the linker group. Out of the groups, it is known that the zinc binding group plays a more important role in the enzyme inhibitory activity and selectivity (Wiest et al., J. Org. Chem. 2013 78: 5051-5055; Methot et al., Bioorg. Med. Chem. Lett. 2008, 18, 973-978).

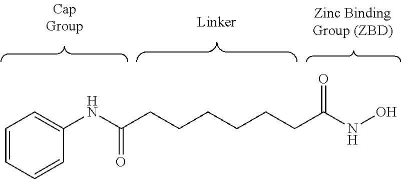 [SMILES: O=C(CCCCCCC(=O)Nc1ccccc1)NO]

Most of said zinc binding group is comprised of hydroxamic acid or benzamide, out of which hydroxamic acid derivatives show a strong HDAC inhibitory effect, but have a problem with low bioavailability and serious off-target activity. Benzamide contains aniline, and thus has a problem in that it may produce toxic metabolites in vivo (Woster et al., Med. Chem. Commun. 2015, online publication).

Accordingly, unlike the non-selective inhibitors having side effects, there is a need to develop a selective HDAC6 inhibitor, which has a zinc binding group with improved bioavailability, while causing no side effects in order to treat cancer, inflammatory diseases, autoimmune diseases, neurological diseases, neurodegenerative disorders and the like.

### RELATED ART REFERENCE

### Patent Documents

International Patent Publication No. WO 2011/091213 (publicized on Jul. 28, 2011): ACY-1215
International Patent Publication No. WO 2011/011186 (publicized on Jan. 27, 2011): Tubastatin
International Patent Publication No. WO 2013/052110 (publicized on Apr. 11, 2013): Sloan-K
International Patent Publication No. WO 2013/041407 (publicized on Mar. 28, 2013): Cellzome
International Patent Publication No. WO 2013/134467 (publicized on Sep. 12, 2013): Kozi
International Patent Publication No. WO 2013/008162 (publicized on Jan. 17, 2013): Novartis
International Patent Publication No. WO 2013/080120 (publicized on Jun. 6, 2013): Novartis
International Patent Publication No. WO 2013/066835 (publicized on May 10, 2013): Tempero
International Patent Publication No. WO 2013/066838 (publicized on May 10, 2013): Tempero
International Patent Publication No. WO 2013/066833 (publicized on May 10, 2013): Tempero
International Patent Publication No. WO 2013/066839 (publicized on May 10, 2013): Tempero

### DISCLOSURE

### Technical Problem

An objective of the present invention is to provide a novel sulfoximine compound having a HDAC6 inhibitory activity, stereoisomers thereof or pharmaceutically acceptable salts thereof.

Another objective of the present invention is to provide a pharmaceutical composition containing a novel sulfoximine compound having a HDAC6 inhibitory activity, stereoisomers thereof or pharmaceutically acceptable salts thereof.

Still another objective of the present invention is to provide a method for preparing a novel sulfoximine compound, stereoisomers thereof or pharmaceutically acceptable salts thereof.

Still another objective of the present invention is to provide a pharmaceutical composition for preventing or treating HDAC-mediated diseases, containing the novel sulfoximine compound, stereoisomers thereof or pharmaceutically acceptable salts thereof as an effective ingredient.

Still another objective of the present invention is to provide a method for preventing or treating HDAC-mediated diseases including administering the novel sulfoximine compound, stereoisomers thereof or pharmaceutically acceptable salts thereof into an individual.

Still another objective of the present invention is to provide a use of the novel sulfoximine compound, stereoisomers thereof or pharmaceutically acceptable salts thereof for preventing or treating HDAC-mediated diseases.

Still another objective of the present invention is to provide a use of the novel sulfoximine compound, stereoisomers thereof or pharmaceutically acceptable salts thereof in preparing a medicament for preventing or treating HDAC-mediated diseases.

### Technical Solution

The present inventors have confirmed a novel compound having a HDAC inhibitory activity and have used the same in preventing and/or treating HDAC-mediated diseases, thereby completing the present invention.

This is described in detail as follows. Every combination of various elements disclosed in the present invention falls within the scope of the present invention. Also, it cannot be seen that the scope of the present invention is limited to the specific description described below.

### Compound

The present invention provides a sulfoximine compound according to any one of (1) to (6) below, stereoisomers thereof or pharmaceutically acceptable salts thereof.
(1) A sulfoximine compound represented by formula I below, stereoisomers thereof or pharmaceutically acceptable salts thereof:

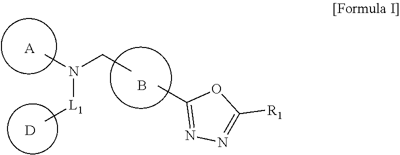

in above formula I,
R₁ is CF₂H or CF₃;
L₁ is —(C₁₋₃ alkylene)-, —C(═O)—, —S(═O)₂—, —S(═O)₂—(C₁₋₃ alkylene)-, —C(═O)N(Rₐ)—,

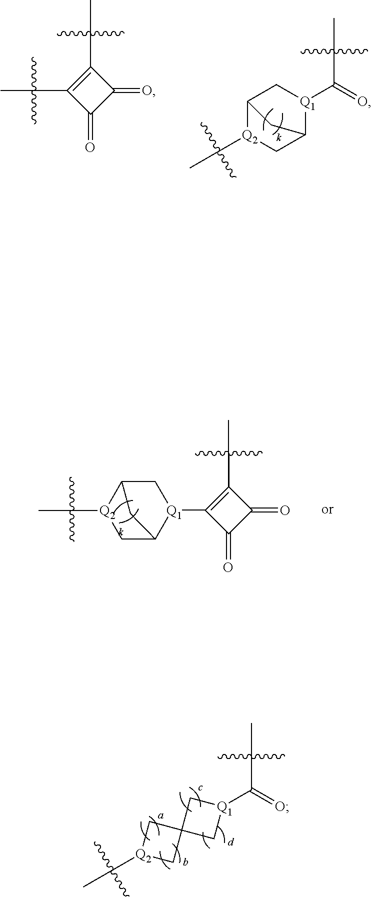

Rₐ is H or C₁₋₆ alkyl;
Q₁ and Q₂ are each independently CH or N;
a, b, c, d and k are each independently 1 or 2;
\textcircled{A} is C₃₋₁₂ cycloalkyl, 3- to 12-membered heterocycloalkyl including in a ring 1 to 3 heteroatoms independently selected from the group consisting of N, O and S, C₆-C₁₂ aryl, —(C₁₋₃ alkylene)C₆-C₁₂ aryl, 5- to 12-membered heteroaryl including in a ring 1 to 3 heteroatoms independently selected from the group consisting of N, O and S, or a 9- to 12-membered fused ring group including a structure in which an aromatic ring group and a non-aromatic ring group are fused;
at least one H of above \textcircled{A} may be each independently substituted with C₁₋₆ alkyl, C₂₋₆ alkenyl, C₂₋₆ alkynyl, C₃₋₁₀ cycloalkyl, C₃₋₁₂ cycloalkenyl, C₁₋₆ alkoxy, 3- to 12-membered heterocycloalkyl including in a ring 1 to 3 heteroatoms independently selected from the group consisting of N, O and S (in which at least one H of heterocycloalkyl may be substituted with —C(═O)(C₁₋₆ alkyl), 5- to 12-membered heteroaryl including in a ring 1 to 3 heteroatoms independently selected from the group consisting of N, O and S, —CF₃, —S(CF₃), halogen or —S(═NH)(═O)R_{c};
R_{c} is H or C₁₋₆ alkyl;
\textcircled{B} is C₆-C₁₂ aryl or 5- to 12-membered heteroaryl including in a ring 1 to 3 heteroatoms independently selected from the group consisting of N, O and S;
at least one H of above \textcircled{B} may be each independently substituted with C₁₋₆ alkyl or halogen;
\textcircled{D} is

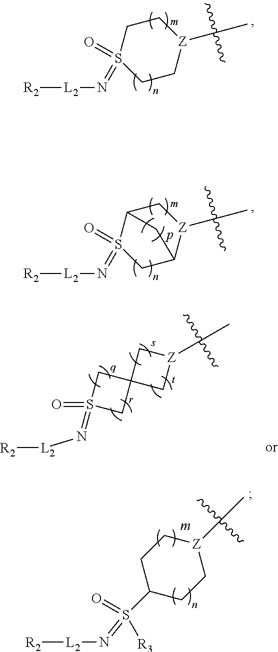

at least one H of above \textcircled{D} may be each independently substituted with C₁₋₆ alkyl or halogen;
Z is CH or N;
m and n are each independently 0, 1 or 2;
p, q, r, s and t are each independently 1 or 2;
L₂ is a single bond, —(C═O)—, —C(═O)O—, —C(═O)O—(C₁₋₃ alkylene)- or —S(═O)₂—;
R₂ is H, C₁₋₆ alkyl, C₆-C₁₂ aryl, CF₃ or —(P═O)(OR_{b})₂;
R_{b} is H or C₁₋₆ alkyl; and
R₃ is —C₁₋₆ alkyl.

In the present invention, in the expression “Cₓ” of a functional group, x may represent the number of carbons (C), and C_{x-y} may mean an integer of carbon number x or more and carbon number y or less.

In the present invention, a “single bond” may mean a case in which adjacent atoms or groups of atoms are directly bonded to each other.

In the present invention, “alkyl” may mean a linear (or straight-chain) saturated hydrocarbon group or a branched (or side-chain) saturated hydrocarbon group unless otherwise specified, and may include at least one selected from methyl, ethyl, n-propyl, isopropyl, n-butyl, sec-butyl, isobutyl, tert-butyl, n-pentyl, n-hexyl, n-heptyl, and the like, but is not limited thereto.

In the present invention, “alkylene” may mean a divalent functional group which is induced from alkyl as defined above, unless otherwise specified. For example, C₁ alkylene is methylene.

In the present invention, “alkenyl” may mean an unsaturated hydrocarbon group including at least one double bond between carbons, unless otherwise specified.

In the present invention, “alkynyl” may mean an unsaturated hydrocarbon group including at least one triple bond between carbons, unless otherwise specified.

In the present invention, “halogen” may mean F, Cl, Br or I, unless otherwise specified.

In the present invention, “aryl” may include a monocyclic aromatic or polycyclic aromatic one, unless otherwise specified, and the monocyclic aromatic one may be phenyl, and the polycyclic aromatic one may be at least one selected from biphenyl, naphthalenyl, etc., but is not limited thereto.

In the present invention, “benzyl” may mean

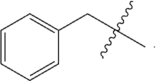 [SMILES: *Cc1ccccc1]

In the present invention, “heteroaryl” may mean a monocyclic or polycyclic hetero ring in which at least one carbon atom is substituted with nitrogen (N), oxygen (O) or sulfur (S) in the aryl. When heteroaryl includes at least two heteroatoms, the two heteroatoms or more may be the same or different from each other. Heteroaryl may include at least one selected from pyridinyl, thiopenyl, triazolyl, tetrazolyl, benzothiazolyl, benzothiophenyl, quinolinyl, indolyl, isoindolyl, benzofuranyl, benzopyrrolyl, furanyl, pyrrolyl, thiazolyl, isothiazolyl, imidazolyl, pyrazolyl, oxazolyl, isooxazolyl, pyrazinyl, pyridazinyl, pyrimidinyl, isoquinolinyl, benzooxazolyl, benzoimidazolyl, purinyl, indolizinyl, chromenyl, and the like, but is not limited thereto.

In the present invention, “cycloalkyl” may mean a saturated hydrocarbon ring having three or more specified carbon atoms including a ring, and the saturated hydrocarbon ring may include all of a monocyclic or polycyclic structure, and may further include a bicyclic or polycyclic structure such as a bridged ring or a spiro structure. Examples of cycloalkyl may include at least one selected from cyclopropyl, cyclobutyl, cyclopentyl, cyclohexyl, cycloheptyl, cyclooctyl, decahydronaphthalenyl, spiro[4.4]nonanyl and bicyclo[2.2.1]heptanyl, and the like, but are not limited thereto.

In the present invention, “heterocycloalkyl” may mean a cyclic group in which at least one carbon atom forming a ring in the cycloalkyl is each independently substituted with a heteroatom selected from the group consisting of N, O, and S. Examples of heterocycloalkyl may include at least one selected from oxiranyl, oxetanyl, tetrahydrofuranyl, tetrahydropyranyl, piperidinyl, piperazinyl, morpholinyl, thiomorpholinyl, pyrrolidinyl,

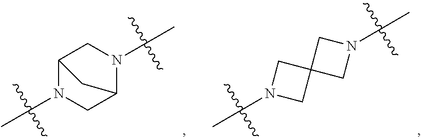 [SMILES: *N1CC2CC1CN2* ; *N1CC2(C1)CN(*)C2]

and the like, but are not limited thereto.

In the present invention, “cycloalkenyl” may mean a functional group in which the defined cycloalkyl structure includes at least one double bond.

In the present invention, “heterocycloalkenyl” may mean a functional group in which at least one carbon atom in the defined cycloalkenyl structure is each independently substituted with a heteroatom selected from the group consisting of N, O, and S.

In the present invention, a fused ring group may include a fused structure between an aromatic ring group and a non-aromatic ring group. The aromatic ring group may include the aryl group or the heteroaryl group as described above, and the non-aromatic ring group may include at least one of the cycloalkyl, heterocycloalkyl, cycloalkenyl, and heterocycloalkenyl as described above. Specifically, the fused ring group may be defined as a structure in which the aryl or heteroaryl is fused with one ring selected from the group consisting of cycloalkyl, heterocycloalkyl, cycloalkenyl, and heterocycloalkenyl as defined above. For example, the fused ring group may include a structure in which any one of aryl and heteroaryl is fused with at least one ring selected from the group consisting of cycloalkyl, heterocycloalkyl, cycloalkenyl, and heterocycloalkenyl (for example,

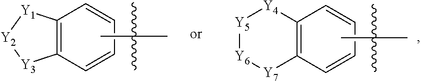

in which Y₁ to Y₇ may be each independently C, N, S or O, hydrogen may be bonded according to the number of bonds of each atom, Y₁—Y₂—Y₃ may be all single bonds or may include one double bond, and Y₄—Y₅—Y₆—Y₇ may be all connected by a single bond or may include at least one double bond), or a structure in which any one of cycloalkyl, heterocycloalkyl, cycloalkenyl and heterocycloalkenyl is fused with at least one ring of aryl and heteroaryl (for example,

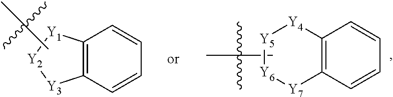

in which Y₁ to Y₇ are the same as defined above).

In the present invention,

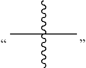

may represent a connected part.
(2) The sulfoximine compound according to above (1), stereoisomers thereof or pharmaceutically acceptable salts thereof,
in which, in above formula I:
L₁ is —C(═O)—, —S(═O)₂—, —S(═O)₂—(C₁₋₃ alkylene)-, —C(═O)N(Rₐ)—,

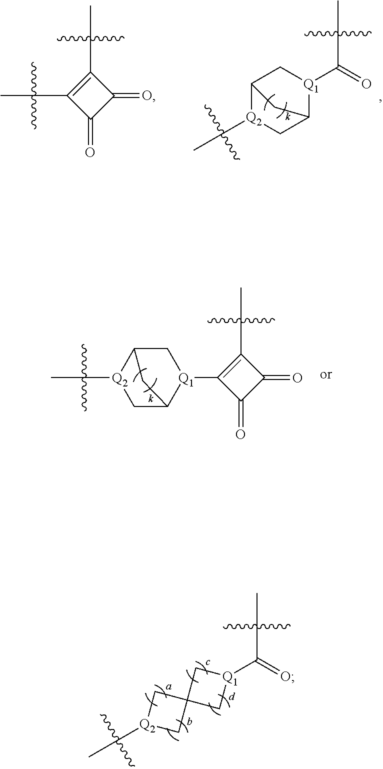

\textcircled{A} is C₆-C₁₂ aryl, —(C₁₋₃ alkylene)C₆-C₁₂ aryl, 5- to 12-membered heteroaryl including in a ring 1 to 3 heteroatoms independently selected from the group consisting of N, O and S, or

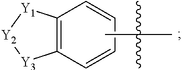

Y₁, Y₂ and Y₃ are each independently —CH₂— or —O—;
at least one H of above \textcircled{A} may be each independently substituted with C₁₋₆ alkyl, C₃₋₁₀ cycloalkyl, C₁₋₆ alkoxy, 3- to 12-membered heterocycloalkyl including in a ring 1 to 3 heteroatoms independently selected from the group consisting of N, O and S {in which at least one H of heterocycloalkyl may be substituted with —C(═O)(C₁₋₆ alkyl)}, 5- to 12-membered heteroaryl including in a ring 1 to 3 heteroatoms independently selected from the group consisting of N, O and S, —CF₃, —S(CF₃), halogen or —S(═NH)(═O)R_{c};
\textcircled{B} is C₆-C₁₂ aryl or 5- to 12-membered heteroaryl including in a ring 1 to 3 heteroatoms independently selected from the group consisting of N, O and S;
at least one H of above \textcircled{B} may be each independently substituted with halogen;
\textcircled{D} is

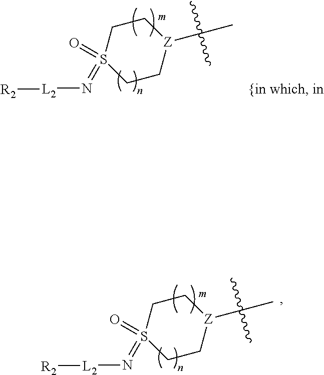

at least one H of

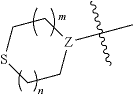

may be each independently substituted with C₁₋₆ alkyl or halogen},

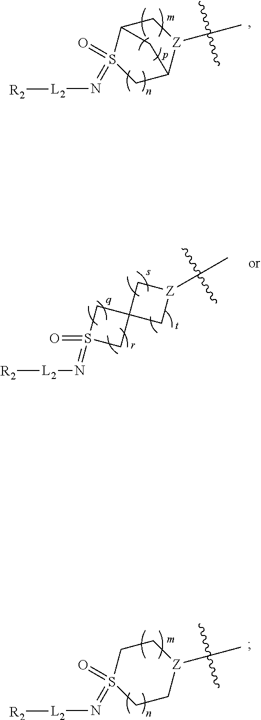

and
R₁, R₂, R₃, Rₐ, R_{b}, R_{c}, Q₁, Q₂, L₂, Z, a, b, c, d, k, m, n, p, q, r, s and t may be the same as defined in above formula I, respectively.
(3) The sulfoximine compound according to above (1) or (2), stereoisomers thereof or pharmaceutically acceptable salts thereof,
in which, the sulfoximine compound according to the present invention may be a compound represented by formula Ia below:

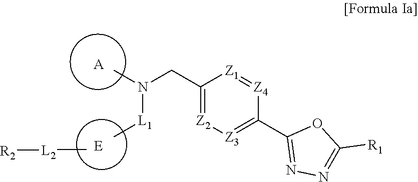

in above formula Ia,
L₁ is —C(═O)—, —S(═O)₂—, —S(═O)₂—(C₁₋₃ alkylene)-, —C(═O)N(Rₐ)—,

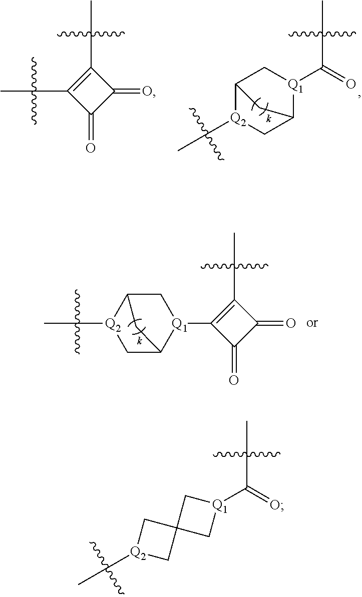

\textcircled{A} is C₆-C₁₂ aryl, —(C₁₋₃ alkylene)C₆-C₁₂ aryl, 5- to 12-membered heteroaryl including in a ring 1 to 3 heteroatoms independently selected from the group consisting of N, O and S, or a 9- to 12-membered fused ring group including a structure in which an aromatic ring group and a non-aromatic ring group are fused;
at least one H of above \textcircled{A} may be each independently substituted with C₁₋₆ alkyl, C₁₋₆ alkoxy, 3- to 12-membered heterocycloalkyl including in a ring 1 to 3 heteroatoms independently selected from the group consisting of N, O and S {in which at least one H of heterocycloalkyl may be substituted with —C(═O)(C₁₋₆ alkyl)}, 5- to 12-membered heteroaryl including in a ring 1 to 3 heteroatoms independently selected from the group consisting of N, O and S, —CF₃, —S(CF₃), halogen or —S(═NH)(═O)R_{c};
Z₁, Z₂, Z₃ and Z₄ are each independently CH, CX or N, in which at least one of Z₁ to Z₄ is CX or N;
X is halogen;
\textcircled{E} is

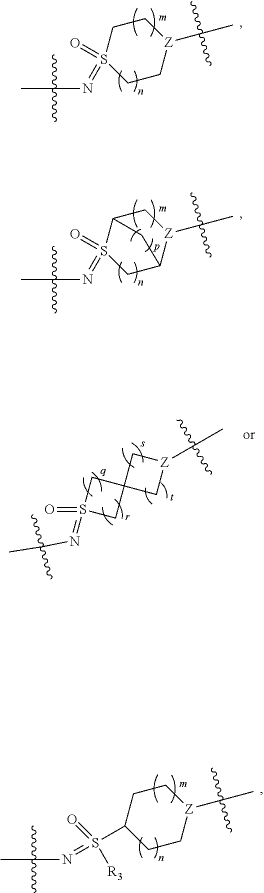

in above

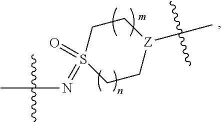

at least one H of

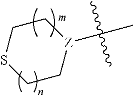

may be substituted with C₁₋₆ alkyl; and
R₁, R₂, R₃, Rₐ, R_{c}, Q₁, Q₂, L₂, Z, k, m, n, p, q, r, s and t are may be the same as defined in above formula I, respectively.

In \textcircled{E} of above formula Ia, Z may be bonded to L₁, and N may be bonded to L₂.
(4) The sulfoximine compound according to above (1), (2) or (3), stereoisomers thereof or pharmaceutically acceptable salts thereof,
in which, the sulfoximine compound according to the present invention may be a compound represented by formula Ib below:

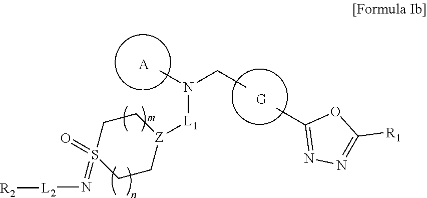

in above formula Ib,

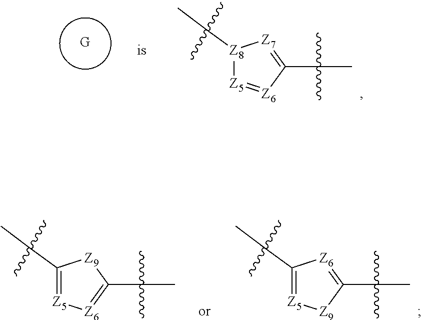

Z₅, Z₆, Z₇ and Z₈ are each independently CH, CX or N, in which at least one of Z₅ to Z₆ is CX or N;
Z₉ is CH₂, CHX, CX₂, NR₇, O or S;
R₇ is H or C₁₋₆ alkyl;
X is halogen;
\textcircled{A}, R₁, R₂, L₂, Z, m and n may be the same as defined in above formula I, respectively.
(5) The sulfoximine compound according to above (1), (2), (3) or (4), stereoisomers thereof or pharmaceutically acceptable salts thereof:
in which, in above formula Ia,
Z₁ is CX or N;
Z₂ is CH, CX or N;
Z₃ and Z₄ are each independently CH or N;
X is halogen;
L₁ is —C(═O)—, —S(═O)₂—, —S(═O)₂—(C₁₋₃ alkylene)-, —C(═O)NH—,

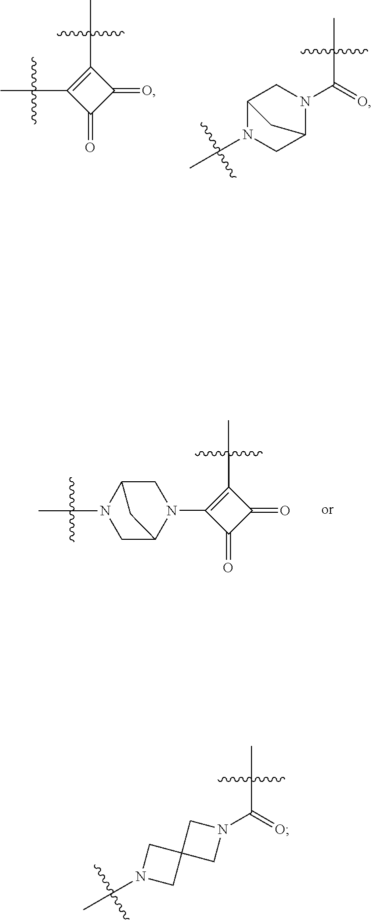 [SMILES: *c1c(*)c(=O)c1=O ; *C(=O)N1CC2CC1CN2* ; *c1c(N2CC3CC2CN3*)c(=O)c1=O ; *C(=O)N1CC2(CN(*)C2)C1]

\textcircled{A} is C₆-C₁₂ aryl, benzyl, 5- to 12-membered heteroaryl including in a ring 1 to 3 heteroatoms independently selected from the group consisting of N, O and S, or

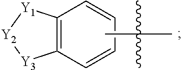

Y₁, Y₂ and Y₃ are each independently —CH₂— or —O—;
at least one H of above \textcircled{A} may be each independently substituted with C₁₋₆ alkyl, C₁₋₆ alkoxy, 3- to 12-membered heterocycloalkyl including in a ring 1 to 3 heteroatoms independently selected from the group consisting of N, O and S {in which at least one H of heterocycloalkyl may be substituted with —C(═O)(C₁₋₆ alkyl)}, 5- to 12-membered heteroaryl including in a ring 1 to 3 heteroatoms independently selected from the group consisting of N, O and S, —CF₃, —S(CF₃), halogen or —S(═NH)(═O)R_{c}; and
R_{c} may be the same as defined in above formula I.
(6) The sulfoximine compound according to above (1), (2), (3), (4) or (5), stereoisomers thereof or pharmaceutically acceptable salts thereof:
in which, in above formula Ia,
Z₁ is CF or N;
Z₂ is CH, CF or N;
Z₃ and Z₄ are each independently CH or N;
L₁ is —C(═O)—, —S(═O)₂—, —S(═O)₂—(C₁₋₃ alkylene)-, —C(═O)NH—,

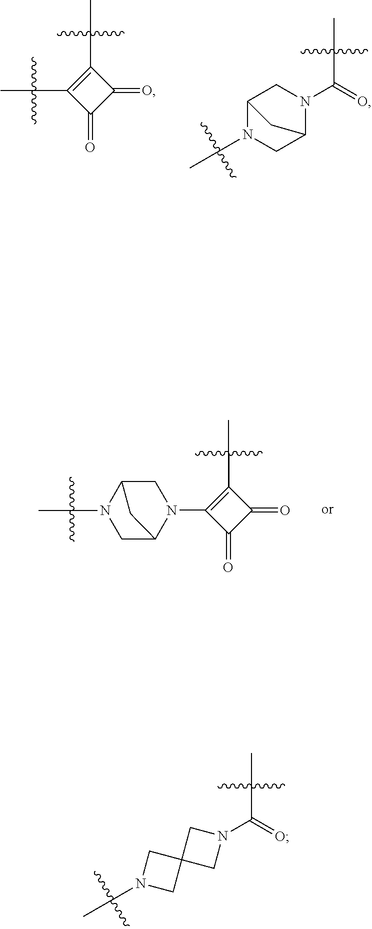 [SMILES: *c1c(*)c(=O)c1=O ; *C(=O)N1CC2CC1CN2* ; *c1c(N2CC3CC2CN3*)c(=O)c1=O ; *C(=O)N1CC2(CN(*)C2)C1]

R₂ is H, C₁₋₆ alkyl, C₆-C₂ aryl, CF₃ or —(P═O)(OR_{b})₂;
R_{b} is C₁₋₆ alkyl;
\textcircled{A} is C₆-C₁₂ aryl, benzyl, 5- to 12-membered heteroaryl including in a ring 1 to 3 heteroatoms independently selected from the group consisting of N, O and S,

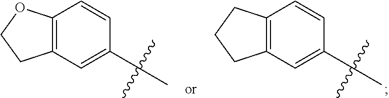 [SMILES: *c1ccc2c(c1)CCO2 ; *c1ccc2c(c1)CCC2]

in above \textcircled{A}, at least one H of C₆-C₁₂ aryl or 5- to 12-membered heteroaryl including in a ring 1 to 3 heteroatoms independently selected from the group consisting of N, O and S may be each independently substituted with C₁₋₆ alkyl, C₁₋₆ alkoxy, 5- to 12-membered heteroaryl including in a ring 1 to 3 heteroatoms independently selected from the group consisting of N, O and S, —CF₃, —S(CF₃), halogen or —S(═NH)(═O)R_{c}; and
R_{c} may be C₁₋₆ alkyl.

The sulfoximine compound represented by formula I above may comprise the group consisting of compounds shown in table 1 below.
(7) The sulfoximine compound according to above (1), (2), (3), (4), (5) or (6), stereoisomers thereof or pharmaceutically acceptable salts thereof,
in which, the sulfoximine compound according to the present invention may be at least one compound selected from the group consisting of compounds shown in table 1 below:

TABLE 1

| Com- | |
|---|---|
| pound | |
| No. | Structure |
| 1 | 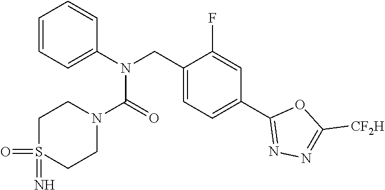 [SMILES: N=S1(=O)CCN(C(=O)N(Cc2ccc(-c3nnc(C(F)F)o3)cc2F)c2ccccc2)CC1] |
| 2 | 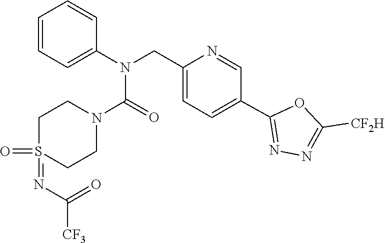 [SMILES: O=C(N1CCS(=O)(=NC(=O)C(F)(F)F)CC1)N(Cc1ccc(-c2nnc(C(F)F)o2)cn1)c1ccccc1] |
| 3 | 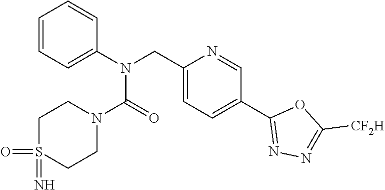 [SMILES: N=S1(=O)CCN(C(=O)N(Cc2ccc(-c3nnc(C(F)F)o3)cn2)c2ccccc2)CC1] |
| 4 | 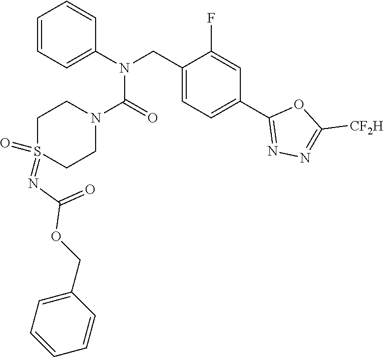 [SMILES: O=C(N=S1(=O)CCN(C(=O)N(Cc2ccc(-c3nnc(C(F)F)o3)cc2F)c2ccccc2)CC1)OCc1ccccc1] |
| 5 | 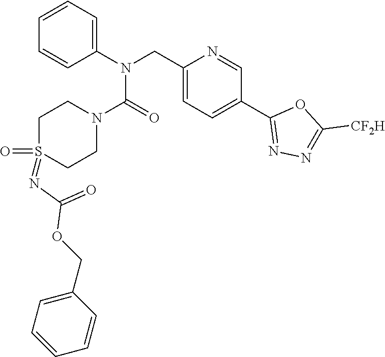 [SMILES: O=C(N=S1(=O)CCN(C(=O)N(Cc2ccc(-c3nnc(C(F)F)o3)cn2)c2ccccc2)CC1)OCc1ccccc1] |
| 6 | 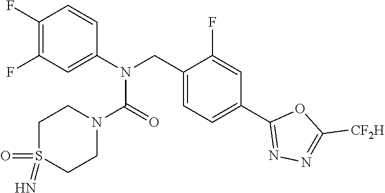 [SMILES: N=S1(=O)CCN(C(=O)N(Cc2ccc(-c3nnc(C(F)F)o3)cc2F)c2ccc(F)c(F)c2)CC1] |
| 7 | 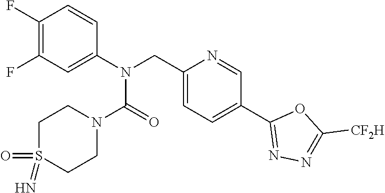 [SMILES: N=S1(=O)CCN(C(=O)N(Cc2ccc(-c3nnc(C(F)F)o3)cn2)c2ccc(F)c(F)c2)CC1] |
| 8 | 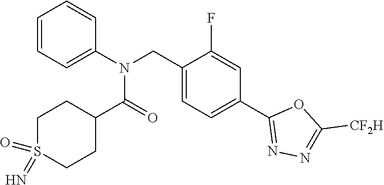 [SMILES: N=S1(=O)CCC(C(=O)N(Cc2ccc(-c3nnc(C(F)F)o3)cc2F)c2ccccc2)CC1] |
| 9 | 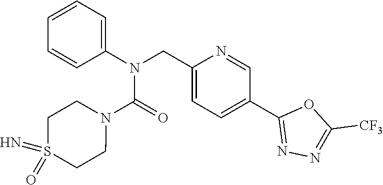 [SMILES: N=S1(=O)CCN(C(=O)N(Cc2ccc(-c3nnc(C(F)(F)F)o3)cn2)c2ccccc2)CC1] |
| 10 | 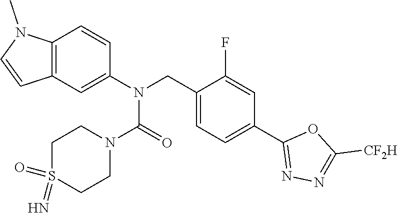 [SMILES: Cn1ccc2cc(N(Cc3ccc(-c4nnc(C(F)F)o4)cc3F)C(=O)N3CCS(=N)(=O)CC3)ccc21] |
| 11 | 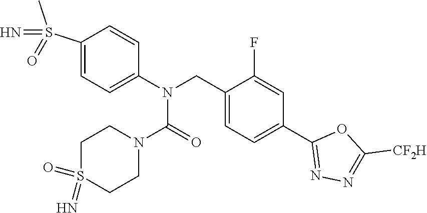 [SMILES: CS(=N)(=O)c1ccc(N(Cc2ccc(-c3nnc(C(F)F)o3)cc2F)C(=O)N2CCS(=N)(=O)CC2)cc1] |
| 12 | 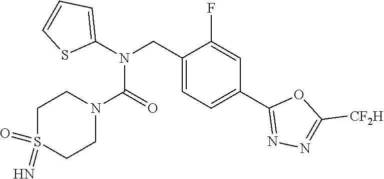 [SMILES: N=S1(=O)CCN(C(=O)N(Cc2ccc(-c3nnc(C(F)F)o3)cc2F)c2cccs2)CC1] |
| 13 | 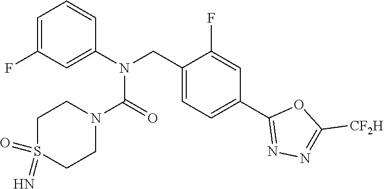 [SMILES: N=S1(=O)CCN(C(=O)N(Cc2ccc(-c3nnc(C(F)F)o3)cc2F)c2cccc(F)c2)CC1] |
| 14 | 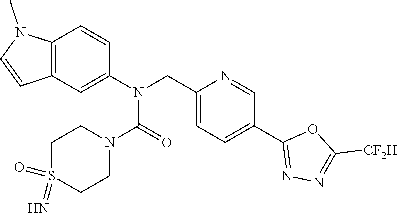 [SMILES: Cn1ccc2cc(N(Cc3ccc(-c4nnc(C(F)F)o4)cn3)C(=O)N3CCS(=N)(=O)CC3)ccc21] |
| 15 | 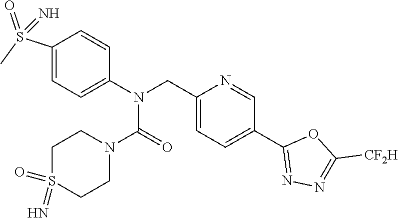 [SMILES: CS(=N)(=O)c1ccc(N(Cc2ccc(-c3nnc(C(F)F)o3)cn2)C(=O)N2CCS(=N)(=O)CC2)cc1] |
| 16 | 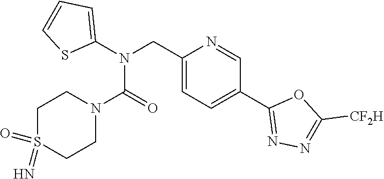 [SMILES: N=S1(=O)CCN(C(=O)N(Cc2ccc(-c3nnc(C(F)F)o3)cn2)c2cccs2)CC1] |
| 17 | 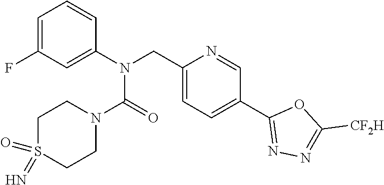 [SMILES: N=S1(=O)CCN(C(=O)N(Cc2ccc(-c3nnc(C(F)F)o3)cn2)c2cccc(F)c2)CC1] |
| 18 | 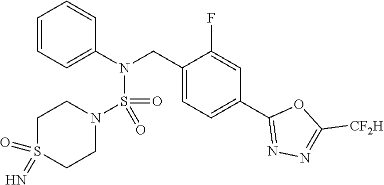 [SMILES: N=S1(=O)CCN(S(=O)(=O)N(Cc2ccc(-c3nnc(C(F)F)o3)cc2F)c2ccccc2)CC1] |
| 19 | 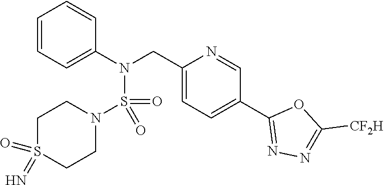 [SMILES: N=S1(=O)CCN(S(=O)(=O)N(Cc2ccc(-c3nnc(C(F)F)o3)cn2)c2ccccc2)CC1] |
| 20 | 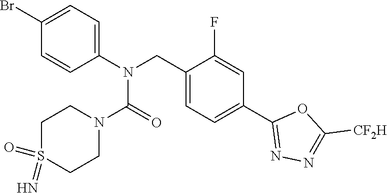 [SMILES: N=S1(=O)CCN(C(=O)N(Cc2ccc(-c3nnc(C(F)F)o3)cc2F)c2ccc(Br)cc2)CC1] |
| 21 | 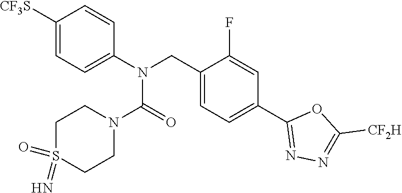 |
| 22 | 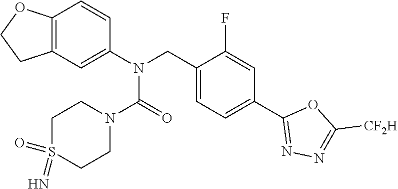 [SMILES: N=S1(=O)CCN(C(=O)N(Cc2ccc(-c3nnc(C(F)F)o3)cc2F)c2ccc3c(c2)CCO3)CC1] |
| 23 | 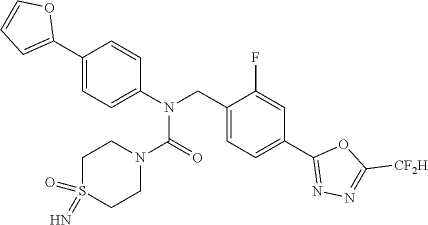 [SMILES: N=S1(=O)CCN(C(=O)N(Cc2ccc(-c3nnc(C(F)F)o3)cc2F)c2ccc(-c3ccco3)cc2)CC1] |
| 24 | 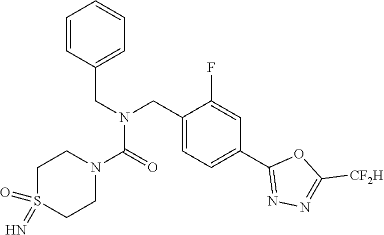 [SMILES: N=S1(=O)CCN(C(=O)N(Cc2ccccc2)Cc2ccc(-c3nnc(C(F)F)o3)cc2F)CC1] |
| 25 | 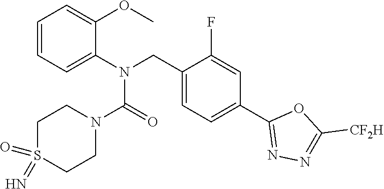 [SMILES: COc1ccccc1N(Cc1ccc(-c2nnc(C(F)F)o2)cc1F)C(=O)N1CCS(=N)(=O)CC1] |
| 26 | 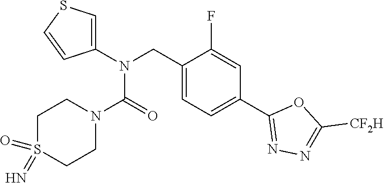 [SMILES: N=S1(=O)CCN(C(=O)N(Cc2ccc(-c3nnc(C(F)F)o3)cc2F)c2ccsc2)CC1] |
| 27 | 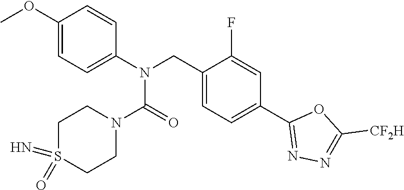 [SMILES: COc1ccc(N(Cc2ccc(-c3nnc(C(F)F)o3)cc2F)C(=O)N2CCS(=N)(=O)CC2)cc1] |
| 28 | 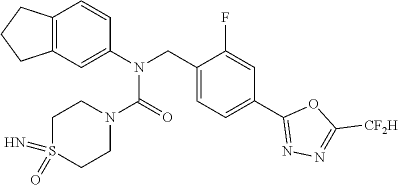 [SMILES: N=S1(=O)CCN(C(=O)N(Cc2ccc(-c3nnc(C(F)F)o3)cc2F)c2ccc3c(c2)CCC3)CC1] |
| 29 | 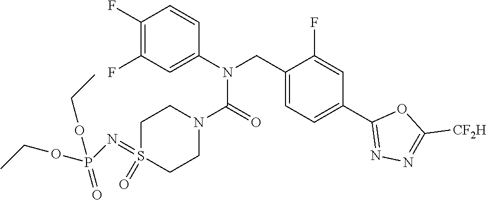 [SMILES: CCOP(=O)(N=S1(=O)CCN(C(=O)N(Cc2ccc(-c3nnc(C(F)F)o3)cc2F)c2ccc(F)c(F)c2)CC1)OCC] |
| 30 | 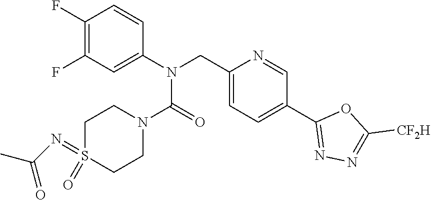 [SMILES: CC(=O)N=S1(=O)CCN(C(=O)N(Cc2ccc(-c3nnc(C(F)F)o3)cn2)c2ccc(F)c(F)c2)CC1] |
| 31 | 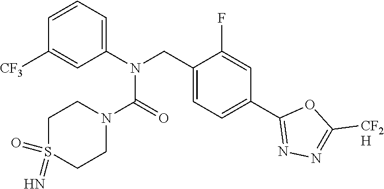 [SMILES: N=S1(=O)CCN(C(=O)N(Cc2ccc(-c3nnc(C(F)F)o3)cc2F)c2cccc(C(F)(F)F)c2)CC1] |
| 32 | 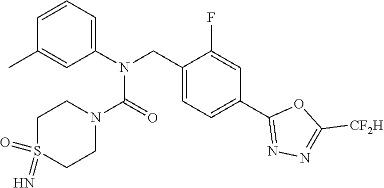 [SMILES: Cc1cccc(N(Cc2ccc(-c3nnc(C(F)F)o3)cc2F)C(=O)N2CCS(=N)(=O)CC2)c1] |
| 33 | 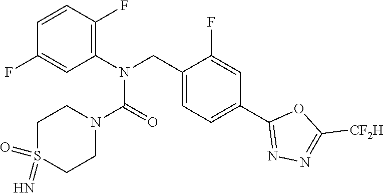 [SMILES: N=S1(=O)CCN(C(=O)N(Cc2ccc(-c3nnc(C(F)F)o3)cc2F)c2cc(F)ccc2F)CC1] |
| 34 | 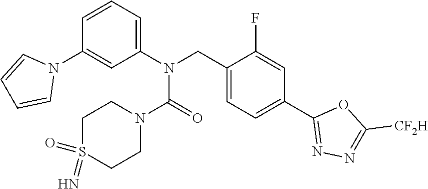 [SMILES: N=S1(=O)CCN(C(=O)N(Cc2ccc(-c3nnc(C(F)F)o3)cc2F)c2cccc(-n3cccc3)c2)CC1] |
| 35 | 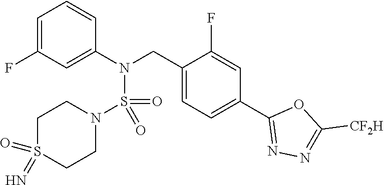 [SMILES: N=S1(=O)CCN(S(=O)(=O)N(Cc2ccc(-c3nnc(C(F)F)o3)cc2F)c2cccc(F)c2)CC1] |
| 36 | 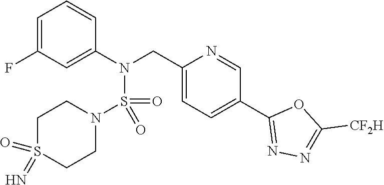 [SMILES: N=S1(=O)CCN(S(=O)(=O)N(Cc2ccc(-c3nnc(C(F)F)o3)cn2)c2cccc(F)c2)CC1] |
| 37 | 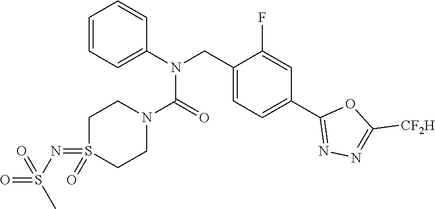 [SMILES: CS(=O)(=O)N=S1(=O)CCN(C(=O)N(Cc2ccc(-c3nnc(C(F)F)o3)cc2F)c2ccccc2)CC1] |
| 38 | 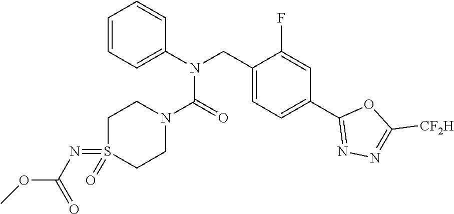 [SMILES: COC(=O)N=S1(=O)CCN(C(=O)N(Cc2ccc(-c3nnc(C(F)F)o3)cc2F)c2ccccc2)CC1] |
| 39 | 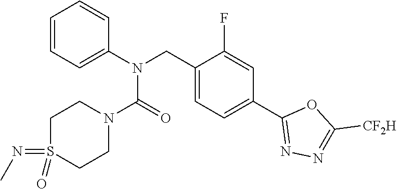 [SMILES: CN=S1(=O)CCN(C(=O)N(Cc2ccc(-c3nnc(C(F)F)o3)cc2F)c2ccccc2)CC1] |
| 40 | 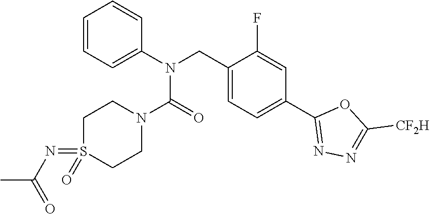 [SMILES: CC(=O)N=S1(=O)CCN(C(=O)N(Cc2ccc(-c3nnc(C(F)F)o3)cc2F)c2ccccc2)CC1] |
| 41 | 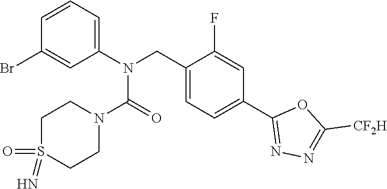 [SMILES: N=S1(=O)CCN(C(=O)N(Cc2ccc(-c3nnc(C(F)F)o3)cc2F)c2cccc(Br)c2)CC1] |
| 42 | 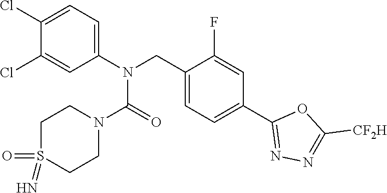 [SMILES: N=S1(=O)CCN(C(=O)N(Cc2ccc(-c3nnc(C(F)F)o3)cc2F)c2ccc(Cl)c(Cl)c2)CC1] |
| 43 | 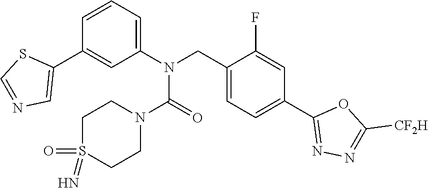 [SMILES: N=S1(=O)CCN(C(=O)N(Cc2ccc(-c3nnc(C(F)F)o3)cc2F)c2cccc(-c3cncs3)c2)CC1] |
| 44 | 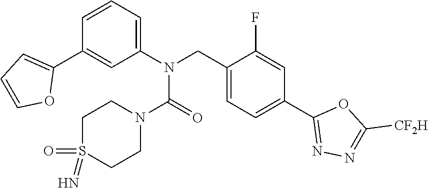 [SMILES: N=S1(=O)CCN(C(=O)N(Cc2ccc(-c3nnc(C(F)F)o3)cc2F)c2cccc(-c3ccco3)c2)CC1] |
| 45 | 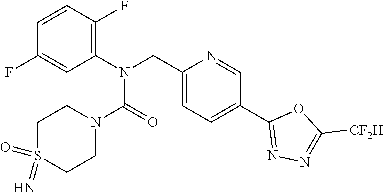 [SMILES: N=S1(=O)CCN(C(=O)N(Cc2ccc(-c3nnc(C(F)F)o3)cn2)c2cc(F)ccc2F)CC1] |
| 46 | 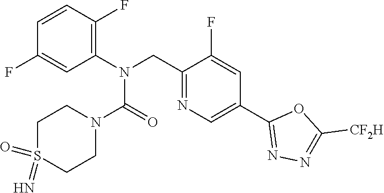 [SMILES: N=S1(=O)CCN(C(=O)N(Cc2ncc(-c3nnc(C(F)F)o3)cc2F)c2cc(F)ccc2F)CC1] |
| 47 | 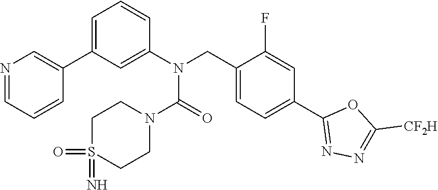 [SMILES: N=S1(=O)CCN(C(=O)N(Cc2ccc(-c3nnc(C(F)F)o3)cc2F)c2cccc(-c3cccnc3)c2)CC1] |
| 48 | 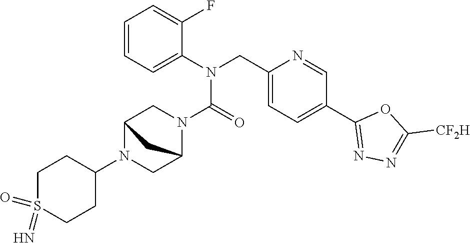 [SMILES: N=S1(=O)CCC(N2C[C@@H]3C[C@H]2CN3C(=O)N(Cc2ccc(-c3nnc(C(F)F)o3)cn2)c2ccccc2F)CC1] |
| 49 | 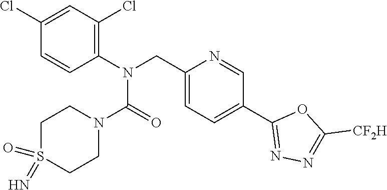 [SMILES: N=S1(=O)CCN(C(=O)N(Cc2ccc(-c3nnc(C(F)F)o3)cn2)c2ccc(Cl)cc2Cl)CC1] |
| 50 | 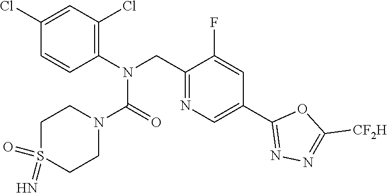 [SMILES: N=S1(=O)CCN(C(=O)N(Cc2ncc(-c3nnc(C(F)F)o3)cc2F)c2ccc(Cl)cc2Cl)CC1] |
| 51 | 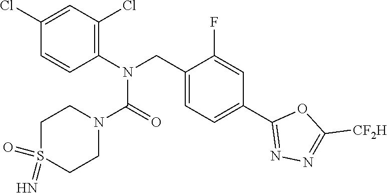 [SMILES: N=S1(=O)CCN(C(=O)N(Cc2ccc(-c3nnc(C(F)F)o3)cc2F)c2ccc(Cl)cc2Cl)CC1] |
| 52 | 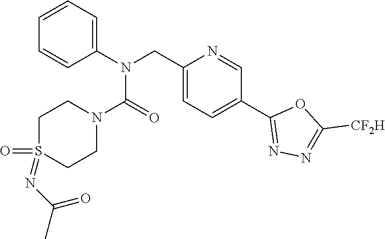 [SMILES: CC(=O)N=S1(=O)CCN(C(=O)N(Cc2ccc(-c3nnc(C(F)F)o3)cn2)c2ccccc2)CC1] |
| 53 | 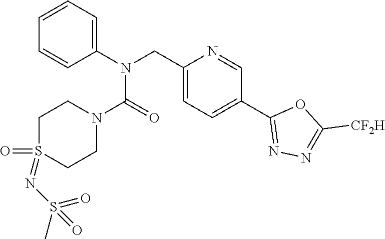 [SMILES: CS(=O)(=O)N=S1(=O)CCN(C(=O)N(Cc2ccc(-c3nnc(C(F)F)o3)cn2)c2ccccc2)CC1] |
| 54 | 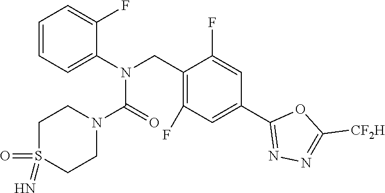 [SMILES: N=S1(=O)CCN(C(=O)N(Cc2c(F)cc(-c3nnc(C(F)F)o3)cc2F)c2ccccc2F)CC1] |
| 55 | 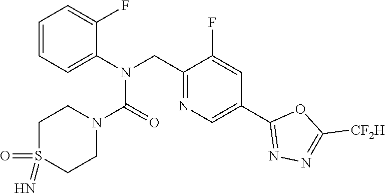 [SMILES: N=S1(=O)CCN(C(=O)N(Cc2ncc(-c3nnc(C(F)F)o3)cc2F)c2ccccc2F)CC1] |
| 56 | 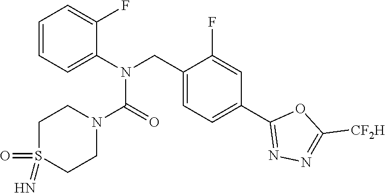 [SMILES: N=S1(=O)CCN(C(=O)N(Cc2ccc(-c3nnc(C(F)F)o3)cc2F)c2ccccc2F)CC1] |
| 57 | 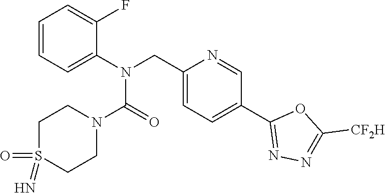 [SMILES: N=S1(=O)CCN(C(=O)N(Cc2ccc(-c3nnc(C(F)F)o3)cn2)c2ccccc2F)CC1] |
| 58 | 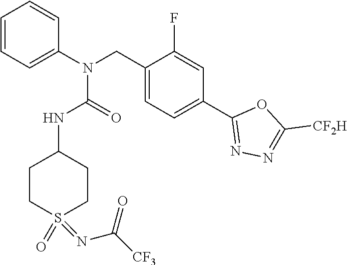 [SMILES: O=C(NC1CCS(=O)(=NC(=O)C(F)(F)F)CC1)N(Cc1ccc(-c2nnc(C(F)F)o2)cc1F)c1ccccc1] |
| 59 | 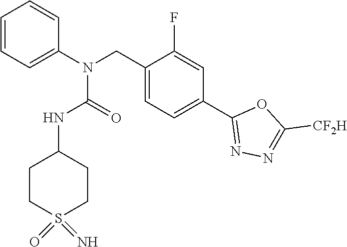 [SMILES: N=S1(=O)CCC(NC(=O)N(Cc2ccc(-c3nnc(C(F)F)o3)cc2F)c2ccccc2)CC1] |
| 60 | 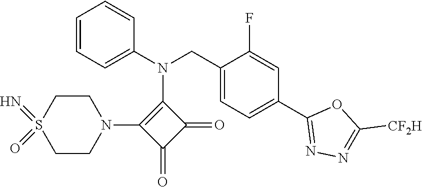 [SMILES: N=S1(=O)CCN(c2c(N(Cc3ccc(-c4nnc(C(F)F)o4)cc3F)c3ccccc3)c(=O)c2=O)CC1] |
| 61 | 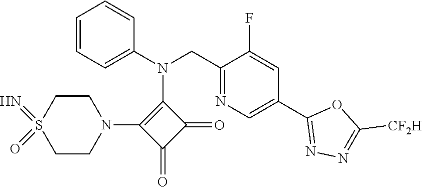 [SMILES: N=S1(=O)CCN(c2c(N(Cc3ncc(-c4nnc(C(F)F)o4)cc3F)c3ccccc3)c(=O)c2=O)CC1] |
| 62 | 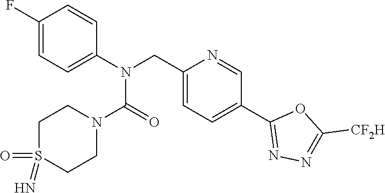 [SMILES: N=S1(=O)CCN(C(=O)N(Cc2ccc(-c3nnc(C(F)F)o3)cn2)c2ccc(F)cc2)CC1] |
| 63 | 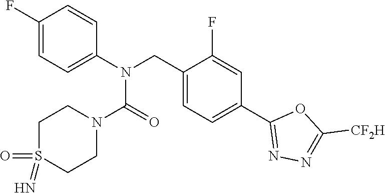 [SMILES: N=S1(=O)CCN(C(=O)N(Cc2ccc(-c3nnc(C(F)F)o3)cc2F)c2ccc(F)cc2)CC1] |
| 64 | 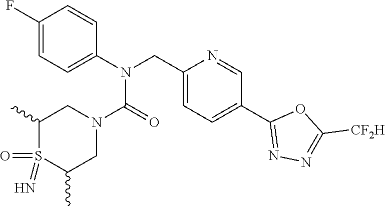 |
| 65 | 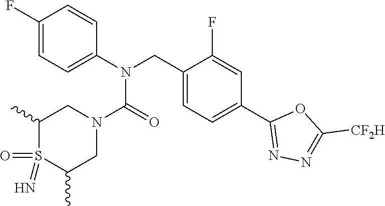 |
| 66 | 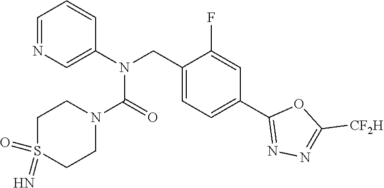 [SMILES: N=S1(=O)CCN(C(=O)N(Cc2ccc(-c3nnc(C(F)F)o3)cc2F)c2cccnc2)CC1] |
| 67 | 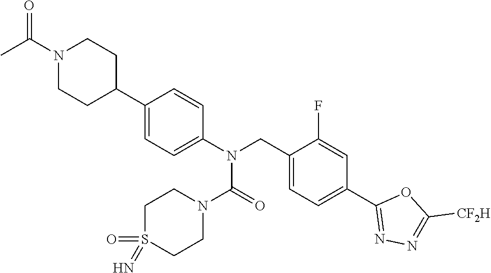 [SMILES: CC(=O)N1CCC(c2ccc(N(Cc3ccc(-c4nnc(C(F)F)o4)cc3F)C(=O)N3CCS(=N)(=O)CC3)cc2)CC1] |
| 68 | 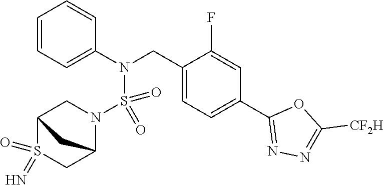 [SMILES: N=S1(=O)C[C@@H]2C[C@H]1CN2S(=O)(=O)N(Cc1ccc(-c2nnc(C(F)F)o2)cc1F)c1ccccc1] |
| 69 | 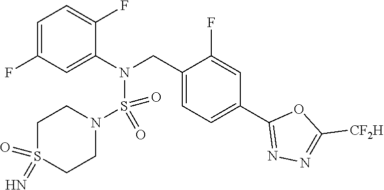 [SMILES: N=S1(=O)CCN(S(=O)(=O)N(Cc2ccc(-c3nnc(C(F)F)o3)cc2F)c2cc(F)ccc2F)CC1] |
| 70 | 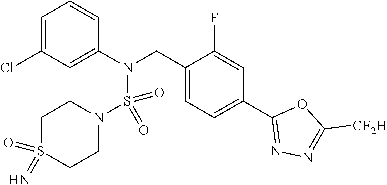 [SMILES: N=S1(=O)CCN(S(=O)(=O)N(Cc2ccc(-c3nnc(C(F)F)o3)cc2F)c2cccc(Cl)c2)CC1] |
| 71 | 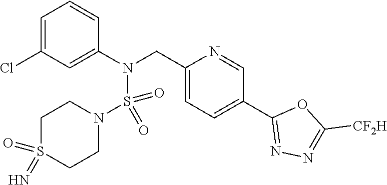 [SMILES: N=S1(=O)CCN(S(=O)(=O)N(Cc2ccc(-c3nnc(C(F)F)o3)cn2)c2cccc(Cl)c2)CC1] |
| 72 | 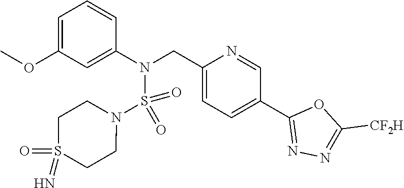 [SMILES: COc1cccc(N(Cc2ccc(-c3nnc(C(F)F)o3)cn2)S(=O)(=O)N2CCS(=N)(=O)CC2)c1] |
| 73 | 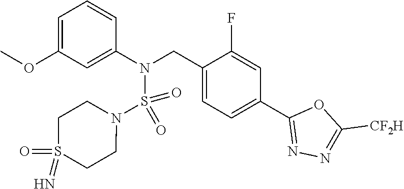 [SMILES: COc1cccc(N(Cc2ccc(-c3nnc(C(F)F)o3)cc2F)S(=O)(=O)N2CCS(=N)(=O)CC2)c1] |
| 74 | 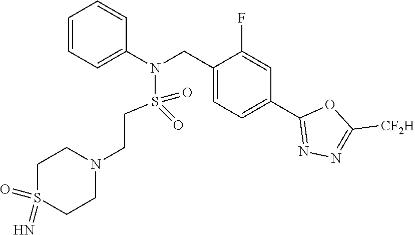 [SMILES: N=S1(=O)CCN(CCS(=O)(=O)N(Cc2ccc(-c3nnc(C(F)F)o3)cc2F)c2ccccc2)CC1] |
| 75 | 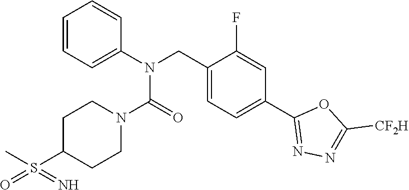 [SMILES: CS(=N)(=O)C1CCN(C(=O)N(Cc2ccc(-c3nnc(C(F)F)o3)cc2F)c2ccccc2)CC1] |
| 76 | 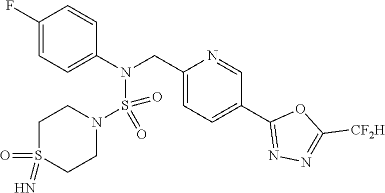 [SMILES: N=S1(=O)CCN(S(=O)(=O)N(Cc2ccc(-c3nnc(C(F)F)o3)cn2)c2ccc(F)cc2)CC1] |
| 77 | 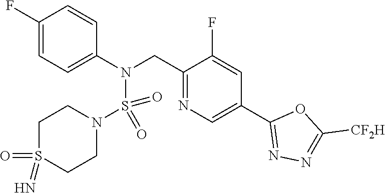 [SMILES: N=S1(=O)CCN(S(=O)(=O)N(Cc2ncc(-c3nnc(C(F)F)o3)cc2F)c2ccc(F)cc2)CC1] |
| 78 | 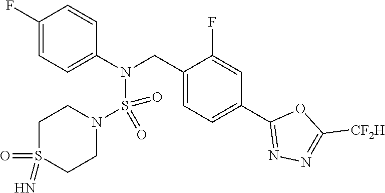 [SMILES: N=S1(=O)CCN(S(=O)(=O)N(Cc2ccc(-c3nnc(C(F)F)o3)cc2F)c2ccc(F)cc2)CC1] |
| 79 | 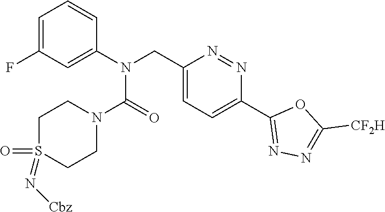 [SMILES: O=C(N=S1(=O)CCN(C(=O)N(Cc2ccc(-c3nnc(C(F)F)o3)nn2)c2cccc(F)c2)CC1)OCc1ccccc1] |
| 80 | 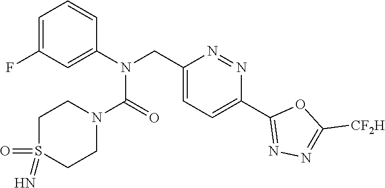 [SMILES: N=S1(=O)CCN(C(=O)N(Cc2ccc(-c3nnc(C(F)F)o3)nn2)c2cccc(F)c2)CC1] |
| 81 | 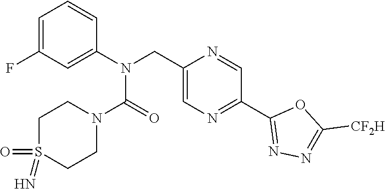 [SMILES: N=S1(=O)CCN(C(=O)N(Cc2cnc(-c3nnc(C(F)F)o3)cn2)c2cccc(F)c2)CC1] |
| 82 | 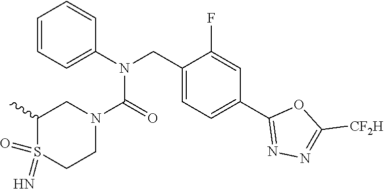 [SMILES: CC1CN(C(=O)N(Cc2ccc(-c3nnc(C(F)F)o3)cc2F)c2ccccc2)CCS1(=N)=O] |
| 83 | 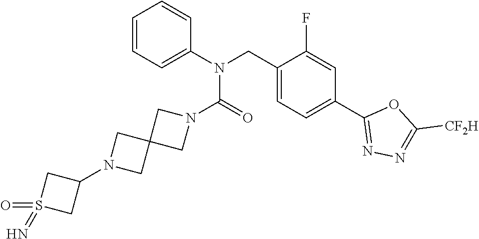 [SMILES: N=S1(=O)CC(N2CC3(CN(C(=O)N(Cc4ccc(-c5nnc(C(F)F)o5)cc4F)c4ccccc4)C3)C2)C1] |
| 84 | 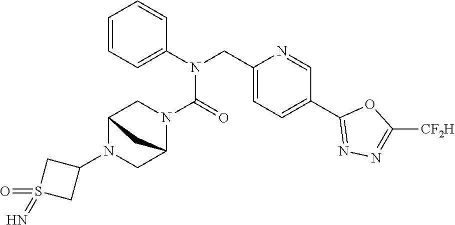 [SMILES: N=S1(=O)CC(N2C[C@@H]3C[C@H]2CN3C(=O)N(Cc2ccc(-c3nnc(C(F)F)o3)cn2)c2ccccc2)C1] |
| 85 | 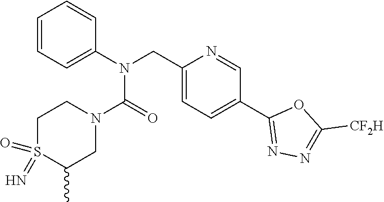 [SMILES: CC1CN(C(=O)N(Cc2ccc(-c3nnc(C(F)F)o3)cn2)c2ccccc2)CCS1(=N)=O] |
| 86 | 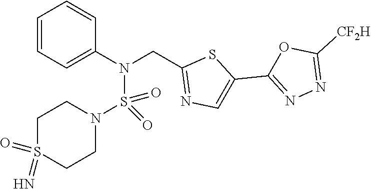 [SMILES: N=S1(=O)CCN(S(=O)(=O)N(Cc2ncc(-c3nnc(C(F)F)o3)s2)c2ccccc2)CC1] |
| 87 | 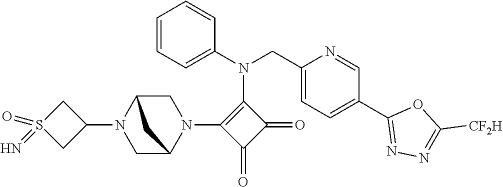 [SMILES: N=S1(=O)CC(N2C[C@@H]3C[C@H]2CN3c2c(N(Cc3ccc(-c4nnc(C(F)F)o4)cn3)c3ccccc3)c(=O)c2=O)C1] |
| 88 | 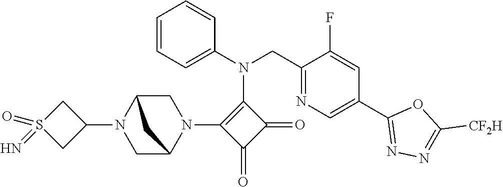 [SMILES: N=S1(=O)CC(N2C[C@@H]3C[C@H]2CN3c2c(N(Cc3ncc(-c4nnc(C(F)F)o4)cc3F)c3ccccc3)c(=O)c2=O)C1] |
| 89 | 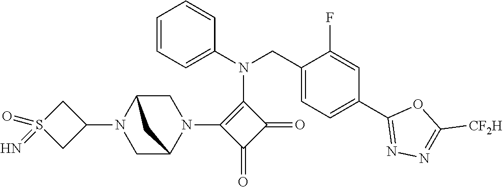 [SMILES: N=S1(=O)CC(N2C[C@@H]3C[C@H]2CN3c2c(N(Cc3ccc(-c4nnc(C(F)F)o4)cc3F)c3ccccc3)c(=O)c2=O)C1] |
| 90 | 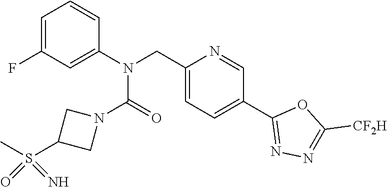 [SMILES: CS(=N)(=O)C1CN(C(=O)N(Cc2ccc(-c3nnc(C(F)F)o3)cn2)c2cccc(F)c2)C1] |
| 91 | 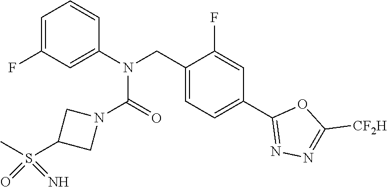 [SMILES: CS(=N)(=O)C1CN(C(=O)N(Cc2ccc(-c3nnc(C(F)F)o3)cc2F)c2cccc(F)c2)C1] |
| 92 | 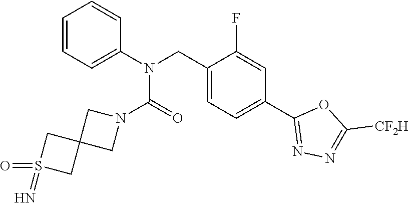 [SMILES: N=S1(=O)CC2(CN(C(=O)N(Cc3ccc(-c4nnc(C(F)F)o4)cc3F)c3ccccc3)C2)C1] |

In the present invention, “pharmaceutically acceptable salts” may mean salts conventionally used in a pharmaceutical industry, for example, inorganic ion salts prepared from calcium, potassium, sodium, magnesium or the like; inorganic acid salts prepared from hydrochloric acid, nitric acid, phosphoric acid, bromic acid, iodic acid, perchloric acid, sulfuric acid or the like; organic acid salts prepared from acetic acid, trifluoroacetic acid, citric acid, maleic acid, succinic acid, oxalic acid, benzoic acid, tartaric acid, fumaric acid, mandelic acid, propionic acid, lactic acid, glycolic acid, gluconic acid, galacturonic acid, glutamic acid, glutaric acid, glucuronic acid, aspartic acid, ascorbic acid, carbonic acid, vanillic acid or the like; sulfonic acid salts prepared from methanesulfonic acid, ethanesulfonic acid, benzenesulfonic acid, p-toluenesulfonic acid, naphthalenesulfonic acid or the like; amino acid salts prepared from glycine, arginine, lysine, or the like; amine salts prepared from trimethylamine, triethylamine, ammonia, pyridine, picoline, or the like; and the like, but types of salt meant in the present invention are not limited to those listed salts.

In the present invention, the sulfoximine compound represented by above formula I may contain at least one asymmetric carbon, and thus may be present as a racemate, a racemic mixture, a single enantiomer, a single diastereomer, and a mixture of diastereomers. Such isomer may be separated by split according to the related art, for example, column chromatography, HPLC or the like. Alternatively, the isomer may be stereospecifically synthesized by using a known array of optically pure starting materials and/or reagents.

In the present invention, “stereoisomer” may include a diastereomer and an enantiomer, in which stereoisomer may include not only an enantiomer, but also a mixture of enantiomers and even a racemate.

### Method for Preparing Compound

A method for preparing a sulfoximine compound represented by formula I according to the present invention, stereoisomers thereof or pharmaceutically acceptable salts thereof may follow reaction formulas 1 to 13 below. A preparation method obtained by modifying the above preparation method to a level obvious to those skilled in the art may also be included in the preparation method of the present invention.

In reaction formulas 1 to 13 below, \textcircled{A} and \textcircled{B} may be the same as defined in formula I.

In reaction formulas 1 to 13 below, “R” and \textcircled{C} may be null or defined as a substituent which may be substituted with at least one of hydrogens of \textcircled{A} in formula I, and R and \textcircled{C}, which are bonded to \textcircled{A}, may be represented one by one, but at least two thereof may be bonded to \textcircled{A} and at least two of R or \textcircled{A} may be the same or different from each other.

In reaction formulas 1 to 13 below, “Halo” may represent a leaving group such as Br, Cl or the like, K may represent H or F, and “PG” may represent a protecting group.

Reaction formulas 1 to 13 below specifically represent a case in which \textcircled{D} of formula I has a

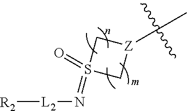

structure (in which n and m each independently represent 1 or 2), but may be also equally applied to a case in which \textcircled{D} has a

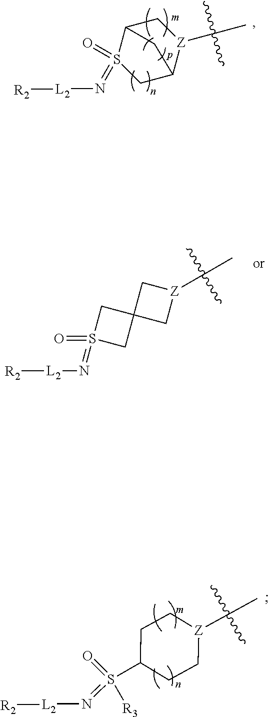

structure (in which n and m each independently represent 0 or 1), as defined in formula I.

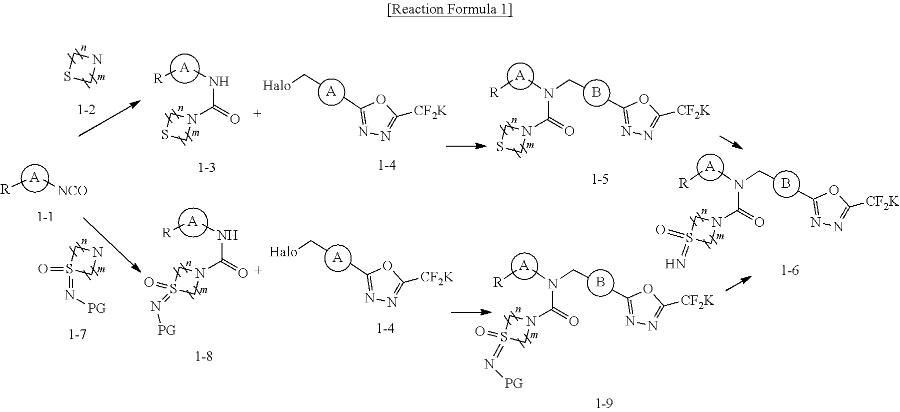

Above reaction formula 1 may represent a method for synthesizing a 1,3,4-oxadiazole compound including a sulfoximine functional group.

According to an upper part of the method of above reaction formula 1, a compound of formula 1-1 including isocyanate may be reacted with a compound of formula 1-2 including a sulfide functional group to prepare a compound of formula 1-3 which is a urea compound. After that, a compound of formula 1-5 may be prepared by a substitution reaction with a compound of formula 1-4, thereby preparing a compound of formula 1-6 into which a sulfoximine functional group is introduced through a bisacetoxyiodobenzene medium and N-source.

In addition, according to a lower part of the method of above reaction formula 1, a compound of formula 1-7, which is a sulfoximine compound having a protecting group included in an imine functional group, may be reacted with a compound of formula 1-1 to prepare a compound of formula 1-8 which is a urea compound. For example, the protecting group may be benzyloxycarbonyl (Cbz). After that, a compound of formula 1-9 may be prepared by a substitution reaction between a compound of formula 1-8 and a compound of formula 1-4, after which a protecting group of the compound of formula 1-9 may be removed to prepare a compound of formula 1-6.

In the present invention, the compounds prepared according to the above reaction formula 1 may include compounds 1, 2, 3, 4, 5, 6, 7, 9, 10, 11, 12, 13, 14, 15, 16, 17, 20, 21, 22, 24, 25, 26, 27, 28, 31, 32, 33, 34, 41, 42, 45, 46, 49, 50, 51, 54, 55, 56, 57, 62, 63, 66, 79, 80, 81, 86 and the like.

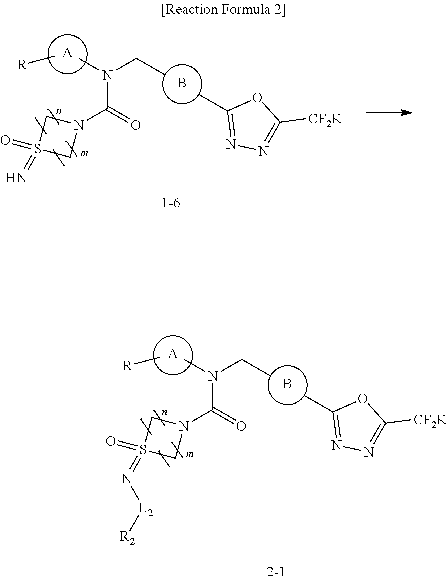

Above reaction formula 2 may represent a method for synthesizing a 1,3,4-oxadiazole compound including a sulfoximine functional group.

According to above reaction formula 2, the compound of formula 1-6 prepared in reaction formula 1 may be subjected to an alkylation reaction, an acetylation reaction, a phosphorylation reaction, and the like, so as to prepare a compound of formula 2-1.

In the present invention, the compounds prepared according to above reaction formula 2 may include compounds 29, 30, 37, 38, 39, 40, 52, 53 and the like.

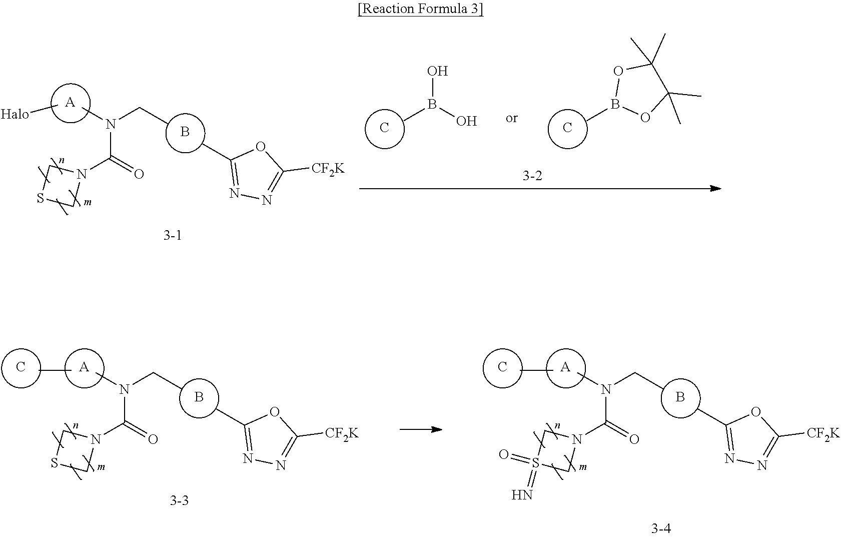

Above reaction formula 3 may represent a method for synthesizing a 1,3,4-oxadiazole compound including a sulfoximine functional group.

According to above reaction formula 3, a compound of formula 3-3 may be prepared through C—C coupling (Suzuki reaction) between a compound of formula 3-1 and a compound of formula 3-2, which may be prepared according to substantially the same method as the preparation of the compound of formula 1-5 described in reaction formula 1, after that a compound of formula 3-4, into which the sulfoximine functional group is introduced through a bisacetoxyiodobenzene medium and N-Source, may be prepared.

In the present invention, the compounds prepared according to above reaction formula 3 may include compounds 23, 43, 44, 47 and the like.

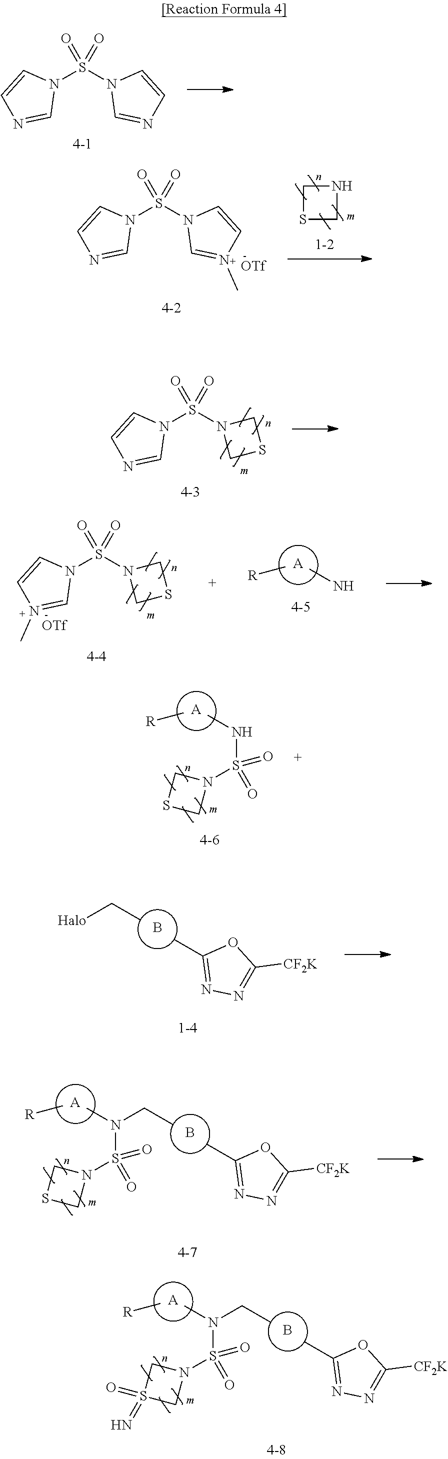

Above reaction formula 4 may represent a method for synthesizing a 1,3,4-oxadiazole sulfamide compound including a sulfoximine functional group.

According to above reaction formula 4, a compound of formula 4-1 may be reacted with methyl triflate (TfOCH₃) to prepare a compound of formula 4-2. After that, the compound of formula 4-2 may be reacted with the compound of formula 1-2 to prepare a compound of formula 4-3. The prepared compound of formula 4-3 may be reacted with methyl triflate to prepare a compound of formula 4-4, and then reacted with a compound of formula 4-5, into which an amine group is introduced, so as to prepare a compound of formula 4-6, into which sulfamide is introduced. After that, a compound of formula 4-7 may be prepared by a substitution reaction with the compound of formula 1-4, thereby preparing a compound of formula 4-8, into which a sulfoximine functional group is introduced through a bisacetoxyiodobenzene medium and N-source.

In the present invention, the compounds prepared according to above reaction formula 4 may include compounds 18, 19, 35, 36, 69, 70, 71, 72, 73, 76, 77, 78 and the like.

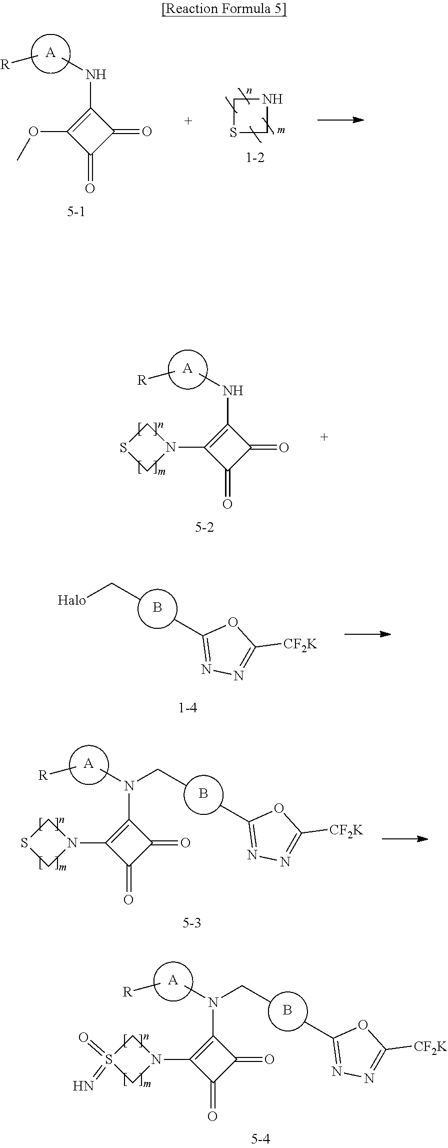

Above reaction formula 5 may represent a method for synthesizing a 1,3,4-oxadiazole cyclobutendione compound including a sulfoximine functional group.

According to above reaction formula 5, a compound of formula 5-1 may be reacted with a compound of formula 1-2 including a sulfide functional group to prepare a compound of formula 5-2 which is a cyclobutendione compound. After that, a compound of formula 5-3 may be prepared by a substitution reaction with the compound of formula 1-4, and then a compound of formula 5-4, into which a sulfoximine functional group is introduced through a bisacetoxyiodobenzene medium and N-source, may be prepared.

In the present invention, the compounds prepared according to above reaction formula may include compounds 60 and 61.

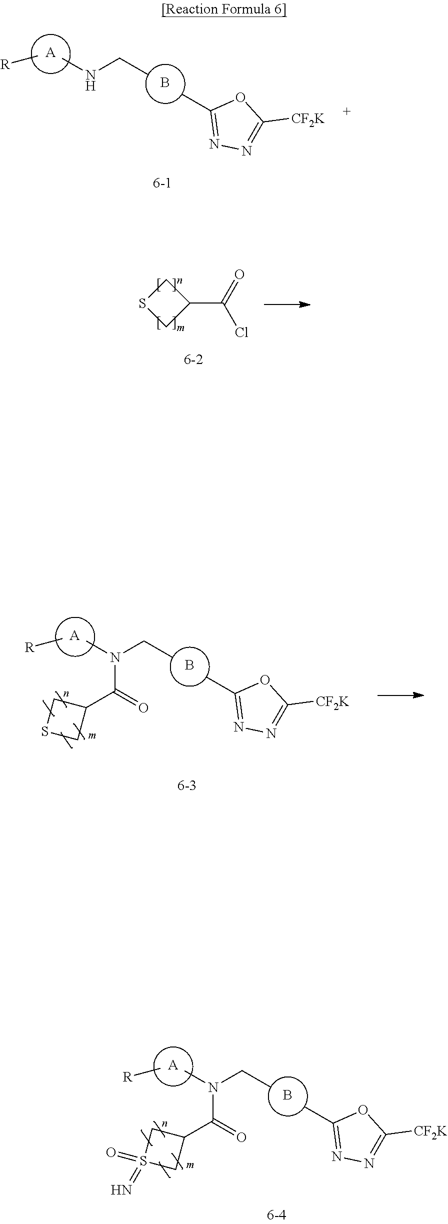

Above reaction formula 6 may represent a method for synthesizing a compound having a 1,3,4-oxadiazole amide structure including a sulfoximine functional group.

According to above reaction formula 6, a compound of formula 6-1 may be reacted with a compound of formula 6-2 to prepare a compound of formula 6-3 which is an amide compound including a sulfide functional group. After that, a compound of formula 6-4, into which a sulfoximine functional group is introduced through a bisacetoxyiodobenzene medium and N-source, may be prepared. The compound prepared according to above reaction formula 6 may include compound 8.

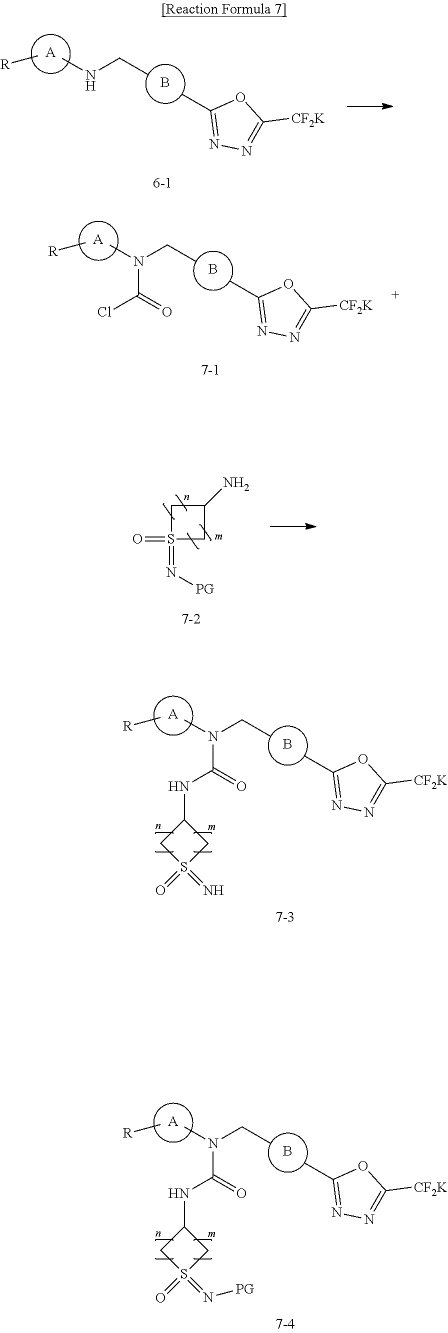

Above reaction formula 7 may represent a method for synthesizing a compound having a 1,3,4-oxadiazole urea structure including a sulfoximine functional group.

According to above reaction formula 7, a compound of formula 7-1 may be prepared through triphosgene with a compound of formula 6-1. After that, a reaction may be made with a compound of formula 7-2, which is a sulfoximine compound having a protecting group included in an S═N functional group, so as to prepare a compound of formula 7-3 and a compound of formula 7-4, from which the protecting group is removed. The compounds prepared according to above reaction formula 7 may include compounds 58, 59, etc.

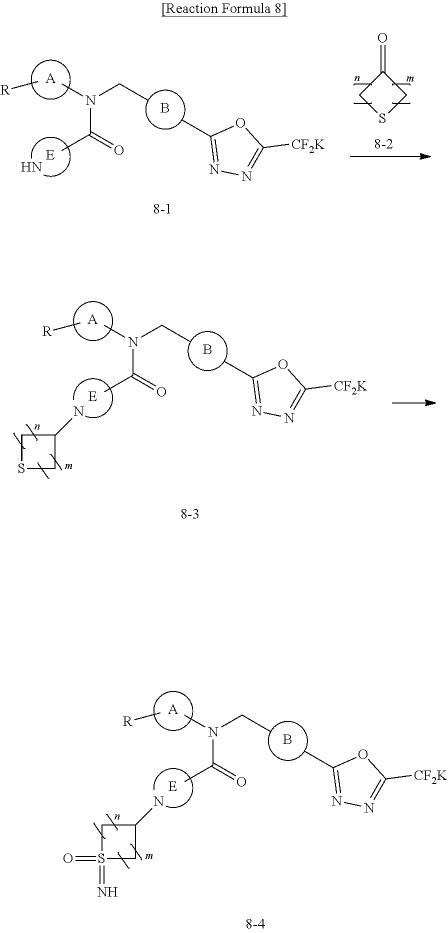

Above reaction formula 8 may represent a method for synthesizing a 1,3,4-oxadiazole compound including a sulfoximine functional group. In above reaction formula 8,

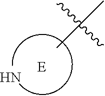

may represent

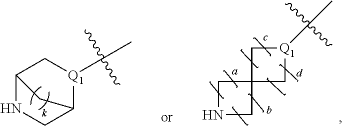

Q₁ may represent CH or N, and k may represent 1 or 2.

According to above reaction formula 8, a compound of formula 8-1 may be subjected to a reductive amination reaction with a compound of formula 8-2 to prepare a compound of formula 8-3 including a sulfide functional group. After that, a compound of formula 8-4, into which a sulfoximine functional group is introduced through a bisacetoxyiodobenzene medium and N-source, may be prepared. The compound prepared according to above reaction formula 8 may include compound 48, 83 and 84.

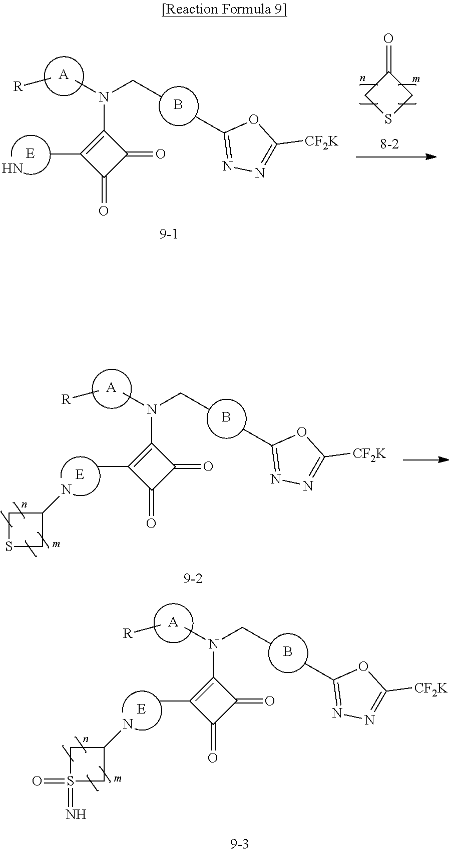

Above reaction formula 9 may represent a method for synthesizing a 1,3,4-oxadiazole cyclobutendione compound including a sulfoximine functional group. In above reaction formula 9,

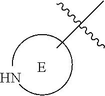

may represent

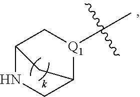

Q₁ may represent CH or N, and k may represent 1 or 2.

According to above reaction formula 9, a compound of formula 9-1 may be subjected to a reductive amination reaction with a compound of formula 8-2 to prepare a compound of formula 9-2 including a sulfide functional group. After that, a compound of formula 9-3, into which a sulfoximine functional group is introduced through a bisacetoxyiodobenzene medium and N-source, may be prepared. The compound prepared according to above reaction formula 9 may include compounds 87, 88 and 89.

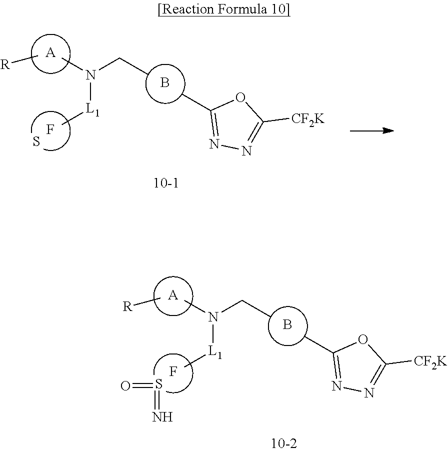

Above reaction formula 10 may represent a method for synthesizing a 1,3,4-oxadiazole compound including a sulfoximine functional group. In above reaction formula 10,

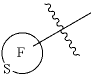

may represent

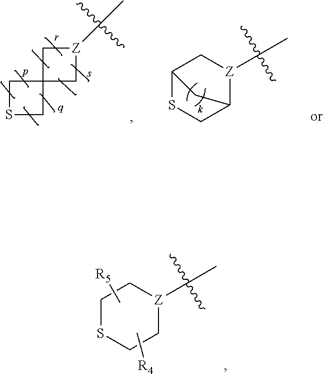

Z may represent CH or N, k may represent 1 or 2, L₁ may represent

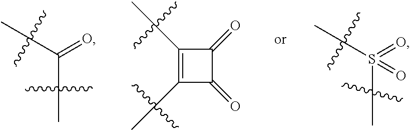

and R₄ and R₅ may each independently represent H, C₁₋₆ alkyl or F.

According to above reaction formula 10, a compound of formula 10-1 may be used to prepare a compound of formula 10-2 into a sulfoximine functional group is introduced through a bisacetoxyiodobenzene medium and N-source. The compound prepared according to above reaction formula 10 may include compounds 64, 65, 68, 82, 85 and 92.

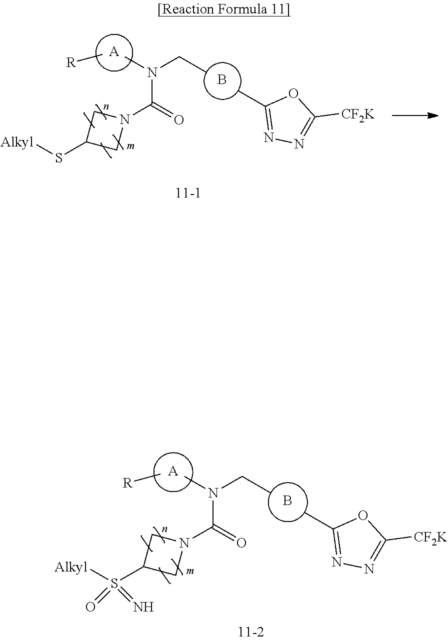

Above reaction formula 11 may represent a method for synthesizing a 1,3,4-oxadiazole compound including a sulfoximine functional group.

According to above reaction formula 11, a compound of formula 11-1 may be used to prepare a compound of formula 11-2 into a sulfoximine functional group is introduced through a bisacetoxyiodobenzene medium and N-source. The compound prepared according to above reaction formula 11 may include compounds 75, 90 and 91.

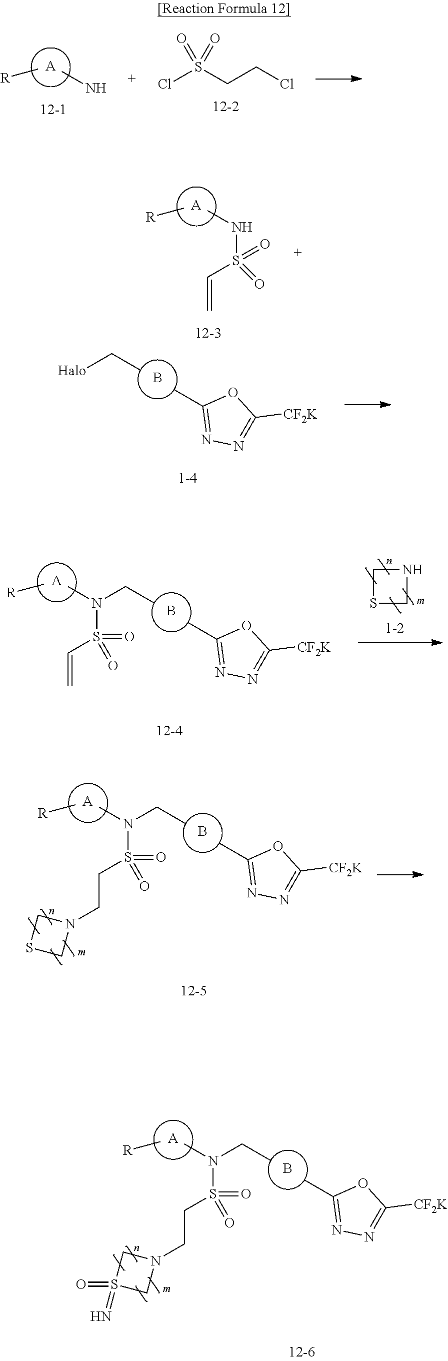

Above reaction formula 12 may represent a method for synthesizing a 1,3,4-oxadiazole compound including a sulfoximine functional group.

According to above reaction formula 12, a compound of formula 12-1 may be reacted with a compound of formula 12-2 to prepare a compound of formula 12-3. After that, the resulting compound may be subjected to a substitution reaction with a compound of formula 1-4 to prepare a compound of formula 12-4, and then reacted with a compound of formula 1-2 including a sulfide functional group to prepare a compound of formula 12-5. After that, a compound of formula 12-6, into which a sulfoximine functional group is introduced through a bisacetoxyiodobenzene medium and N-source, may be prepared. The compound prepared according to above reaction formula 12 may include compound 74.

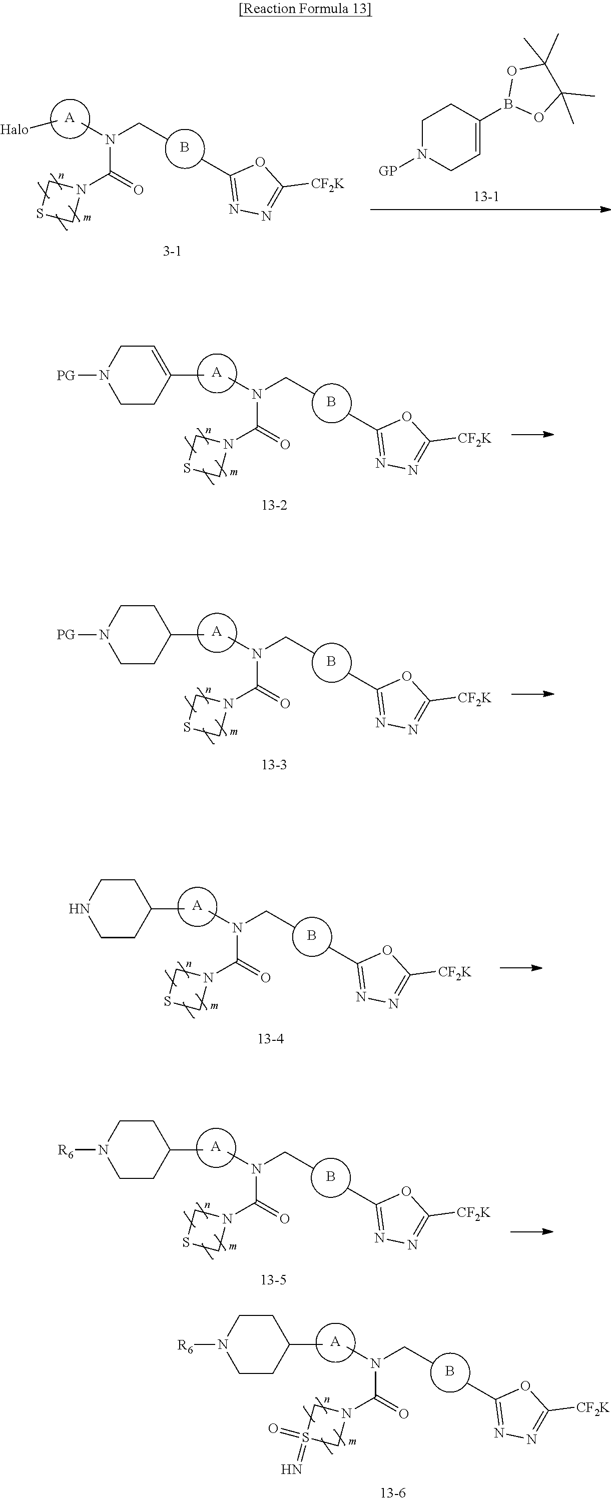

Above reaction formula 13 may represent a method for synthesizing a 1,3,4-oxadiazole compound including a sulfoximine functional group. In above reaction formula 13, R₆ may represent an acetyl group.

According to above reaction formula 13, a compound of formula 13-2 having a tetrahydropyridine structure including a protecting group may be prepared through C—C coupling (Suzuki reaction) between a compound of formula 13-1 and a compound of formula 3-1, which may be prepared according to substantially the same method as the preparation of the compound of formula 1-5 described in reaction formula 1. After that, a compound of formula 13-3, in which tetrahydropyridine of compound of formula 13-2 is converted to piperidine through a reduction reaction, may be prepared. Then, the protecting group may be removed to prepare a compound of formula 13-4, and then an acylation reaction and an alkylation reaction may be performed to prepare a compound of formula 13-5. After that, a compound of formula 13-6, into which a sulfoximine functional group is introduced through a bisacetoxyiodobenzene medium and N-source, may be prepared. The compound prepared according to above reaction formula 13 may include compound 67.

### Pharmaceutical Composition and Use Thereof

The pharmaceutical composition according to the present invention may include a sulfoximine compound according to above (1), (2), (3), (4), (5), (6) or (7) of the present invention, stereoisomers thereof or pharmaceutically acceptable salts thereof.

The pharmaceutical composition according to the present invention may include a sulfoximine compound according to above (1), (2), (3), (4), (5), (6) or (7) of the present invention, stereoisomers thereof or pharmaceutically acceptable salts thereof as an active ingredient, and used in preventing or treating HDAC-mediated diseases.

The pharmaceutical composition according to the present invention may include a sulfoximine compound represented by formula I, stereoisomers thereof or pharmaceutically acceptable salts thereof as an active ingredient, and used in preventing or treating HDAC-mediated diseases.

Above HDAC-mediated diseases may include infectious diseases, neoplasm, endocrinopathy, nutritional and metabolic diseases, mental and behavioral disorders, neurological diseases, eye and ocular adnexal diseases, circulatory diseases, respiratory diseases, digestive troubles, renal failure, skin and subcutaneous tissue diseases, musculoskeletal system and connective tissue diseases, or teratosis, deformities and chromosomal aberration.

In the present invention, the HDAC-mediated diseases may be a disease associated with HDAC6 activity, that is, a HDAC6-mediated disease.

The HDAC6-mediated diseases may include cancer, inflammatory diseases, autoimmune diseases, neurological or degenerative neurological diseases, specifically, lung cancer, colon cancer, breast cancer, prostate cancer, liver cancer, brain cancer, ovarian cancer, gastric cancer, skin cancer, pancreatic cancer, glioma, glioblastoma carcinoma, leukemia, lymphoma, multiple myeloma, solid cancer, Wilson's disease, spinocerebellar ataxia, prion disease, Parkinson's disease, Huntington's disease, amyotrophic lateral sclerosis, amyloidosis, Alzheimer's disease, alcoholic liver disease, spinal muscular atrophy, rheumatoid arthritis, osteoarthritis, Charcot-Marie-Tooth disease (CMT), heart failure (HF), pulmonary arterial hypertension (PAH) or idiopathic pulmonary fibrosis (IPF), in addition to symptoms or diseases related to abnormal functions of the HDAC.

The endocrinopathy, nutritional and metabolic diseases may include Wilson's disease, amyloidosis or diabetes, the mental and behavioral disorders may include depression or Rett syndrome, and the neurological diseases may include central nervous system atrophy, neurodegenerative disease, movement disorder, neuropathy, motor neuron disease, central nervous system demyelinating disease, or Charcot-Marie-Tooth disease (CMT), the eye and ocular adnexal diseases may include uveitis, the renal failure (RF) may include acute renal failure or chronic renal failure, the skin and subcutaneous tissue diseases may include psoriasis, the musculoskeletal system and connective tissue diseases may include rheumatoid arthritis, osteoarthritis or systemic lupus erythematosus, the teratosis, deformities and chromosomal aberration may include autosomal dominant polycystic kidney disease, the infectious diseases may include prion disease, the neoplasm may include benign tumor or malignant tumor, the circulatory diseases may include atrial fibrillation (AF), stroke, heart failure (HF) or pulmonary arterial hypertension (PAH), the respiratory diseases may include asthma or idiopathic pulmonary fibrosis (IPF), and the digestive diseases may include alcoholic liver disease, inflammatory bowel disease, Crohn's disease or ulcerative bowel disease.

The pharmaceutical composition of the present invention may further include at least one type of a pharmaceutically acceptable carrier, in addition to the sulfoximine compound according to above (1), (2), (3), (4), (5), (6) or (7) of the present invention, optical isomers thereof or pharmaceutically acceptable salts thereof. In this case, the pharmaceutically acceptable carrier may include one which is conventionally used in formulating a preparation, specifically including, but not limited thereto, lactose, dextrose, sucrose, sorbitol, mannitol, starch, acacia rubber, calcium phosphate, alginate, gelatin, calcium silicate, microcrystalline cellulose, polyvinyl pyrrolidone, cellulose, water, syrup, methyl cellulose, methyl hydroxybenzoate, propyl hydroxybenzoate, talc, magnesium stearate, mineral oil, or the like. In addition, the pharmaceutical composition of the present invention may further include lubricant, humectant, a sweetening agent, a flavoring agent, emulsifier, a suspending agent, preservative, or the like, in addition to the above ingredients.

Furthermore, the pharmaceutical composition of the present invention may be formulated into an oral dosage form such as tablet, powder, granule, pill, capsule, suspension, emulsion, liquid for internal use, emulsion, syrup, etc., as well as a form of external application, suppository and sterile solution for injection, and thus may be prepared in a unit dose form or prepared by being inserted into a multi-dose container. The preparations may be prepared according to a conventional method used for formulation in the art or a method disclosed in Remington's Pharmaceutical Science (19th ed., 1995), and may be formulated into various preparations depending on each disease or ingredient.

The pharmaceutical composition of the present invention may be orally or parenterally administered (for example, applied intravenously, hypodermically, intraperitoneally or locally) according to an intended method, in which a dosage thereof varies depending on a patient's condition and weight, a degree of disease, a drug form, and an administration route and time, but may be appropriately selected by those skilled in the art. Specifically, a daily dosage of the compound according to above (1), (2), (3), (4), (5), (6) or (7) of the present invention may be about 0.1 to about 1000 mg/kg, preferably about 5 to about 100 mg/kg, and may be administered at one time a day or several times a day by dividing the daily dosage of the compound.

In addition to the sulfoximine compound according to above (1), (2), (3), (4), (5), (6) or (7) of the present invention, stereoisomers thereof or pharmaceutically acceptable salts thereof, the pharmaceutical composition of the present invention may further include at least one ingredient which may exhibit the same or similar medicinal effects or may bring synergy to medicinal effects in combination.

The present invention may provide a method for preventing or treating histone deacetylase-mediated diseases, including administering the sulfoximine compound according to above (1), (2), (3), (4), (5), (6) or (7) of the present invention, stereoisomers thereof or pharmaceutically acceptable salts thereof into an individual.

The sulfoximine compound according to above (1), (2), (3), (4), (5), (6) or (7) of the present invention, stereoisomers thereof or pharmaceutically acceptable salts thereof may be administered in a therapeutically effective amount thereof.

In the present invention, the “therapeutically effective amount” may refer to an amount enough to treat a disease at a reasonable benefit/risk ratio applicable to medical treatment, and a level of effective dose may be determined according to factors including a patient's disease type, severity, activity of a drug, sensitivity to the drug, an administration time, an administration route and excretion rate, a treatment period and a concurrently used drug, as well as other factors well known in a medical field. Specifically, it may refer to an amount effective for preventing or treating HDAC6-mediated diseases.

In the present invention, “individual” may refer to a subject, whose disease needs to be prevented or treated, and more specifically to a mammal such as a human, monkey, mouse, dog, cat, horse, cow, etc., but is not limited thereto.

In the present invention, the term “prevention” may refer to all the acts, which inhibit or delay the occurrence of a disease by administering the sulfoximine compound according to above (1), (2), (3), (4), (5), (6) or (7) of the present invention, stereoisomers thereof or pharmaceutically acceptable salts thereof.

In the present invention, the term “treatment” may refer to all the acts, by which a symptom of the disease gets better or takes a favorable turn by administering the sulfoximine compound according to above (1), (2), (3), (4), (5), (6) or (7) of the present invention, stereoisomers thereof or pharmaceutically acceptable salts thereof.

The present invention may provide a use of the sulfoximine compound according to above (1), (2), (3), (4), (5), (6) or (7) of the present invention, stereoisomers thereof or pharmaceutically acceptable salts thereof for preventing or treating HDAC-mediated diseases.

The present invention may provide a use of the sulfoximine compound according to above (1), (2), (3), (4), (5), (6) or (7) of the present invention, stereoisomers thereof or pharmaceutically acceptable salts thereof in preparing a medicament for preventing or treating histone deacetylase-mediated diseases.

Matters mentioned in the sulfoximine compound, use, composition and therapeutic method of the present invention may be equally applied, if not contradictory to each other. Besides, the terms and abbreviations used in the present specification may have their original meanings, unless defined otherwise.

### Advantageous Effects

The sulfoximine compound of the present invention, stereoisomers thereof or pharmaceutically acceptable salts thereof can selectively inhibit HDAC6, and thus have a remarkably excellent effect on preventing or treating HDAC-mediated diseases. Thus, the sulfoximine compound of the present invention, stereoisomers thereof or pharmaceutically acceptable salts thereof can be advantageously used in preventing or treating HDAC-mediated diseases.

### MODE FOR INVENTION

Hereinafter, the present invention will be described in more detail through preparation examples and exemplary examples. However, the following preparation examples and exemplary examples are provided for the purpose of illustrating the present invention, and thus the present invention is not limited to the preparation examples and exemplary examples.

### Preparation Example

Each of the compounds according to the present invention was synthesized by a method described below, and a conventional method derived by combining the following specific synthesis methods may be used by those skilled in the art.

Each of the compounds used in the synthesis was purchased from a supplier outside or synthesized by using an organic synthesis method apparent to those skilled in the art, and was used without a separate purification process. The compound of each example was identified through 400 MHz ¹H-NMR (Agilent, 400-MR), LC-Mass (Waters, SQD2) analysis.

### Example 1: Synthesis of Compound 1, N-(4-(5-(difluoromethyl)-1,3,4-oxadiazol-2-yl)-2-fluorobenzyl)-1-imino-N-phenylthiomorpholin-4-carboxamide 1-oxide

### [Step 1] Synthesis of N-phenylthiomorpholin-4-carboxamide

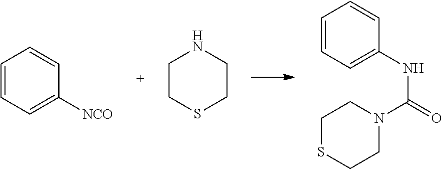

Isocyanatobenzene (100.00%, 2.000 g, 16.790 mmol) and thiomorpholine (100.00%, 1.000 equiv., 16.790 mmol) were dissolved in dichloromethane (10 mL) at room temperature, after which the resulting solution was stirred at the same temperature for four hours. Solvent was removed from the reaction mixture under reduced pressure, after which hexane (20 mL) and ethyl acetate (10 mL) were added into the resulting concentrate and stirred to filter out a precipitated solid, which was then washed with hexane and dried to obtain the title compound (3.000 g, 80.38%) in a white solid form.

### [Step 2] Synthesis of N-(4-(5-(difluoromethyl)-1,3,4-oxadiazol-2-yl)-2-fluorobenzyl)-N-phenylthiomorpholin-4-carboxamide

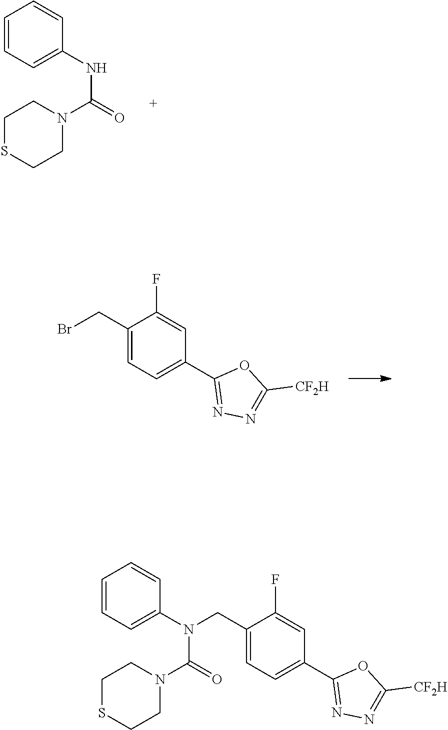 [SMILES: O=C(Nc1ccccc1)N1CCSCC1 ; Fc1cc(-c2nnc(C(F)F)o2)ccc1CBr ; O=C(N1CCSCC1)N(Cc1ccc(-c2nnc(C(F)F)o2)cc1F)c1ccccc1]

N-phenylthiomorpholin-4-carboxamide (100.00%, 2.270 g, 10.210 mmol) prepared in step 1 was dissolved in N,N-dimethylformamide (10 mL) at 0° C., after which sodium hydride (100.00%, 1.500 equiv., 15.320 mmol) was added to the resulting solution and stirred at the same temperature for 30 minutes. 2-(4-(bromomethyl)-3-fluorophenyl)-5-(difluoromethyl)-1,3,4-oxadiazole (100.00%, 1.000 equiv., 10.210 mmol) was added into the reaction mixture and further stirred at room temperature for two hours. Solvent was removed from the reaction mixture under reduced pressure, after which saturated aqueous solution of sodium hydrogen carbonate was poured into the resulting concentrate and extracted with dichloromethane. An organic layer was washed with saturated aqueous solution of sodium chloride, dehydrated with anhydrous magnesium sulfate, filtered, and concentrated under reduced pressure. The resulting concentrate was purified via column chromatography (SiO₂, 40 g cartridge; dichloromethane/methanol=0 to 10%) and concentrated to obtain the title compound (2.940 g, 64.19%) in a colorless oil form.

### [Step 3] Synthesis of Compound 1

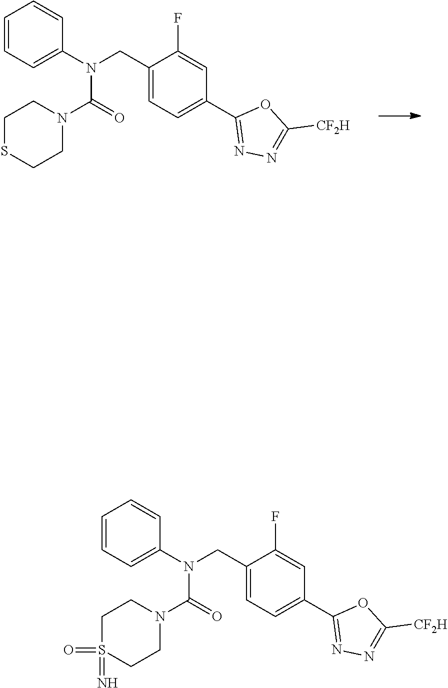 [SMILES: O=C(N1CCSCC1)N(Cc1ccc(-c2nnc(C(F)F)o2)cc1F)c1ccccc1 ; N=S1(=O)CCN(C(=O)N(Cc2ccc(-c3nnc(C(F)F)o3)cc2F)c2ccccc2)CC1]

N-(4-(5-(difluoromethyl)-1,3,4-oxadiazol-2-yl)-2-fluorobenzyl)-N-phenylthiomorpholin-4-carboxamide (100.00%, 2.940 g, 6.555 mmol) prepared in step 2, ammonium carbamate (100.00%, 2.000 equiv., 13.110 mmol) and (diacetoxyiodo)benzene (100.00%, 2.500 equiv., 16.390 mmol) were dissolved in methanol (50 ML) at room temperature, after which the resulting solution was stirred overnight at the same temperature. Solvent was removed from the reaction mixture under reduced pressure, after which saturated aqueous solution of sodium hydrogen carbonate was poured into the resulting concentrate and extracted with dichloromethane. An organic layer was washed with saturated aqueous solution of sodium chloride, dehydrated with anhydrous magnesium sulfate, filtered, and concentrated under reduced pressure. The resulting concentrate was purified via column chromatography (SiO₂, 40 g cartridge; dichloromethane/methanol=0 to 10%) and concentrated to obtain the title compound (2.500 g, 79.54%) in a colorless oil form.

¹H NMR (400 MHz, CDCl₃) δ 7.86 (dd, J=8.0, 1.5 Hz, 1H), 7.74 (dd, J=10.1, 1.5 Hz, 1H), 7.67 (t, J=7.6 Hz, 1H), 7.36 (t, J=7.8 Hz, 2H), 7.23-7.19 (m, 1H), 7.13-7.11 (m, 2H), 7.03 (s, 0.25H), 6.90 (s, 0.5H), 6.77 (s, 0.25H), 4.92 (s, 2H), 3.79-3.72 (m, 2H), 3.67-3.60 (m, 2H), 2.80 (t, J=5.2 Hz, 4H), 2.46 (s, 1H); LRMS (ES) m/z 480.9 (M⁺+1)

### Example 2: Synthesis of Compound 2, N-((5-(5-(difluoromethyl)-1,3,4-oxadiazol-2-yl)pyridin-2-yl)methyl)-N-phenyl-1-((2,2,2-trifluoroacetyl)imino)thiomorpholin-4-carboxamide 1-oxide

### [Step 1] Synthesis of N-phenyl-1-((2,2,2-trifluoroacetyl)imino)thiomorpholin-4-carboxamide 1-oxide

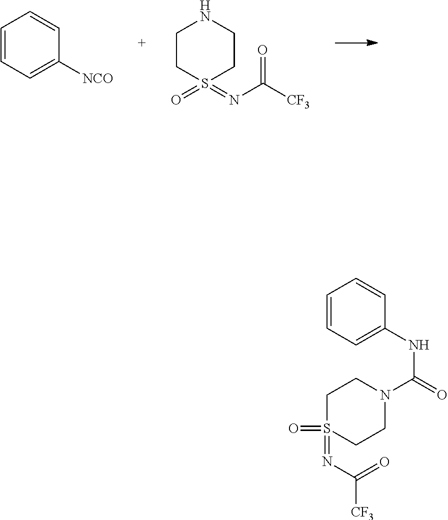

Isocyanatobenzene (0.590 g, 4.953 mmol) and 2,2,2-trifluoro-N-(1-oxidothiomorpholin-1-ylidene)acetamide (1.140 g, 4.953 mmol) were dissolved in dichloromethane (10 mL) at room temperature, after which the resulting solution was stirred at the same temperature for five hours. Solvent was removed from the resulting mixture under reduced pressure, after which the resulting concentrate was purified via column chromatography (SiO₂, 12 g cartridge; ethyl acetate/hexane=0 to 30%) and concentrated to obtain the title compound (0.160 g, 9.2%) in a white solid form.

### [Step 2] Synthesis of Compound 2

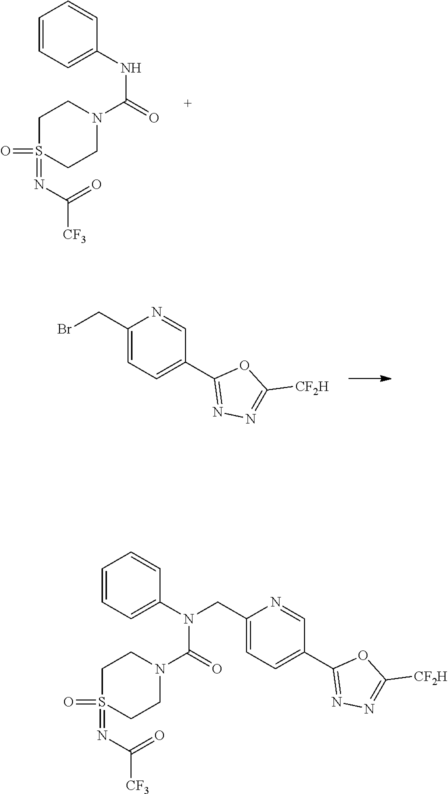 [SMILES: O=C(Nc1ccccc1)N1CCS(=O)(=NC(=O)C(F)(F)F)CC1 ; FC(F)c1nnc(-c2ccc(CBr)nc2)o1 ; O=C(N1CCS(=O)(=NC(=O)C(F)(F)F)CC1)N(Cc1ccc(-c2nnc(C(F)F)o2)cn1)c1ccccc1]

N-phenyl-1-((2,2,2-trifluoroacetyl)imino)thiomorpholin-4-carboxamide 1-oxide (0.120 g, 0.344 mmol) prepared in step 1 was dissolved in N,N-dimethylformamide (10 mL) at 0° C., after which sodium hydride (60.00%, 0.014 g, 0.344 mmol) was added to the resulting solution and stirred at the same temperature for 30 minutes. 2-(6-(bromomethyl)pyridin-3-yl)-5-(difluoromethyl)-1,3,4-oxadiazole (0.100 g, 0.344 mmol) was added into the reaction mixture and further stirred at room temperature for three hours. Water was poured into the reaction mixture and extracted with ethyl acetate. An organic layer was washed with saturated aqueous solution of sodium chloride, dehydrated with anhydrous sodium sulfate, filtered, and concentrated under reduced pressure. The resulting concentrate was purified via column chromatography (SiO₂, 12 g cartridge; ethyl acetate/hexane=0 to 50%) and concentrated to obtain the title compound (0.052 g, 27.1%) in a colorless oil form.

¹H NMR (400 MHz, CDCl₃) δ 9.25 (d, J=1.6 Hz, 1H), 8.43 (dd, J=8.0, 2.0 Hz, 1H), 7.54 (d, J=8.0 Hz, 1H), 7.39 (t, J=7.8 Hz, 2H), 7.25-7.19 (m, 3H), 7.08 (s, 0.25H), 6.95 (s, 0.5H), 6.82 (s, 0.25H), 5.13 (s, 2H), 3.98-3.94 (m, 2H), 3.74-3.69 (m, 2H), 3.53-3.37 (m, 4H); LRMS (ES) m/z 559.9 (M⁺+1)

### Example 3: Synthesis of Compound 3, N-((5-(5-(difluoromethyl)-1,3,4-oxadiazol-2-yl)pyridin-2-yl)methyl)-1-imino-N-phenylthiomorpholin-4-carboxamide 1-oxide

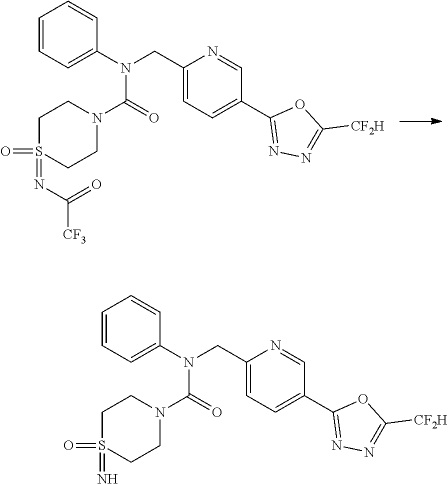 [SMILES: O=C(N1CCS(=O)(=NC(=O)C(F)(F)F)CC1)N(Cc1ccc(-c2nnc(C(F)F)o2)cn1)c1ccccc1 ; N=S1(=O)CCN(C(=O)N(Cc2ccc(-c3nnc(C(F)F)o3)cn2)c2ccccc2)CC1]

N-((5-(5-(difluoromethyl)-1,3,4-oxadiazol-2-yl)pyridin-2-yl)methyl)-N-phenyl-1-((2,2,2-trifluoroacetyl)imino)thiomorpholin-4-carboxamide 1-oxide (0.052 g, 0.093 mmol) prepared in step 2 of example 2 and potassium carbonate (0.051 g, 0.372 mmol) were dissolved in methanol (5 mL) at room temperature, after which the resulting solution was stirred at the same temperature for one hour. Solvent was removed from the resulting mixture under reduced pressure, after which the resulting concentrate was purified via column chromatography (SiO₂, 12 g cartridge; methanol/dichloromethane=0 to 10%) and concentrated to obtain the title compound (0.013 g, 30.2%) in a yellow oil form.

¹H NMR (400 MHz, CDCl₃) δ 9.26 (d, J=1.6 Hz, 1H), 8.38 (dd, J=8.2, 2.2 Hz, 1H), 7.57 (d, J=8.0 Hz, 1H), 7.39-7.32 (m, 2H), 7.21-7.17 (m, 3H), 7.07 (s, 0.25H), 6.95 (s, 0.5H), 6.82 (s, 0.25H), 5.11 (s, 2H), 3.81-3.63 (m, 4H), 3.02-2.91 (m, 4H), 2.50 (brs, 1H); LRMS (ES) m/z 463.9 (M⁺+1)

### Example 4: Synthesis of Compound 4, Benzyl (4-((4-(5-(difluoromethyl)-1,3,4-oxadiazol-2-yl)-2-fluorobenzyl)(phenyl)carbamoyl)-1-oxidothiomorpholin-1-ylidene)carbamate

### [Step 1] Synthesis of benzyl (1-oxido-4-(phenylcarbamoyl)thiomorpholin-1-ylidene)carbamate

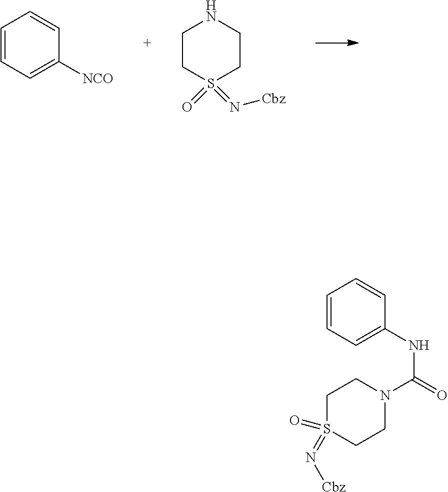

Benzyl (1-oxidothiomorpholin-1-ylidene)carbamate (100.00%, 2.000 g, 7.454 mmol) and isocyanatobenzene (100.00%, 1.000 equiv., 7.454 mmol) were dissolved in diethyl ether (50 mL) at room temperature, after which the resulting solution was stirred at the same temperature for five hours. A precipitated solid was filtered, washed with hexane, and dried to obtain the title compound (2.65 g, 91.76%, in a yellow solid form).

### [Step 2] Synthesis of Compound 4

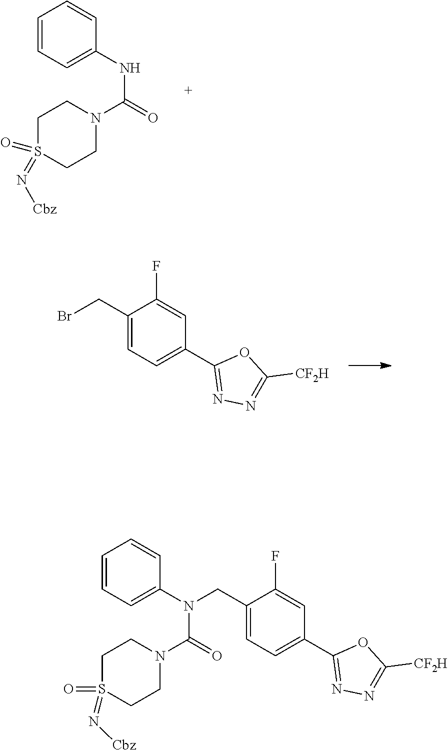 [SMILES: O=C(N=S1(=O)CCN(C(=O)Nc2ccccc2)CC1)OCc1ccccc1 ; Fc1cc(-c2nnc(C(F)F)o2)ccc1CBr ; O=C(N=S1(=O)CCN(C(=O)N(Cc2ccc(-c3nnc(C(F)F)o3)cc2F)c2ccccc2)CC1)OCc1ccccc1]

The title compound (2.08 g, 49.56%, colorless oil) was obtained according to substantially the same reaction as described in step 2 of example 1 except for using benzyl (1-oxido-4-(phenylcarbamoyl)thiomorpholin-1-ylidene)carbamate (100.00%, 2.650 g, 6.840 mmol) prepared in step 1 instead of N-phenylthiomorpholin-4-carboxamide.

¹H NMR (400 MHz, CDCl₃) δ 7.87 (d, J=8.0 Hz, 1H), 7.74 (d, J=10.0 Hz, 1H), 7.66 (t, J=7.6 Hz, 1H), 7.39-7.28 (m, 7H), 7.25-7.21 (m, 1H), 7.12 (d, J=8.0 Hz, 2H), 7.05 (s, 0.25H), 6.92 (s, 0.5H), 6.79 (s, 0.25H), 5.09 (s, 2H), 4.93 (s, 2H), 3.90-3.85 (m, 2H), 3.89-3.85 (m, 2H), 3.56-3.50 (m, 2H), 3.42-3.38 (m, 2H); LRMS (ES) m/z 614.9 (M⁺+1)

### Example 5: Synthesis of Compound 5, Benzyl (4-(((5-(5-(difluoromethyl)-1,3,4-oxadiazol-2-yl)pyridin-2-yl)methyl)(phenyl)carbamoyl)-1-oxidothiomorpholin-1-ylidene)carbamate

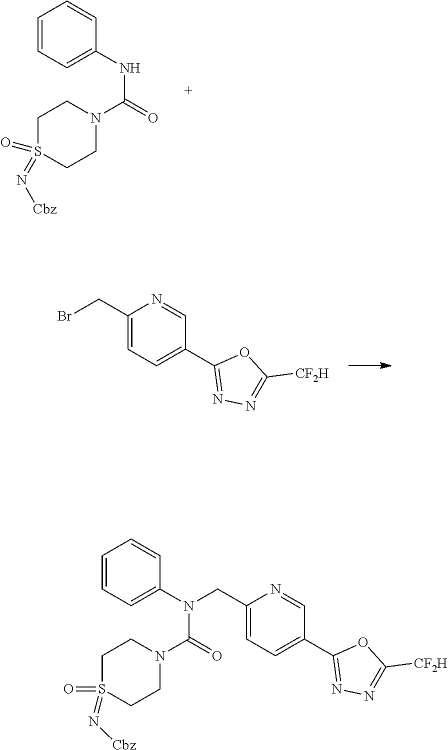 [SMILES: O=C(N=S1(=O)CCN(C(=O)Nc2ccccc2)CC1)OCc1ccccc1 ; FC(F)c1nnc(-c2ccc(CBr)nc2)o1 ; O=C(N=S1(=O)CCN(C(=O)N(Cc2ccc(-c3nnc(C(F)F)o3)cn2)c2ccccc2)CC1)OCc1ccccc1]

The title compound (0.12 g, 15.58%, white solid) was obtained according to substantially the same reaction as described in step 2 of example 2 except for using benzyl (1-oxido-4-(phenylcarbamoyl)thiomorpholin-1-ylidene)carbamate instead of N-phenyl-1-((2,2,2-trifluoroacetyl)imino)thiomorpholin-4-carboxamide 1-oxide.

¹H NMR (400 MHz, CDCl₃) δ 9.25 (d, J=1.6 Hz, 1H), 8.40 (dd, J=8.2, 2.2 Hz, 1H), 7.54 (d, J=8.4 Hz, 1H), 7.41-7.30 (m, 7H), 7.22-7.18 (m, 3H), 7.08 (s, 0.25H), 6.95 (s, 0.5H), 6.82 (s, 0.25H), 5.11 (s, 2H), 5.10 (s, 2H), 3.92-3.87 (m, 2H), 3.57-3.50 (m, 4H), 3.28-3.22 (m, 2H); LRMS (ES) m/z 597.9 (M⁺+1)

### Example 6: Synthesis of Compound 6, N-(4-(5-(difluoromethyl)-1,3,4-oxadiazol-2-yl)-2-fluorobenzyl)-N-(3,4-difluorophenyl)-1-iminothiomorpholin-4-carboxamide 1-oxide

### [Step 1] Synthesis of N-(3,4-difluorophenyl)thiomorpholin-4-carboxamide

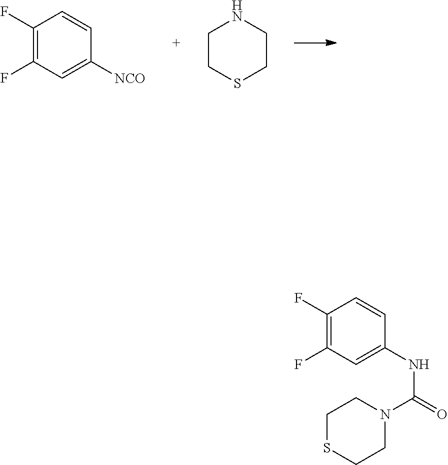

The title compound (1.500 g, 90.07%) was obtained in a white solid form according to substantially the same reaction as described in step 1 of example 1 except for using 1,2-difluoro-4-isocyanato-benzene instead of isocyanatobenzene.

### [Step 2] Synthesis of N-((4-(5-(difluoromethyl)-1,3,4-oxadiazol-2-yl)-2-fluorobenzyl)-N-(3,4-difluorophenyl)thiomorpholin-4-carboxamide

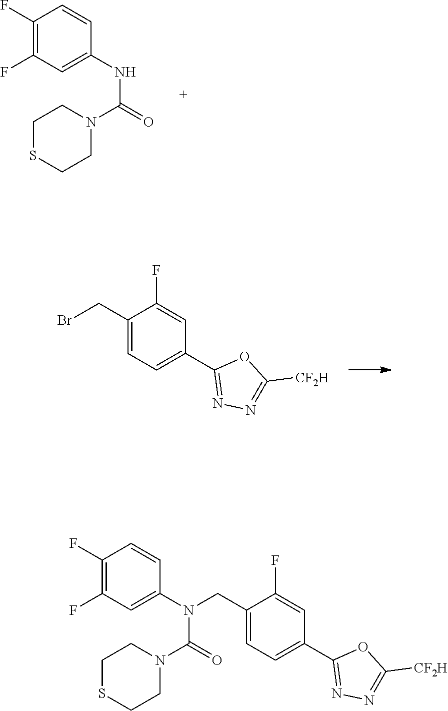 [SMILES: O=C(Nc1ccc(F)c(F)c1)N1CCSCC1 ; Fc1cc(-c2nnc(C(F)F)o2)ccc1CBr ; O=C(N1CCSCC1)N(Cc1ccc(-c2nnc(C(F)F)o2)cc1F)c1ccc(F)c(F)c1]

The title compound (0.300 g, 53.32%) was obtained in a white foam solid form according to substantially the same reaction as described in step 2 of example 1 except for using N-(3,4-difluorophenyl)thiomorpholin-4-carboxamide (100.00%, 0.300 g, 1.161 mmol) prepared in step 1 instead of N-phenylthiomorpholin-4-carboxamide.

### [Step 3] Synthesis of Compound 6

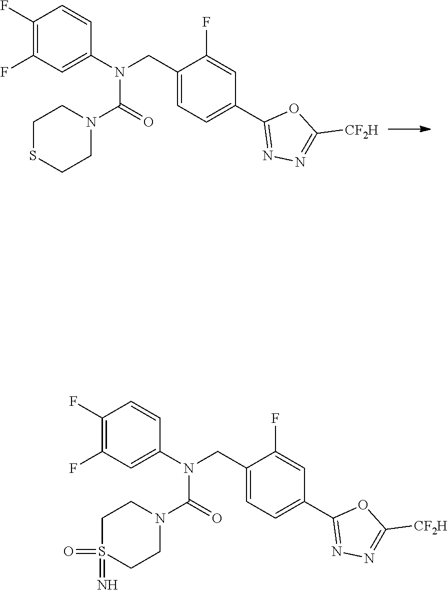 [SMILES: O=C(N1CCSCC1)N(Cc1ccc(-c2nnc(C(F)F)o2)cc1F)c1ccc(F)c(F)c1 ; N=S1(=O)CCN(C(=O)N(Cc2ccc(-c3nnc(C(F)F)o3)cc2F)c2ccc(F)c(F)c2)CC1]

The title compound (0.22 g, 68.91%) was obtained in a colorless oil form according to substantially the same reaction as described in step 3 of example 1 except for using N-((4-(5-(difluoromethyl)-1,3,4-oxadiazol-2-yl)-2-fluorobenzyl)-N-(3,4-difluorophenyl)thiomorpholin-4-carboxamide prepared in step 2 instead of N-(4-(5-(difluoromethyl)-1,3,4-oxadiazol-2-yl)-2-fluorobenzyl)-N-phenylthiomorpholin-4-carboxamide.

¹H NMR (400 MHz, CDCl₃) δ 7.90 (dd, J=8.0, 1.6 Hz, 1H), 7.78 (dd, J=10.1, 1.5 Hz, 1H), 7.68 (t, J=7.6 Hz, 1H), 7.20-7.14 (m, 1H), 7.06 (s, 0.25H), 7.03-6.98 (m, 1H), 6.93 (s, 0.5H), 6.89-6.85 (m, 1H), 6.80 (s, 0.25H), 4.90 (s, 2H), 3.79-3.62 (m, 4H), 2.93-2.90 (m, 4H), 2.50 (brs, 1H); LRMS (ES) m/z 516.9 (M⁺+1)

### Example 7: Synthesis of Compound 7, N-((5-(5-(difluoromethyl)-1,3,4-oxadiazol-2-yl)pyridin-2-yl)methyl)-N-(3,4-difluorophenyl)-1-iminothiomorpholin-4-carboxamide 1-oxide

### [Step 1] Synthesis of N-((5-(5-(difluoromethyl)-1,3,4-oxadiazol-2-yl)pyridin-2-yl)methyl)-N-(3,4-difluorophenyl)thiomorpholin-4-carboxamide

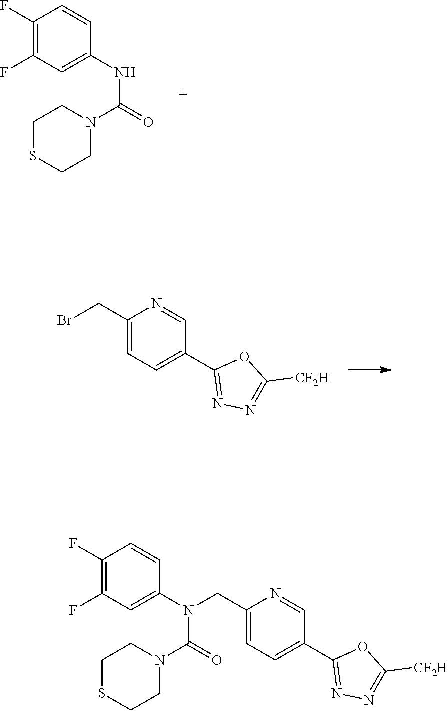 [SMILES: O=C(Nc1ccc(F)c(F)c1)N1CCSCC1 ; FC(F)c1nnc(-c2ccc(CBr)nc2)o1 ; O=C(N1CCSCC1)N(Cc1ccc(-c2nnc(C(F)F)o2)cn1)c1ccc(F)c(F)c1]

The title compound (0.220 g, 40.53%) was obtained in a colorless oil form according to substantially the same reaction as described in step 2 of example 6 except for using 2-(6-(bromomethyl)pyridin-3-yl)-5-(difluoromethyl)-1,3,4-oxadiazole instead of 2-(4-(bromomethyl)-3-fluorophenyl)-5-(difluoromethyl)-1,3,4-oxadiazole in step 2 of example 6.

### [Step 2] Synthesis of Compound 7

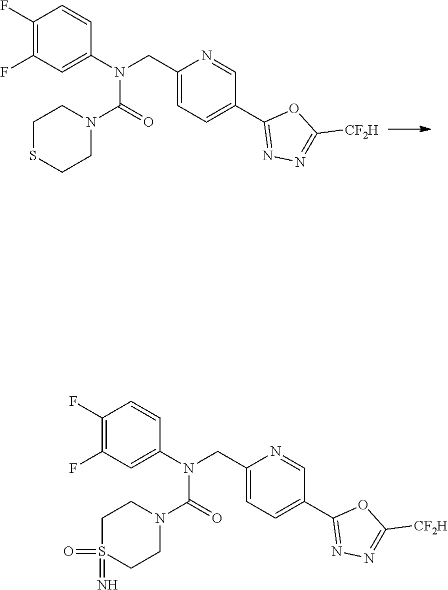 [SMILES: O=C(N1CCSCC1)N(Cc1ccc(-c2nnc(C(F)F)o2)cn1)c1ccc(F)c(F)c1 ; N=S1(=O)CCN(C(=O)N(Cc2ccc(-c3nnc(C(F)F)o3)cn2)c2ccc(F)c(F)c2)CC1]

The title compound (0.15 g, 64.82%) was obtained in a colorless oil form according to substantially the same reaction as described in step 3 of example 6 by using N-((5-(5-(difluoromethyl)-1,3,4-oxadiazol-2-yl)pyridin-2-yl)methyl)-N-(3,4-difluorophenyl)thiomorpholin-4-carboxamide obtained from step 1.

¹H NMR (400 MHz, CDCl₃) 9.25 (d, J=1.6 Hz, 1H), 8.40 (dd, J=8.2, 2.2 Hz, 1H), 7.54 (d, J=8.2 Hz, 1H), 7.18-7.08 (m, 2H), 7.08 (s, 0.25H), 6.97-6.93 (m, 1H), 6.96 (s, 0.5H), 6.82 (s, 0.25H), 5.06 (s, 2H), 3.79-3.61 (m, 4H), 3.07-2.98 (m, 4H), 2.40 (brs, 1H); LRMS (ES) m/z 499.9 (M⁺+1)

### Example 8: Synthesis of Compound 8, N-(4-(5-(difluoromethyl)-1,3,4-oxadiazol-2-yl)-2-fluorobenzyl)-1-imino-N-phenyltetrahydro-2H-thiopyran-4-carboxamide 1-oxide

### [Step 1] Synthesis of N-(4-(5-(difluoromethyl)-1,3,4-oxadiazol-2-yl)-2-fluorobenzyl)-N-phenyltetrahydro-2H-thiopyran-4-carboxamide

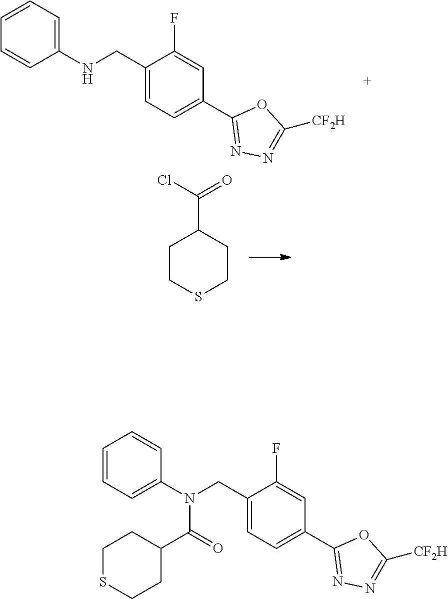

N-((4-(5-(difluoromethyl)-1,3,4-oxadiazol-2-yl)-2-fluoro-phenyl)methyl)aniline (100.00%, 0.600 g, 1.879 mmol) was dissolved in N,N-dimethylformamide (10 mL) at 0° C., after which sodium hydride (60.00%, 1.500 equiv., 2.819 mmol) was added to the resulting solution and stirred at the same temperature for 30 minutes. Tetrahydrothiopyran-4-carbonyl chloride (100.00%, 1.000 equiv., 1.879 mmol) was added into the reaction mixture and further stirred at room temperature for two hours. Solvent was removed from the reaction mixture under reduced pressure, after which saturated aqueous solution of sodium hydrogen carbonate was poured into the resulting concentrate and extracted with dichloromethane. An organic layer was washed with saturated aqueous solution of sodium chloride, dehydrated with anhydrous magnesium sulfate, filtered, and concentrated under reduced pressure. The resulting concentrate was purified via column chromatography (SiO₂, 12 g cartridge; dichloromethane/methanol=0 to 10%) and concentrated to obtain the title compound (0.510 g, 60.65%) in a colorless oil form.

### [Step 2] Synthesis of Compound 8

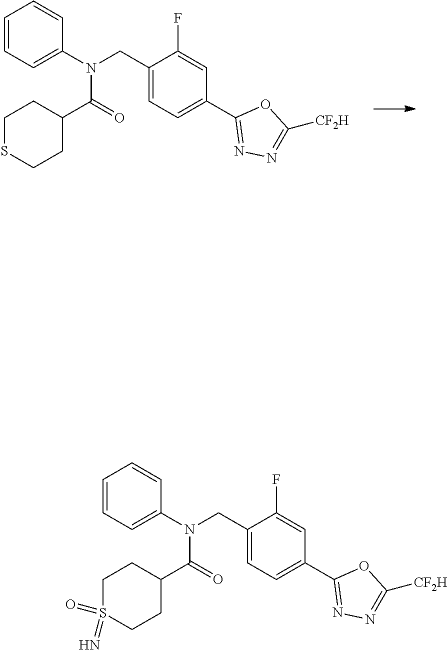 [SMILES: O=C(C1CCSCC1)N(Cc1ccc(-c2nnc(C(F)F)o2)cc1F)c1ccccc1 ; N=S1(=O)CCC(C(=O)N(Cc2ccc(-c3nnc(C(F)F)o3)cc2F)c2ccccc2)CC1]

The title compound (0.120 g, 22.01%) was obtained in a colorless oil form according to substantially the same reaction as described in step 3 of example 1 except for using N-(4-(5-(difluoromethyl)-1,3,4-oxadiazol-2-yl)-2-fluorobenzyl)-N-phenyltetrahydro-2H-thiopyran-4-carboxamide prepared in step 1 instead of N-(4-(5-(difluoromethyl)-1,3,4-oxadiazol-2-yl)-2-fluorobenzyl)-N-phenylthiomorpholin-4-carboxamide.

¹H NMR (400 MHz, CDCl₃) δ 7.87 (d, J=8.0 Hz, 1H), 7.73 (dd, J=9.8, 1.5 Hz, 1H), 7.56-7.51 (m, 1H), 7.41-7.39 (m, 3H), 7.05-7.03 (m, 2H), 7.05 (s, 0.25H), 6.92 (s, 0.5H), 6.79 (s, 0.25H), 5.04 (d, J=2.2 Hz, 2H), 3.30-3.26 (m, 2H), 2.82-2.79 (m, 2H), 2.52-2.50 (m, 1H), 2.41-2.36 (m, 2H), 2.20 (brs, 1H), 2.09-2.05 (m, 2H); LRMS (ES) m/z 479.9 (M⁺+1)

### Example 9: Synthesis of Compound 9, 1-imino-N-phenyl-N-((5-(5-(trifluoromethyl)-1,3,4-oxadiazol-2-yl)pyridin-2-yl)methyl)thiomorpholine-4-carboxamide 1-oxide

### [Step 1] Synthesis of N-phenyl-N-((5-(5-(trifluoromethyl)-1,3,4-oxadiazol-2-yl)pyridin-2-yl)methyl)thiomorpholine-4-carboxamide

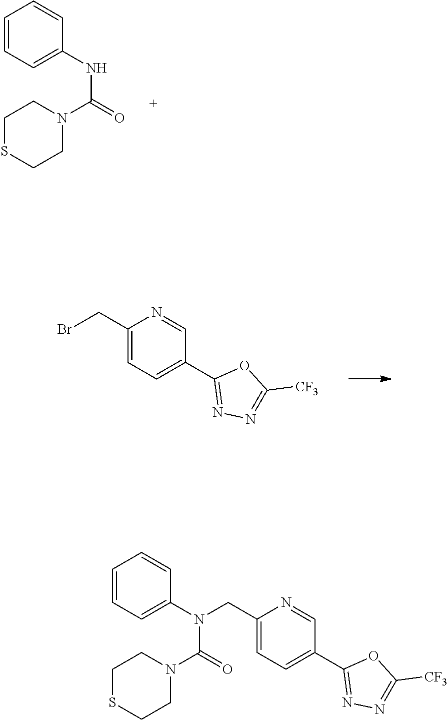 [SMILES: O=C(Nc1ccccc1)N1CCSCC1 ; FC(F)(F)c1nnc(-c2ccc(CBr)nc2)o1 ; O=C(N1CCSCC1)N(Cc1ccc(-c2nnc(C(F)(F)F)o2)cn1)c1ccccc1]

N-phenylthiomorpholin-4-carboxamide (100.00%, 0.050 g, 0.225 mmol) was dissolved in N,N-dimethylformamide (10 mL) at 0° C., after which sodium hydride (100.00%, 1.500 equiv., 0.337 mmol) was added to the resulting solution and stirred at the same temperature for 30 minutes. 2-(6-(bromomethyl)pyridin-3-yl)-5-(trifluoromethyl)-1,3,4-oxadiazole (100.00%, 1.000 equiv, 0.225 mmol) was added into the reaction mixture and further stirred at room temperature for two hours. Solvent was removed from the reaction mixture under reduced pressure, after which saturated aqueous solution of sodium hydrogen carbonate was poured into the resulting concentrate and extracted with dichloromethane. An organic layer was washed with saturated aqueous solution of sodium chloride, dehydrated with anhydrous magnesium sulfate, filtered, and concentrated under reduced pressure. The resulting concentrate was purified via column chromatography (SiO₂, 12 g cartridge; hexane/ethyl acetate=0 to 30%) and concentrated to obtain the title compound (0.005 g, 4.95%) in a colorless oil form.

### [Step 2] Synthesis of Compound 9

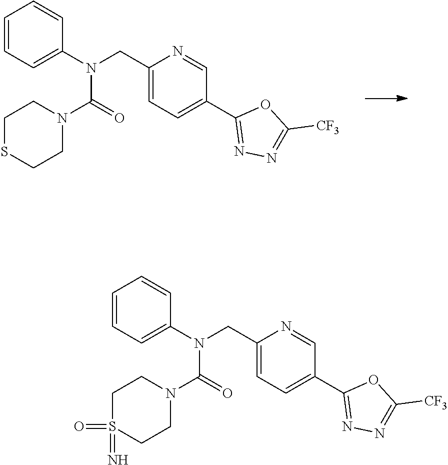 [SMILES: O=C(N1CCSCC1)N(Cc1ccc(-c2nnc(C(F)(F)F)o2)cn1)c1ccccc1 ; N=S1(=O)CCN(C(=O)N(Cc2ccc(-c3nnc(C(F)(F)F)o3)cn2)c2ccccc2)CC1]

The title compound (0.010 g, 20.78%) was obtained in a colorless oil form according to substantially the same reaction as described in step 3 of example 1 except for using N-phenyl-N-((5-(5-(trifluoromethyl)-1,3,4-oxadiazol-2-yl)pyridin-2-yl)methyl)thiomorpholin-4-carboxamide prepared in step 1 instead of N-(4-(5-(difluoromethyl)-1,3,4-oxadiazol-2-yl)-2-fluorobenzyl)-N-phenylthiomorpholin-4-carboxamide.

¹H NMR (400 MHz, CDCl₃) δ 9.26 (d, J=1.6 Hz, 1H), 8.39 (dd, J=8.2, 2.2 Hz, 1H), 7.39-7.35 (m, 2H), 7.22-7.18 (m, 3H), 5.12 (s, 2H), 3.82-3.76 (m, 2H), 3.70-3.63 (m, 2H), 2.98-2.94 (m, 4H), 2.50 (brs, 1H); LRMS (ES) m/z 481.9 (M⁺+1)

### Examples 10 to 13: Synthesis of Compounds 10 to 13

Compounds 10 to 13 according to examples 10 to 13 were prepared in accordance with the same reactions as described in steps 1, 2 and 3 of example 1, respectively, except for using the reactants of table 2 below instead of isocyanatobenzene in step 1 of example 1. Table 2 shows the properties and yields of the products prepared in each of steps 1 to 3 in examples 10 to 13.

TABLE 2

| | | Yield/property | Yield/property | Yield/property |
|---|---|---|---|---|
| | | of product | of product | of product |
| | | obtained from | obtained from | obtained from |
| Classification | Reactant in step 1 | step 1 | step 2 | step 3 |
| Example 10 | 5-isocyanato-1-methyl-1H- | 0.700 g, 77.87% | 0.072 g, 26.36% | 0.040 g, 52.32% |
| | indole | White solid | Colorless oil | Colorless oil |
| Example 11 | (4- | 1.100 g, 67.71% | 0.085 g, 30.75% | 0.040 g, 41.82% |
| | isocyanatophenyl)(methyl)sulfan | White solid | Colorless oil | Colorless oil |
| Example 12 | 2-isocyanatothiophene | 0.300 g, 92.39% | 0.080 g, 40.18% | 0.030 g, 32.96% |
| | | White solid | Colorless oil | Colorless oil |
| Example 13 | 1-fluoro-3-isocyanatobenzene | 1.300 g, 74.18% | 0.167 g, 57.36% | 0.110 g, 69.68% |
| | | White solid | Colorless oil | Colorless oil |

The names of compounds 10 to 13 prepared in each of examples 10 to 13, and the NMR and LC-Mass analysis results thereof are shown in table 3 below.

TABLE 3

| | Compound | | |
|---|---|---|---|
| Classification | No. | Compound name | NMR/LRMS |
| Example 10 | 10 | N-(4-(5-(difluoromethyl)- | ¹H NMR (400 MHz, CDCl₃) δ 7.87 (dd, J = |
| | | 1,3,4-oxadiazol-2-yl)-2- | 8.0, 1.6 Hz, 1H), 7.74-7.68 (m, 2H), 7.37 |
| | | fluorobenzyl)-1-imino-N- | (d, J = 2.0 Hz, 1H), 7.29-7.27 (m, 1H), 7.12 |
| | | (1-methyl-1H-indol-5- | (d, J = 3.1 Hz, 1H), 7.05 (s, 0.25H), 6.96 (dd, |
| | | yl)thiomorpholin-4- | J = 8.7, 2.1 Hz, 1H), 6.92 (s, 0.5H), 6.79 (s, |
| | | carboxamide 1-oxide | 0.25H), 6.44 (dd, J = 3.1, 0.7 Hz, 1H), 4.97 |
| | | | (s, 2H), 3.84-3.78 (m, 2H), 3.70-3.64 (m, |
| | | | 2H), 2.74 (t, J = 5.2 Hz, 4H), 2.41 (brs, 1H); |
| | | | LRMS (ES) m/z 534.0 (M⁺ + 1) |
| Example 11 | 11 | N-(4-(5-(difluoromethyl)- | ¹H NMR (400 MHz, CDCl₃) δ 8.01 (d, J = |
| | | 1,3,4-oxadiazol-2-yl)-2- | 1.9 Hz, 2H), 7.90 (dd, J = 8.0, 1.5 Hz, 1H), |
| | | fluorobenzyl)-1-imino-N- | 7.80 (dd, J = 10.2, 1.5 Hz, 1H), 7.68 (t, J = |
| | | (4-(S- | 7.7 Hz, 1H), 7.31-7.29 (m, 2H), 7.06 (s, |
| | | methylsulfonimidoyl)phe- | 0.25H), 6.93 (s, 0.5H), 6.80 (s, 0.25H), 5.02 |
| | | nyl)thiomorpholin-4- | (s, 2H), 3.80-3.63 (m, 4H), 3.12 (s, 3H), |
| | | carboxamide 1-oxide | 2.96-2.94 (m, 4H), 2.80 (brs, 1H), 2.60 (brs, |
| | | | 1H); LRMS (ES) m/z 557.9 (M⁺ + 1) |
| Example 12 | 12 | N-(4-(5-(difluoromethyl)- | ¹H NMR (400 MHz, CDCl₃) δ 7.69 (dd, J = |
| | | 1,3,4-oxadiazol-2-yl)-2- | 164.9, 164.9 Hz, 1H), 7.80 (dd, J = 10.0, 1.6 |
| | | fluorobenzyl)-1-imino-N- | Hz, 1H), 7.63 (t, J = 7.6 Hz, 1H), 7.08-7.06 |
| | | (thiophen-2- | (m, 1H), 7.06 (s, 0.25H), 6.93 (s, 0.5H), 6.86 |
| | | yl)thiomorpholin-4- | (dd, J = 5.6, 3.7 Hz, 1H), 6.80 (s, 0.25H), |
| | | carboxamide 1-oxide | 6.68 (dd, J = 3.7, 1.3 Hz, 1H), 4.92 (s, 2H), |
| | | | 4.13-3.72 (m, 4H), 2.90-2.85 (m, 4H), 2.50 |
| | | | (brs, 1H); LRMS (ES) m/z 486.9 (M⁺ + 1) |
| Example 13 | 13 | N-(4-(5-(difluoromethyl)- | ¹H NMR (400 MHz, CDCl₃) δ 7.89 (dd, J = |
| | | 1,3,4-oxadiazol-2-yl)-2- | 8.0, 1.6 Hz, 1H), 7.77 (dd, J = 10.1, 1.6 Hz, |
| | | fluorobenzyl)-N-(3- | 1H), 7.67 (t, J = 7.6 Hz, 1H), 7.36-7.30 (m, |
| | | fluorophenyl)-1- | 1H), 7.05 (s, 0.25H), 6.95-6.87 (m, 3H), |
| | | iminothiomorpholin-4- | 6.91 (s, 0.5H), 6.79 (s, 0.25H), 4.94 (s, 2H), |
| | | carboxamide 1-oxide | 3.81-3.62 (m, 4H), 2.93-2.86 (m, 4H), 2.50 |
| | | | (brs, 1H); LRMS (ES) m/z 500.0 (M⁺ + 1) |

### Examples 14 to 17: Synthesis of Compounds 14 to 17

Compounds 14 to 17 according to examples 14 to 17 were prepared in accordance with the same reactions as described in steps 1, 2 and 3 of examples 10 to 13, respectively, except for using 2-(6-(bromomethyl)pyridin-3-yl)-5-(difluoromethyl)-1,3,4-oxadiazole instead of 2-(4-(bromomethyl)-3-fluorophenyl)-5-(difluoromethyl)-1,3,4-oxadiazole in step 2 of examples 10 to 13, respectively. Table 4 shows the properties and yields of the products prepared in each of steps 1 to 3 in examples 14 to 17.

TABLE 4

| | Yield/property of product | Yield/property of product | Yield/property of product |
|---|---|---|---|
| Classification | obtained from step 1 | obtained from step 2 | obtained from step 3 |
| Example 14 | (Same as product in | 0.137 g, 51.92% | 0.110 g, 75.46% |
| | step 1 of example 10) | Colorless oil | Colorless oil |
| Example 15 | (Same as product in | 0.080 g, 29.97% | 0.030 g, 33.19% |
| | step 1 of example 11) | Colorless oil | Colorless oil |
| Example 16 | (Same as product in | 0.085 g, 32.14% | 0.030 g, 32.96% |
| | step 1 of example 12) | Colorless oil | Colorless oil |
| Example 17 | (Same as product in | 0.129 g, 45.99% | 0.110 g, 79.75% |
| | step 1 of example 13) | Colorless oil | Colorless oil |

The names of compounds 14 to 17 prepared in each of examples 14 to 17, and the NMR and LC-Mass analysis results thereof are shown in table 5 below.

TABLE 5

| | Compound | | |
|---|---|---|---|
| Classification | No. | Compound name | NMR/LRMS |
| Example 14 | 14 | N-((5-(5- | ¹H NMR (400 MHz, CDCl₃) δ 9.24 (dd, J = 2.1, |
| | | (difluoromethyl)- | 0.6 Hz, 1H), 8.36 (dd, J = 8.2, 2.2 Hz, 1H), 7.62 |
| | | 1,3,4-oxadiazol-2- | (d, J = 8.2 Hz, 1H), 7.45 (d, J = 1.9 Hz, 1H), |
| | | yl)pyridin-2- | 7.30-7.28 (m, 1H), 7.11-7.06 (m, 2H), 7.08 |
| | | yl)methyl)-1-imino-N- | (s, 0.25H), 6.94 (s, 0.5H), 6.81 (s, 0.25H), 6.44 |
| | | (1-methyl-1H-indol-5- | (dd, J = 3.1, 0.7 Hz, 1H), 5.13 (s, 2H), 3.82- |
| | | yl)thiomorpholin-4- | 3.66 (m, 4H), 3.50 (s, 3H), 2.90-2.87 (m, 4H), |
| | | carboxamide 1-oxide | 2.50 (brs, 1H); LRMS (ES) m/z 516.8 (M⁺ + 1) |
| Example 15 | 15 | N-((5-(5-(difluoromethyl)- | ¹H NMR (400 MHz, CDCl₃) δ 9.27 (d, J = 1.7 |
| | | 1,3,4-oxadiazol-2- | Hz, 1H), 8.44 (dd, J = 8.2, 2.2 Hz, 1H), 8.00- |
| | | yl)pyridin-2- | 7.96 (m, 2H), 7.57-7.54 (m, 1H), 7.32-7.30 |
| | | yl)methyl)-1-imino-N- | (m, 2H), 7.09 (s, 0.25H), 6.96 (s, 0.5H), 6.83 (s, |
| | | (4-(S-methyl- | 0.25H), 5.16 (s, 2H), 3.85-3.68 (m, 4H), 3.14- |
| | | sulfonimidoyl)phenyl)thio- | 3.03 (m, 4H), 3.12 (s, 3H), 2.60 (brs, 2H); |
| | | morpholin-4-carboxamide | LRMS (ES) m/z 540.9 (M⁺ + 1) |
| | | 1-oxide | |
| Example 16 | 16 | N-((5-(5-(difluoromethyl)- | ¹H NMR (400 MHz, CDCl₃) δ 9.26 (d, J = 1.7 |
| | | 1,3,4-oxadiazol-2- | Hz, 1H), 8.38 (dd, J = 8.2, 2.2 Hz, 1H), 7.53 (d, |
| | | yl)pyridin-2- | J = 8.2 Hz, 1H), 7.08 (s, 0.25H), 7.02 (dd, J = |
| | | yl)methyl)-1-imino- | 5.6, 1.3 Hz, 1H), 6.95 (s, 0.5H), 6.84-6.82 (m, |
| | | N-(thiophen- | 1H), 6.82 (s, 0.25H), 6.71 (dd, J = 3.7, 1.3 Hz, |
| | | 2-yl)thiomorpholin- | 1H), 5.07 (s, 2H), 3.91-3.72 (m, 4H), 3.01- |
| | | 4-carboxamide 1-oxide | 2.88 (m, 4H), 2.60 (s, 1H); LRMS (ES) m/z |
| | | | 469.9 (M⁺ + 1) |
| Example 17 | 17 | N-((5-(5- | ¹H NMR (400 MHz, CDCl₃) δ 9.23-9.22 (m, |
| | | (difluoromethyl)- | 1H), 8.37 (dd, J = 8.2, 2.2 Hz, 1H), 7.53 (d, J = |
| | | 1,3,4-oxadiazol-2- | 8.2 Hz, 1H), 7.32-7.26 (m, 1H), 7.08 (s, |
| | | yl)pyridin-2- | 0.25H), 6.96-6.84 (m, 3H), 6.92 (s, 0.5H), 6.84 |
| | | yl)methyl-N-(3- | (s, 0.25H), 5.09 (s, 2H), 3.80-3.61 (m, 4H), |
| | | fluorophenyl)-1- | 3.06-3.00 (m, 4H), 2.40 (s, 1H); LRMS (ES) |
| | | iminothiomorpholin-4- | m/z 481.9 (M⁺ + 1) |
| | | carboxamide 1-oxide | |

### Example 18: Synthesis of Compound 18, N-(4-(5-(difluoromethyl)-1,3,4-oxadiazol-2-yl)-2-fluorobenzyl)-1-imino-N-phenylthiomorpholin-4-sulfonamide 1-oxide

### [Step 1] Synthesis of 1-((1H-imidazol-1-yl)sulfonyl)-3-methyl-1H-imidazol-3-ium trifluoromethanesulfonate

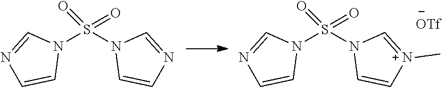

1,1′-sulfonylbis(1H-imidazole) (100.00%, 3.000 g, 15.136 mmol) and methyl trifluoromethanesulfonate (100.00% solution, 1.4903 mL, 13.623 mmol) were dissolved in dichloromethane (10 mL) at room temperature, after which the resulting solution was stirred at the same temperature for three days. Solvent was removed from the reaction mixture under reduced pressure, after which hexane (20 mL) and ethyl acetate (10 mL) were added into the resulting concentrate and stirred to filter out a precipitated solid, which was then washed with hexane and dried to obtain the title compound (4.000 g, 72.94%) in a white solid form.

### [Step 2] Synthesis of 4-((1H-imidazol-1-yl)sulfonyl)thiomorpholine

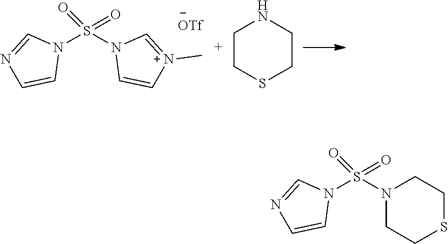

1-((1H-imidazol-1-yl)sulfonyl)-3-methyl-1H-imidazol-3-ium trifluoromethanesulfonate (100.00%, 4.000 g, 11.040 mmol) prepared in step 1 and thiomorpholine (100.00%, 1.100 equiv., 12.140 mmol) were dissolved in acetonitrile (50 mL) at room temperature, after which the resulting solution was stirred overnight at the same temperature. Solvent was removed from the reaction mixture under reduced pressure, after which saturated aqueous solution of sodium hydrogen carbonate was poured into the resulting concentrate and extracted with dichloromethane. An organic layer was washed with saturated aqueous solution of sodium chloride, dehydrated with anhydrous magnesium sulfate, filtered, and concentrated under reduced pressure. The resulting concentrate was purified via column chromatography (SiO₂, 40 g cartridge; hexane/ethyl acetate=0 to 30%) and concentrated to obtain the title compound (0.500 g, 19.41%) in a white solid form.

### [Step 3] Synthesis of 3-methyl-1-(thiomorpholinosulfonyl)-1H-imidazol-3-ium trifluoromethanesulfonate

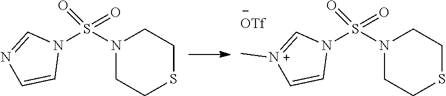

4-((1H-imidazol-1-yl)sulfonyl)thiomorpholine (100.00%, 2.260 g, 9.687 mmol) prepared in step 2 and methyl triflate (100.00%, 1.100 equiv., 10.660 mmol) were dissolved in dichloromethane (50 mL) at room temperature, after which the resulting solution was stirred overnight at the same temperature. Solvent was removed from the reaction mixture under reduced pressure, after which dichloromethane (20 mL) and methanol (10 mL) were added into the resulting concentrate and stirred to filter out a precipitated solid, which was then washed with hexane and dried to obtain the title compound (1.500 g, 38.96%) in a white solid form.

### [Step 4] Synthesis of N-phenylthiomorpholin-4-sulfonamide

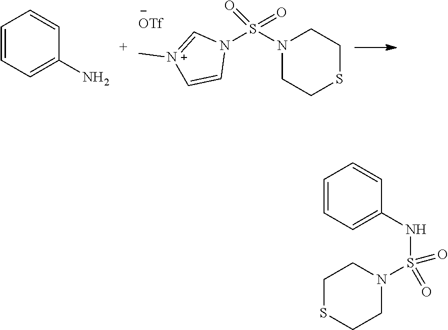

3-methyl-1-(thiomorpholinosulfonyl)-1H-imidazol-3-ium trifluoromethanesulfonate (100.00%, 1.500 g, 3.775 mmol) prepared in step 3 and aniline (100.00%, 1.000 equiv., 3.775 mmol) were dissolved in acetonitrile (30 mL) at 80° C., after which the resulting solution was stirred overnight at the same temperature and then a reaction was finished by lowering the temperature to room temperature. Solvent was removed from the reaction mixture under reduced pressure, after which saturated aqueous solution of sodium hydrogen carbonate was poured into the resulting concentrate and extracted with dichloromethane. An organic layer was washed with saturated aqueous solution of sodium chloride, dehydrated with anhydrous magnesium sulfate, filtered, and concentrated under reduced pressure. The resulting concentrate was purified via column chromatography (SiO₂, 40 g cartridge; hexane/ethyl acetate=0 to 50%) and concentrated to obtain the title compound (0.800 g, 82.02%) in a white solid form.

### [Step 5] Synthesis of N-(4-(5-(difluoromethyl)-1,3,4-oxadiazol-2-yl)-2-fluorobenzyl)-N-phenylthiomorpholin-4-sulfonamide

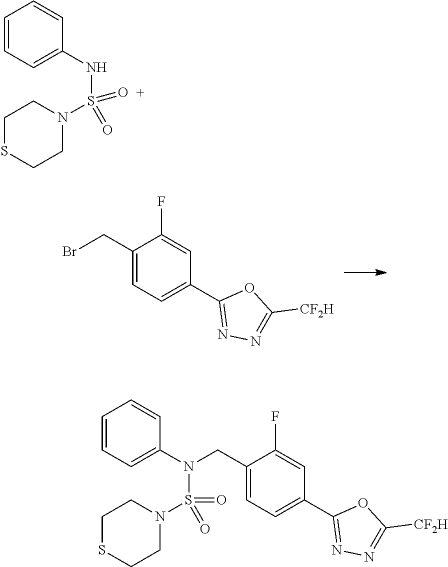 [SMILES: O=S(=O)(Nc1ccccc1)N1CCSCC1 ; Fc1cc(-c2nnc(C(F)F)o2)ccc1CBr ; O=S(=O)(N1CCSCC1)N(Cc1ccc(-c2nnc(C(F)F)o2)cc1F)c1ccccc1]

The title compound (0.204 g, 47.93%) was obtained in a colorless oil form according to substantially the same reaction as described in step 2 of example 1 except for using N-phenylthiomorpholin-4-sulfonamide prepared in step 4 instead of N-phenylthiomorpholin-4-carboxamide.

### [Step 6] Synthesis of Compound 18

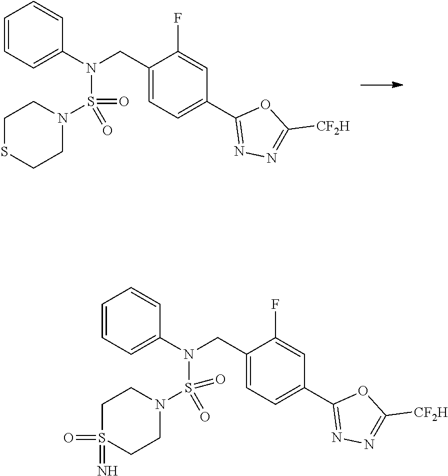 [SMILES: O=S(=O)(N1CCSCC1)N(Cc1ccc(-c2nnc(C(F)F)o2)cc1F)c1ccccc1 ; N=S1(=O)CCN(S(=O)(=O)N(Cc2ccc(-c3nnc(C(F)F)o3)cc2F)c2ccccc2)CC1]

The title compound (0.150 g, 69.11%) was obtained in a colorless oil form according to substantially the same reaction as described in step 3 of example 1 except for using N-(4-(5-(difluoromethyl)-1,3,4-oxadiazol-2-yl)-2-fluorobenzyl)-N-phenylthiomorpholin-4-sulfonamide prepared in step 5 instead of N-(4-(5-(difluoromethyl)-1,3,4-oxadiazol-2-yl)-2-fluorobenzyl)-N-phenylthiomorpholin-4-carboxamide.

¹H NMR (400 MHz, CDCl₃) δ 7.81 (dd, J=8.0, 1.5 Hz, 1H), 7.73 (dd, J=9.9, 1.5 Hz, 1H), 7.47 (t, J=7.6 Hz, 1H), 7.36-7.26 (m, 5H), 7.04 (s, 0.25H), 6.91 (s, 0.5H), 6.78 (s, 0.25H), 4.92 (s, 2H), 3.76-3.60 (m, 4H), 3.10-3.04 (m, 4H), 2.60 (brs, 1H); LRMS (ES) m/z 516.9 (M⁺+1)

### Example 19: Synthesis of Compound 19, N-((5-(5-(difluoromethyl)-1,3,4-oxadiazol-2-yl)pyridin-2-yl)methyl)-1-imino-N-phenylthiomorpholin-4-sulfonamide 1-oxide

### [Step 1] Synthesis of N-((5-(5-(difluoromethyl)-1,3,4-oxadiazol-2-yl)pyridin-2-yl)methyl)-N-phenylthiomorpholin-4-sulfonamide

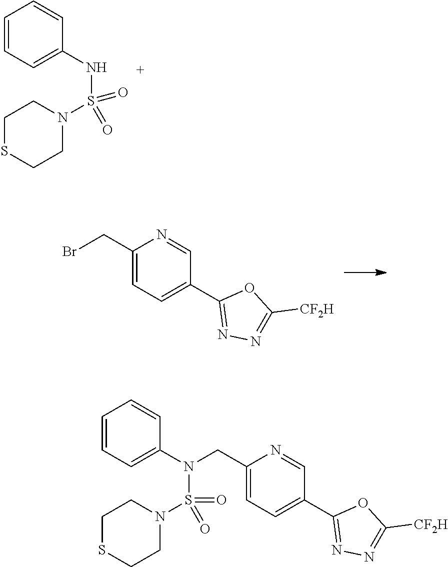 [SMILES: O=S(=O)(Nc1ccccc1)N1CCSCC1 ; FC(F)c1nnc(-c2ccc(CBr)nc2)o1 ; O=S(=O)(N1CCSCC1)N(Cc1ccc(-c2nnc(C(F)F)o2)cn1)c1ccccc1]

The title compound (0.300 g, 78.96%) was obtained in a colorless oil form according to substantially the same reaction as described in step 5 of example 18 except for using 2-(6-(bromomethyl)pyridin-3-yl)-5-(difluoromethyl)-1,3,4-oxadiazole instead of 2-(4-(bromomethyl)-3-fluorophenyl)-5-(difluoromethyl)-1,3,4-oxadiazole in step 5 of example 18.

### [Step 2] Synthesis of Compound 19

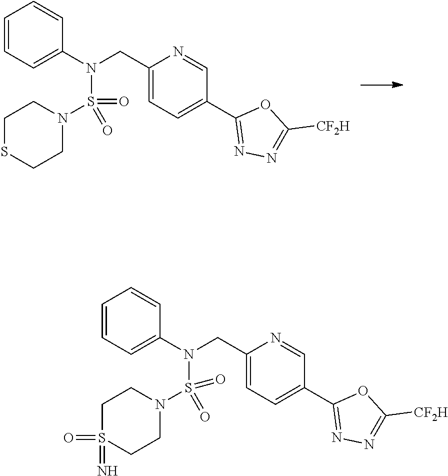 [SMILES: O=S(=O)(N1CCSCC1)N(Cc1ccc(-c2nnc(C(F)F)o2)cn1)c1ccccc1 ; N=S1(=O)CCN(S(=O)(=O)N(Cc2ccc(-c3nnc(C(F)F)o3)cn2)c2ccccc2)CC1]

The title compound (0.250 g, 78.15%) was obtained in a colorless oil form according to substantially the same reaction as described in step 3 of example 1 except for using N-((5-(5-(difluoromethyl)-1,3,4-oxadiazol-2-yl)pyridin-2-yl)methyl)-N-phenylthiomorpholin-4-sulfonamide prepared in step 1.

¹H NMR (400 MHz, CDCl₃) δ 9.25-9.24 (m, 1H), 8.36 (dd, J=8.2, 2.2 Hz, 1H), 7.57 (d, J=8.2 Hz, 1H), 7.42-7.28 (m, 5H), 7.08 (s, 0.25H), 6.95 (s, 0.5H), 6.82 (s, 0.25H), 5.06 (s, 2H), 3.84-3.68 (m, 4H), 3.11-3.03 (m, 4H), 2.50 (brs, 1H); LRMS (ES) m/z 499.9 (M⁺+1)

### Examples 20 to 22: Synthesis of Compounds 20 to 22

Compounds 20 to 22 according to examples 20 to 22 were prepared in accordance with the same reactions as described in steps 1, 2 and 3 of example 1, respectively, except for using the reactants of Table 6 below instead of isocyanatobenzene in step 1 of example 1. Table 6 shows the properties and yields of the products prepared in each of steps 1 to 3 in examples 20 to 22.

TABLE 6

| | | Yield/property | Yield/property | Yield/property |
|---|---|---|---|---|
| | | of product | of product | of product |
| | | obtained from | obtained from | obtained from |
| Classification | Reactant in step 1 | step 1 | step 2 | step 3 |
| Example 20 | 1-bromo-4-isocyanato- | 1.480 g, 97.30% | 1.170 g, 66.82% | 0.068 g, 64.22% |
| | benzene | White solid | White solid | White solid |
| Example 21 | (4-isocyanato- | 0.950 g, 92.23% | 0.478 g, 40.14% | 0.039 g, 36.91% |
| | phenyl)(trifluoro- | Light yellow oil | Colorless oil | White solid |
| | methyl)sulfan | | | |
| Example 22 | 5-isocyanato-2,3- | 0.187 g, 57.01% | 0.813 g, 54.90% | 0.060 g, 56.43% |
| | dihydrobenzofuran | White solid | White solid | White solid |

The names of compounds 20 to 22 prepared in each of examples 20 to 22, and the NMR and LC-Mass analysis results thereof are shown in table 7 below.

TABLE 7

| | Compound | | |
|---|---|---|---|
| Classification | No. | Compound name | NMR/LRMS |
| Example 20 | 20 | N-(4-bromophenyl)-N-(4- | ¹H NMR (400 MHz, CDCl₃) δ 7.89 (d, J = |
| | | (5-(difluoromethyl)-1,3,4- | 8.0 Hz, 1H), 7.77 (d, J = 8.6 Hz, 1H), 7.68 |
| | | oxadiazol-2-yl)-2- | (t, J = 7.7 Hz, 1H), 7.49 (d, J = 8.7 Hz, 2H), |
| | | fluorobenzyl)-1- | 7.06-6.80 (m, 3H), 5.32 (s, 2H), 3.79-3.73 |
| | | iminothiomorpholin-4- | (m, 2H), 3.69-3.63 (m, 2H), 2.91-2.88 (m, |
| | | carboxamide 1-oxide | 4H); LRMS (ES) m/z 558.8 (M⁺ + 1) |
| Example 21 | 21 | N-(4-(5-(difluoromethyl)- | ¹H NMR (400 MHz, CDCl₃) δ 7.90 (d, J = |
| | | 1,3,4-oxadiazol-2-y1)-2- | 8.0 Hz, 1H), 7.79 (d, J = 8.6 Hz, 1H), 7.69- |
| | | fluorobenzyl)-1-imino-N- | 7.65 (m, 3H), 7.20 (d, J = 8.7 Hz, 2H), 6.93 |
| | | (4-((trifluoro- | (t, J = 51.7 Hz, 1H), 4.99 (s, 2H), 3.79-3.74 |
| | | methyl)thio)phenyl)thio- | (m, 2H), 3.69-3.64 (m, 2H), 2.92-2.89 (m, |
| | | morpholin-4- | 4H); LRMS (ES) m/z 580.8 (M⁺ + 1). |
| | | carboxamide 1-oxide | |
| Example 22 | 22 | N-(4-(5-(difluoromethyl)- | ¹H NMR (400 MHz, CDCl₃) δ 7.89 (d, J = |
| | | 1,3,4-oxadiazol-2-y1)-2- | 8.0 Hz, 1H), 7.76 (d, J = 8.5 Hz, 1H), 7.68 |
| | | fluorobenzyl)-N-(2,3- | (t, J = 7.6 Hz, 1H), 7.06-6.80 (m, 3H), 6.73 |
| | | dihydrobenzofuran-5-yl)- | (d, J = 8.4 Hz, 1H), 4.87 (s, 2H), 4.62 (t, J = |
| | | 1-iminothiomorpholin-4- | 8.7 Hz, 2H), 3.81-3.76 (m, 2H), 3.70-3.64 |
| | | carboxamide 1-oxide | (m, 2H), 3.21 (t, J = 8.7 Hz, 2H), 2.85 (t, J = |
| | | | 5.2 Hz, 4H); LRMS (ES) m/z 522.9 (M⁺ + 1). |

### Example 23: Synthesis of Compound 23, N-(4-(5-(difluoromethyl)-1,3,4-oxadiazol-2-yl)-2-fluorobenzyl)-N-(4-(furan-2-yl)phenyl)-1-iminothiomorpholin-4-carboxamide 1-oxide

### [Step 1] Synthesis of N-(4-(5-(difluoromethyl)-1,3,4-oxadiazol-2-yl)-2-fluorobenzyl)-N-(4-(furan-2-yl)phenyl)thiomorpholin-4-carboxamide

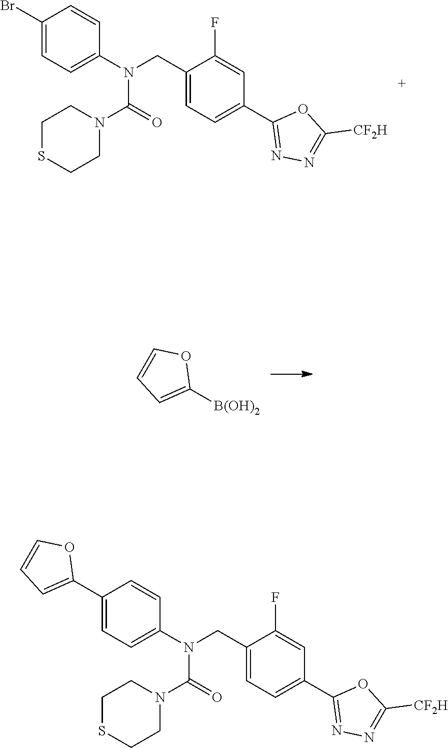 [SMILES: O=C(N1CCSCC1)N(Cc1ccc(-c2nnc(C(F)F)o2)cc1F)c1ccc(Br)cc1 ; OB(O)c1ccco1 ; O=C(N1CCSCC1)N(Cc1ccc(-c2nnc(C(F)F)o2)cc1F)c1ccc(-c2ccco2)cc1]

N-(4-bromophenyl)-N-((4-(5-(difluoromethyl)-1,3,4-oxadiazol-2-yl)-2-fluoro-benzyl)thiomorpholin-4-carboxamide (100.00%, 0.200 g, 0.379 mmol), 2-furylboronic acid (100.00%, 0.051 g, 0.456 mmol), (1,1′-bis(di-tert-butylphosphino)ferrocene)dichloropalladium(II) (Pd(dtbpf)Cl₂, 100.00%, 0.012 g, 0.018 mmol) and cesium carbonate (100.00%, 0.247 g, 0.758 mmol) were mixed in 1,4-dioxane (2 mL)/water (0.5 mL) at room temperature, after which the resulting mixture was irradiated with microwave, then heated at 100° C. for 30 minutes, and then a reaction was finished by lowering the temperature to room temperature. Solvent was removed from the reaction mixture under reduced pressure, after which saturated aqueous solution of sodium hydrogen carbonate was poured into the resulting concentrate, and extraction was performed with dichloromethane, then filtered via a plastic filter to remove a solid residue and an aqueous solution layer therefrom, and then concentrated under reduced pressure. The resulting concentrate was purified via column chromatography (SiO₂, 4 g cartridge; ethyl acetate/hexane=0 to 20%) and concentrated to obtain the title compound (0.110 g, 56.38%) in a light yellow solid form.

### [Step 2] Synthesis of Compound 23

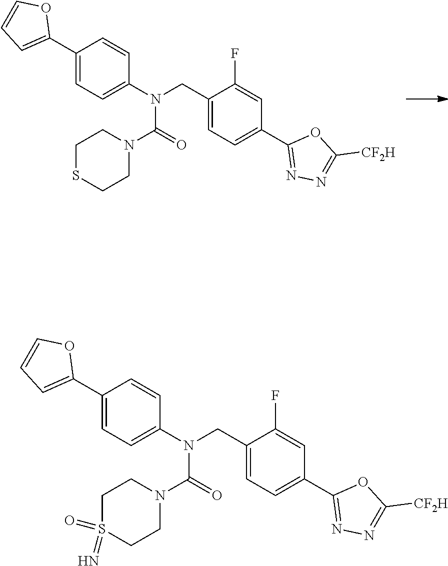 [SMILES: O=C(N1CCSCC1)N(Cc1ccc(-c2nnc(C(F)F)o2)cc1F)c1ccc(-c2ccco2)cc1 ; N=S1(=O)CCN(C(=O)N(Cc2ccc(-c3nnc(C(F)F)o3)cc2F)c2ccc(-c3ccco3)cc2)CC1]

N-(4-(5-(difluoromethyl)-1,3,4-oxadiazol-2-yl)-2-fluorobenzyl)-N-(4-(furan-2-yl)phenyl)thiomorpholin-4-carboxamide (100.00%, 0.100 g, 0.194 mmol) prepared in step 1, iodobenzene diacetate (100.00%, 0.153 g, 0.475 mmol) and ammonium carbamate (100.00%, 0.030 g, 0.384 mmol) were dissolved in methanol (3 mL) at room temperature, after which the resulting solution was stirred at the same temperature. Solvent was removed from the resulting mixture under reduced pressure, after which the resulting concentrate was purified via column chromatography (SiO₂, 4 g cartridge; ethyl acetate=100%) and concentrated to obtain the title compound (0.038 g, 35.84%) in a yellow solid form.

¹H NMR (400 MHz, CDCl₃) δ 7.89 (dd, J=8.0, 1.6 Hz, 1H), 7.76 (d, J=8.5 Hz, 1H), 7.72-7.65 (m, 3H), 7.49 (d, J=1.2 Hz, 1H), 7.15 (d, J=8.7 Hz, 2H), 6.92 (t, J=51.7 Hz, 1H), 6.67-6.66 (m, 1H), 6.50-6.49 (m, 1H), 4.97 (s, 2H), 3.82-3.77 (m, 2H), 3.72-3.67 (m, 2H), 2.88 (t, J=5.2 Hz, 4H); LRMS (ES) m/z 546.8 (M⁺+1).

### Examples 24 to 28: Synthesis of Compounds 24 to 28

Compounds 24 to 28 according to examples 24 to 28 were prepared in accordance with the same reactions as described in steps 1, 2 and 3 of example 1, respectively, except for using the reactants of Table 8 below instead of isocyanatobenzene in step 1 of example 1. Table 8 shows the properties and yields of the products prepared in each of steps 1 to 3 in examples 24 to 28.

TABLE 8

| | | Yield/property of | Yield/property of | Yield/property of |
|---|---|---|---|---|
| | | product | product | product |
| | | obtained from | obtained from | obtained from |
| Classification | Reactant in step 1 | step 1 | step 2 | step 3 |
| Example 24 | Isocyanatomethylbenzene | 0.753 g, 84.86% | 0.040 g, 10.22% | 0.017 g, 39.83% |
| | | White solid | White solid | White solid |
| Example 25 | 1-isocyanato-2-methoxy- | 0.696 g, 82.29% | 0.134 g, 35.33% | 0.063 g, 44.15% |
| | benzene | White solid | White solid | Light yellow solid |
| Example 26 | 3-isocyanatothiophene | 0.395 g, 43.31% | 0.167 g, 41.94% | 0.079 g, 44.28% |
| | | White solid | White solid | Light yellow oil |
| Example 27 | 1-isocyanato-4-methoxy- | 0.700 g, 82.76% | 0.195 g, 51.41% | 0.100 g, 48.16% |
| | benzene | White solid | Colorless oil | Colorless oil |
| Example 28 | 5-isocyanato-2,3- | 0.700 g, 84.93% | 0.190 g, 68.04% | 0.100 g, 47.49% |
| | dihydro-1h-indene | White solid | Colorless oil | Colorless oil |

The names of compounds 24 to 28 prepared in each of examples 24 to 28, and the NMR and LC-Mass analysis results thereof are shown in table 9 below.

TABLE 9

| | Compound | | |
|---|---|---|---|
| Classification | No. | Compound name | NMR/LRMS |
| Example 24 | 24 | N-benzyl-N-(4-(5- | ¹H NMR (400 MHz, CDCl₃) δ 7.91 (dd, J = |
| | | (difluoromethyl)-1,3,4- | 7.9, 1.6 Hz, 1H), 7.83 (dd, J = 10.1, 1.6 |
| | | oxadiazol-2-y1)-2- | Hz, 1H), 7.42-7.35 (m, 4H), 7.18 (d, J = |
| | | fluorobenzyl)-1- | 8.2 Hz, 2H), 6.95 (t, J = 51.7 Hz, 1H), 4.46 |
| | | iminothiomorpholin-4- | (s, 2H), 4.42 (s, 2H), 3.85-3.82 (m, 2H), |
| | | carboxamide 1-oxide | 3.81-3.78 (m, 2H), 3.14-3.13 (m, 4H); |
| | | | LRMS (ES) m/z 494.9 (M⁺ + 1). |
| Example 25 | 25 | N-(4-(5-(difluoromethyl)- | ¹H NMR (400 MHz, CDCl₃) δ 7.87 (dd, J = |
| | | 1,3,4-oxadiazol-2-y1)-2- | 8.1, 1.5 Hz, 1H), 7.81 (t, J = 7.5 Hz, 1H), |
| | | fluorobenzyl)-1-imino-N- | 7.69 (dd, J = 9.9, 1.4 Hz, 1H), 7.29-7.25 |
| | | (2-methoxy- | (m, 1H), 7.05-6.79 (m, 4H), 4.85 (s, 2H), |
| | | phenyl)thiomorpholin- | 3.85 (s, 3H), 3.79-3.73 (m, 2H), 3.65- |
| | | 4-carboxamide 1-oxide | 3.59 (m, 2H), 2.72-2.67 (m, 4H); LRMS |
| | | | (ES) m/z 510.8 (M⁺ + 1). |
| Example 26 | 26 | N-(4-(5-(difluoromethyl)- | ¹H NMR (400 MHz, CDCl₃) δ 7.88 (d, J = |
| | | 1,3,4-oxadiazol-2-yl)-2- | 8.0 Hz, 1H), 7.79 (d, J = 8.8 Hz, 1H), 7.63 |
| | | fluorobenzyl)-1-imino-N- | (t, J = 7.6 Hz, 1H), 7.34-7.32 (m, 1H), |
| | | (thiophen-3- | 6.93-6.80 (m, 3H), 4.92 (s, 2H), 3.83- |
| | | yl)thiomorpholin-4- | 3.77 (m, 2H), 3.72-3.66 (m, 2H), 2.92- |
| | | carboxamide 1-oxide | 2.90 (m, 4H); LRMS (ES) m/z 486.8 (M⁺ + 1). |
| Example 27 | 27 | N-(4-(5-(difluoromethyl)- | ¹H NMR (400 MHz, CDCl₃) δ 7.87 (dd, J = |
| | | 1,3,4-oxadiazol-2-y1)-2- | 8.0, 1.6 Hz, 1H), 7.74 (dd, J = 10.0, 1.6 |
| | | fluorobenzyl)-1-imino-N- | Hz, 1H), 7.68 (t, J = 7.6 Hz, 1H), 7.05- |
| | | (4-methoxy- | 7.00 (m, 2H), 7.01 (s, 0.25H), 6.92 (s, |
| | | phenyl)thiomorpholin- | 0.5H), 6.88-6.84 (m, 2H), 6.79 (s, 0.25H), |
| | | 4-carboxamide 1-oxide | 4.88 (s, 2H), 3.80-3.50 (m, 4H), 3.80 (s, |
| | | | 3H), 2.83-2.80 (m, 4H), 2.50 (brs, 1H); |
| | | | LRMS (ES) m/z 510.9 (M⁺ + 1) |
| Example 28 | 28 | N-(4-(5-(difluoromethyl)- | ¹H NMR (400 MHz, CDCl₃) δ 7.88 (dd, J = |
| | | 1,3,4-oxadiazol-2-y1)-2- | 8.0, 1.6 Hz, 1H), 7.77 (dd, J = 10.0, 1.6 |
| | | fluorobenzyl)-N-(2,3- | Hz, 1H), 7.67 (t, J = 7.6 Hz, 1H), 7.18 (d, |
| | | dihydro-1H-inden-5-yl)-1- | J = 8.0 Hz, 1H), 7.05 (s, 0.25H), 7.00 (d, J = |
| | | iminothiomorpholin-4- | 1.6 Hz, 1H), 6.92 (s, 0.5H), 6.87 (dd, J = |
| | | carboxamide 1-oxide | 8.0, 2.0 Hz, 1H), 6.80 (s, 0.25H), 4.91 (s, |
| | | | 2H), 3.82-3.62 (m, 4H), 2.91-2.87 (m, |
| | | | 4H), 2.84-2.82 (m, 4H), 2.50 (s, 1H), 2.14- |
| | | | 2.10 (m, 2H); LRMS (ES) m/z 520.9 (M⁺ + 1) |

### Example 29: Synthesis of Compound 29, Diethyl (4-((4-(5-(difluoromethyl)-1,3,4-oxadiazol-2-yl)-2-fluorobenzyl)(3,4-difluorophenyl)carbamoyl)-1-oxidothiomorpholin-1-ylidene)phosphoramidate

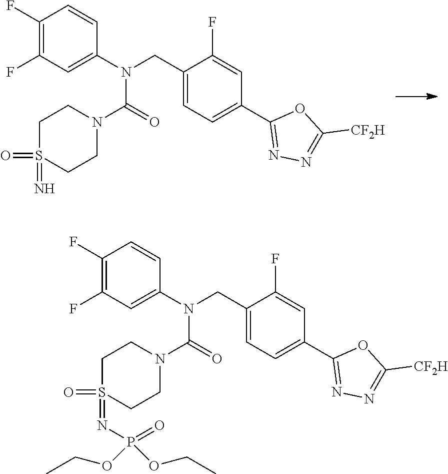 [SMILES: N=S1(=O)CCN(C(=O)N(Cc2ccc(-c3nnc(C(F)F)o3)cc2F)c2ccc(F)c(F)c2)CC1 ; CCOP(=O)(N=S1(=O)CCN(C(=O)N(Cc2ccc(-c3nnc(C(F)F)o3)cc2F)c2ccc(F)c(F)c2)CC1)OCC]

N-(4-(5-(difluoromethyl)-1,3,4-oxadiazol-2-yl)-2-fluorobenzyl)-N-(3,4-difluorophenyl)-1-iminothiomorpholin-4-carboxamide 1-oxide (100.00%, 0.080 g, 0.155 mmol) obtained according to substantially the same reactions as described in example 6, 1-ethoxyphosphoryloxyethane (100.00%, 1.000 equiv., 0.155 mmol), iodine (100.00%, 0.100 equiv., 0.016 mmol) and hydrogen peroxide (100.00%, 1.000 equiv., 0.155 mmol) were dissolved in dichloromethane (10 mL) at room temperature, after which the resulting solution was stirred overnight at the same temperature. Saturated aqueous solution of sodium hydrogen carbonate was poured into the reaction mixture and extracted with dichloromethane. An organic layer was washed with saturated aqueous solution of sodium chloride, dehydrated with anhydrous magnesium sulfate, filtered, and concentrated under reduced pressure. The resulting concentrate was purified via column chromatography (SiO₂, 12 g cartridge; dichloromethane/methanol=0 to 10%) and concentrated to obtain the title compound (0.040 g, 39.56%) in a colorless oil form.

¹H NMR (400 MHz, CDCl₃) δ 7.90 (dd, J=8.0, 1.6 Hz, 1H), 7.78 (dd, J=10.4, 1.6 Hz, 1H), 7.69-7.65 (m, 1H), 7.21-7.14 (m, 1H), 7.06 (s, 0.25H), 7.03-6.97 (m, 1H), 6.93 (s, 0.5H), 6.89-6.86 (m, 1H), 6.80 (s, 0.25H), 4.90 (s, 2H), 4.29-4.25 (m, 2H), 4.06-4.02 (m, 4H), 3.91-3.87 (m, 2H), 3.60-3.54 (m, 2H), 3.40-3.35 (m, 2H), 3.05-3.00 (m, 2H), 1.33-1.30 (m, 6H); LRMS (ES) m/z 652.9 (M⁺+1)

### Example 30: Synthesis of Compound 30, 1-(acetylimino)-N-((5-(5-(difluoromethyl)-1,3,4-oxadiazol-2-yl)pyridin-2-yl)methyl)-N-(3,4-difluorophenyl)thiomorpholin-4-carboxamide 1-oxide

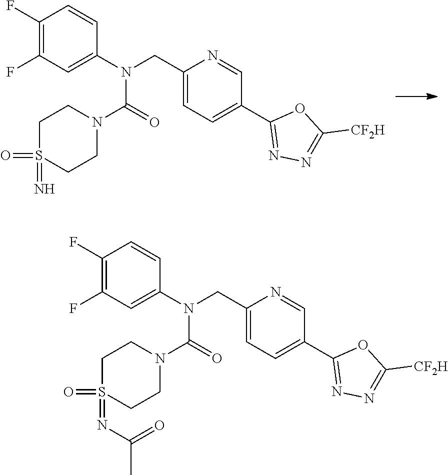 [SMILES: N=S1(=O)CCN(C(=O)N(Cc2ccc(-c3nnc(C(F)F)o3)cn2)c2ccc(F)c(F)c2)CC1 ; CC(=O)N=S1(=O)CCN(C(=O)N(Cc2ccc(-c3nnc(C(F)F)o3)cn2)c2ccc(F)c(F)c2)CC1]

N-((5-(5-(difluoromethyl)-1,3,4-oxadiazol-2-yl)pyridin-2-yl)methyl)-N-(3,4-difluorophenyl)-1-iminothiomorpholin-4-carboxamide 1-oxide (100.00%, 0.034 g, 0.068 mmol) obtained according to substantially the same reactions as described in example 7, acetyl chloride (100.00%, 2.000 equiv., 0.136 mmol) and triethylamine (100.00%, 1.500 equiv., 0.102 mmol) were dissolved in dichloromethane (5 mL) at room temperature, after which the resulting solution was stirred overnight at the same temperature. Saturated aqueous solution of sodium hydrogen carbonate was poured into the reaction mixture and extracted with dichloromethane. An organic layer was washed with saturated aqueous solution of sodium chloride, dehydrated with anhydrous magnesium sulfate, filtered, and concentrated under reduced pressure. The resulting concentrate was purified via column chromatography (SiO₂, 12 g cartridge; dichloromethane/methanol=0 to 10%) and concentrated to obtain the title compound (0.015 g, 40.68%) in a colorless oil form.

¹H NMR (400 MHz, CDCl₃) δ 9.27 (dd, J=2.0, 0.4 Hz, 1H), 8.43 (dd, J=8.2, 2.2 Hz, 1H), 7.54 (dd, J=8.4, 0.4 Hz, 1H), 7.22-7.05 (m, 2H), 7.09 (s, 0.25H), 7.00-6.94 (m, 1H), 6.96 (s, 0.5H), 6.83 (s, 0.25H), 5.08 (s, 2H), 3.90-3.80 (m, 2H), 3.60-3.50 (m, 4H), 3.30-3.20 (m, 2H), 2.09 (s, 3H); LRMS (ES) m/z 541.9 (M⁺+1)

### Examples 31 to 34: Synthesis of Compounds 31 to 34

Compounds 31 to 34 according to examples 31 to 34 were prepared in accordance with the same reactions as described in steps 1, 2 and 3 of example 1, respectively, except for using the reactants of Table 10 below instead of isocyanatobenzene in step 1 of example 1. Table 10 shows the properties and yields of the products prepared in each of steps 1 to 3 in examples 31 to 34.

TABLE 10

| | | Yield/property of | Yield/property of | Yield/property of |
|---|---|---|---|---|
| | | product | product | product |
| | | obtained from | obtained from | obtained from |
| Classification | Reactant in step 1 | step 1 | step 2 | step 3 |
| Example 31 | 1-isocyanato-3- | 0.436 g, 56.21% | 0.092 g, 51.71% | 0.065 g, 64.55% |
| | (trifluoromethyl)- | White solid | Light yellow solid | White solid |
| | benzene | | | |
| Example 32 | 1-isocyanato-3- | 0.738 g, 83.17% | 0.125 g, 63.86% | 0.110 g, 82.47% |
| | methyl-benzene | White solid | Colorless oil | White solid |
| Example 33 | 1,4-difluoro-2- | 0.583 g, 70.01% | 0.101 g, 53.86% | 0.090 g, 83.73% |
| | isocyanato-benzene | White solid | Colorless oil | White solid |
| Example 34 | 1-(3- | 0.633 g, 81.14% | 0.030 g, 16.79% | 0.005 g, 15.71% |
| | isocyanatophenyl)-1H- | White solid | White solid | White solid |
| | pyrrole | | | |

The names of compounds 31 to 34 prepared in each of examples 31 to 34, and the NMR and LC-Mass analysis results thereof are shown in table 11 below.

TABLE 11

| | Compound | | |
|---|---|---|---|
| Classification | No. | Compound name | NMR/LRMS |
| Example 31 | 31 | N-(4-(5-(difluoromethyl)- | ¹H NMR (400 MHz, CDCl₃) δ 7.91 (d, J = |
| | | 1,3,4-oxadiazol-2-y1)-2- | 8.0 Hz, 1H), 7.79 (d, J = 10.2 Hz, 1H), 7.69 |
| | | fluorobenzyl)-1-imino-N- | (t, J = 7.6 Hz, 1H), 7.54-7.42 (m, 2H), |
| | | (3-(trifluoro- | 7.42 (s, 1H), 7.34-7.06 (m, 1H), 6.93 (t, J = |
| | | methyl)phenyl)thio- | 51.7 Hz, 1H), 4.98 (s, 2H), 3.79-3.73 |
| | | morpholin-4-carboxamide | (m, 2H), 3.68-3.62 (m, 2H), 2.89 (t, J = |
| | | 1-oxide | 5.2 Hz, 4H); LRMS (ES) m/z 548.7 (M⁺ + 1). |
| Example 32 | 32 | N-(4-(5-(difluoromethyl)- | ¹H NMR (400 MHz, CDCl₃) δ 7.88 (d, J = |
| | | 1,3,4-oxadiazol-2-y1)-2- | 8.0 Hz, 1H), 7.76 (d, J = 10.1 Hz, 1H), 7.67 |
| | | fluorobenzyl)-1-imino-N- | (t, J = 7.6 Hz, 1H), 7.24 (t, J = 7.8 Hz, 1H), |
| | | (m-tolyl)thiomorpholin-4- | 7.06-6.80 (m, 4H), 4.93 (s, 2H), 3.80- |
| | | carboxamide 1-oxide | 3.76 (m, 2H), 3.69-3.64 (m, 2H), 2.83 (t, |
| | | | J = 5.2 Hz, 4H), 2.35 (s, 3H); LRMS (ES) |
| | | | m/z 494.8 (M⁺ + 1). |
| Example 33 | 33 | N-(4-(5-(difluoromethyl)- | ¹H NMR (400 MHz, CDCl₃) δ 7.89 (dd, J = |
| | | 1,3,4-oxadiazol-2-y1)-2- | 8.0, 1.5 Hz, 1H), 7.79-7.72 (m, 2H), |
| | | fluorobenzyl)-N-(2,5- | 7.16-7.10 (m, 1H), 7.06-6.80 (m, 3H), |
| | | difluorophenyl)-1- | 4.90 (s, 2H), 3.78-3.74 (m, 2H), 3.69- |
| | | iminothiomorpholin-4- | 3.64 (m, 2H), 2.88 (t, J = 5.2 Hz, 4H); |
| | | carboxamide 1-oxide | LRMS (ES) m/z 516.8 (M⁺ + 1). |
| Example 34 | 34 | N-(3-(1H-pyrrol-1- | ¹H NMR (400 MHz, CDCl₃) δ 7.91 (dd, J = |
| | | yl)phenyl)-N-(4-(5- | 8.0, 1.5 Hz, 1H), 7.79 (dd, J = 10.1, 1.5 |
| | | (difluoromethyl)-1,3,4- | Hz, 1H), 7.72 (t, J = 7.6 Hz, 1H), 7.41 (t, J = |
| | | oxadiazol-2-y1)-2- | 8.0 Hz, 1H), 7.27-7.25 (m, 1H), 7.21- |
| | | fluorobenzyl)-1- | 7.20 (m, 1H), 7.06-6.39 (m, 4H), 6.39 (t, |
| | | iminothiomorpholin-4- | J = 2.2 Hz, 2H), 5.00 (s, 2H), 3.83-3.78 |
| | | carboxamide 1-oxide | (m, 2H), 3.73-3.66 (m, 2H), 2.91 (t, J = |
| | | | 5.2 Hz, 4H); LRMS (ES) m/z 545.8 (M⁺ + 1). |

### Example 35: Synthesis of Compound 35, N-(4-(5-(difluoromethyl)-1,3,4-oxadiazol-2-yl)-2-fluorobenzyl)-N-(3-fluorophenyl)-1-iminothiomorpholin-4-sulfonamide 1-oxide

### [Step 1] Synthesis of N-(3-fluorophenyl)thiomorpholin-4-sulfonamide

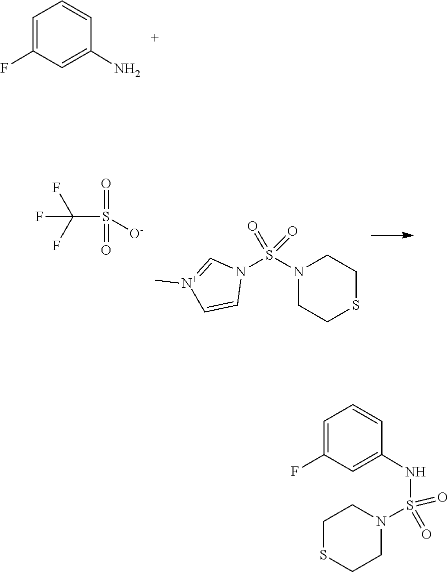 [SMILES: Nc1cccc(F)c1 ; O=S(=O)([O-])C(F)(F)F ; C[n+]1ccn(S(=O)(=O)N2CCSCC2)c1 ; O=S(=O)(Nc1cccc(F)c1)N1CCSCC1]

3-methyl-1-(thiomorpholinosulfonyl)-1H-imidazol-3-ium trifluoromethanesulfonate (100.00%, 0.580 g, 1.459 mmol) prepared in step 3 of example 18 and 3-fluoroaniline (100.00% solution, 0.14 mL, 1.462 mmol) were dissolved in acetonitrile (10 mL) at room temperature, after which the resulting solution was heated under reflux for 18 hours, and a reaction was finished by lowering the temperature to room temperature. Solvent was removed from the reaction mixture under reduced pressure, after which saturated aqueous solution of sodium hydrogen carbonate was poured into the resulting concentrate, and extraction was performed with dichloromethane, then filtered via a plastic filter to remove a solid residue and an aqueous solution layer therefrom, and then concentrated under reduced pressure. The resulting concentrate was purified via column chromatography (SiO₂, 12 g cartridge; ethyl acetate/hexane=5 to 20%) and concentrated to obtain the title compound (0.253 g, 62.72%) in a light yellow solid form.

### [Step 2] Synthesis of N-[4-[5-(difluoromethyl)-1,3,4-oxadiazol-2-yl]-2-fluorobenzyl]-N-(3-fluorophenyl)thiomorpholin-4-sulfonamide

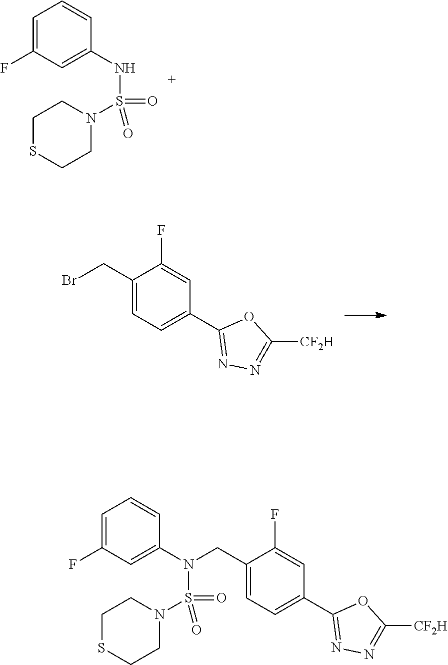 [SMILES: O=S(=O)(Nc1cccc(F)c1)N1CCSCC1 ; Fc1cc(-c2nnc(C(F)F)o2)ccc1CBr ; O=S(=O)(N1CCSCC1)N(Cc1ccc(-c2nnc(C(F)F)o2)cc1F)c1cccc(F)c1]

The title compound (0.153 g, 70.13%) was obtained in a white solid form according to substantially the same reaction as described in step 2 of example 1 except for using N-(3-fluorophenyl)thiomorpholin-4-sulfonamide prepared in step 1.

### [Step 3] Synthesis of Compound 35

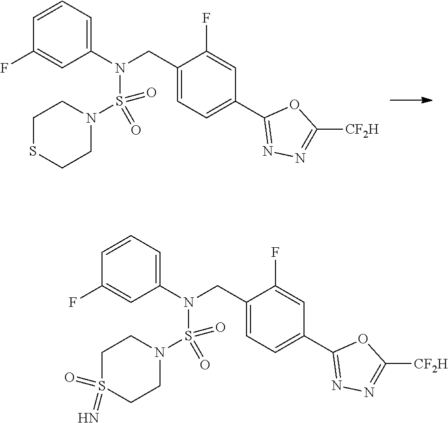 [SMILES: O=S(=O)(N1CCSCC1)N(Cc1ccc(-c2nnc(C(F)F)o2)cc1F)c1cccc(F)c1 ; N=S1(=O)CCN(S(=O)(=O)N(Cc2ccc(-c3nnc(C(F)F)o3)cc2F)c2cccc(F)c2)CC1]

The title compound (0.151 g, 92.96%) was obtained in a white solid form according to substantially the same reaction as described in step 3 of example 1 except for using N-[4-[5-(difluoromethyl)-1,3,4-oxadiazol-2-yl]-2-fluorobenzyl]-N-(3-fluorophenyl)thiomorpholin-4-sulfonamide prepared in step 2.

¹H NMR (400 MHz, CDCl₃) δ 7.86 (dd, J=8.0, 1.5 Hz, 1H), 7.79 (dd, J=9.9, 1.5 Hz, 1H), 7.49 (t, J=7.6 Hz, 1H), 7.37-7.31 (m, 1H), 7.09-6.80 (m, 4H), 0.94 (s, 2H), 3.82-3.76 (m, 2H), 3.73-3.67 (m, 2H), 3.19-3.09 (m, 4H); LRMS (ES) m/z 534.7 (M⁺+1).

### Example 36: Synthesis of Compound 36, N-((5-(5-(difluoromethyl)-1,3,4-oxadiazol-2-yl)pyridin-2-yl)methyl)-N-(3-fluorophenyl)-1-iminothiomorpholin-4-sulfonamide 1-oxide

### [Step 1] Synthesis of N-[[5-[5-(difluoromethyl)-1,3,4-oxadiazol-2-yl]pyridin-2-yl]methyl]-N-(3-fluorophenyl)thiomorpholin-4-sulfonamide

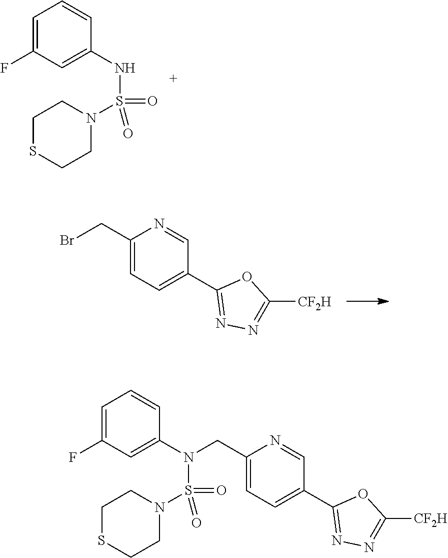 [SMILES: O=S(=O)(Nc1cccc(F)c1)N1CCSCC1 ; FC(F)c1nnc(-c2ccc(CBr)nc2)o1 ; O=S(=O)(N1CCSCC1)N(Cc1ccc(-c2nnc(C(F)F)o2)cn1)c1cccc(F)c1]

The title compound (0.208 g, 98.68%) was obtained in a colorless oil form according to substantially the same reaction as described in step 2 of example 35 except for using 2-(6-(bromomethyl)pyridin-3-yl)-5-(difluoromethyl)-1,3,4-oxadiazole instead of 2-(4-(bromomethyl)-3-fluorophenyl)-5-(difluoromethyl)-1,3,4-oxadiazole.

### [Step 2] Synthesis of Compound 36

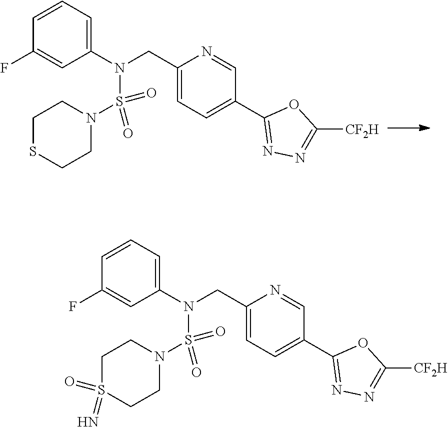 [SMILES: O=S(=O)(N1CCSCC1)N(Cc1ccc(-c2nnc(C(F)F)o2)cn1)c1cccc(F)c1 ; N=S1(=O)CCN(S(=O)(=O)N(Cc2ccc(-c3nnc(C(F)F)o3)cn2)c2cccc(F)c2)CC1]

The title compound (0.105 g, 47.5%) was obtained in a white solid form according to the same reaction as described in step 3 of example 1 except for using N-[[5-[5-(difluoromethyl)-1,3,4-oxadiazol-2-yl]pyridin-2-yl]methyl]-N-(3-fluorophenyl)thiomorpholin-4-sulfonamide obtained from step 1.

¹H NMR (400 MHz, CDCl₃) δ 9.29 (d, J=1.6 Hz, 1H), 8.39 (dd, J=8.2, 2.2 Hz, 1H), 7.55 (d, J=8.0 Hz, 1H), 7.38-7.32 (m, 1H), 7.23-7.20 (m, 2H), 7.08-6.83 (m, 2H), 5.06 (s, 2H), 3.86-3.82 (m, 2H), 3.78-3.74 (m, 2H); LRMS (ES) m/z 517.7 (M⁺+1).

### Example 37: Synthesis of Compound 37, N-(4-(5-(difluoromethyl)-1,3,4-oxadiazol-2-yl)-2-fluorobenzyl)-1-((methylsulfonyl)imino)-N-phenylthiomorpholin-4-carboxamide 1-oxide

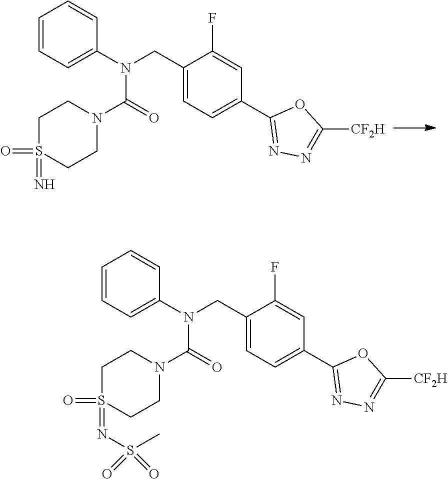 [SMILES: N=S1(=O)CCN(C(=O)N(Cc2ccc(-c3nnc(C(F)F)o3)cc2F)c2ccccc2)CC1 ; CS(=O)(=O)N=S1(=O)CCN(C(=O)N(Cc2ccc(-c3nnc(C(F)F)o3)cc2F)c2ccccc2)CC1]

N-(4-(5-(difluoromethyl)-1,3,4-oxadiazol-2-yl)-2-fluorobenzyl)-1-imino-N-phenylthiomorpholin-4-carboxamide 1-oxide (100.00%, 0.200 g, 0.417 mmol) obtained according to substantially the same reactions as described in example 1, methanesulfonyl chloride (100.00%, 1.500 equiv., 0.626 mmol) and triethylamine (100.00%, 2.000 equiv., 0.834 mmol) were dissolved in dichloromethane (10 mL) at room temperature, after which the resulting solution was stirred overnight at the same temperature. Saturated aqueous solution of sodium hydrogen carbonate was poured into the reaction mixture and extracted with dichloromethane. An organic layer was washed with saturated aqueous solution of sodium chloride, dehydrated with anhydrous magnesium sulfate, filtered, and concentrated under reduced pressure. The resulting concentrate was purified via column chromatography (SiO₂, 12 g cartridge; dichloromethane/methanol=0 to 10%) and concentrated to obtain the title compound (0.180 g, 77.39%) in a colorless oil form.

¹H NMR (400 MHz, CDCl₃) δ 7.87 (dd, J=8.0, 1.6 Hz, 1H), 7.75 (dd, J=10.0, 1.6 Hz, 1H), 7.65 (t, J=7.6 Hz, 1H), 7.38 (t, J=7.8 Hz, 2H), 7.28-7.24 (m, 1H), 7.13-7.11 (m, 2H), 7.05 (s, 0.25H), 6.92 (s, 0.5H), 6.79 (s, 0.25H), 4.94 (s, 2H), 4.00-3.96 (m, 2H), 3.57-3.44 (m, 4H), 3.08 (s, 3H), 3.06-3.01 (m, 2H); LRMS (ES) m/z 558.9 (M⁺+1)

### Example 38: Synthesis of Compound 38, Methyl (4-((4-(5-(difluoromethyl)-1,3,4-oxadiazol-2-yl)-2-fluorobenzyl)(phenyl)carbamoyl)-1-oxidothiomorpholin-1-ylidene)carbamate

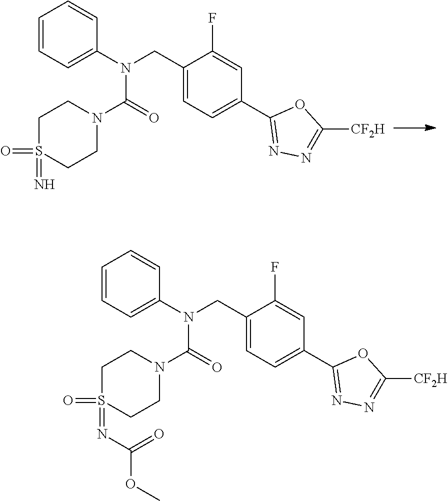 [SMILES: N=S1(=O)CCN(C(=O)N(Cc2ccc(-c3nnc(C(F)F)o3)cc2F)c2ccccc2)CC1 ; COC(=O)N=S1(=O)CCN(C(=O)N(Cc2ccc(-c3nnc(C(F)F)o3)cc2F)c2ccccc2)CC1]

N-(4-(5-(difluoromethyl)-1,3,4-oxadiazol-2-yl)-2-fluorobenzyl)-1-imino-N-phenylthiomorpholin-4-carboxamide 1-oxide (100.00%, 0.200 g, 0.417 mmol) obtained according to substantially the same reactions as described in example 1, methyl carbonochloridate (100.00%, 1.500 equiv., 0.626 mmol) and triethylamine (100.00%, 2.000 equiv., 0.834 mmol) were dissolved in dichloromethane (5 mL) at room temperature, after which the resulting solution was stirred overnight at the same temperature. Saturated aqueous solution of sodium hydrogen carbonate was poured into the reaction mixture and extracted with dichloromethane. An organic layer was washed with saturated aqueous solution of sodium chloride, dehydrated with anhydrous magnesium sulfate, filtered, and concentrated under reduced pressure. The resulting concentrate was purified via column chromatography (SiO₂, 12 g cartridge; dichloromethane/methanol=0 to 10%) and concentrated to obtain the title compound (0.150 g, 66.91%) in a colorless oil form.

¹H NMR (400 MHz, CDCl₃) δ 7.88 (dd, J=8.0, 1.6 Hz, 1H), 7.76 (dd, J=10.0, 1.6 Hz, 1H), 7.67 (t, J=7.6 Hz, 1H), 7.40-7.36 (m, 2H), 7.26-7.23 (m, 1H), 7.14-7.12 (m, 2H), 7.05 (s, 0.25H), 6.92 (s, 0.5H), 6.79 (s, 0.25H), 4.94 (s, 2H), 3.93-3.92 (m, 2H), 3.58 (s, 3H), 3.56-3.52 (m, 2H), 3.43-3.38 (m, 2H), 3.05-2.99 (m, 2H); LRMS (ES) m/z 538.9 (M⁺+1)

### Example 39: Synthesis of Compound 39, N-(4-(5-(difluoromethyl)-1,3,4-oxadiazol-2-yl)-2-fluorobenzyl)-1-(methylimino)-N-phenylthiomorpholin-4-carboxamide 1-oxide

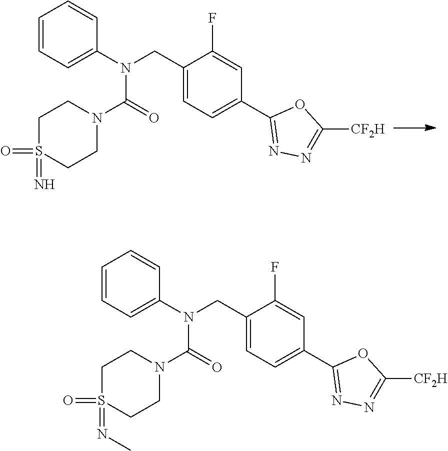 [SMILES: N=S1(=O)CCN(C(=O)N(Cc2ccc(-c3nnc(C(F)F)o3)cc2F)c2ccccc2)CC1 ; CN=S1(=O)CCN(C(=O)N(Cc2ccc(-c3nnc(C(F)F)o3)cc2F)c2ccccc2)CC1]

N-(4-(5-(difluoromethyl)-1,3,4-oxadiazol-2-yl)-2-fluorobenzyl)-1-imino-N-phenylthiomorpholin-4-carboxamide 1-oxide (100.00%, 0.200 g, 0.417 mmol) obtained according to substantially the same reactions as described in example 1, sodium carbonate (100.00%, 5.000 equiv., 2.086 mmol) and trimethyloxonium tetrafluoroborate (100.00%, 1.000 equiv., 0.417 mmol) were dissolved in dichloromethane (5 mL) at room temperature, after which the resulting solution was stirred overnight at the same temperature. Saturated aqueous solution of sodium hydrogen carbonate was poured into the reaction mixture and extracted with dichloromethane. An organic layer was washed with saturated aqueous solution of sodium chloride, dehydrated with anhydrous magnesium sulfate, filtered, and concentrated under reduced pressure. The resulting concentrate was purified via column chromatography (SiO₂, 12 g cartridge; dichloromethane/methanol=0 to 10%) and concentrated to obtain the title compound (0.015 g, 7.29%) in a colorless oil form.

¹H NMR (400 MHz, CDCl₃) δ 7.88 (dd, J=8.0, 1.2 Hz, 1H), 7.75 (dd, J=10.0, 1.6 Hz, 1H), 7.68 (t, J=7.6 Hz, 1H), 7.39-7.35 (m, 2H), 7.24-7.20 (m, 1H), 7.14-7.12 (m, 2H), 7.05 (s, 0.25H), 6.92 (s, 0.5H), 6.79 (s, 0.25H), 4.94 (s, 2H), 3.82-3.76 (m, 2H), 3.58-3.51 (m, 2H), 2.87-2.78 (m, 4H), 2.74 (s, 3H); LRMS (ES) m/z 494.9 (M⁺+1)

### Example 40: Synthesis of Compound 40, 1-(acetylimino)-N-(4-(5-(difluoromethyl)-1,3,4-oxadiazol-2-yl)-2-fluorobenzyl)-N-phenylthiomorpholin-4-carboxamide 1-oxide

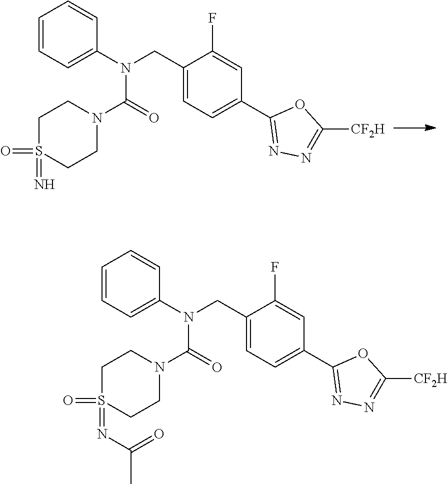 [SMILES: N=S1(=O)CCN(C(=O)N(Cc2ccc(-c3nnc(C(F)F)o3)cc2F)c2ccccc2)CC1 ; CC(=O)N=S1(=O)CCN(C(=O)N(Cc2ccc(-c3nnc(C(F)F)o3)cc2F)c2ccccc2)CC1]

The title compound (0.020 g, 36.78%) was obtained in a colorless oil form according to substantially the same reaction as described in example 30 except for using N-(4-(5-(difluoromethyl)-1,3,4-oxadiazol-2-yl)-2-fluorobenzyl)-1-imino-N-phenylthiomorpholin-4-carboxamide 1-oxide obtained in substantially the same manner as in example 1, instead of N-((5-(5-(difluoromethyl)-1,3,4-oxadiazol-2-yl)pyridin-2-yl)methyl)-N-(3,4-difluorophenyl)-1-iminothiomorpholin-4-carboxamide 1-oxide.

¹H NMR (400 MHz, CDCl₃) δ 7.88 (dd, J=8.0, 1.6 Hz, 1H), 7.76 (dd, J=10.0, 1.6 Hz, 1H), 7.68 (t, J=7.6 Hz, 1H), 7.41-7.37 (m, 2H), 7.27-7.23 (m, 1H), 7.15-7.12 (m, 2H), 7.05 (s, 0.25H), 6.92 (s, 0.5H), 6.79 (s, 0.25H), 4.95 (s, 2H), 3.88-3.81 (m, 2H), 3.60-3.55 (m, 2H), 3.49-3.39 (m, 2H), 3.01-2.97 (m, 2H), 2.05 (s, 3H); LRMS (ES) m/z 522.9 (M⁺+1)

### Examples 41 and 42: Synthesis of Compounds 41 and 42

Compounds 41 and 42 according to examples 41 and 42 were prepared in accordance with the same reactions as described in steps 1, 2 and 3 of example 1, respectively, except for using the reactants of Table 12 below instead of isocyanatobenzene in step 1 of example 1. Table 12 shows the properties and yields of the products prepared in each of steps 1 to 3 in examples 41 and 42.

TABLE 12

| | | Yield/property of | Yield/property of | Yield/property of |
|---|---|---|---|---|
| | | product | product | product |
| | Reactant | obtained from | obtained from | obtained from |
| Classification | in step 1 | step 1 | step 2 | step 3 |
| Example 41 | 1-bromo-3- | 2.500 g, 82.18% | 2.500 g, 71.39% | 0.080 g, 75.56% |
| | isocyanato-benzene | White solid | Colorless oil | Colorless oil |
| Example 42 | 1,2-dichloro-4- | 0.600 g, 77.48% | 0.065 g, 18.29% | 0.020 g, 29.03% |
| | isocyanato-benzene | White solid | Colorless oil | Colorless oil |

The names of compounds 41 and 42 prepared in each of examples 41 and 42, and the NMR and LC-Mass analysis results thereof are shown in table 13 below.

TABLE 13

| | Compound | | |
|---|---|---|---|
| Classification | No. | Compound name | NMR/LRMS |
| Example 41 | 41 | N-(3-bromophenyl)-N-(4- | ¹H NMR (400 MHz, CDCl₃) δ 7.90 (dd, J = |
| | | (5-(difluoromethyl)-1,3,4- | 8.0, 1.6 Hz, 1H), 7.79 (dd, J = 10.4, 1.6 |
| | | oxadiazol-2-yl)-2- | Hz, 1H), 7.67 (t, J = 7.6 Hz, 1H), 7.37- |
| | | fluorobenzyl)-1- | 7.33 (m, 2H), 7.26-7.24 (m, 1H), 7.08- |
| | | iminothiomorpholin-4- | 7.06 (m, 1H), 7.08 (s, 0.25H), 6.93 (s, |
| | | carboxamide 1-oxide | 0.5H), 6.80 (s, 0.25H), 4.93 (s, 2H), 3.80- |
| | | | 3.63 (m, 4H), 2.93-3.86 (m, 4H), 2.55 |
| | | | (brs, 1H); LRMS (ES) m/z 560.9 (M⁺ + 1) |
| Example 42 | 42 | N-(3,4-dichlorophenyl)-N- | ¹H NMR (400 MHz, CDCl₃) δ 7.91 (dd, J = |
| | | (4-(5-(difluoromethyl)- | 8.0, 1.6 Hz, 1H), 7.80 (dd, J = 10.0, 1.6 |
| | | 1,3,4-oxadiazol-2-yl)-2- | Hz, 1H), 7.67 (t, J = 7.8 Hz, 1H), 7.45- |
| | | fluorobenzyl)-1- | 7.42 (m, 1H), 7.28-7.25 (m, 1H), 7.06 (s, |
| | | iminothiomorpholin-4- | 0.25H), 6.99 (dd, J= 8.6, 2.6 Hz, 1H), 6.93 |
| | | carboxamide 1-oxide | (s, 0.5H), 6.80 (s, 0.25H), 4.92 (s, 2H), |
| | | | 3.78-3.63 (m, 4H), 2.95-2.92 (m, 4H), |
| | | | 2.55 (brs, 1H); LRMS (ES) m/z 549.9 (M⁺ + 1) |

### Example 43: Synthesis of Compound 43, N-(4-(5-(difluoromethyl)-1,3,4-oxadiazol-2-yl)-2-fluorobenzyl)-1-imino-N-(3-(thiazole-5-yl)phenyl)thiomorpholin-4-carboxamide 1-oxide

### [Step 1] Synthesis of N-(4-(5-(difluoromethyl)-1,3,4-oxadiazol-2-yl)-2-fluorobenzyl)-N-(3-(thiazol-5-yl)phenyl)thiomorpholin-4-carboxamide

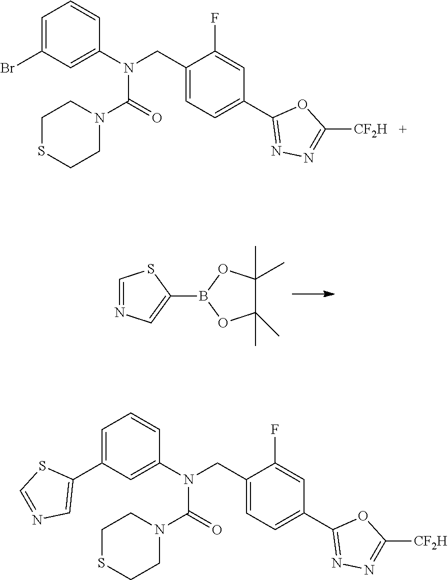 [SMILES: O=C(N1CCSCC1)N(Cc1ccc(-c2nnc(C(F)F)o2)cc1F)c1cccc(Br)c1 ; CC1(C)OB(c2cncs2)OC1(C)C ; O=C(N1CCSCC1)N(Cc1ccc(-c2nnc(C(F)F)o2)cc1F)c1cccc(-c2cncs2)c1]

N-(3-bromophenyl)-N-(4-(5-(difluoromethyl)-1,3,4-oxadiazol-2-yl)-2-fluorobenzyl)thiomorpholin-4-carboxamide (100.00%, 0.200 g, 0.379 mmol), 5-(4,4,5,5-tetramethyl-1,3,2-dioxaborolan-2-yl)thiazole (100.00%, 1.500 equiv., 0.569 mmol), (1,1′-bis(di-tert-butylphosphino)ferrocene)palladium(II) dichloride (100.00%, 0.100 equiv., 0.038 mmol) and cesium carbonate (100.00%, 2.500 equiv, 0.948 mmol) were mixed in 1,4-dioxane (10 mL)/water (3 mL), after which the resulting mixture was irradiated with microwave, then heated at 100° C. for 30 minutes, and then a reaction was finished by lowering the temperature to room temperature. Solvent was removed from the reaction mixture under reduced pressure, after which saturated aqueous solution of sodium hydrogen carbonate was poured into the resulting concentrate and extracted with dichloromethane. An organic layer was washed with saturated aqueous solution of sodium chloride, dehydrated with anhydrous magnesium sulfate, filtered, and concentrated under reduced pressure. The resulting concentrate was purified via column chromatography (SiO₂, 12 g cartridge; dichloromethane/methanol=0 to 30%) and concentrated to obtain the title compound (0.013 g, 6.45%) in a colorless oil form.

### [Step 2] Synthesis of Compound 43

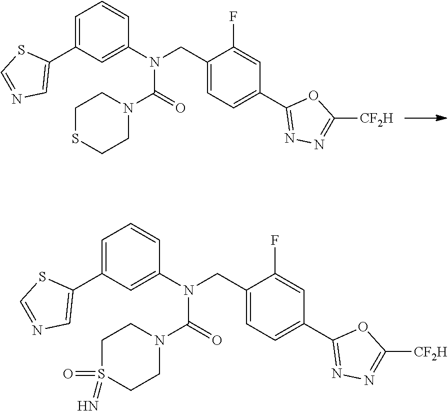 [SMILES: O=C(N1CCSCC1)N(Cc1ccc(-c2nnc(C(F)F)o2)cc1F)c1cccc(-c2cncs2)c1 ; N=S1(=O)CCN(C(=O)N(Cc2ccc(-c3nnc(C(F)F)o3)cc2F)c2cccc(-c3cncs3)c2)CC1]

The title compound (0.004 g, 29.07%) was obtained in a colorless oil form according to substantially the same reaction as described in step 3 of example 1 except for using N-(4-(5-(difluoromethyl)-1,3,4-oxadiazol-2-yl)-2-fluorobenzyl)-N-(3-(thiazol-5-yl)phenyl)thiomorpholin-4-carboxamide prepared in step 1 instead of N-(4-(5-(difluoromethyl)-1,3,4-oxadiazol-2-yl)-2-fluorobenzyl)-N-phenylthiomorpholin-4-carboxamide.

¹H NMR (400 MHz, CDCl₃) δ 8.81 (s, 1H), 8.07 (s, 1H), 7.91 (dd, J=8.0, 1.6 Hz, 1H), 7.79 (dd, J=10.2, 1.4 Hz, 1H), 7.72 (t, J=7.8 Hz, 1H), 7.43-7.39 (m, 2H), 7.35 (s, 1H), 7.11-7.09 (m, 1H), 7.05 (s, 0.25H), 6.93 (s, 0.5H), 6.80 (s, 0.25H), 5.00 (s, 2H), 3.83-3.50 (m, 4H), 2.91-2.88 (m, 4H), 2.50 (brs, 1H); LRMS (ES) m/z 563.9 (M⁺+1)

### Example 44: Synthesis of Compound 44, N-(4-(5-(difluoromethyl)-1,3,4-oxadiazol-2-yl)-2-fluorobenzyl)-N-(3-(furan-2-yl)phenyl)-1-iminothiomorpholin-4-carboxamide 1-oxide

### [Step 1] Synthesis of N-(4-(5-(difluoromethyl)-1,3,4-oxadiazol-2-yl)-2-fluorobenzyl)-N-(3-(furan-2-yl)phenyl)thiomorpholin-4-carboxamide

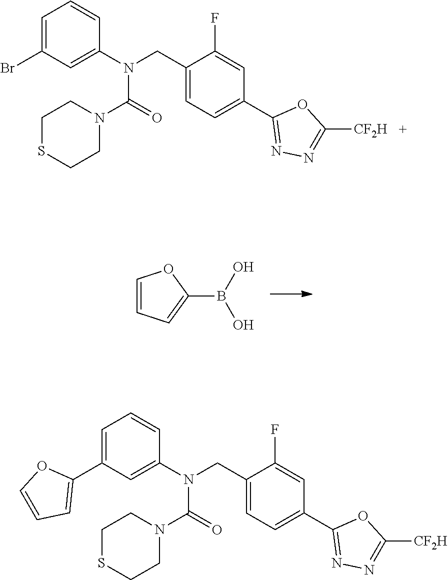 [SMILES: O=C(N1CCSCC1)N(Cc1ccc(-c2nnc(C(F)F)o2)cc1F)c1cccc(Br)c1 ; OB(O)c1ccco1 ; O=C(N1CCSCC1)N(Cc1ccc(-c2nnc(C(F)F)o2)cc1F)c1cccc(-c2ccco2)c1]

The title compound (0.030 g, 15.38%) was obtained in a colorless oil form according to substantially the same reaction as described in step 1 of example 43 except for using 2-furylboronic acid instead of 5-(4,4,5,5-tetramethyl-1,3,2-dioxaborolan-2-yl)thiazole.

### [Step 2] Synthesis of Compound 44

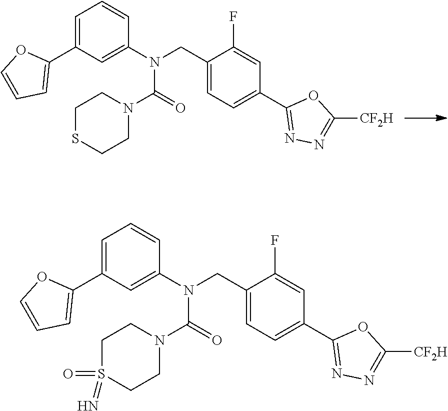 [SMILES: O=C(N1CCSCC1)N(Cc1ccc(-c2nnc(C(F)F)o2)cc1F)c1cccc(-c2ccco2)c1 ; N=S1(=O)CCN(C(=O)N(Cc2ccc(-c3nnc(C(F)F)o3)cc2F)c2cccc(-c3ccco3)c2)CC1]

The title compound (0.010 g, 36.28%) was obtained in a colorless oil form according to substantially the same reaction as described in step 2 of example 43 except for using N-(4-(5-(difluoromethyl)-1,3,4-oxadiazol-2-yl)-2-fluorobenzyl)-N-(3-(furan-2-yl)phenyl)thiomorpholin-4-carboxamide obtained from step 1.

¹H NMR (400 MHz, CDCl₃) δ 7.89 (dd, J=8.0, 1.6 Hz, 1H), 7.78 (dd, J=10.0, 1.6 Hz, 1H), 7.70 (t, J=7.6 Hz, 1H), 7.51-7.48 (m, 3H), 7.37 (t, J=7.8 Hz, 1H), 7.05 (s, 0.25H), 7.01-6.99 (m, 1H), 6.92 (s, 0.5H), 6.79 (s, 0.25H), 6.68-6.67 (m, 1H), 6.51-6.50 (m, 1H), 4.98 (s, 2H), 3.85-3.66 (m, 4H), 2.90-2.86 (m, 4H), 2.55 (brs, 1H); LRMS (ES) m/z 546.9 (M⁺+1)

### Example 45: Synthesis of Compound 45, N-((5-(5-(difluoromethyl)-1,3,4-oxadiazol-2-yl)pyridin-2-yl)methyl)-N-(2,5-difluorophenyl)-1-iminothiomorpholin-4-carboxamide 1-oxide

### [Step 1] Synthesis of N-[[5-[5-(difluoromethyl)-1,3,4-oxadiazol-2-yl]pyridin-2-yl]methyl]-N-(2,5-difluorophenyl)thiomorpholin-4-carboxamide

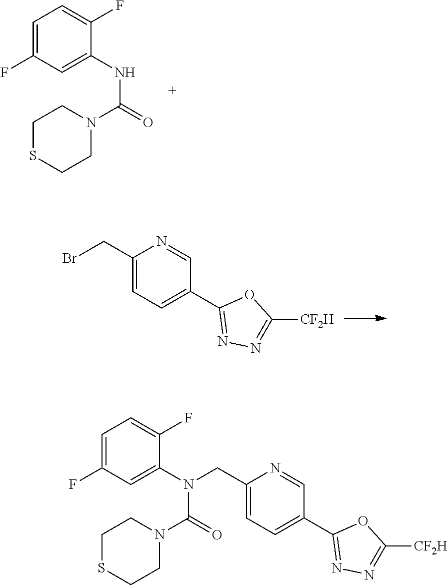 [SMILES: O=C(Nc1cc(F)ccc1F)N1CCSCC1 ; FC(F)c1nnc(-c2ccc(CBr)nc2)o1 ; O=C(N1CCSCC1)N(Cc1ccc(-c2nnc(C(F)F)o2)cn1)c1cc(F)ccc1F]

The title compound (0.074 g, 40.89%) was obtained in a white solid form according to substantially the same reaction as described in step 1 of example 7 except for using N-(2,5-difluorophenyl)thiomorpholin-4-carboxamide instead of N-(3,4-difluorophenyl)thiomorpholin-4-carboxamide in step 1 of example 7.

### [Step 2] Synthesis of Compound 45

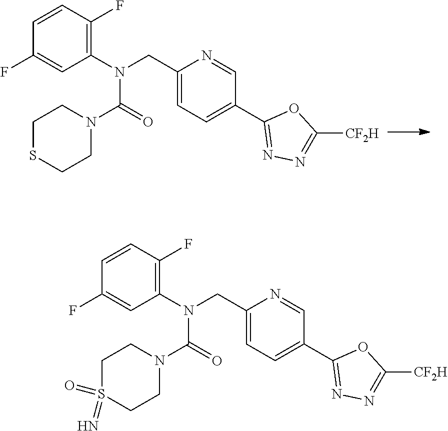 [SMILES: O=C(N1CCSCC1)N(Cc1ccc(-c2nnc(C(F)F)o2)cn1)c1cc(F)ccc1F ; N=S1(=O)CCN(C(=O)N(Cc2ccc(-c3nnc(C(F)F)o3)cn2)c2cc(F)ccc2F)CC1]

The title compound (0.047 g, 59.56%) was obtained in a white solid form according to substantially the same reaction as described in step 2 of example 7 except for using N-[[5-[5-(difluoromethyl)-1,3,4-oxadiazol-2-yl]pyridin-2-yl]methyl]-N-(2,5-difluorophenyl)thiomorpholin-4-carboxamide obtained from step 1.

¹H NMR (400 MHz, CDCl₃) δ 9.24 (d, J=1.6 Hz, 1H), 8.39 (dd, J=8.2, 2.2 Hz, 1H), 7.61 (d, J=8.2 Hz, 1H), 7.16-6.83 (m, 4H), 5.02 (s, 2H), 3.82-3.76 (m, 2H), 3.71-3.65 (m, 2H), 2.97-2.93 (m, 4H); LRMS (ES) m/z 499.9 (M⁺+1).

### Example 46: Synthesis of Compound 46, N-((5-(5-(difluoromethyl)-1,3,4-oxadiazol-2-yl)-3-fluoropyridin-2-yl)methyl)-N-(2,5-difluorophenyl)-1-iminothiomorpholin-4-carboxamide 1-oxide

### [Step 1] Synthesis of N-[[5-[5-(difluoromethyl)-1,3,4-oxadiazol-2-yl]-3-fluoro-2-pyridyl]methyl]-N-(2,5-difluorophenyl)thiomorpholin-4-carboxamide

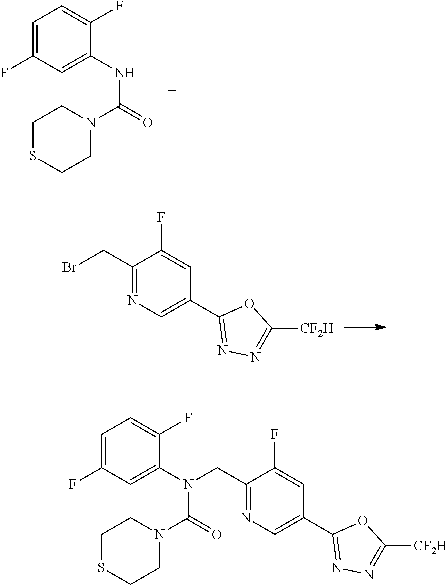 [SMILES: O=C(Nc1cc(F)ccc1F)N1CCSCC1 ; Fc1cc(-c2nnc(C(F)F)o2)cnc1CBr ; O=C(N1CCSCC1)N(Cc1ncc(-c2nnc(C(F)F)o2)cc1F)c1cc(F)ccc1F]

The title compound (0.057 g, 30.33%) was obtained in a white solid form according to substantially the same reaction as described in step 1 of example 45 except for using 2-(6-(bromomethyl)-5-fluoropyridine-3-yl)-5-(difluoromethyl)-1,3,4-oxadiazole instead of 2-(6-(bromomethyl)pyridin-3-yl)-5-(difluoromethyl)-1,3,4-oxadiazole in step 1 of example 45.

### [Step 2] Synthesis of Compound 46

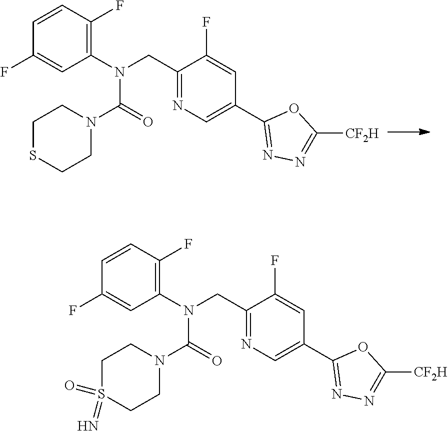 [SMILES: O=C(N1CCSCC1)N(Cc1ncc(-c2nnc(C(F)F)o2)cc1F)c1cc(F)ccc1F ; N=S1(=O)CCN(C(=O)N(Cc2ncc(-c3nnc(C(F)F)o3)cc2F)c2cc(F)ccc2F)CC1]

The title compound (0.048 g, 79.16%) was obtained in a light yellow solid form according to substantially the same reaction as described in step 2 of example 7 except for using N-[[5-[5-(difluoromethyl)-1,3,4-oxadiazol-2-yl]-3-fluoro-2-pyridyl]methyl]-N-(2,5-difluorophenyl)thiomorpholin-4-carboxamide obtained from step 1.

¹H NMR (400 MHz, CDCl₃) δ 9.10 (s, 1H), 8.12 (dd, J=9.3, 1.7 Hz, 1H), 7.17-6.84 (m, 4H), 5.09 (s, 2H), 3.82-3.76 (m, 2H), 3.70-3.64 (m, 2H), 3.11-3.00 (m, 4H), 3.11-3.00 (m, 4H); LRMS (ES) m/z 517.8 (M⁺+1).

### Example 47: Synthesis of Compound 47, N-(4-(5-(difluoromethyl)-1,3,4-oxadiazol-2-yl)-2-fluorobenzyl)-1-imino-N-(3-(pyridin-3-yl)phenyl)thiomorpholin-4-carboxamide 1-oxide

### [Step 1] Synthesis of N-(4-(5-(difluoromethyl)-1,3,4-oxadiazol-2-yl)-2-fluorobenzyl)-N-(3-(pyridin-3-yl)phenyl)thiomorpholin-4-carboxamide

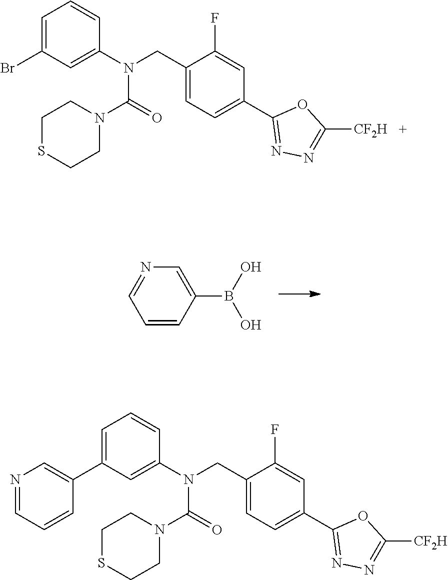 [SMILES: O=C(N1CCSCC1)N(Cc1ccc(-c2nnc(C(F)F)o2)cc1F)c1cccc(Br)c1 ; OB(O)c1cccnc1 ; O=C(N1CCSCC1)N(Cc1ccc(-c2nnc(C(F)F)o2)cc1F)c1cccc(-c2cccnc2)c1]

The title compound (0.113 g, 28.35%) was obtained in a colorless oil form according to substantially the same reaction as described in step 1 of example 44 except for using 3-pyridylboronic acid instead of 2-furylboronic acid.

### [Step 2] Synthesis of Compound 47

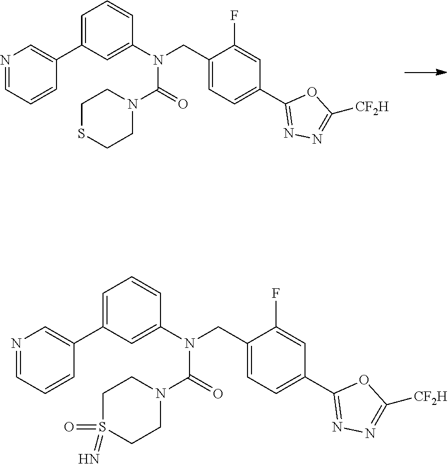 [SMILES: O=C(N1CCSCC1)N(Cc1ccc(-c2nnc(C(F)F)o2)cc1F)c1cccc(-c2cccnc2)c1 ; N=S1(=O)CCN(C(=O)N(Cc2ccc(-c3nnc(C(F)F)o3)cc2F)c2cccc(-c3cccnc3)c2)CC1]

The title compound (0.080 g, 66.84%) was obtained in a colorless oil form according to substantially the same reaction as described in step 2 of example 44 except for using N-(4-(5-(difluoromethyl)-1,3,4-oxadiazol-2-yl)-2-fluorobenzyl)-N-(3-(pyridin-3-yl)phenyl)thiomorpholin-4-carboxamide prepared from step 1.

¹H NMR (400 MHz, CDCl₃) δ 8.78 (d, J=1.6 Hz, 1H), 8.63 (dd, J=4.8, 1.6 Hz, 1H), 7.89 (dd, J=8.0, 1.6 Hz, 1H), 7.82-7.70 (m, 3H), 7.49-7.36 (m, 4H), 7.16-7.13 (m, 1H), 7.05 (s, 0.25H), 6.92 (s, 0.5H), 6.79 (s, 0.25H), 5.01 (s, 2H), 3.83-3.64 (m, 4H), 2.92-2.85 (m, 4H), 2.50 (brs, 1H); LRMS (ES) m/z 557.9 (M⁺+1)

### Example 48: Synthesis of Compound 48, (1S,4S)—N-((5-(5-(difluoromethyl)-1,3,4-oxadiazol-2-yl)pyridin-2-yl)methyl)-N-(2-fluorophenyl)-5-(1-imino-1-oxidotetrahydro-2H-thiopyran-4-yl)-2,5-diazabicyclo[2.2.1]heptan-2-carboxamide

### [Step 1] Synthesis of (1S,4S)—N-((5-(5-(difluoromethyl)-1,3,4-oxadiazol-2-yl)-2-pyridyl)methyl)-N-(2-fluorophenyl)-5-(tetrahydro-2H-thiopyran-4-yl)-2,5-diazabicyclo[2.2.1]heptan-2-carboxamide

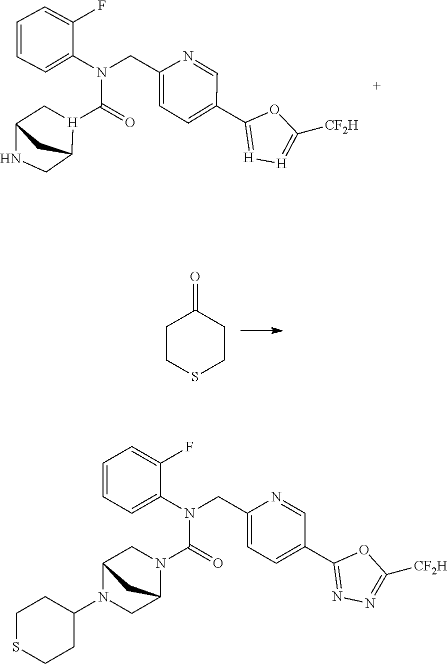

(1S,4S)—N-((5-(5-(difluoromethyl)-1,3,4-oxadiazol-2-yl)pyridin-2-yl)methyl)-N-(2-fluorophenyl)-2,5-diazabicyclo[2.2.1]heptan-2-carboxamide (100.00%, 0.300 g, 0.675 mmol), tetrahydrothiopyran-4-one (100.00%, 1.000 equiv., 0.675 mmol) and sodium triacetoxyborohydride (100.00%, 2.000 equiv., 1.350 mmol) were dissolved in dichloromethane (10 mL) at room temperature, after which the resulting solution was stirred overnight at the same temperature. Saturated aqueous solution of sodium hydrogen carbonate was poured into the reaction mixture and extracted with dichloromethane. An organic layer was washed with saturated aqueous solution of sodium chloride, dehydrated with anhydrous magnesium sulfate, filtered, and concentrated under reduced pressure. The resulting concentrate was purified via column chromatography (SiO₂, 12 g cartridge; dichloromethane/methanol=0 to 10%) and concentrated to obtain the title compound (0.284 g, 77.25%) in a colorless oil form.

### [Step 2] Synthesis of Compound 48

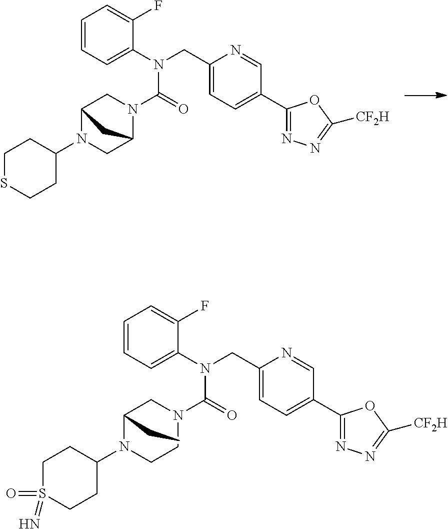

The title compound (0.200 g, 66.63%) was obtained in a colorless oil form according to substantially the same reaction as described in step 3 of example 1 except for using (1S,4S)—N-((5-(5-(difluoromethyl)-1,3,4-oxadiazol-2-yl)-2-pyridyl)methyl)-N-(2-fluorophenyl)-5-(tetrahydro-2H-thiopyran-4-yl)-2,5-diazabicyclo[2.2.1]heptan-2-carboxamide prepared in step 1 instead of N-(4-(5-(difluoromethyl)-1,3,4-oxadiazol-2-yl)-2-fluorobenzyl)-N-phenylthiomorpholin-4-carboxamide.

¹H NMR (400 MHz, CDCl₃) δ 9.16 (d, J=1.6 Hz, 1H), 8.35 (dd, J=8.2, 2.2 Hz, 1H), 7.73 (d, J=8.0 Hz, 1H), 7.26-7.17 (m, 2H), 7.11-7.07 (m, 2H), 7.07 (s, 0.25H), 6.94 (s, 0.5H), 6.81 (s, 0.25H), 5.09-4.95 (m, 2H), 4.34 (s, 1H), 3.40 (s, 1H), 3.37-3.27 (m, 2H), 3.05-2.90 (m, 2H), 2.88-2.82 (m, 2H), 2.65-2.64 (m, 1H), 2.58-2.52 (m, 2H), 2.50 (brs, 1H), 2.22-2.12 (m, 2H), 2.06-1.85 (m, 2H), 1.78-1.60 (m, 2H); LRMS (ES) m/z 576.9 (M⁺+1)

### Examples 49 to 51: Synthesis of Compounds 49 to 51

Compounds 49 to 51 according to examples 49 to 51 were prepared in accordance with the same reactions as described in steps 1, 2 and 3 of example 1, respectively, except for using 2,4-dichloro-1-isocyanato-benzene instead of isocyanatobenzene in step 1 of example 1, and using the reactant of Table 14 below instead of 2-(4-(bromomethyl)-3-fluorophenyl)-5-(difluoromethyl)-1,3,4-oxadiazole in step 2. Table 14 shows the properties and yields of the products prepared in each of steps 1 to 3 in examples 49 to 51.

TABLE 14

| | | Yield/property of | Yield/property of | Yield/property of |
|---|---|---|---|---|
| | | product | product | product |
| | Reactant | obtained from | obtained from | obtained from |
| Classification | in step 2 | step 1 | step 2 | step 3 |
| Example 49 | 2-(6- | 0.740 g, 95.55% | 0.088 g, 51.21% | 0.044 g, 47.08% |
| | (bromomethyl)pyridin- | White solid | Yellow solid | Light yellow solid |
| | 3-y1)-5- | | | |
| | (difluoromethyl)-1,3,4- | | | |
| | oxadiazole | | | |
| Example 50 | 2-(6-(bromomethyl)-5- | Same as step 1 | 0.041 g, 23.04% | 0.023 g, 52.92% |
| | fluoropyridin-3-y1)-5- | of example 49 | Yellow solid | White solid |
| | (difluoromethyl)-1,3,4- | | | |
| | oxadiazole | | | |
| Example 51 | 2-[4-(bromomethyl)-3- | Same as step 1 | 0.072 g, 40.52% | 0.054 g, 70.76% |
| | fluoro-phenyl]-5- | of example 49 | White solid | White solid |
| | (difluoromethyl)-1,3,4- | | | |
| | oxadiazole | | | |

The names of compounds 49 to 51 prepared in each of examples 49 to 51, and the NMR and LC-Mass analysis results thereof are shown in table 15 below.

TABLE 15

| | Compound | | |
|---|---|---|---|
| Classification | No. | Compound name | NMR/LRMS |
| Example 49 | 49 | N-(2,4-dichlorophenyl)-N- | ¹H NMR (400 MHz, CDCl₃) δ 9.22 (d, J = |
| | | ((5-(5-(difluoromethyl)- | 1.7 Hz, 1H), 8.38 (dd, J = 8.2, 2.2 Hz, 1H), |
| | | 1,3,4-oxadiazol-2- | 7.63 (d, J = 8.2 Hz, 1H), 7.50 (d, J = 2.0 |
| | | yl)pyridin-2-yl)methyl)-1- | Hz, 1H), 7.34-7.30 (m, 2H), 6.95 (t, J = |
| | | iminothiomorpholin-4- | 51.7 Hz, 1H), 4.95 (s, 2H), 3.76-3.70 (m, |
| | | carboxamide 1-oxide | 2H), 3.65-3.59 (m, 2H), 2.96-2.91 (m, |
| | | | 4H), 2.58 (brs, 1H); LRMS (ES) m/z 533.8 |
| | | | (M⁺ + 1). |
| Example 50 | 50 | N-(2,4-dichlorophenyl)-N- | ¹H NMR (400 MHz, CDCl₃) δ 9.10 (s, |
| | | ((5-(5-(difluoromethyl)- | 1H), 8.10 (dd, J= 9.3, 1.7 Hz, 1H), 7.53 (d, |
| | | 1,3,4-oxadiazol-2-y1)-3- | J = 2.3 Hz, 1H), 7.38 (d, J = 8.6 Hz, 1H), |
| | | fluoropyridin-2-yl)methyl)- | 7.32-7.30 (m, 1H), 6.96 (t, J = 51.6 Hz, |
| | | 1-iminothiomorpholin-4- | 1H), 5.01 (s, 2H), 3.74-3.70 (m, 2H), 3.65- |
| | | carboxamide 1-oxide | 3.59 (m, 2H), 3.10-3.05 (m, 4H), 2.58 |
| | | | (brs, 1H); LRMS (ES) m/z 551.8 (M⁺ + 1). |
| Example 51 | 51 | N-(2,4-dichlorophenyl)-N- | ¹H NMR (400 MHz, CDCl₃) δ 7.89 (dd, J = |
| | | (4-(5-(difluoromethyl)- | 8.0, 1.4 Hz, 1H), 7.82 (t, J = 7.5 Hz, 1H), |
| | | 1,3,4-oxadiazol-2-y1)-2- | 7.71 (dd, J = 9.9, 1.3 Hz, 1H), 7.51 (d, J = |
| | | fluorobenzyl)-1- | 2.4 Hz, 1H), 7.24 (dd, J = 8.5, 2.4 Hz, 1H), |
| | | iminothiomorpholin-4- | 7.06-6.80 (m, 2H), 4.87 (s, 2H), 3.75- |
| | | carboxamide 1-oxide | 3.68 (m, 2H), 3.64-3.58 (m, 2H), 2.85 (t, |
| | | | J = 5.2 Hz, 4H); LRMS (ES) m/z 550.7 |
| | | | (M⁺ + 1). |

### Example 52: Synthesis of Compound 52, 1-(acetylimino)-N-((5-(5-(difluoromethyl)-1,3,4-oxadiazol-2-yl)pyridin-2-yl)methyl)-N-phenylthiomorpholin-4-carboxamide 1-oxide

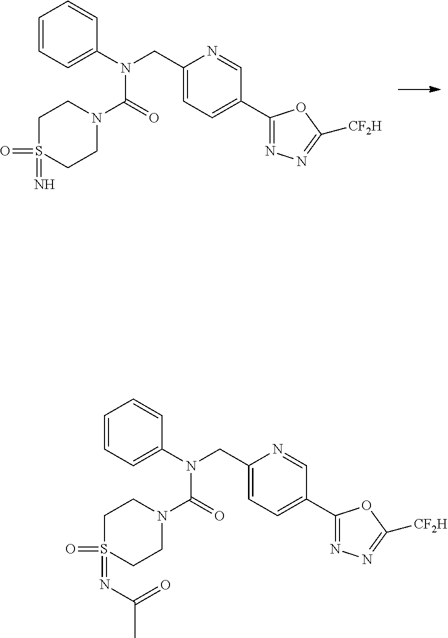 [SMILES: N=S1(=O)CCN(C(=O)N(Cc2ccc(-c3nnc(C(F)F)o3)cn2)c2ccccc2)CC1 ; CC(=O)N=S1(=O)CCN(C(=O)N(Cc2ccc(-c3nnc(C(F)F)o3)cn2)c2ccccc2)CC1]

The title compound (0.100 g, 57.30%) was obtained in a colorless oil form according to substantially the same reactions as described in example 30 except for using N-((5-(5-(difluoromethyl)-1,3,4-oxadiazol-2-yl)pyridin-2-yl)methyl)-1-imino-N-phenylthiomorpholin-4-carboxamide 1-oxide obtained according to substantially the same reaction as described in example 3, instead of N-(4-(5-(difluoromethyl)-1,3,4-oxadiazol-2-yl)-2-fluorobenzyl)-N-(3,4-difluorophenyl)-1-iminothiomorpholin-4-carboxamide 1-oxide.

¹H NMR (400 MHz, CDCl₃) δ 9.26 (d, J=1.6 Hz, 1H), 8.41 (dd, J=8.2, 2.2 Hz, 1H), 7.55 (d, J=8.0 Hz, 1H), 7.40-7.36 (m, 2H), 7.23-7.19 (m, 3H), 7.08 (s, 0.25H), 6.95 (s, 0.5H), 6.82 (s, 0.25H), 5.12 (s, 2H), 3.87-3.83 (m, 2H), 3.60-3.55 (m, 4H), 3.23-3.17 (m, 2H), 2.10 (s, 3H); LRMS (ES) m/z 505.9 (M⁺+1)

### Example 53: Synthesis of Compound 53, N-((5-(5-(difluoromethyl)-1,3,4-oxadiazol-2-yl)pyridin-2-yl)methyl)-1-((methylsulfonyl)imino)-N-phenylthiomorpholin-4-carboxamide 1-oxide

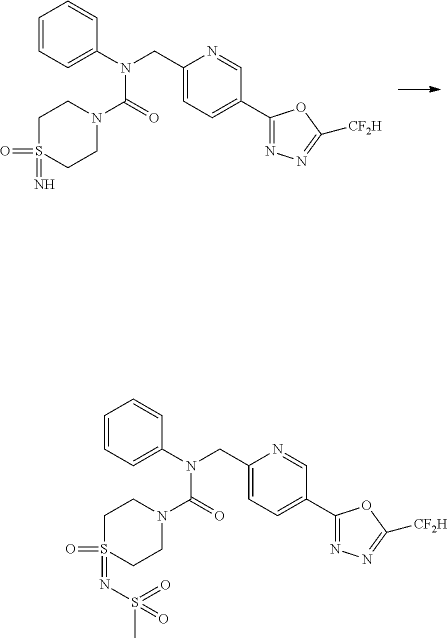 [SMILES: N=S1(=O)CCN(C(=O)N(Cc2ccc(-c3nnc(C(F)F)o3)cn2)c2ccccc2)CC1 ; CS(=O)(=O)N=S1(=O)CCN(C(=O)N(Cc2ccc(-c3nnc(C(F)F)o3)cn2)c2ccccc2)CC1]

The title compound (0.110 g, 47.05%) was obtained in a colorless oil form according to substantially the same reaction as described in example 37 except for using N-((5-(5-(difluoromethyl)-1,3,4-oxadiazol-2-yl)pyridin-2-yl)methyl)-1-imino-N-phenylthiomorpholin-4-carboxamide 1-oxide obtained according to substantially the same reaction as described in example 3, instead of N-(4-(5-(difluoromethyl)-1,3,4-oxadiazol-2-yl)-2-fluorobenzyl)-N-(3,4-difluorophenyl)-1-iminothiomorpholin-4-carboxamide 1-oxide.

¹H NMR (400 MHz, CDCl₃) δ 9.26 (d, J=1.6 Hz, 1H), 8.41 (dd, J=8.2, 2.2 Hz, 1H), 7.54 (d, J=8.2 Hz, 1H), 7.39-7.35 (m, 2H), 7.23-7.17 (m, 2H), 7.08 (s, 0.25H), 6.96 (s, 0.5H), 6.83 (s, 0.25H), 5.13 (s, 2H), 4.04-3.98 (m, 2H), 3.62-3.51 (m, 4H), 3.33-3.28 (m, 2H), 3.11 (s, 3H); LRMS (ES) m/z 541.9 (M⁺+1)

### Examples 54 to 57: Synthesis of Compounds 54 to 57

Compounds 54 to 57 according to examples 54 to 57 were prepared in accordance with the same reactions as described in steps 1, 2 and 3 of example 1, respectively, except for using 1-fluoro-2-isocyanato-benzene instead of isocyanatobenzene in step 1 of example 1, and using the reactant of table 16 below instead of 2-(4-(bromomethyl)-3-fluorophenyl)-5-(difluoromethyl)-1,3,4-oxadiazole in step 2. Table 16 shows the properties and yields of the products prepared in each of steps 1 to 3 in examples 54 to 57.

TABLE 16

| | | Yield/property of | Yield/property of | Yield/property of |
|---|---|---|---|---|
| | | product | product | product |
| | Reactant | obtained from | obtained from | obtained from |
| Classification | in step 2 | step 1 | step 2 | step 3 |
| Example 54 | 2-(4-(bromomethyl)-3,5- | 1.200 g, 68.44% | 0.078 g, 38.69% | 0.057 g, 68.67% |
| | difluoro-phenyl)-5- | White solid | White solid | White solid |
| | (difluoromethyl)-1,3,4- | | | |
| | oxadiazole | | | |
| Example 55 | 2-(6-(bromomethyl)-5- | Same as step 1 | 0.041 g, 49.3%, | 0.041 g, 49.29% |
| | fluoropyridin-3-yl)-5- | of example 54 | White solid | White solid |
| | (difluoromethyl)-1,3,4- | | | |
| | oxadiazole | | | |
| Example 56 | 2-(4-(bromomethyl)-3- | Same as step 1 | 0.147 g, 75.74% | 0.137 g, 73.81% |
| | fluoro-phenyl)-5- | of example 54 | White solid | White solid |
| | (difluoromethyl)-1,3,4- | | | |
| | oxadiazole | | | |
| Example 57 | 2-(6- | Same as step 1 | 0.054 g, 28.87% | 0.036 g, 62.35% |
| | (bromomethyl)pyridin- | of example 54 | Light yellow oil | Light yellow solid |
| | 3-y1)-5- | | | |
| | (difluoromethyl)-1,3,4- | | | |
| | oxadiazole | | | |

The names of compounds 54 to 57 prepared in each of examples 54 to 57, and the NMR and LC-Mass analysis results thereof are shown in table 17 below.

TABLE 17

| | Compound | | |
|---|---|---|---|
| Classification | No. | Compound name | NMR/LRMS |
| Example 54 | 54 | N-(4-(5-(difluoromethyl)- | ¹H NMR (400 MHz, CDCl₃) δ 7.64 (d, J = |
| | | 1,3,4-oxadiazol-2-y1)-2,6- | 7.2 Hz, 2H), 7.32-7.28 (m, 1H), 7.19- |
| | | difluorobenzyl)-N-(2- | 7.14 (m, 3H), 6.94 (t, J = 51.6 Hz, 1H), |
| | | fluorophenyl)-1- | 4.89 (s, 2H), 3.75-3.71 (m, 2H), 3.64- |
| | | iminothiomorpholin-4- | 3.58 (m, 2H), 2.75 (t, J = 5.1 Hz, 4H); |
| | | carboxamide 1-oxide | LRMS (ES) m/z 516.8 (M⁺ + 1). |
| Example 55 | 55 | N-((5-(5-(difluoromethyl)- | ¹H NMR (400 MHz, CDCl₃) δ 9.10 (s, |
| | | 1,3,4-oxadiazol-2-y1)-3- | 1H), 8.10 (dd, J = 9.3, 1.7 Hz, 1H), 7.34- |
| | | fluoropyridin-2-yl)methyl- | 7.25 (m, 3H), 7.20-7.15 (m, 2H), 6.96 (t, |
| | | N-(2-fluorophenyl)-1- | J = 51.6 Hz, 1H), 5.10 (s, 2H), 3.80-3.75 |
| | | iminothiomorpholin-4- | (m, 2H), 3.69-3.63 (m, 2H), 3.01-2.98 |
| | | carboxamide 1-oxide | (m, 4H); LRMS (ES) m/z 499.8 (M⁺ + 1). |
| Example 56 | 56 | N-(4-(5-(difluoromethyl)- | ¹H NMR (400 MHz, CDCl₃) δ 7.87 (dd, J = |
| | | 1,3,4-oxadiazol-2-yl)-2- | 8.0, 1.6 Hz, 1H), 7.78 (t, J = 7.6 Hz, |
| | | fluorobenzyl)-N-(2- | 1H), 7.70 (dd, J = 10.0, 1.6 Hz, 1H), 7.29- |
| | | fluorophenyl)-1- | 7.25 (m, 1H), 7.18-7.13 (m, 3H), 6.92 (t, |
| | | iminothiomorpholin-4- | J = 51.7 Hz, 1H), 4.90 (s, 2H), 3.79-3.73 |
| | | carboxamide 1-oxide | (m, 2H), 3.67-3.61 (m, 2H), 2.79 (t, J = |
| | | | 5.2 Hz, 4H); LRMS (ES) m/z 498.8 (M⁺ + 1). |
| Example 57 | 57 | N-((5-(5-(difluoromethyl)- | ¹H NMR (400 MHz, CDCl₃) δ 9.21 (d, J = |
| | | 1,3,4-oxadiazol-2- | 1.7 Hz, 1H), 8.37 (dd, J = 8.2, 2.2 Hz, 1H), |
| | | yl)pyridin-2-yl)methyl-N- | 7.65 (d, J = 8.2 Hz, 1H), 7.33-7.22 (m, |
| | | (2-fluorophenyl)-1- | 2H), 7.19-7.13 (m, 2H), 6.95 (t, J = 51.6 |
| | | iminothiomorpholin-4- | Hz, 1H), 5.02 (s, 2H), 3.80-3.76 (m, 2H), |
| | | carboxamide 1-oxide | 3.68-3.64 (m, 2H), 2.89-2.88 (m, 4H); |
| | | | LRMS (ES) m/z 481.7 (M⁺ + 1). |

### Example 58: Synthesis of Compound 58, N-(4-(3-(4-(5-(difluoromethyl)-1,3,4-oxadiazol-2-yl)-2-fluorobenzyl)-3-phenylureido)-1-oxidotetrahydro-2H-thiopyran-1-yliden)-2,2,2-trifluoroacetamide

### Example 59: Synthesis of Compound 59, 1-(4-(5-(difluoromethyl)-1,3,4-oxadiazol-2-yl)-2-fluorobenzyl)-3-(1-imino-1-oxidotetrahydro-2H-thiopyran-4-yl)-1-phenylurea

### [Step 1] Synthesis of (4-(5-(difluoromethyl)-1,3,4-oxadiazol-2-yl)-2-fluorobenzyl)(phenyl)carbamic chloride

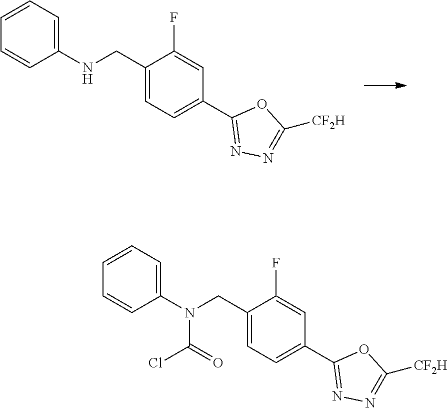 [SMILES: Fc1cc(-c2nnc(C(F)F)o2)ccc1CNc1ccccc1 ; O=C(Cl)N(Cc1ccc(-c2nnc(C(F)F)o2)cc1F)c1ccccc1]

N-(4-(5-(difluoromethyl)-1,3,4-oxadiazol-2-yl)-2-fluorobenzyl)aniline (1.380 g, 4.322 mmol) and N,N-diisopropylethylamine (0.753 mL, 4.322 mmol) were dissolved in dichloromethane (20 mL), after which triphosgene (0.898 g, 3.025 mmol) was added to the resulting solution at 0° C., stirred at the same temperature for 30 minutes, and further stirred at room temperature for 18 hours. Water was poured into the reaction mixture and extracted with dichloromethane. An organic layer was washed with saturated aqueous solution of sodium chloride, dehydrated with anhydrous sodium sulfate, filtered, and concentrated under reduced pressure. The resulting concentrate was purified via column chromatography (SiO₂, 12 g cartridge; ethyl acetate/hexane=0 to 30%) and concentrated to obtain the title compound (0.880 g, 53.3%) in a white solid form.

### [Step 2] Synthesis of Compounds 58 and 59

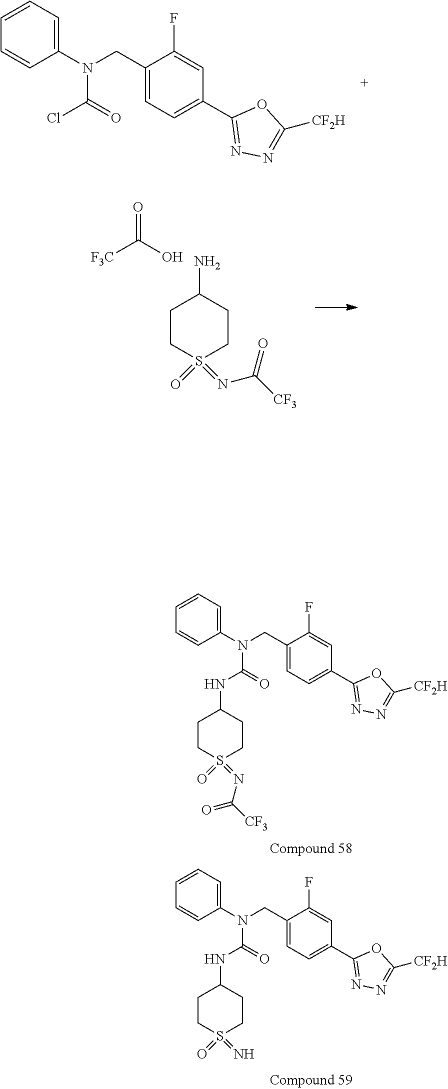

N-(4-amino-1-oxidotetrahydro-2H-thiopyran-1-ylidene)-2,2,2-trifluoroacetamide 2,2,2-trifluoroacetate (0.500 g, 1.396 mmol) was dissolved in N,N-dimethylformamide (10 mL) at 0° C., after which sodium hydride (60.00%, 0.140 g, 3.489 mmol) was added to the resulting solution and stirred at the same temperature for 30 minutes. (4-(5-(difluoromethyl)-1,3,4-oxadiazol-2-yl)-2-fluorobenzyl)(phenyl)carbamic chloride (0.586 g, 1.535 mmol) prepared in step 1 was added to the reaction mixture and further stirred at room temperature for three hours. Water was poured into the reaction mixture and extracted with ethyl acetate. An organic layer was washed with saturated aqueous solution of sodium chloride, dehydrated with anhydrous sodium sulfate, filtered, and concentrated under reduced pressure. The resulting concentrate was purified via column chromatography (SiO₂, 12 g cartridge; ethyl acetate/hexane=0 to 100%) and concentrated to obtain each of compound 58 (0.110 g, 13.4%) and compound 59 (0.160 g, 23.2%) in a white solid form.

<Compound 58> ¹H NMR (400 MHz, CDCl₃) δ 7.86 (dd, J=8.0, 1.2 Hz, 1H), 7.72 (dd, J=9.8, 1.4 Hz, 1H), 7.58 (t, J=7.6 Hz, 1H), 7.43-7.34 (m, 3H), 7.13-7.12 (m, 2H), 7.05 (s, 0.25H), 6.92 (s, 0.5H), 6.79 (s, 0.25H), 5.02 (s, 2H), 4.39 (d, J=7.2 Hz, 1H), 4.11-4.02 (m, 1H), 3.89-3.85 (m, 2H), 3.35-3.28 (m, 2H), 2.38-2.33 (m, 2H), 1.92-1.83 (m, 2H); LRMS (ES) m/z 590.9 (M⁺+1)

<Compound 59> ¹H NMR (400 MHz, CDCl₃) δ 7.84 (d, J=7.6 Hz, 1H), 7.68 (d, J=9.6 Hz, 1H), 7.58 (t, J=7.6 Hz, 1H), 7.39-7.30 (m, 3H), 7.11-7.08 (m, 2H), 7.04 (s, 0.25H), 6.90 (s, 0.5H), 6.78 (s, 0.25H), 4.97 (s, 2H), 4.34 (d, J=7.6 Hz, 1H), 3.90-3.93 (m, 1H), 3.18-3.00 (m, 4H), 2.80 (brs, 1H), 2.18-2.14 (m, 2H), 1.93-1.87 (m, 2H); LRMS (ES) m/z 494.9 (M⁺+1)

### Example 60: Synthesis of Compound 60, 3-((4-(5-(difluoromethyl)-1,3,4-oxadiazol-2-yl)-2-fluorobenzyl)(phenyl)amino)-4-(1-imino-1-oxidothiomorpholino)cyclobut-3-en-1,2-dione

### [Step 1] Synthesis of 3-(phenylamino)-4-thiomorpholinocyclobut-3-en-1,2-dione

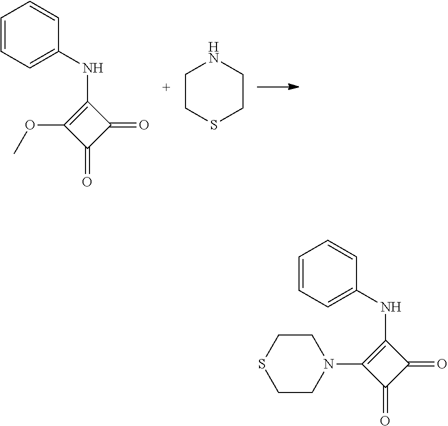

3-methoxy-4-(phenylamino)cyclobut-3-en-1,2-dione (100.00%, 2.000 g, 9.843 mmol), thiomorpholine (100.00%, 1.200 equiv., 11.812 mmol) and N,N-diisopropylethylamine (100.00%, 3.000 equiv., 29.529 mmol) were dissolved in methanol (30 mL) at room temperature, after which the resulting solution was stirred at the same temperature for three days. A precipitated solid was filtered, washed with hexane, and dried to obtain the title compound (2.000 g, 74.08%) in a white solid form.

### [Step 2] Synthesis of 3-((4-(5-(difluoromethyl)-1,3,4-oxadiazol-2-yl)-2-fluorobenzyl)(phenyl)amino)-4-thiomorpholinocyclobut-3-en-1,2-dione

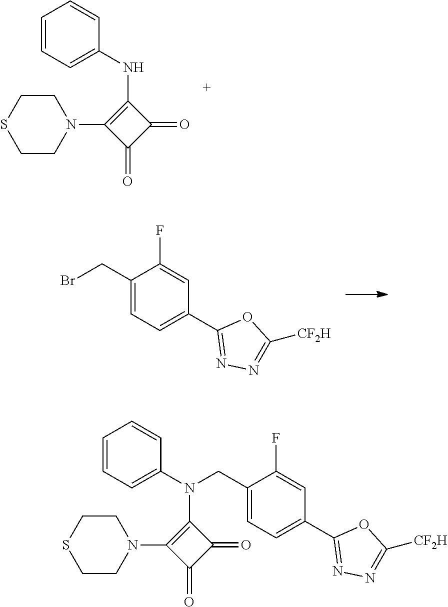 [SMILES: O=c1c(Nc2ccccc2)c(N2CCSCC2)c1=O ; Fc1cc(-c2nnc(C(F)F)o2)ccc1CBr ; O=c1c(N2CCSCC2)c(N(Cc2ccc(-c3nnc(C(F)F)o3)cc2F)c2ccccc2)c1=O]

3-(phenylamino)-4-thiomorpholinocyclobut-3-en-1,2-dione (100.00%, 0.300 g, 1.094 mmol) prepared in step 1 was dissolved in N,N-dimethylformamide (10 mL) at 0° C., after which sodium hydride (100.00%, 1.500 equiv., 1.641 mmol) was added to the resulting solution and stirred at the same temperature for 30 minutes. 2-(4-(bromomethyl)-3-fluorophenyl)-5-(difluoromethyl)-1,3,4-oxadiazole (100.00%, 1.000 equiv., 1.094 mmol) was added into the reaction mixture and further stirred at room temperature for two hours. Saturated aqueous solution of sodium hydrogen carbonate was poured into the reaction mixture, and extracted with ethyl acetate. An organic layer was washed with saturated aqueous solution of sodium chloride, dehydrated with anhydrous magnesium sulfate, filtered, and concentrated under reduced pressure. The resulting concentrate was purified via column chromatography (SiO₂, 12 g cartridge; hexane/ethyl acetate=0 to 50%) and concentrated to obtain the title compound (0.300 g, 54.81%) in a white solid form.

### [Step 3] Synthesis of Compound 60

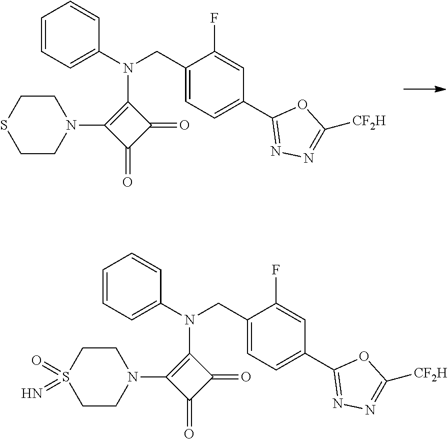 [SMILES: O=c1c(N2CCSCC2)c(N(Cc2ccc(-c3nnc(C(F)F)o3)cc2F)c2ccccc2)c1=O ; N=S1(=O)CCN(c2c(N(Cc3ccc(-c4nnc(C(F)F)o4)cc3F)c3ccccc3)c(=O)c2=O)CC1]

3-((4-(5-(difluoromethyl)-1,3,4-oxadiazol-2-yl)-2-fluorobenzyl)(phenyl)amino)-4-thiomorpholinocyclobut-3-en-1,2-dione (100.00%, 0.150 g, 0.300 mmol) prepared in step 2, iodobenzene diacetate (100.00%, 2.500 equiv., 0.749 mmol) and ammonium carbamate (100.00%, 2.000 equiv., 0.599 mmol) were dissolved in methanol (10 mL) at room temperature, after which the resulting solution was stirred overnight at the same temperature. A precipitated solid was filtered, washed with hexane, and dried to obtain the title compound (0.080 g, 50.22%) in a white solid form.

¹H NMR (400 MHz, CDCl₃) δ 7.86-7.81 (m, 2H), 7.67-7.63 (m, 1H), 7.66 (s, 0.25H), 7.53 (s, 0.5H), 7.42-7.38 (m, 2H), 7.40 (s, 0.25H), 7.25-7.18 (m, 3H), 5.57 (s, 2H), 3.80 (s, 1H), 3.56-3.36 (m, 4H), 3.02-2.93 (m, 4H); LRMS (ES) m/z 532.9 (M⁺+1)

### Example 61: Synthesis of Compound 61, 3-(((5-(5-(difluoromethyl)-1,3,4-oxadiazol-2-yl)-3-fluoropyridin-2-yl)methyl)(phenyl)amino)-4-(1-imino-1-oxidothiomorpholino)cyclobut-3-en-1,2-dione

### [Step 1] Synthesis of 3-(((5-(5-(difluoromethyl)-1,3,4-oxadiazol-2-yl)-3-fluoropyridin-2-yl)methyl)(phenyl)amino)-4-thiomorpholinocyclobut-3-en-1,2-dione

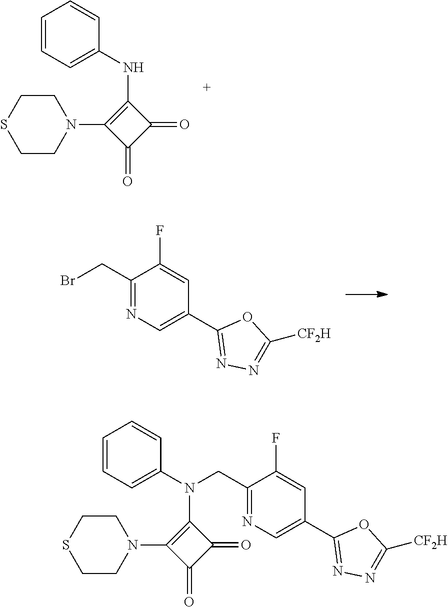 [SMILES: O=c1c(Nc2ccccc2)c(N2CCSCC2)c1=O ; Fc1cc(-c2nnc(C(F)F)o2)cnc1CBr ; O=c1c(N2CCSCC2)c(N(Cc2ncc(-c3nnc(C(F)F)o3)cc2F)c2ccccc2)c1=O]

The title compound (0.128 g, 35.01%) was obtained in a colorless oil form according to substantially the same reaction as described in step 2 of example 60 except for using 2-(6-(bromomethyl)-5-fluoro-3-pyridyl)-5-(difluoromethyl)-1,3,4-oxadiazole instead of 2-(4-(bromomethyl)-3-fluorophenyl)-5-(difluoromethyl)-1,3,4-oxadiazole.

### [Step 2] Synthesis of Compound 61

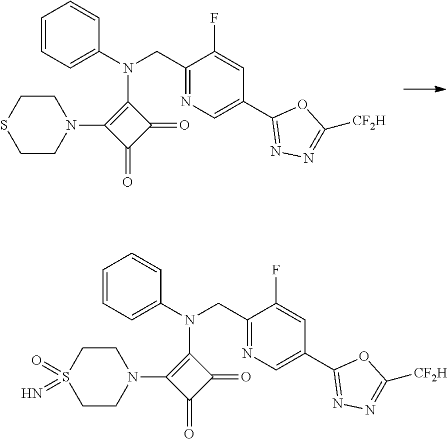 [SMILES: O=c1c(N2CCSCC2)c(N(Cc2ncc(-c3nnc(C(F)F)o3)cc2F)c2ccccc2)c1=O ; N=S1(=O)CCN(c2c(N(Cc3ncc(-c4nnc(C(F)F)o4)cc3F)c3ccccc3)c(=O)c2=O)CC1]

The title compound (0.05 g, 51.09%) was obtained in a colorless oil form according to substantially the same reaction as described in step 3 of example 60 except for using 3-(((5-(5-(difluoromethyl)-1,3,4-oxadiazol-2-yl)-3-fluoropyridin-2-yl)methyl)(phenyl)amino)-4-thiomorpholinocyclobut-3-en-1,2-dione.

¹H NMR (400 MHz, CDCl₃) δ 8.99 (s, 1H), 8.40 (dd, J=10.0, 1.6 Hz, 1H), 7.70 (s, 0.25H), 7.57 (s, 0.5H), 7.45 (s, 0.25H), 7.40 (t, J=8.0 Hz, 2H), 7.19-7.17 (m, 3H), 5.68 (s, 2H), 3.66 (s, 1H), 3.60-3.40 (m, 4H), 3.05-2.97 (m, 4H); LRMS (ES) m/z 533.9 (M⁺+1)

### Example 62: Synthesis of Compound 62, N-[[5-[5-(difluoromethyl)-1,3,4-oxadiazol-2-yl]-2-pyridyl]methyl]-N-(4-fluorophenyl)-1-imino-1-oxo-1,4-thiazinan-4-carboxamide

### [Step 1] Synthesis of N-(4-fluorophenyl)thiomorpholin-4-carboxamide

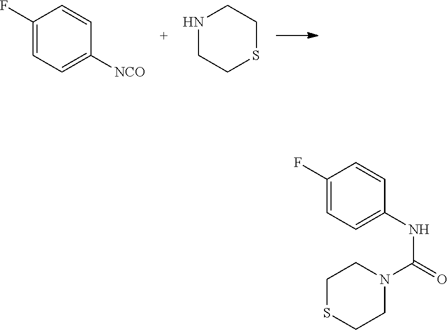

1-fluoro-4-isocyanato-benzene (100.00%, 0.500 g, 3.647 mmol) and thiomorpholine (100.00%, 0.376 g, 3.644 mmol) were dissolved in diethyl ether (20 mL) at room temperature, and then the resulting solution was stirred at the same temperature for 18 hours. The resulting precipitated solid was filtered, washed with diethyl ether, and dried to obtain a title compound (0.667 g, 76.12%) in a white solid form.

### [Step 2] Synthesis of N-[[5-[5-(difluoromethyl)-1,3,4-oxadiazol-2-yl]-2-pyridyl]methyl]-N-(4-fluorophenyl)thiomorpholin-4-carboxamide

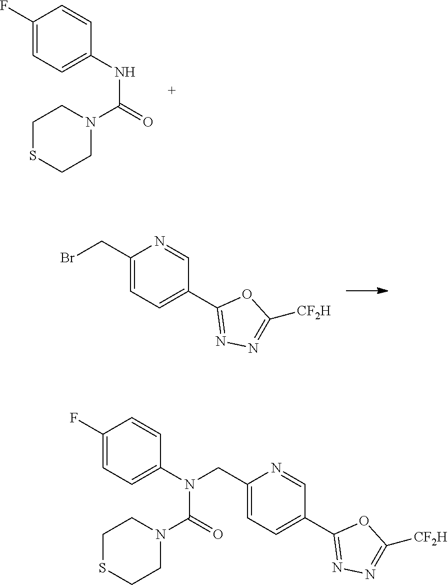 [SMILES: O=C(Nc1ccc(F)cc1)N1CCSCC1 ; FC(F)c1nnc(-c2ccc(CBr)nc2)o1 ; O=C(N1CCSCC1)N(Cc1ccc(-c2nnc(C(F)F)o2)cn1)c1ccc(F)cc1]

N-(4-fluorophenyl)thiomorpholin-4-carboxamide (100.00%, 0.100 g, 0.416 mmol) and sodium hydride (60.00%, 0.018 g, 0.450 mmol) were dissolved in N,N-dimethylformamide (2 mL) at 0° C., and then 2-[6-(bromomethyl)-3-pyridyl]-5-(difluoromethyl)-1,3,4-oxadiazole (100.00%, 0.121 g, 0.417 mmol) was added into the resulting solution and stirred at room temperature for 18 hours. Solvent was removed from the reaction mixture under reduced pressure, and then water was poured into the resulting concentrate, extracted with dichloromethane, filtered via a plastic filter to remove a solid residue and an aqueous solution layer therefrom, and concentrated under reduced pressure. The resulting concentrate was purified via column chromatography (SiO₂, 12 g cartridge; ethyl acetate/hexane=5 to 40%), and concentrated to obtain a title compound (0.040 g, 21.39%) in a light yellow oil form.

### [Step 3] Synthesis of Compound 62

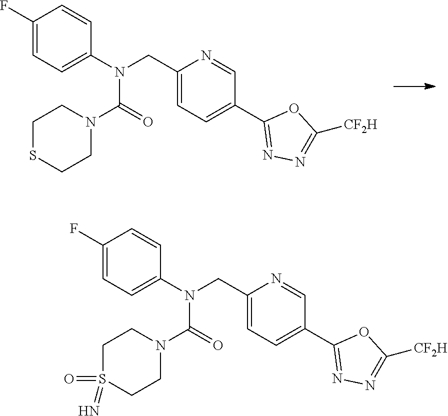 [SMILES: O=C(N1CCSCC1)N(Cc1ccc(-c2nnc(C(F)F)o2)cn1)c1ccc(F)cc1 ; N=S1(=O)CCN(C(=O)N(Cc2ccc(-c3nnc(C(F)F)o3)cn2)c2ccc(F)cc2)CC1]

N-[[5-[5-(difluoromethyl)-1,3,4-oxadiazol-2-yl]-2-pyridyl]methyl]-N-(4-fluorophenyl)thiomorpholin-4-carboxamide (100.00%, 0.040 g, 0.089 mmol), iodobenzene diacetate (100.00%, 0.072 g, 0.224 mmol) and ammonium carbamate (100.00%, 0.014 g, 0.179 mmol) were dissolved in methanol (3 mL) at room temperature, and then the resulting solution was stirred at 50° C. for 18 hours, after which a temperature was lowered to room temperature to terminate the reaction. Solvent was removed from the resulting mixture under reduced pressure, and then the resulting concentrate was purified via column chromatography (SiO₂, 4 g cartridge; methanol/dichloromethane=0 to 5%) and concentrated to obtain a title compound (0.010 g, 23.38%) in a light yellow solid form.

¹H NMR (400 MHz, CDCl₃) δ 7.89 (d, J=8.0 Hz, 1H), 7.77 (d, J=8.6 Hz, 1H), 7.68 (t, J=7.7 Hz, 1H), 7.49 (d, J=8.7 Hz, 2H), 7.06-6.80 (m, 3H), 5.32 (s, 2H), 3.79-3.73 (m, 2H), 3.69-3.63 (m, 2H), 2.91-2.88 (m, 4H); LRMS (ES) m/z 481.8 (M⁺+1).

### Example 63: Synthesis of Compound 63, N-[[4-[5-(difluoromethyl)-1,3,4-oxadiazol-2-yl]-2-fluoro-phenyl]methyl]-N-(4-fluorophenyl)-1-imino-1-oxo-1,4-thiazinan-4-carboxamide

### [Step 1] Synthesis of N-(4-fluorophenyl)thiomorpholin-4-carboxamide

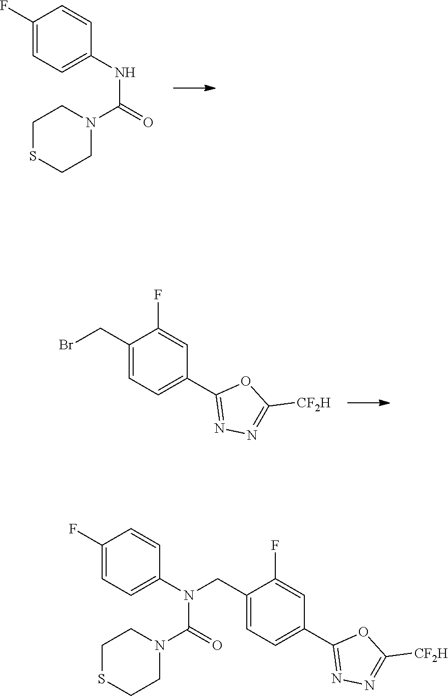 [SMILES: O=C(Nc1ccc(F)cc1)N1CCSCC1 ; Fc1cc(-c2nnc(C(F)F)o2)ccc1CBr ; O=C(N1CCSCC1)N(Cc1ccc(-c2nnc(C(F)F)o2)cc1F)c1ccc(F)cc1]

N-(4-fluorophenyl)thiomorpholin-4-carboxamide (100.00%, 0.100 g, 0.416 mmol) and sodium hydride (60.00%, 0.018 g, 0.450 mmol) were dissolved in N,N-dimethylformamide (2 mL) at 0° C., and then 2-[4-(bromomethyl)-3-fluoro-phenyl]-5-(difluoromethyl)-1,3,4-oxadiazole (100.00%, 0.128 g, 0.417 mmol) was added into the resulting solution and stirred at room temperature for 18 hours. Solvent was removed from the reaction mixture under reduced pressure, and then water was poured into the resulting concentrate, extracted with dichloromethane, filtered via a plastic filter to remove a solid residue and an aqueous solution layer therefrom, and concentrated under reduced pressure. The resulting concentrate was purified via column chromatography (SiO₂, 4 g cartridge; ethyl acetate/hexane=5 to 40%), and concentrated to obtain a title compound (0.033 g, 17.00%) in a white solid form.

### [Step 3] Synthesis of Compound 63

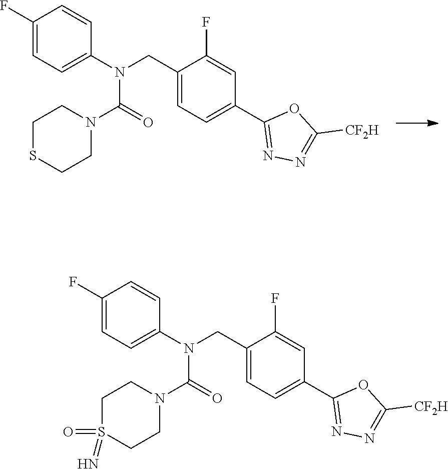 [SMILES: O=C(N1CCSCC1)N(Cc1ccc(-c2nnc(C(F)F)o2)cc1F)c1ccc(F)cc1 ; N=S1(=O)CCN(C(=O)N(Cc2ccc(-c3nnc(C(F)F)o3)cc2F)c2ccc(F)cc2)CC1]

N-[[4-[5-(difluoromethyl)-1,3,4-oxadiazol-2-yl]-2-fluoro-phenyl]methyl]-N-(4-fluorophenyl)thiomorpholin-4-carboxamide (100.00%, 0.033 g, 0.071 mmol), iodobenzene diacetate (100.00%, 0.057 g, 0.177 mmol) and ammonium carbamate (100.00%, 0.011 g, 0.141 mmol) were dissolved in methanol (3 mL) at room temperature, and then the resulting solution was stirred at 50° C. for 18 hours, after which a temperature was lowered to room temperature to terminate the reaction. Solvent was removed from the resulting mixture under reduced pressure, and then the resulting concentrate was purified via column chromatography (SiO₂, 4 g cartridge; methanol/dichloromethane=0 to 5%) and concentrated to obtain a title compound (0.025 g, 71.02%) in a light yellow solid form.

¹H NMR (400 MHz, CDCl₃) δ 7.90 (d, J=7.92 Hz, 1H), 7.77 (dd, J=10.1, 1.3 Hz, 1H), 7.13-6.80 (m, 5H), 4.91 (s, 2H), 3.79-3.75 (m, 2H), 3.67-3.63 (m, 2H), 2.88-2.87 (m, 4H); LRMS (ES) m/z 498.8 (M⁺+1)

### Example 64: Synthesis of Compound 64, N-[[5-[5-(difluoromethyl)-1,3,4-oxadiazol-2-yl]-2-pyridyl]methyl]-N-(4-fluorophenyl)-1-imino-2,6-dimethyl-1-oxo-1,4-thiazinan-4-carboxamide

### [Step 1] Synthesis of N-(4-fluorophenyl)-2,6-dimethyl-thiomorpholin-4-carboxamide

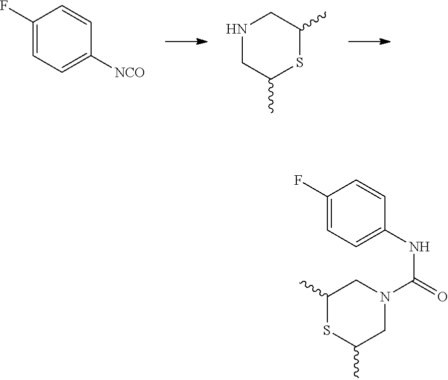

1-fluoro-4-isocyanato-benzene (100.00% solution, 0.4098 mL, 3.647 mmol) and 2,6-dimethylthiomorpholine (100.00%, 0.376 g, 2.865 mmol) were dissolved in diethyl ether (10 mL) at room temperature, and then the resulting solution was stirred at the same temperature for 18 hours. The resulting precipitated solid was filtered, washed with diethyl ether, and dried to obtain a title compound (0.568 g, 58.03%) in a light orange colored solid form.

### [Step 2] Synthesis of N-[[5-[5-(difluoromethyl)-1,3,4-oxadiazol-2-yl]-2-pyridyl]methyl]-N-(4-fluorophenyl)-2,6-dimethyl-thiomorpholin-4-carboxamide

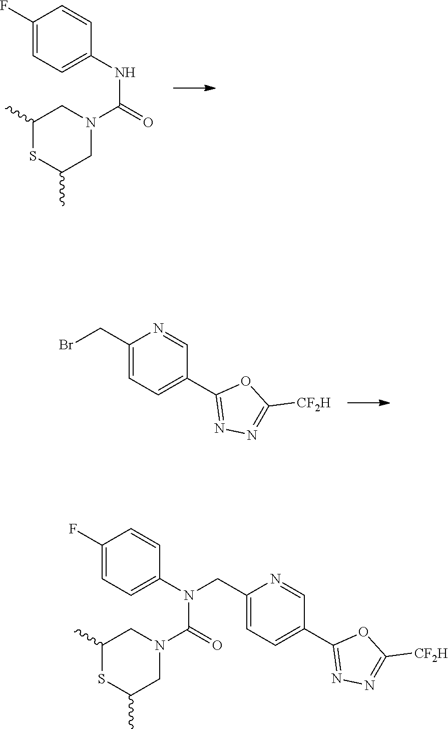

N-(4-fluorophenyl)-2,6-dimethyl-thiomorpholin-4-carboxamide (100.00%, 0.100 g, 0.373 mmol) and sodium hydride (60.00%, 0.016 g, 0.400 mmol) were dissolved in N,N-dimethylformamide (2 mL) at 0° C., and then 2-[6-(bromomethyl)-3-pyridyl]-5-(difluoromethyl)-1,3,4-oxadiazole (100.00%, 0.108 g, 0.372 mmol) was added into the resulting solution and stirred at room temperature for 18 hours. Solvent was removed from the reaction mixture under reduced pressure, and then water was poured into the resulting concentrate, extracted with dichloromethane, filtered via a plastic filter to remove a solid residue and an aqueous solution layer therefrom, and concentrated under reduced pressure. The resulting concentrate was purified via column chromatography (SiO₂, 4 g cartridge; ethyl acetate/hexane=5 to 40%), and concentrated to obtain a title compound (0.050 g, 28.10%) in a light yellow oil form.

### [Step 3] Synthesis of Compound 64

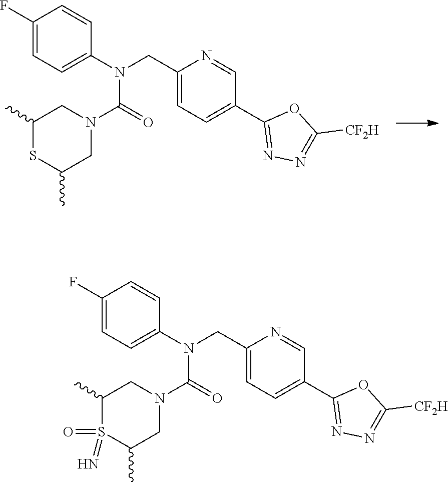

N-[[5-[5-(difluoromethyl)-1,3,4-oxadiazol-2-yl]-2-pyridyl]methyl]-N-(4-fluorophenyl)-2,6-dimethyl-thiomorpholin-4-carboxamide (100.00%, 0.050 g, 0.105 mmol), iodobenzene diacetate (100.00%, 0.084 g, 0.261 mmol) and ammonium carbamate (100.00%, 0.016 g, 0.205 mmol) were dissolved in methanol (3 mL) at room temperature, and then the resulting solution was stirred at 50° C. for 18 hours, after which a temperature was lowered to room temperature to terminate the reaction. Solvent was removed from the resulting mixture under reduced pressure, and then the resulting concentrate was purified via column chromatography (SiO₂, 4 g cartridge; ethyl acetate/hexane=50 to 100%) and concentrated to obtain a title compound (0.033 g, 61.98%) in a light yellow solid form.

¹H NMR (400 MHz, CDCl₃) δ 9.25 (d, J=1.8 Hz, 1H), 8.36 (dd, J=8.1, 2.3 Hz, 1H), 7.60 (d, J=8.12 Hz, 1H), 7.28-7.21 (m, 2H), 7.09-7.06 (m, 2H), 7.05-6.83 (m, 1H), 5.18-4.86 (m, 2H), 3.97-3.92 (m, 0.6H), 3.79-3.74 (m, 0.6H), 3.69 (dd, J=14.2, 2.8 Hz, 0.6H), 3.52-3.47 (m, 0.6H), 3.31-3.25 (m, 0.6H), 3.03-2.93 (m, 3H), 1.25-1.21 (m, 6H); LRMS (ES) m/z 509.9 (M⁺+1)

### Example 65: Synthesis of Compound 65, N-[[4-[5-(difluoromethyl)-1,3,4-oxadiazol-2-yl]-2-fluoro-phenyl]methyl]-N-(4-fluorophenyl)-1-imino-2,6-dimethyl-1-oxo-1,4-thiazinan-4-carboxamide

### [Step 1] Synthesis of N-[[4-[5-(difluoromethyl)-1,3,4-oxadiazol-2-yl]-2-fluoro-phenyl]methyl]-N-(4-fluorophenyl)-2,6-dimethyl-thiomorpholin-4-carboxamide

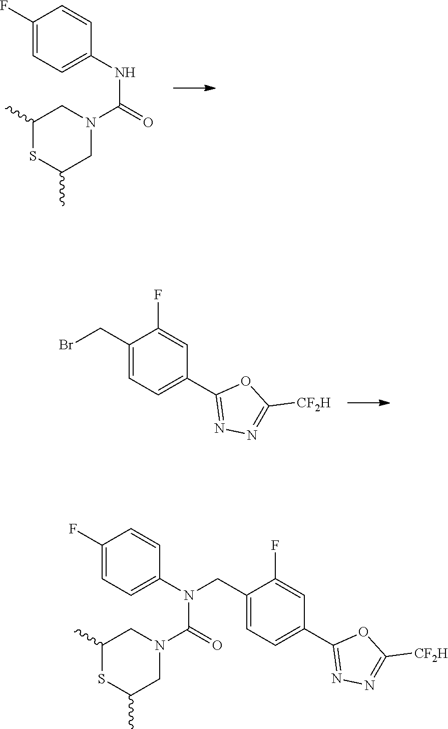

N-(4-fluorophenyl)-2,6-dimethyl-thiomorpholin-4-carboxamide (100.00%, 0.100 g, 0.373 mmol) and sodium hydride (60.00%, 0.016 g, 0.400 mmol) were dissolved in N,N-dimethylformamide (2 mL) at 0° C., and then 2-[4-(bromomethyl)-3-fluoro-phenyl]-5-(difluoromethyl)-1,3,4-oxadiazole (100.00%, 0.114 g, 0.371 mmol) was added into the resulting solution and stirred at room temperature for 18 hours. Solvent was removed from the reaction mixture under reduced pressure, and then water was poured into the resulting concentrate, extracted with dichloromethane, filtered via a plastic filter to remove a solid residue and an aqueous solution layer therefrom, and concentrated under reduced pressure. The resulting concentrate was purified via column chromatography (SiO₂, 4 g cartridge; ethyl acetate/hexane=5 to 40%), and concentrated to obtain a title compound (0.146 g, 79.24%) in a colorless oil form.

### [Step 2] Synthesis of Compound 65

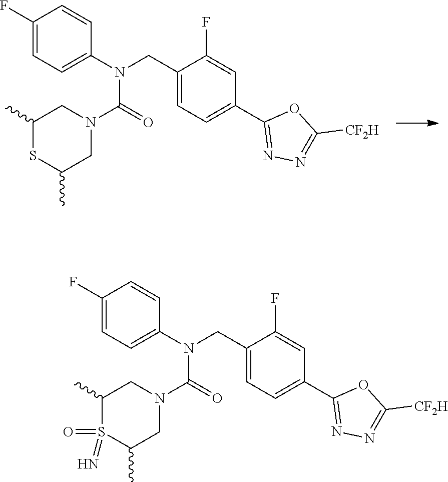

N-[[4-[5-(difluoromethyl)-1,3,4-oxadiazol-2-yl]-2-fluoro-phenyl]methyl]-N-(4-fluorophenyl)-2,6-dimethyl-thiomorpholin-4-carboxamide (100.00%, 0.146 g, 0.295 mmol), iodobenzene diacetate (100.00%, 0.238 g, 0.739 mmol) and ammonium carbamate (100.00%, 0.046 g, 0.589 mmol) were dissolved in methanol (3 mL) at room temperature, and then the resulting solution was stirred at 50° C. for 18 hours, after which a temperature was lowered to room temperature to terminate the reaction. Solvent was removed from the resulting mixture under reduced pressure, and then the resulting concentrate was purified via column chromatography (SiO₂, 4 g cartridge; ethyl acetate/hexane=50 to 100%) and concentrated to obtain a title compound (0.126 g, 81.21%) in a white solid form.

¹H NMR (400 MHz, CDCl₃) δ 7.88 (d, J=8.0 Hz, 1H), 7.78-7.69 (m, 2H), 7.13-7.06 (m, 4H), 7.05-6.80 (m, 1H), 5.00-4.73 (m, 2H), 3.98 (d, J=15.0 Hz, 0.6H), 3.76 (d, J=14.2 Hz, 0.6H), 3.68 (d, J=14.0 Hz, 0.6H), 3.31-3.26 (m, 0.6H), 2.99-2.93 (m, 1H), 2.81-2.77 (m, 2H), 2.14-2.04 (m, 0.6H), 1.24-1.18 (m, 6H); LRMS (ES) m/z 526.9 (M⁺+1)

### Example 66: Synthesis of Compound 66, N-[[4-[5-(difluoromethyl)-1,3,4-oxadiazol-2-yl]-2-fluoro-phenyl]methyl]-1-imino-1-oxo-N-(3-pyridyl)-1,4-thiazinan-4-carboxamide

### [Step 1] Synthesis of benzyl N-[l-oxo-4-(3-pyridylcarbamoyl)-1,4-thiazinan-1-ylidene]carbamate

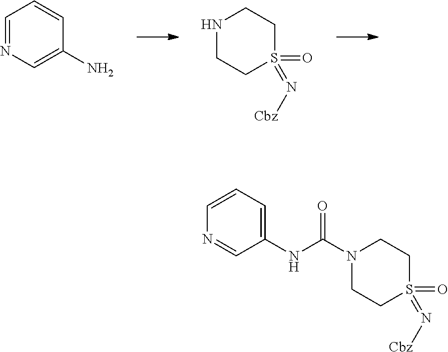 [SMILES: Nc1cccnc1 ; O=C(N=S1(=O)CCNCC1)OCc1ccccc1 ; O=C(N=S1(=O)CCN(C(=O)Nc2cccnc2)CC1)OCc1ccccc1]

Pyridin-3-amine (100.00%, 0.200 g, 2.125 mmol) and di(imidazol-1-yl)methanone (100.00%, 0.448 g, 2.763 mmol) were dissolved in tetrahydrofuran (5 mL), and then the resulting solution was stirred at room temperature for one hour, after which benzyl N-(1-oxo-1,4-thiazinan-1-ylidene)carbamate (100.00%, 0.684 g, 2.549 mmol) was added thereto and further stirred at the same temperature for 18 hours. Solvent was removed from the resulting mixture under reduced pressure, and then the resulting concentrate was purified via column chromatography (SiO₂, 4 g cartridge; ethyl acetate/hexane=50 to 100%) and concentrated to obtain a title compound (0.112 g, 13.57%) in a light yellow solid form.

### [Step 2] Synthesis of benzyl N-[4-[[4-[5-(difluoromethyl)-1,3,4-oxadiazol-2-yl]-2-fluoro-phenyl]methyl-(3-pyridyl)carbamoyl]-1-oxo-1,4-thiazinan-1-ylidene]carbamate

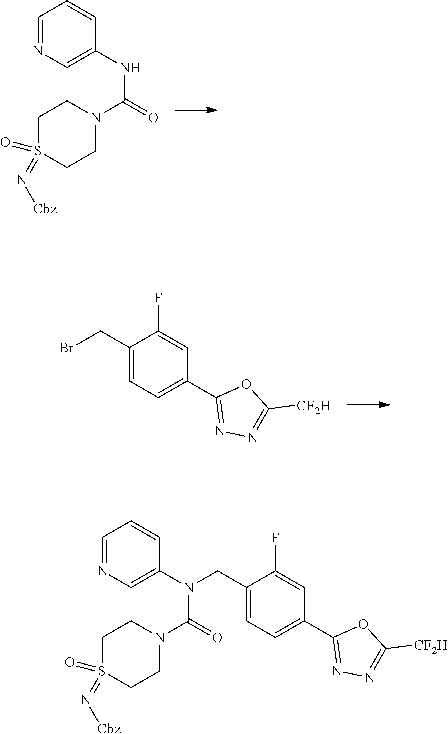 [SMILES: O=C(N=S1(=O)CCN(C(=O)Nc2cccnc2)CC1)OCc1ccccc1 ; Fc1cc(-c2nnc(C(F)F)o2)ccc1CBr ; O=C(N=S1(=O)CCN(C(=O)N(Cc2ccc(-c3nnc(C(F)F)o3)cc2F)c2cccnc2)CC1)OCc1ccccc1]

Benzyl N-[1-oxo-4-(3-pyridylcarbamoyl)-1,4-thiazinan-1-ylidene]carbamate (100.00%, 0.100 g, 0.258 mmol) and sodium hydride (60.00%, 0.011 g, 0.275 mmol) were dissolved in N,N-dimethylformamide (3 mL) at 0° C., and then 2-[4-(bromomethyl)-3-fluoro-phenyl]-5-(difluoromethyl)-1,3,4-oxadiazole (100.00%, 0.079 g, 0.257 mmol) was added into the resulting solution and stirred at room temperature for three hours. Solvent was removed from the reaction mixture under reduced pressure, and then N-dichloromethane aqueous solution was poured into the resulting concentrate, extracted with dichloromethane, filtered via a plastic filter to remove a solid residue and an aqueous solution layer therefrom, and concentrated under reduced pressure. The resulting concentrate was purified via column chromatography (SiO₂, 4 g cartridge; ethyl acetate/hexane=50 to 100%), and concentrated to obtain a title compound (0.030 g, 18.96%) in a light yellow oil form.

### [Step 3] Synthesis of Compound 66

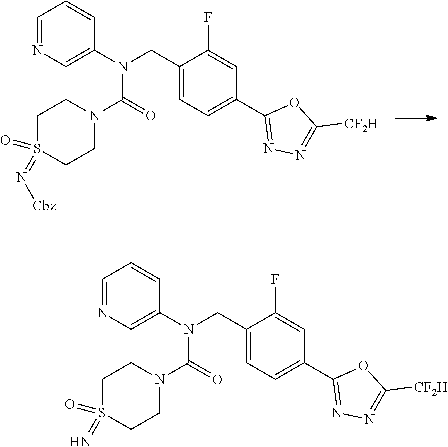 [SMILES: O=C(N=S1(=O)CCN(C(=O)N(Cc2ccc(-c3nnc(C(F)F)o3)cc2F)c2cccnc2)CC1)OCc1ccccc1 ; N=S1(=O)CCN(C(=O)N(Cc2ccc(-c3nnc(C(F)F)o3)cc2F)c2cccnc2)CC1]

Benzyl N-[4-[[4-[5-(difluoromethyl)-1,3,4-oxadiazol-2-yl]-2-fluoro-phenyl]methyl-(3-pyridyl)carbamoyl]-1-oxo-1,4-thiazinan-1-ylidene]carbamate (100.00%, 0.030 g, 0.049 mmol) was dissolved in methanol (5 mL) and stirred at room temperature, and then 10%-Pd/C (5 mg) was slowly added thereinto at the same temperature, and stirred for three hours in the presence of a hydrogen balloon attached thereto at the same temperature. The reaction mixture was filtered via a celite pad to remove a solid therefrom, and then solvent was removed from the resulting filtrate without the solid under reduced pressure, and the obtained product was used without an additional purification process (0.022 g, 93.80%, light yellow solid).

¹H NMR (400 MHz, CDCl₃) δ 78.50-8.47 (m, 1H), 7.91 (dd, J=8.0, 1.6 Hz, 1H), 7.79 (dd, J=10.2, 1.5 Hz, 1H), 7.71 (t, J=7.6 Hz, 1H), 7.51-7.48 (m, 1H), 7.36-7.33 (m, 1H), 7.06-6.80 (m, 1H), 4.97 (s, 2H), 3.77-3.72 (m, 2H), 3.68-3.63 (m, 2H), 2.90 (t, J=5.2 Hz, 4H); LRMS (ES) m/z 481.7 (M⁺+1)

### Example 67: Synthesis of Compound 67, N-(4-(1-acetylpiperidin-4-yl)phenyl)-N-(4-(5-(difluoromethyl)-1,3,4-oxadiazol-2-yl)-2-fluorobenzyl)thiomorpholin-4-carboxamide

### [Step 1] Synthesis of tert-butyl 4-[4-[[4-[5-(difluoromethyl)-1,3,4-oxadiazol-2-yl]-2-fluoro-phenyl]methyl-(thiomorpholin-4-carbonyl)amino]phenyl]-3,6-dihydro-2H-pyridin-1-carboxylate

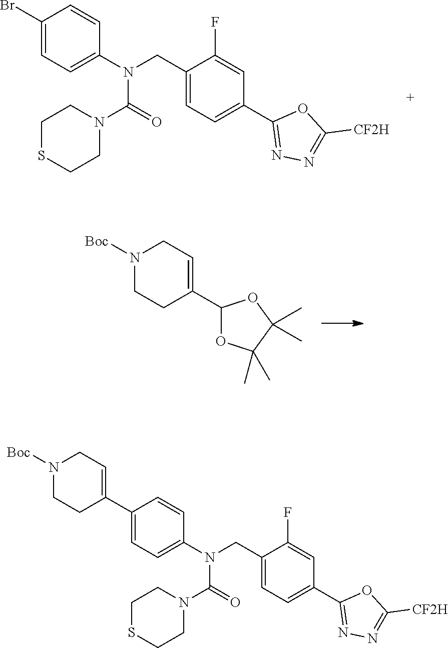 [SMILES: O=C(N1CCSCC1)N(Cc1ccc(-c2nnc(C(F)F)o2)cc1F)c1ccc(Br)cc1 ; CC(C)(C)OC(=O)N1CC=C(C2OC(C)(C)C(C)(C)O2)CC1 ; CC(C)(C)OC(=O)N1CC=C(c2ccc(N(Cc3ccc(-c4nnc(C(F)F)o4)cc3F)C(=O)N3CCSCC3)cc2)CC1]

N-(4-bromophenyl)-N-[[4-[5-(difluoromethyl)-1,3,4-oxadiazol-2-yl]-2-fluoro-phenyl]methyl]thiomorpholin-4-carboxamide (100.00%, 1.710 g, 3.242 mmol), tert-butyl 4-(4,4,5,5-tetramethyl-1,3,2-dioxaborolan-2-yl)-3,6-dihydro-2H-pyridin-1-carboxylate (100.00%, 1.200 g, 3.881 mmol), [1,1′-bis(di-tert-butylphosphino)ferrocene]dichloropalladium(II) (Pd(dtbpf)Cl₂, 100.00%, 0.106 g, 0.163 mmol), and cesium carbonate (100.00%, 2.110 g, 6.476 mmol) were mixed in 1,4-dioxane (12 mL)/water (3 mL) at room temperature, and then the resulting mixture was irradiated with microwave and heated at 100° C. for 30 minutes, after which a temperature was lowered to room temperature to terminate the reaction. Solvent was removed from the reaction mixture under reduced pressure, and then a saturated aqueous sodium hydrogen carbonate solution was poured into the resulting concentrate and extracted with dichloromethane. An organic layer was washed with saturated aqueous sodium chloride solution, dehydrated with anhydrous magnesium sulfate, filtered, and concentrated under reduced pressure. The resulting concentrate was purified via column chromatography (SiO₂, 40 g cartridge; ethyl acetate/hexane=10 to 40%) and concentrated to obtain a title compound (0.950 g, 46.53%) in a brown solid form.

### [Step 2] Tert-butyl 4-[4-[[4-[5-(difluoromethyl)-1,3,4-oxadiazol-2-yl]-2-fluoro-phenyl]methyl-(thiomorpholin-4-carbonyl)amino]phenyl]piperidin-1-carboxylate

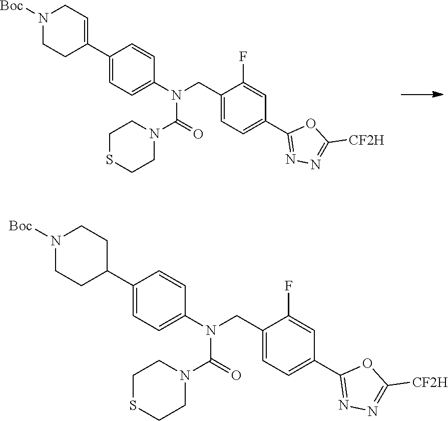 [SMILES: CC(C)(C)OC(=O)N1CC=C(c2ccc(N(Cc3ccc(-c4nnc(C(F)F)o4)cc3F)C(=O)N3CCSCC3)cc2)CC1 ; CC(C)(C)OC(=O)N1CCC(c2ccc(N(Cc3ccc(-c4nnc(C(F)F)o4)cc3F)C(=O)N3CCSCC3)cc2)CC1]

Tert-butyl 4-[4-[[4-[5-(difluoromethyl)-1,3,4-oxadiazol-2-yl]-2-fluoro-phenyl]methyl-(thiomorpholin-4-carbonyl)amino]phenyl]-3,6-dihydro-2H-pyridin-1-carboxylate (100.00%, 0.950 g, 1.509 mmol) was dissolved in methanol (50 mL) and stirred at room temperature, and then 10%-Pd/C (100 mg) was slowly added into the resulting solution at the same temperature and stirred at 50° C. for 18 hours in the presence of a hydrogen balloon attached thereto, and a temperature was lowered to room temperature to terminate the reaction. The reaction mixture was filtered via a celite pad to remove a solid therefrom, and then solvent was removed from the resulting filtrate without the solid under reduced pressure. Then, the resulting concentrate was purified via column chromatography (SiO₂, 12 g cartridge; ethyl acetate/hexane=10 to 40%), and concentrated to obtain a title compound (0.341 g, 35.78%) in a white solid form.

### [Step 3] N-[[4-[5-(difluoromethyl)-1,3,4-oxadiazol-2-yl]-2-fluoro-phenyl]methyl]-N-[4-(4-piperidyl)phenyl]thiomorpholin-4-carboxamide

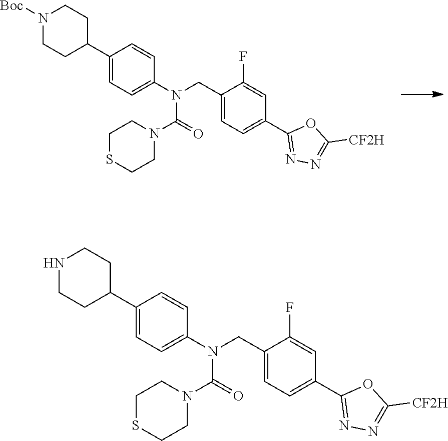 [SMILES: CC(C)(C)OC(=O)N1CCC(c2ccc(N(Cc3ccc(-c4nnc(C(F)F)o4)cc3F)C(=O)N3CCSCC3)cc2)CC1 ; O=C(N1CCSCC1)N(Cc1ccc(-c2nnc(C(F)F)o2)cc1F)c1ccc(C2CCNCC2)cc1]

Tert-butyl 4-[4-[[4-[5-(difluoromethyl)-1,3,4-oxadiazol-2-yl]-2-fluoro-phenyl]methyl-(thiomorpholin-4-carbonyl)amino]phenyl]piperidin-1-carboxylate (100.00%, 0.341 g, 0.540 mmol) and trifluoroacetic acid (100.00% solution, 0.3 mL, 3.920 mmol) were dissolved in dichloromethane (3 mL) at room temperature, and then the resulting solution was stirred at the same temperature for 18 hours. A saturated aqueous sodium hydrogen carbonate solution was poured into the reaction mixture and extracted with dichloromethane. An organic layer was washed with saturated aqueous sodium chloride solution, dehydrated with anhydrous magnesium sulfate, filtered, and concentrated under reduced pressure. The obtained product was used without a further purification process (0.287 g, 100.00%, light orange-colored solid).

### [Step 4] N-[4-(1-acetyl-4-piperidyl)phenyl]-N-[[4-[5-(difluoromethyl)-1,3,4-oxadiazol-2-yl]-2-fluoro-phenyl]methyl]thiomorpholin-4-carboxamide

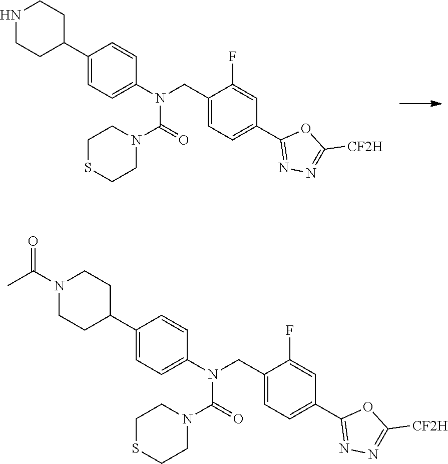 [SMILES: O=C(N1CCSCC1)N(Cc1ccc(-c2nnc(C(F)F)o2)cc1F)c1ccc(C2CCNCC2)cc1 ; CC(=O)N1CCC(c2ccc(N(Cc3ccc(-c4nnc(C(F)F)o4)cc3F)C(=O)N3CCSCC3)cc2)CC1]

N-[[4-[5-(difluoromethyl)-1,3,4-oxadiazol-2-yl]-2-fluoro-phenyl]methyl]-N-[4-(4-piperidyl)phenyl]thiomorpholin-4-carboxamide (100.00%, 0.100 g, 0.188 mmol), acetyl chloride (100.00% solution, 0.02 mL, 0.281 mmol) and triethylamine (100.00% solution, 0.05 mL, 0.359 mmol) were dissolved in dichloromethane (3 mL) at 0° C., and then the resulting solution was stirred at room temperature for three hours. A saturated aqueous sodium hydrogen carbonate solution was poured into the reaction mixture, extracted with dichloromethane, filtered via a plastic filter to remove a solid residue and an aqueous solution layer therefrom, and concentrated under reduced pressure. The resulting concentrate was purified via column chromatography (SiO₂, 4 g cartridge; ethyl acetate/hexane=50 to 100%), and concentrated to obtain a title compound (0.092 g, 85.26%) in a light yellow oil form.

### [Step 5] Synthesis of Compound 67

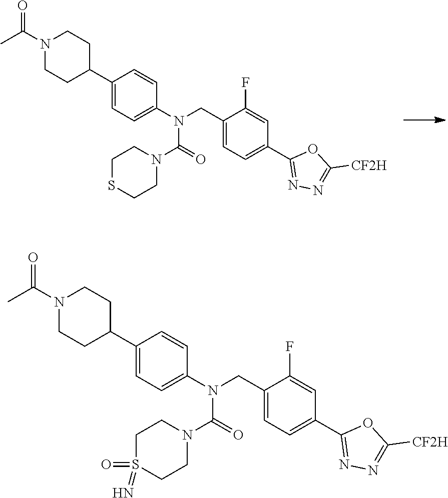 [SMILES: CC(=O)N1CCC(c2ccc(N(Cc3ccc(-c4nnc(C(F)F)o4)cc3F)C(=O)N3CCSCC3)cc2)CC1 ; CC(=O)N1CCC(c2ccc(N(Cc3ccc(-c4nnc(C(F)F)o4)cc3F)C(=O)N3CCS(=N)(=O)CC3)cc2)CC1]

N-[4-(1-acetyl-4-piperidyl)phenyl]-N-[[4-[5-(difluoromethyl)-1,3,4-oxadiazol-2-yl]-2-fluoro-phenyl]methyl]thiomorpholin-4-carboxamide (100.00%, 0.092 g, 0.160 mmol), iodobenzene diacetate (100.00%, 0.129 g, 0.401 mmol), and ammonium carbamate (100.00%, 0.025 g, 0.320 mmol) were dissolved in methanol (3 mL) at room temperature, and then the resulting solution was stirred at 50° C. for six hours, after which a temperature was lowered to room temperature to terminate the reaction. Solvent was removed from the resulting mixture under reduced pressure, and then the resulting concentrate was purified via column chromatography (SiO₂, 4 g cartridge; methanol/dichloromethane=0 to 5%) and concentrated to obtain a title compound (0.062 g, 63.94%) in a white solid form.

¹H NMR (400 MHz, CDCl₃) δ 7.85 (d, J=7.88 Hz, 1H), 7.73 (d, J=10.0 Hz, 1H), 7.65 (t, J=7.6 Hz, 1H), 7.17 (d, J=8.16 Hz, 2H), 7.06-6.80 (m, 3H), 4.90 (s, 2H), 4.79-4.76 (m, 1H), 3.95-3.92 (m, 1H), 3.74-3.73 (m, 2H), 3.62-3.61 (m, 2H), 3.16 (t, J=12.9 Hz, 1H), 2.88-2.79 (m, 4H), 2.75-2.69 (m, 1H), 2.64-2.58 (m, 1H), 2.12 (s, 3H), 1.90-1.83 (m, 2H), 1.61-1.52 (m, 2H); LRMS (ES) m/z 605.9 (M⁺+1)

### Example 68: Synthesis of Compound 68, (1S,4S)—N-[[4-[5-(difluoromethyl)-1,3,4-oxadiazol-2-yl]-2-fluoro-phenyl]methyl]-2-imino-2-oxo-N-phenyl-2lambda6-thia-5-azabicyclo[2.2.1]heptan-5-carboxamide

### [Step 1] Synthesis of (1S,4S)—N-phenyl-2-thia-5-azabicyclo[2.2.1]heptan-5-carboxamide

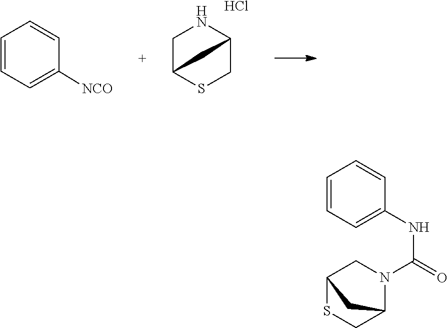

Isocyanatobenzene (100.00%, 0.500 g, 4.197 mmol), (1S,4S)-2-thia-5-azabicyclo[2.2.1]heptane; hydrochloride (100.00%, 1.000 equiv., 4.197 mmol) and N,N-diisopropylethylamine (100.00%, 2.000 equiv., 8.395 mmol) were dissolved in dichloromethane (20 mL) at room temperature and the resulting solution was stirred overnight at the same temperature. Solvent was removed from the reaction mixture under reduced pressure, and then dichloromethane (20 mL) and methanol (10 mL) were added into the resulting concentrate and stirred to filter out a precipitated solid, which was then washed with hexane and dried to obtain a title compound (0.700 g, 71.18%) in a white solid form.

### [Step 2] Synthesis of (1S,4S)—N-[[4-[5-(difluoromethyl)-1,3,4-oxadiazol-2-yl]-2-fluoro-phenyl]methyl]-N-phenyl-2-thia-5-azabicyclo[2.2.1]heptan-5-carboxamide

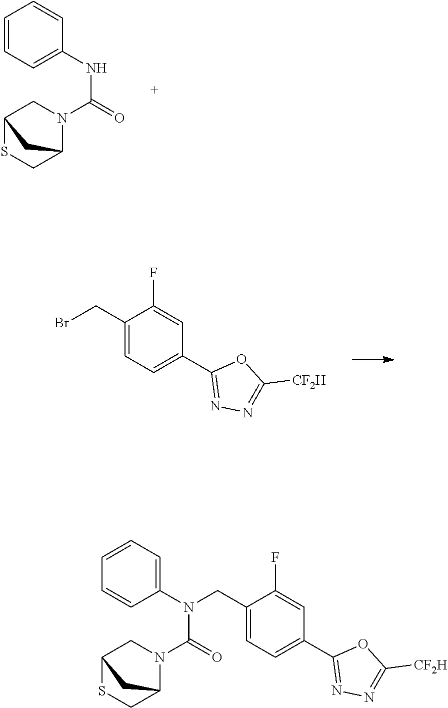 [SMILES: O=C(Nc1ccccc1)N1C[C@@H]2C[C@H]1CS2 ; Fc1cc(-c2nnc(C(F)F)o2)ccc1CBr ; O=C(N(Cc1ccc(-c2nnc(C(F)F)o2)cc1F)c1ccccc1)N1C[C@@H]2C[C@H]1CS2]

(1S,4S)—N-phenyl-2-thia-5-azabicyclo[2.2.1]heptan-5-carboxamide (100.00%, 0.210 g, 0.896 mmol) prepared in step 1 was dissolved in N,N-dimethylformamide (10 mL) at 0° C., and then sodium hydride (100.00%, 1.500 equiv., 1.344 mmol) was added to the resulting solution and stirred at the same temperature for 30 minutes. 2-[4-(bromomethyl)-3-fluoro-phenyl]-5-(difluoromethyl)-1,3,4-oxadiazole (100.00%, 1.000 equiv., 0.896 mmol) was added into the reaction mixture and further stirred at room temperature for two hours. Solvent was removed from the reaction mixture under reduced pressure, and then a saturated aqueous sodium hydrogen carbonate solution was poured into the resulting concentrate and extracted with dichloromethane. An organic layer was washed with saturated aqueous sodium chloride solution, dehydrated with anhydrous magnesium sulfate, filtered, and concentrated under reduced pressure. The resulting concentrate was purified via column chromatography (SiO₂, 12 g cartridge; dichloromethane/methanol=0 to 10%), and concentrated to obtain a title compound (0.021 g, 5.09%) in a colorless oil form.

### [Step 3] Synthesis of Compound 68

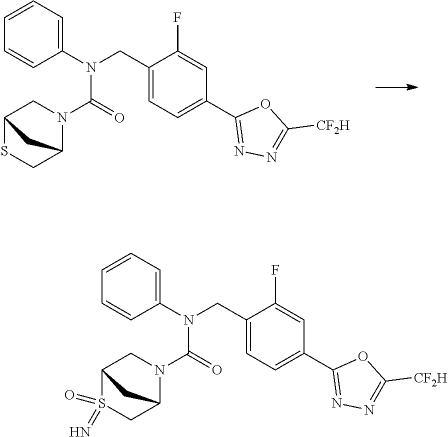 [SMILES: O=C(N(Cc1ccc(-c2nnc(C(F)F)o2)cc1F)c1ccccc1)N1C[C@@H]2C[C@H]1CS2 ; N=S1(=O)C[C@@H]2C[C@H]1CN2C(=O)N(Cc1ccc(-c2nnc(C(F)F)o2)cc1F)c1ccccc1]

(1S,4S)—N-[[4-[5-(difluoromethyl)-1,3,4-oxadiazol-2-yl]-2-fluoro-phenyl]methyl]-N-phenyl-2-thia-5-azabicyclo[2.2.1]heptan-5-carboxamide (100.00%, 0.076 g, 0.165 mmol) prepared in step 2, (diacetoxyiodo)benzene (100.00%, 2.500 equiv., 0.413 mmol) and ammonium carbamate (100.00%, 2.000 equiv., 0.330 mmol) were dissolved in methanol (10 mL) at room temperature, and then the resulting solution was stirred overnight at the same temperature. Solvent was removed from the reaction mixture under reduced pressure, and then a saturated aqueous sodium hydrogen carbonate solution was poured into the resulting concentrate and extracted with dichloromethane. An organic layer was washed with saturated aqueous sodium chloride solution, dehydrated with anhydrous magnesium sulfate, filtered, and concentrated under reduced pressure. The resulting concentrate was purified via column chromatography (SiO₂, 12 g cartridge; dichloromethane/methanol=0 to 10%), and concentrated to obtain a title compound (0.030 g, 36.98%) in a colorless oil form.

¹H NMR (400 MHz, CDCl₃) δ 7.91-7.88 (m, 1H), 7.80-7.71 (m, 2H), 7.35-7.31 (m, 2H), 7.23-7.19 (m, 1H), 7.14-7.12 (m, 2H), 7.05 (s, 0.25H), 6.92 (s, 0.5H), 6.79 (s, 0.25H), 5.21-5.15 (m, 1H), 4.87-4.81 (m, 1H), 4.69 (s, 1H), 3.68-3.65 (m, 1H), 3.56-3.51 (m, 1H), 3.44 (s, 1H), 2.69-2.63 (m, 1H), 2.60-2.55 (m, 1H), 2.25-2.20 (m, 1H), 1.90 (brs, 1H); LRMS (ES) m/z 492.9 (M⁺+1)

### Example 69: Synthesis of Compound 69, N-[[4-[5-(difluoromethyl)-1,3,4-oxadiazol-2-yl]-2-fluoro-phenyl]methyl]-N-(2,5-difluorophenyl)-1-imino-1-oxo-1,4-thiazinan-4-sulfonamide

### [Step 1] Synthesis of N-(2,5-difluorophenyl)thiomorpholin-4-sulfonamide

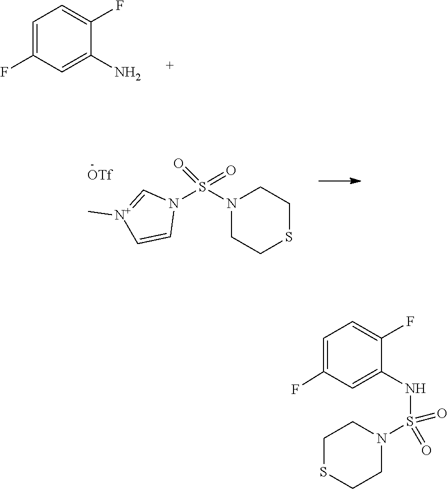 [SMILES: Nc1cc(F)ccc1F ; C[n+]1ccn(S(=O)(=O)N2CCSCC2)c1 ; O=S(=O)(Nc1cc(F)ccc1F)N1CCSCC1]

4-(3-methylimidazol-3-ium-1-yl)sulfonylthiomorpholine; trifluoromethanesulfonate (100.00%, 1.000 g, 2.516 mmol) and 2,5-difluoroaniline (100.00%, 1.100 equiv., 2.768 mmol) were dissolved in acetonitrile (30 mL) at 80° C., and then the resulting solution was stirred overnight at the same temperature and a temperature was lowered to room temperature to terminate the reaction. A saturated aqueous sodium hydrogen carbonate solution was poured into the reaction mixture, and extracted with ethyl acetate. An organic layer was washed with saturated aqueous sodium chloride solution, dehydrated with anhydrous magnesium sulfate, filtered, and concentrated under reduced pressure. The resulting concentrate was purified via column chromatography (SiO₂, 12 g cartridge; hexane/ethyl acetate=0 to 50%) and concentrated to obtain a title compound (0.500 g, 67.52%) in a white solid form.

### [Step 2] Synthesis of N-[[4-[5-(difluoromethyl)-1,3,4-oxadiazol-2-yl]-2-fluoro-phenyl]methyl]-N-(2,5-difluorophenyl)thiomorpholin-4-sulfonamide

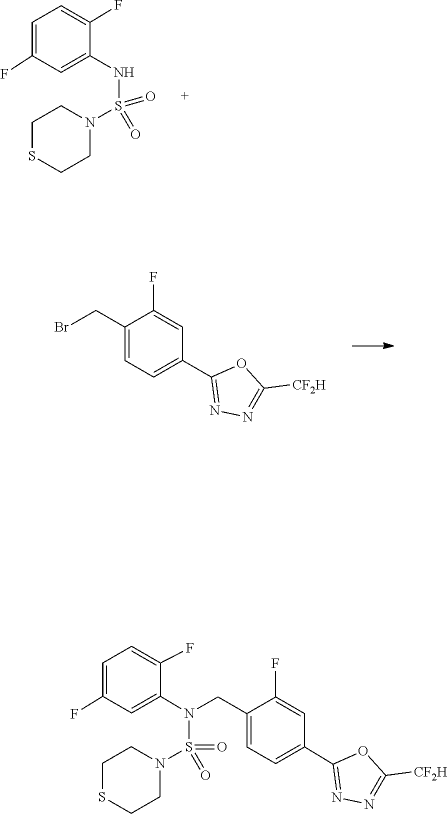 [SMILES: O=S(=O)(Nc1cc(F)ccc1F)N1CCSCC1 ; Fc1cc(-c2nnc(C(F)F)o2)ccc1CBr ; O=S(=O)(N1CCSCC1)N(Cc1ccc(-c2nnc(C(F)F)o2)cc1F)c1cc(F)ccc1F]

N-(2,5-difluorophenyl)thiomorpholin-4-sulfonamide (100.00%, 0.080 g, 0.272 mmol) prepared in step 1 was dissolved in N,N-dimethylformamide (10 mL) at 0° C., and then sodium hydride (100.00%, 1.500 equiv., 0.408 mmol) was added to the resulting solution and stirred at the same temperature for 30 minutes. 2-[4-(bromomethyl)-3-fluoro-phenyl]-5-(difluoromethyl)-1,3,4-oxadiazole (100.00%, 1.100 equiv., 0.299 mmol) was added into the reaction mixture and further stirred at room temperature for two hours. A saturated aqueous sodium hydrogen carbonate solution was poured into the reaction mixture, and extracted with ethyl acetate. An organic layer was washed with saturated aqueous sodium chloride solution, dehydrated with anhydrous magnesium sulfate, filtered, and concentrated under reduced pressure. The resulting concentrate was purified via column chromatography (SiO₂, 12 g cartridge; dichloromethane/methanol=0 to 10%), and concentrated to obtain a title compound (0.100 g, 70.68%) in a colorless oil form.

### [Step 3] Synthesis of Compound 69

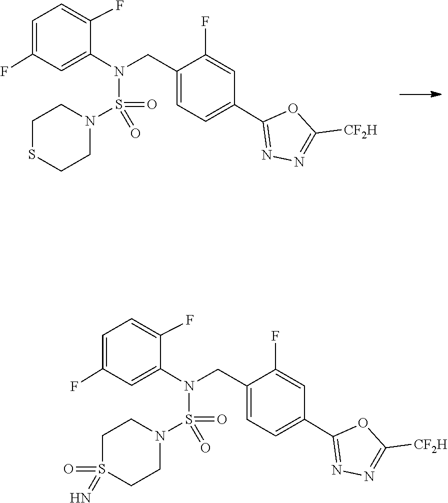 [SMILES: O=S(=O)(N1CCSCC1)N(Cc1ccc(-c2nnc(C(F)F)o2)cc1F)c1cc(F)ccc1F ; N=S1(=O)CCN(S(=O)(=O)N(Cc2ccc(-c3nnc(C(F)F)o3)cc2F)c2cc(F)ccc2F)CC1]

N-[[4-[5-(difluoromethyl)-1,3,4-oxadiazol-2-yl]-2-fluoro-phenyl]methyl]-N-(2,5-difluorophenyl)thiomorpholin-4-sulfonamide (100.00%, 0.100 g, 0.192 mmol) prepared in step 2, ammonium carbamate (100.00%, 2.000 equiv., 0.384 mmol) and (diacetoxyiodo)benzene (100.00%, 2.500 equiv., 0.480 mmol) were dissolved in methanol (10 mL) at room temperature, and then the resulting solution was stirred overnight at the same temperature. Solvent was removed from the reaction mixture under reduced pressure, and then a saturated aqueous sodium hydrogen carbonate solution was poured into the resulting concentrate and extracted with dichloromethane. An organic layer was washed with saturated aqueous sodium chloride solution, dehydrated with anhydrous magnesium sulfate, filtered, and concentrated under reduced pressure. The resulting concentrate was purified via column chromatography (SiO₂, 12 g cartridge; dichloromethane/methanol=0 to 10%), and concentrated to obtain a title compound (0.050 g, 47.19%) in a colorless oil form.

¹H NMR (400 MHz, CDCl₃) δ 7.87 (dd, J=8.0, 1.6 Hz, 1H), 7.78 (dd, J=9.6, 1.6 Hz, 1H), 7.52 (t, J=7.6 Hz, 1H), 7.14-7.03 (m, 3H), 7.02 (s, 0.25H), 7.69 (s, 0.5H), 6.80 (s, 0.25H), 4.90 (s, 2H), 3.92-3.73 (m, 4H), 3.22-3.10 (m, 4H), 2.14 (brs, 1H); LRMS (ES) m/z 552.9 (M⁺+1).

### Example 70: Synthesis of Compound 70, N-(3-chlorophenyl)-N-[[4-[5-(difluoromethyl)-1,3,4-oxadiazol-2-yl]-2-fluoro-phenyl]methyl]-1-imino-1-oxo-1,4-thiazinan-4-sulfonamide

### [Step 1] Synthesis of N-(3-chlorophenyl)thiomorpholin-4-sulfonamide

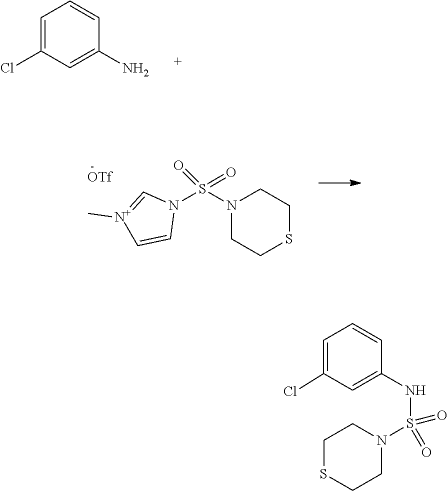 [SMILES: Nc1cccc(Cl)c1 ; C[n+]1ccn(S(=O)(=O)N2CCSCC2)c1 ; O=S(=O)(Nc1cccc(Cl)c1)N1CCSCC1]

4-(3-methylimidazol-3-ium-1-yl)sulfonylthiomorpholine; trifluoromethanesulfonate (100.00%, 0.991 g, 2.494 mmol) and 3-chloroaniline (100.00%, 1.100 equiv., 2.743 mmol) were dissolved in acetonitrile (20 mL) at 80° C., and then the resulting solution was stirred overnight at the same temperature and a temperature was lowered to room temperature to terminate the reaction. Solvent was removed from the resulting mixture under reduced pressure, and then the resulting concentrate was purified via column chromatography (SiO₂, 12 g cartridge; dichloromethane/methanol=0 to 10%) and concentrated to obtain a title compound (0.560 g, 76.70%) in a white solid form.

### [Step 2] Synthesis of N-(3-chlorophenyl)-N-[[4-[5-(difluoromethyl)-1,3,4-oxadiazol-2-yl]-2-fluoro-phenyl]methyl]thiomorpholin-4-sulfonamide

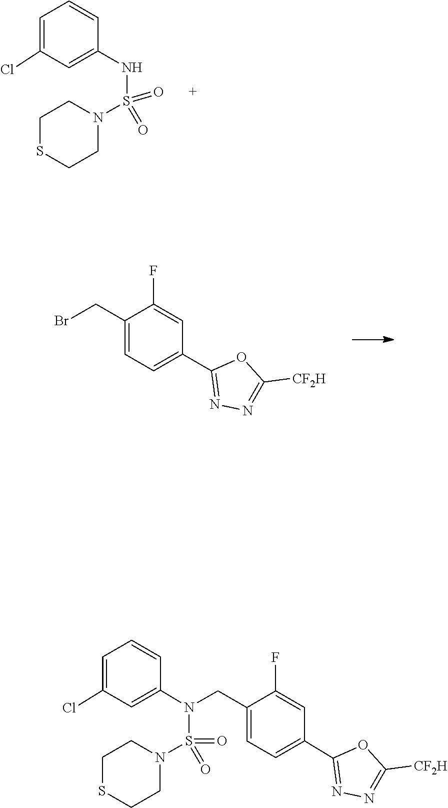 [SMILES: O=S(=O)(Nc1cccc(Cl)c1)N1CCSCC1 ; Fc1cc(-c2nnc(C(F)F)o2)ccc1CBr ; O=S(=O)(N1CCSCC1)N(Cc1ccc(-c2nnc(C(F)F)o2)cc1F)c1cccc(Cl)c1]

N-(3-chlorophenyl)thiomorpholin-4-sulfonamide (100.00%, 0.260 g, 0.888 mmol) prepared in step 1, 2-[4-(bromomethyl)-3-fluoro-phenyl]-5-(difluoromethyl)-1,3,4-oxadiazole (100.00%, 1.100 equiv., 0.977 mmol), potassium carbonate (100.00%, 1.500 equiv., 1.332 mmol) and potassium iodide (100.00%, 0.100 equiv., 0.089 mmol) were dissolved in N,N-dimethylformamide (10 mL) at room temperature, and then the resulting solution was stirred overnight at the same temperature. A saturated aqueous sodium hydrogen carbonate solution was poured into the reaction mixture, and extracted with ethyl acetate. An organic layer was washed with saturated aqueous sodium chloride solution, dehydrated with anhydrous magnesium sulfate, filtered, and concentrated under reduced pressure. The resulting concentrate was purified via column chromatography (SiO₂, 12 g cartridge; dichloromethane/methanol=0 to 10%), and concentrated to obtain a title compound (0.260 g, 56.42%) in a colorless oil form.

### [Step 3] Synthesis of Compound 70

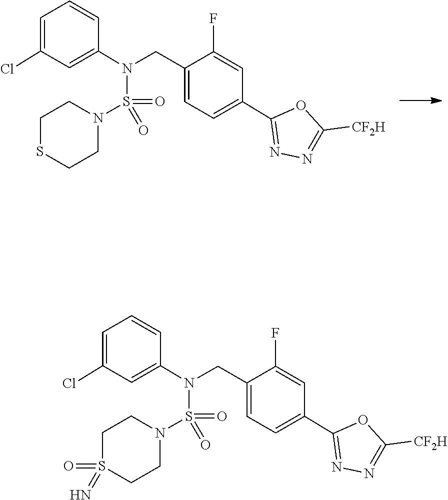 [SMILES: O=S(=O)(N1CCSCC1)N(Cc1ccc(-c2nnc(C(F)F)o2)cc1F)c1cccc(Cl)c1 ; N=S1(=O)CCN(S(=O)(=O)N(Cc2ccc(-c3nnc(C(F)F)o3)cc2F)c2cccc(Cl)c2)CC1]

N-(3-chlorophenyl)-N-[[4-[5-(difluoromethyl)-1,3,4-oxadiazol-2-yl]-2-fluoro-phenyl]methyl]thiomorpholin-4-sulfonamide (100.00%, 0.260 g, 0.501 mmol) prepared in step 2, ammonium carbamate (100.00%, 2.000 equiv., 1.002 mmol) and (diacetoxyiodo)benzene (100.00%, 2.500 equiv., 1.252 mmol) were dissolved in methanol (10 mL) at room temperature, and then the resulting solution was stirred overnight at the same temperature. Solvent was removed from the reaction mixture under reduced pressure, and then a saturated aqueous sodium hydrogen carbonate solution was poured into the resulting concentrate and extracted with dichloromethane. An organic layer was washed with saturated aqueous sodium chloride solution, dehydrated with anhydrous magnesium sulfate, filtered, and concentrated under reduced pressure. The resulting concentrate was purified via column chromatography (SiO₂, 12 g cartridge; dichloromethane/methanol=0 to 10%), and concentrated to obtain a title compound (0.200 g, 72.59%) in a colorless oil form.

¹H NMR (400 MHz, CDCl₃) δ 7.86 (dd, J=8.0, 1.2 Hz, 1H), 7.78 (dd, J=10.0, 1.6 Hz, 1H), 7.47 (t, J=7.6 Hz, 1H), 7.34-7.27 (m, 3H), 7.18-7.15 (m, 1H), 7.06 (s, 0.25H), 6.93 (s, 0.5H), 6.80 (s, 0.25H), 4.92 (s, 2H), 3.82-3.66 (m, 4H), 3.20-3.09 (m, 4H), 2.10 (brs, 1H); LRMS (ES) m/z 550.9 (M⁺+1).

### Example 71: Synthesis of Compound 71, N-(3-chlorophenyl)-N-[[5-[5-(difluoromethyl)-1,3,4-oxadiazol-2-yl]-2-pyridyl]methyl]-1-imino-1-oxo-1,4-thiazinan-4-sulfonamide

### [Step 1] Synthesis of N-(3-chlorophenyl)-N-[[5-[5-(difluoromethyl)-1,3,4-oxadiazol-2-yl]-2-pyridyl]methyl]thiomorpholin-4-sulfonamide

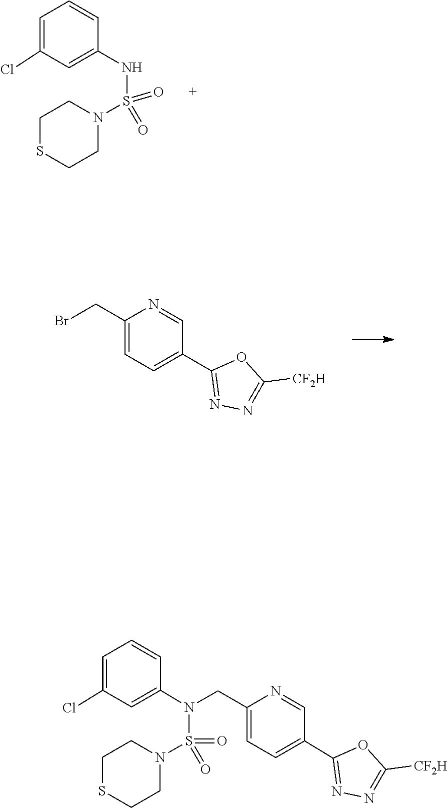 [SMILES: O=S(=O)(Nc1cccc(Cl)c1)N1CCSCC1 ; FC(F)c1nnc(-c2ccc(CBr)nc2)o1 ; O=S(=O)(N1CCSCC1)N(Cc1ccc(-c2nnc(C(F)F)o2)cn1)c1cccc(Cl)c1]

N-(3-chlorophenyl)thiomorpholin-4-sulfonamide (100.00%, 0.240 g, 0.820 mmol), 2-[6-(bromomethyl)-3-pyridyl]-5-(difluoromethyl)-1,3,4-oxadiazole (100.00%, 1.100 equiv., 0.902 mmol), potassium carbonate (100.00%, 1.500 equiv., 1.230 mmol) and potassium iodide (100.00%, 0.100 equiv., 0.082 mmol) were dissolved in N,N-dimethylformamide (10 mL) at room temperature, and then the resulting solution was stirred overnight at the same temperature. A saturated aqueous sodium hydrogen carbonate solution was poured into the reaction mixture, and extracted with ethyl acetate. An organic layer was washed with saturated aqueous sodium chloride solution, dehydrated with anhydrous magnesium sulfate, filtered, and concentrated under reduced pressure. The resulting concentrate was purified via column chromatography (SiO₂, 12 g cartridge; dichloromethane/methanol=0 to 10%), and concentrated to obtain a title compound (0.219 g, 53.22%) in a colorless oil form.

### [Step 2] Synthesis of Compound 71

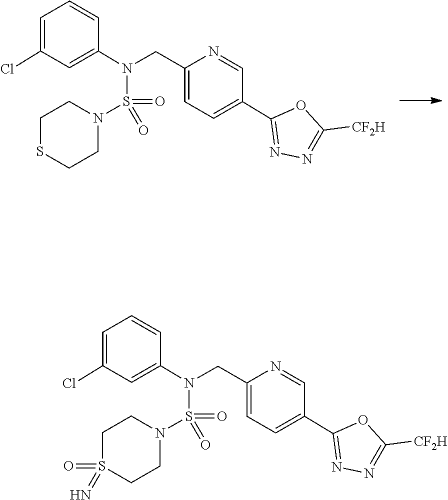 [SMILES: O=S(=O)(N1CCSCC1)N(Cc1ccc(-c2nnc(C(F)F)o2)cn1)c1cccc(Cl)c1 ; N=S1(=O)CCN(S(=O)(=O)N(Cc2ccc(-c3nnc(C(F)F)o3)cn2)c2cccc(Cl)c2)CC1]

N-(3-chlorophenyl)-N-[[5-[5-(difluoromethyl)-1,3,4-oxadiazol-2-yl]-2-pyridyl]methyl]thiomorpholin-4-sulfonamide (100.00%, 0.219 g, 0.436 mmol) prepared in step 1, ammonium carbamate (100.00%, 2.000 equiv., 0.873 mmol) and (diacetoxyiodo)benzene (100.00%, 2.500 equiv., 1.091 mmol) were dissolved in methanol (10 mL) at room temperature, and then the resulting solution was stirred overnight at the same temperature. Solvent was removed from the reaction mixture under reduced pressure, and then a saturated aqueous sodium hydrogen carbonate solution was poured into the resulting concentrate and extracted with dichloromethane. An organic layer was washed with saturated aqueous sodium chloride solution, dehydrated with anhydrous magnesium sulfate, filtered, and concentrated under reduced pressure. The resulting concentrate was purified via column chromatography (SiO₂, 12 g cartridge; dichloromethane/methanol=0 to 10%), and concentrated to obtain a title compound (0.150 g, 64.51%) in a colorless oil form.

¹H NMR (400 MHz, CDCl₃) δ 9.29 (t, J=1.0 Hz, 1H), 8.39 (dd, J=8.2, 2.2 Hz, 1H), 7.55 (d, J=8.4 Hz, 1H), 7.48-7.47 (m, 1H), 7.32-7.29 (m, 3H), 7.09 (s, 0.25H), 6.96 (s, 0.5H), 6.83 (s, 0.25H), 5.04 (s, 2H), 3.87-3.74 (m, 4H), 3.15-3.11 (m, 4H), 2.20 (brs, 1H); LRMS (ES) m/z 533.9 (M⁺+1).

### Example 72: Synthesis of Compound 72, N-[[5-[5-(difluoromethyl)-1,3,4-oxadiazol-2-yl]-2-pyridyl]methyl]-1-imino-N-(3-methoxyphenyl)-1-oxo-1,4-thiazinan-4-sulfonamide

### [Step 1] Synthesis of N-(3-methoxyphenyl)thiomorpholin-4-sulfonamide

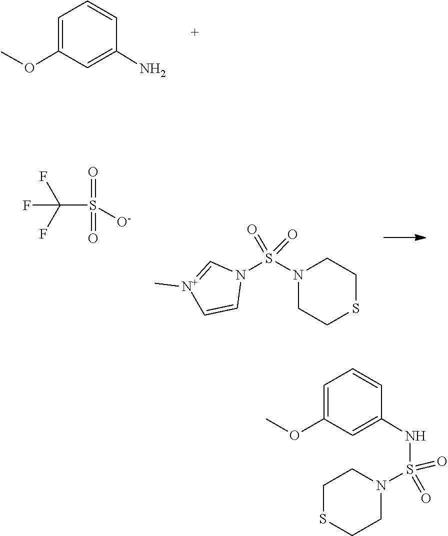

4-(3-methylimidazol-3-ium-1-yl)sulfonylthiomorpholine; trifluoromethanesulfonate salt (100.00%, 0.580 g, 1.459 mmol) and 3-methoxyaniline (100.00% solution, 0.14 mL, 1.246 mmol) were dissolved in acetonitrile (10 mL) at room temperature, and then the resulting solution was heated under reflux for 18 hours, and a temperature was lowered to room temperature to terminate the reaction. Solvent was removed from the reaction mixture under reduced pressure, and then saturated aqueous sodium hydrogen carbonate solution was poured into the resulting concentrate, extracted with dichloromethane, filtered via a plastic filter to remove a solid residue and an aqueous solution layer therefrom, and concentrated under reduced pressure. The resulting concentrate was purified via column chromatography (SiO₂, 12 g cartridge; ethyl acetate/hexane=5 to 20%), and concentrated to obtain a title compound (0.151 g, 35.87%) in a brown oil form.

### [Step 2] Synthesis of N-[[5-[5-(difluoromethyl)-1,3,4-oxadiazol-2-yl]-2-pyridyl]methyl]-N-(3-methoxyphenyl)thiomorpholin-4-sulfonamide

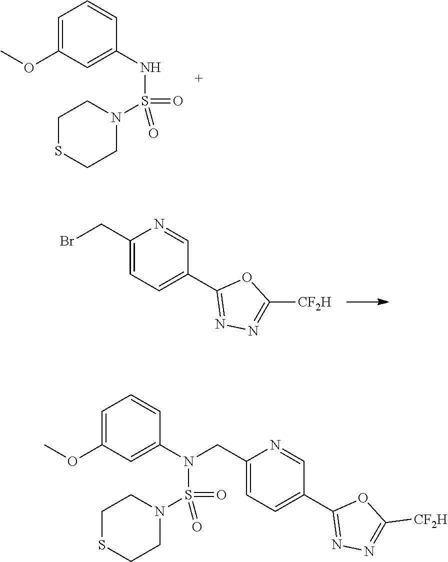 [SMILES: COc1cccc(NS(=O)(=O)N2CCSCC2)c1 ; FC(F)c1nnc(-c2ccc(CBr)nc2)o1 ; COc1cccc(N(Cc2ccc(-c3nnc(C(F)F)o3)cn2)S(=O)(=O)N2CCSCC2)c1]

N-(3-methoxyphenyl)thiomorpholin-4-sulfonamide (100.00%, 0.075 g, 0.260 mmol) and sodium hydride (60.00%, 0.013 g, 0.325 mmol) were dissolved in N,N-dimethylformamide (3 mL) at 0° C., and then 2-[6-(bromomethyl)-3-pyridyl]-5-(difluoromethyl)-1,3,4-oxadiazole (100.00%, 0.086 g, 0.296 mmol) was added into the resulting solution and stirred at room temperature for 18 hours. Solvent was removed from the reaction mixture under reduced pressure, and then saturated aqueous sodium hydrogen carbonate solution was poured into the resulting concentrate, extracted with dichloromethane, filtered via a plastic filter to remove a solid residue and an aqueous solution layer therefrom, and concentrated under reduced pressure. The resulting concentrate was purified via column chromatography (SiO₂, 4 g cartridge; ethyl acetate/hexane=10 to 50%), and concentrated to obtain a title compound (0.106 g, 81.93%) in a light yellow oil form.

### [Step 3] Synthesis of Compound 72

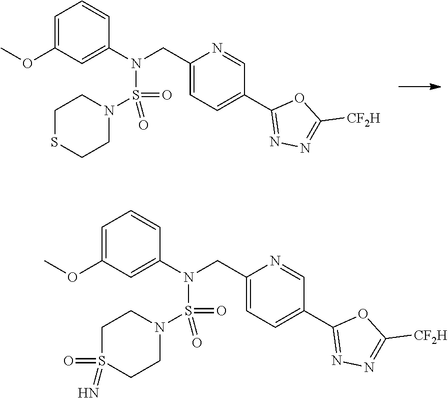 [SMILES: COc1cccc(N(Cc2ccc(-c3nnc(C(F)F)o3)cn2)S(=O)(=O)N2CCSCC2)c1 ; COc1cccc(N(Cc2ccc(-c3nnc(C(F)F)o3)cn2)S(=O)(=O)N2CCS(=N)(=O)CC2)c1]

N-[[5-[5-(difluoromethyl)-1,3,4-oxadiazol-2-yl]-2-pyridyl]methyl]-N-(3-methoxyphenyl)thiomorpholin-4-sulfonamide (100.00%, 0.106 g, 0.213 mmol), iodobenzene diacetate (100.00%, 0.172 g, 0.534 mmol) and ammonium carbamate (100.00%, 0.033 g, 0.423 mmol) were dissolved in methanol (3 mL) at room temperature, and then the resulting solution was stirred at 50° C. for six hours, after which a temperature was lowered to room temperature to terminate the reaction. Solvent was removed from the resulting mixture under reduced pressure, and then the resulting concentrate was purified via column chromatography (SiO₂, 4 g cartridge; methanol/dichloromethane=0 to 5%) and concentrated to obtain a title compound (0.057 g, 50.61%) in a light orange-colored solid form.

¹H NMR (400 MHz, CDCl₃) δ 9.25 (d, J=1.60 Hz, 1H), 8.36 (dd, J=8.2, 2.2 Hz, 1H), 7.58 (d, J=8.00 Hz, 1H), 7.26-7.24 (m, 1H), 7.08-6.82 (m, 4H), 5.05 (s, 2H), 3.85-3.79 (m, 5H), 3.75-3.71 (m, 2H), 3.12-3.07 (m, 4H); LRMS (ES) m/z 529.8 (M⁺+1).

### Example 73: Synthesis of Compound 73, N-[[4-[5-(difluoromethyl)-1,3,4-oxadiazol-2-yl]-2-fluoro-phenyl]methyl]-1-imino-N-(3-methoxyphenyl)-1-oxo-1,4-thiazinan-4-sulfonamide

### [Step 1] Synthesis of N-[[4-[5-(difluoromethyl)-1,3,4-oxadiazol-2-yl]-2-fluoro-phenyl]methyl]-N-(3-methoxyphenyl)thiomorpholin-4-sulfonamide

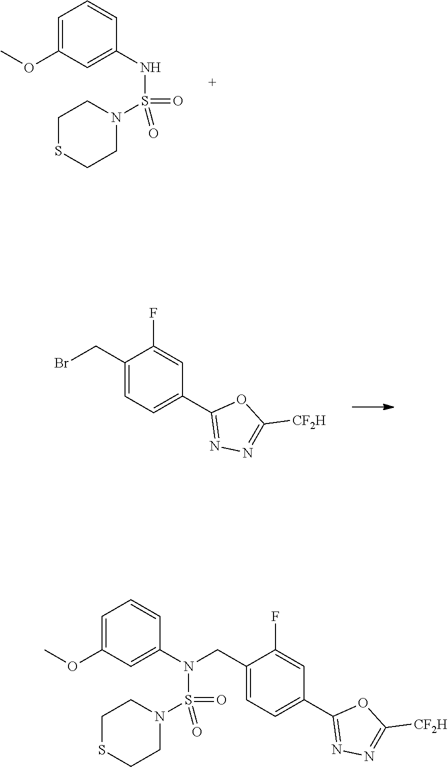 [SMILES: COc1cccc(NS(=O)(=O)N2CCSCC2)c1 ; Fc1cc(-c2nnc(C(F)F)o2)ccc1CBr ; COc1cccc(N(Cc2ccc(-c3nnc(C(F)F)o3)cc2F)S(=O)(=O)N2CCSCC2)c1]

N-(3-methoxyphenyl)thiomorpholin-4-sulfonamide (100.00%, 0.075 g, 0.260 mmol) and sodium hydride (60.00%, 0.013 g, 0.325 mmol) were dissolved in N,N-dimethylformamide (3 mL) at 0° C., and then 2-[4-(bromomethyl)-3-fluoro-phenyl]-5-(difluoromethyl)-1,3,4-oxadiazole (100.00%, 0.091 g, 0.296 mmol) was added into the resulting solution and stirred at room temperature for 18 hours. Solvent was removed from the reaction mixture under reduced pressure, and then saturated aqueous sodium hydrogen carbonate solution was poured into the resulting concentrate, extracted with dichloromethane, filtered via a plastic filter to remove a solid residue and an aqueous solution layer therefrom, and concentrated under reduced pressure. The resulting concentrate was purified via column chromatography (SiO₂, 4 g cartridge; ethyl acetate/hexane=10 to 50%), and concentrated to obtain a title compound (0.085 g, 63.53%) in a light yellow oil form.

### [Step 2] Synthesis of Compound 73

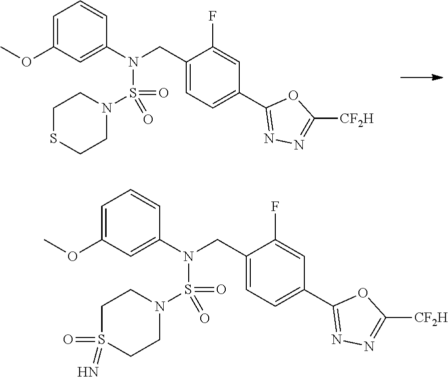 [SMILES: COc1cccc(N(Cc2ccc(-c3nnc(C(F)F)o3)cc2F)S(=O)(=O)N2CCSCC2)c1 ; COc1cccc(N(Cc2ccc(-c3nnc(C(F)F)o3)cc2F)S(=O)(=O)N2CCS(=N)(=O)CC2)c1]

N-[[4-[5-(difluoromethyl)-1,3,4-oxadiazol-2-yl]-2-fluoro-phenyl]methyl]-N-(3-methoxyphenyl)thiomorpholin-4-sulfonamide (100.00%, 0.085 g, 0.165 mmol), iodobenzene diacetate (100.00%, 0.143 g, 0.444 mmol) and ammonium carbamate (100.00%, 0.028 g, 0.359 mmol) were dissolved in methanol (3 mL) at room temperature, and then the resulting solution was stirred at 50° C. for six hours, after which a temperature was lowered to room temperature to terminate the reaction. Solvent was removed from the resulting mixture under reduced pressure, and then the resulting concentrate was purified via column chromatography (SiO₂, 4 g cartridge; methanol/dichloromethane=0 to 5%) and concentrated to obtain a title compound (0.051 g, 56.58%) in a white solid form.

¹H NMR (400 MHz, CDCl₃) δ 7.85 (dd, J=8.0, 1.5 Hz, 1H), 7.77 (dd, J=9.9, 1.5 Hz, 1H), 7.51 (t, J=7.64 Hz, 1H), 7.26-7.24 (m, 1H), 7.05-6.79 (m, 4H), 4.94 (s, 2H), 3.78-3.75 (m, 5H), 3.72-3.65 (m, 2H), 3.13-3.09 (m, 4H); LRMS (ES) m/z 546.5 (M⁺+1).

### Example 74: Synthesis of Compound 74, N-[[4-[5-(difluoromethyl)-1,3,4-oxadiazol-2-yl]-2-fluoro-phenyl]methyl]-2-(1-imino-1-oxo-1,4-thiazinan-4-yl)-N-phenyl-ethanesulfonamide

### [Step 1] Synthesis of N-phenylethenesulfonamide

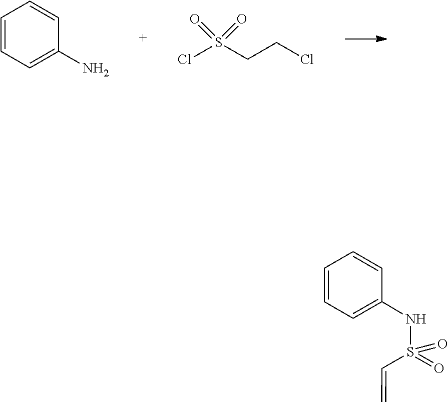

Aniline (100.00%, 1.000 g, 10.740 mmol), 2-chloroethanesulfonyl chloride (100.00%, 1.000 equiv., 10.740 mmol), and triethylamine (100.00%, 2.000 equiv., 21.480 mmol) were dissolved in dichloromethane (50 mL) at room temperature, and then the resulting solution was stirred overnight at the same temperature. A saturated aqueous sodium hydrogen carbonate solution was poured into the reaction mixture and extracted with dichloromethane. An organic layer was washed with saturated aqueous sodium chloride solution, dehydrated with anhydrous magnesium sulfate, filtered, and concentrated under reduced pressure. The resulting concentrate was purified via column chromatography (SiO₂, 40 g cartridge; hexane/ethyl acetate=0 to 30%) and concentrated to obtain a title compound (0.738 g, 37.51%) as a colorless oil form.

### [Step 2] Synthesis of N-[[4-[5-(difluoromethyl)-1,3,4-oxadiazol-2-yl]-2-fluoro-phenyl]methyl]-N-phenyl-ethenesulfonamide

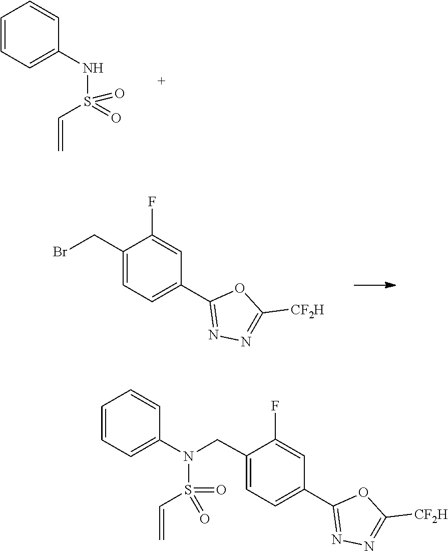 [SMILES: C=CS(=O)(=O)Nc1ccccc1 ; Fc1cc(-c2nnc(C(F)F)o2)ccc1CBr ; C=CS(=O)(=O)N(Cc1ccc(-c2nnc(C(F)F)o2)cc1F)c1ccccc1]

N-phenylethenesulfonamide (100.00%, 0.738 g, 4.028 mmol) prepared in step 1, 2-[4-(bromomethyl)-3-fluoro-phenyl]-5-(difluoromethyl)-1,3,4-oxadiazole (100.00%, 1.000 equiv., 4.028 mmol), potassium carbonate (100.00%, 1.500 equiv., 6.042 mmol) and potassium iodide (100.00%, 0.100 equiv., 0.403 mmol) were dissolved in N,N-dimethylformamide (30 mL) at room temperature, and then the resulting solution was stirred overnight at the same temperature. A saturated aqueous sodium hydrogen carbonate solution was poured into the reaction mixture, and extracted with ethyl acetate. An organic layer was washed with saturated aqueous sodium chloride solution, dehydrated with anhydrous magnesium sulfate, filtered, and concentrated under reduced pressure. The resulting concentrate was purified via column chromatography (SiO₂, 40 g cartridge; hexane/ethyl acetate=0 to 50%) and concentrated to obtain a title compound (1.530 g, 92.79%) as a colorless oil form.

### [Step 3] Synthesis of N-[[4-[5-(difluoromethyl)-1,3,4-oxadiazol-2-yl]-2-fluoro-phenyl]methyl]-N-phenyl-2-thiomorpholino-ethanesulfonamide

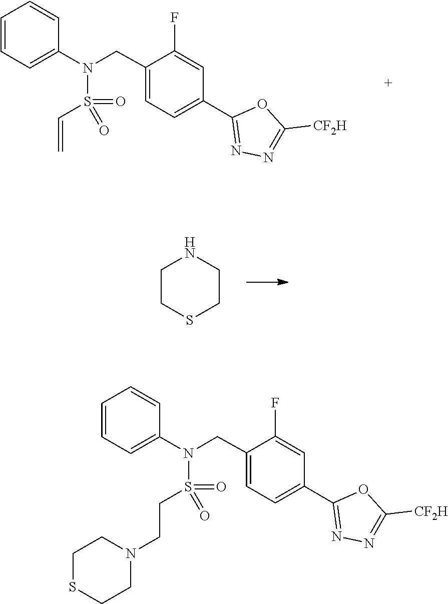 [SMILES: C=CS(=O)(=O)N(Cc1ccc(-c2nnc(C(F)F)o2)cc1F)c1ccccc1 ; C1CSCCN1 ; O=S(=O)(CCN1CCSCC1)N(Cc1ccc(-c2nnc(C(F)F)o2)cc1F)c1ccccc1]

N-[[4-[5-(difluoromethyl)-1,3,4-oxadiazol-2-yl]-2-fluoro-phenyl]methyl]-N-phenyl-ethenesulfonamide (100.00%, 1.530 g, 3.737 mmol) prepared in step 2, thiomorpholine (100.00%, 1.300 equiv., 4.858 mmol) and N,N-diisopropylethylamine (100.00%, 2.000 equiv., 7.474 mmol) were dissolved in dichloromethane (30 mL) at room temperature, and then the resulting solution was stirred overnight at the same temperature. A saturated aqueous sodium hydrogen carbonate solution was poured into the reaction mixture and extracted with dichloromethane. An organic layer was washed with saturated aqueous sodium chloride solution, dehydrated with anhydrous magnesium sulfate, filtered, and concentrated under reduced pressure. The resulting concentrate was purified via column chromatography (SiO₂, 40 g cartridge; hexane/ethyl acetate=0 to 100%) and concentrated to obtain a title compound (0.917 g, 47.87%) as a colorless oil form.

### [Step 4] Synthesis of N-[[4-[5-(difluoromethyl)-1,3,4-oxadiazol-2-yl]-2-fluoro-phenyl]methyl]-2-(1-oxo-1,4-thiazinan-4-yl)-N-phenyl-ethanesulfonamide

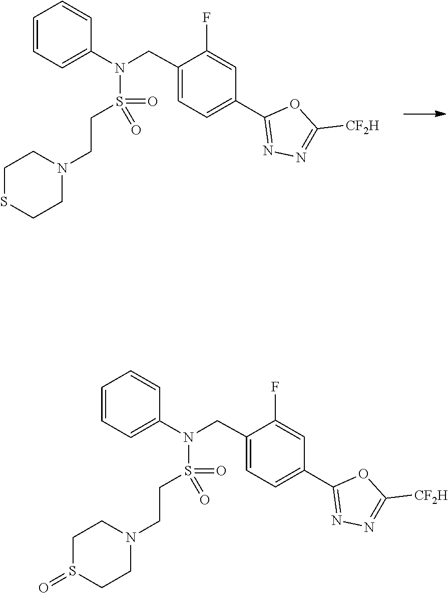 [SMILES: O=S(=O)(CCN1CCSCC1)N(Cc1ccc(-c2nnc(C(F)F)o2)cc1F)c1ccccc1 ; O=S1CCN(CCS(=O)(=O)N(Cc2ccc(-c3nnc(C(F)F)o3)cc2F)c2ccccc2)CC1]

N-[[4-[5-(difluoromethyl)-1,3,4-oxadiazol-2-yl]-2-fluoro-phenyl]methyl]-N-phenyl-2-thiomorpholino-ethanesulfonamide (100.00%, 0.900 g, 1.756 mmol) prepared in step 3, (diacetoxyiodo)benzene (100.00%, 2.500 equiv., 4.389 mmol) and ammonium carbamate (100.00%, 2.000 equiv., 3.512 mmol) were dissolved in methanol (10 mL) at room temperature, and then the resulting solution was stirred overnight at the same temperature. Solvent was removed from the reaction mixture under reduced pressure, and then a saturated aqueous sodium hydrogen carbonate solution was poured into the resulting concentrate and extracted with dichloromethane. An organic layer was washed with saturated aqueous sodium chloride solution, dehydrated with anhydrous magnesium sulfate, filtered, and concentrated under reduced pressure. The resulting concentrate was purified via column chromatography (SiO₂, 12 g cartridge; dichloromethane/methanol=0 to 10%), and concentrated to obtain a title compound (0.239 g, 25.75%) in a colorless oil form.

### [Step 5] Synthesis of Compound 74

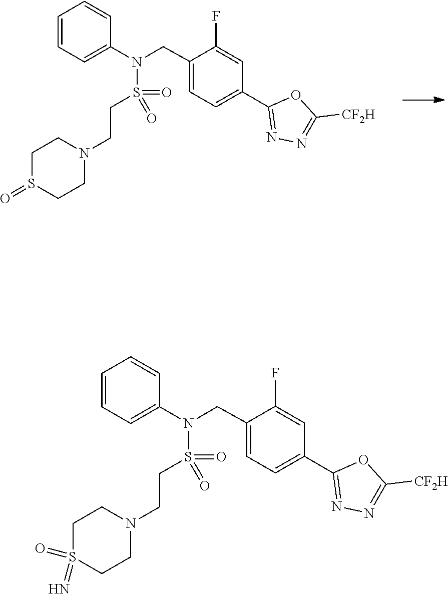 [SMILES: O=S1CCN(CCS(=O)(=O)N(Cc2ccc(-c3nnc(C(F)F)o3)cc2F)c2ccccc2)CC1 ; N=S1(=O)CCN(CCS(=O)(=O)N(Cc2ccc(-c3nnc(C(F)F)o3)cc2F)c2ccccc2)CC1]

N-[[4-[5-(difluoromethyl)-1,3,4-oxadiazol-2-yl]-2-fluoro-phenyl]methyl]-2-(1-oxo-1,4-thiazinan-4-yl)-N-phenyl-ethanesulfonamide (100.00%, 0.239 g, 0.452 mmol) prepared in step 4, ammonium carbamate (100.00%, 2.000 equiv., 0.904 mmol) and (diacetoxyiodo)benzene (100.00%, 2.500 equiv., 1.130 mmol) were dissolved in methanol (10 mL) at room temperature, and then the resulting solution was stirred overnight at the same temperature. Solvent was removed from the reaction mixture under reduced pressure, and then a saturated aqueous sodium hydrogen carbonate solution was poured into the resulting concentrate and extracted with dichloromethane. An organic layer was washed with saturated aqueous sodium chloride solution, dehydrated with anhydrous magnesium sulfate, filtered, and concentrated under reduced pressure. The resulting concentrate was purified via column chromatography (SiO₂, 12 g cartridge; dichloromethane/methanol=0 to 10%), and concentrated to obtain a title compound (0.030 g, 12.21%) in a colorless oil form.

¹H NMR (400 MHz, CDCl₃) δ 7.87 (dd, J=8.0, 1.4 Hz, 1H), 7.75 (dd, J=9.9, 1.5 Hz, 1H), 7.63 (t, J=7.7 Hz, 1H), 7.40-7.31 (m, 5H), 7.05 (s, 0.25H), 6.92 (s, 0.5H), 6.79 (s, 0.25H), 5.05 (s, 2H), 3.31-3.27 (m, 2H), 3.17-3.03 (m, 10H), 2.52 (s, 1H); LRMS (ES) m/z 444.3 (M⁺+1).

### Example 75: Synthesis of Compound 75, N-[[4-[5-(difluoromethyl)-1,3,4-oxadiazol-2-yl]-2-fluoro-phenyl]methyl]-4-(methylsulfonimidoyl)-N-phenyl-piperidin-1-carboxamide

### [Step 1] Synthesis of 4-methylsulfyl-N-phenyl-piperidin-1-carboxamide

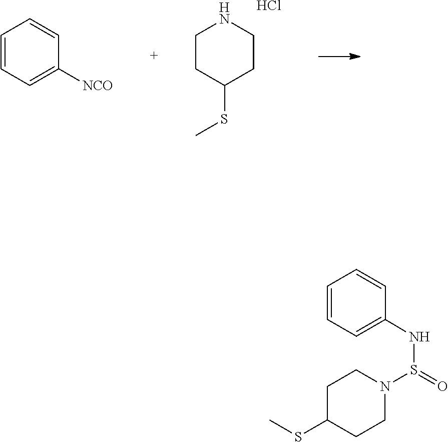

4-methylsulfylpiperidine; hydrochloride (100.00%, 0.500 g, 2.982 mmol), isocyanatobenzene (100.00%, 1.000 equiv., 2.982 mmol) and N,N-diisopropylethylamine (100.00%, 1.000 equiv., 2.982 mmol) were dissolved in dichloromethane (10 mL) at room temperature, and then the resulting solution was stirred overnight at the same temperature. Solvent was removed from the reaction mixture under reduced pressure, and then dichloromethane (20 mL) and methanol (10 mL) were added into the resulting concentrate and stirred to filter out a precipitated solid, which was then washed with hexane and dried to obtain a title compound (0.400 g, 53.58%) in a white solid form.

### [Step 2] Synthesis of N-[[4-[5-(difluoromethyl)-1,3,4-oxadiazol-2-yl]-2-fluoro-phenyl]methyl]-4-methylsulfyl-N-phenyl-piperidin-1-carboxamide

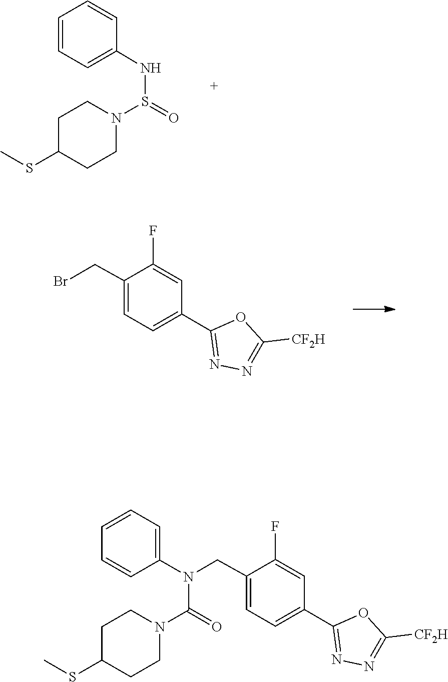 [SMILES: CSC1CCN(S(=O)Nc2ccccc2)CC1 ; Fc1cc(-c2nnc(C(F)F)o2)ccc1CBr ; CSC1CCN(C(=O)N(Cc2ccc(-c3nnc(C(F)F)o3)cc2F)c2ccccc2)CC1]

4-methylsulfyl-N-phenyl-piperidin-1-carboxamide (100.00%, 0.240 g, 0.959 mmol) prepared in step 1 was dissolved in N,N-dimethylformamide (10 mL) at 0° C., and then sodium hydride (100.00%, 1.500 equiv., 1.438 mmol) was added to the resulting solution and stirred at the same temperature for 30 minutes. 2-[4-(bromomethyl)-3-fluoro-phenyl]-5-(difluoromethyl)-1,3,4-oxadiazole (100.00%, 1.100 equiv., 1.054 mmol) was added into the reaction mixture and further stirred at room temperature for two hours. A saturated aqueous sodium hydrogen carbonate solution was poured into the reaction mixture, and extracted with ethyl acetate. An organic layer was washed with saturated aqueous sodium chloride solution, dehydrated with anhydrous magnesium sulfate, filtered, and concentrated under reduced pressure. The resulting concentrate was purified via column chromatography (SiO₂, 12 g cartridge; dichloromethane/methanol=0 to 10%), and concentrated to obtain a title compound (0.025 g, 5.47%) in a colorless oil form.

### [Step 3] Synthesis of Compound 75

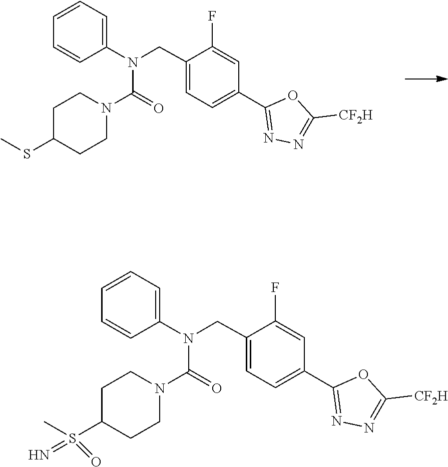 [SMILES: CSC1CCN(C(=O)N(Cc2ccc(-c3nnc(C(F)F)o3)cc2F)c2ccccc2)CC1 ; CS(=N)(=O)C1CCN(C(=O)N(Cc2ccc(-c3nnc(C(F)F)o3)cc2F)c2ccccc2)CC1]

N-[[4-[5-(difluoromethyl)-1,3,4-oxadiazol-2-yl]-2-fluoro-phenyl]methyl]-4-methylsulfyl-N-phenyl-piperidin-1-carboxamide (100.00%, 0.025 g, 0.052 mmol) prepared in step 2, ammonium carbamate (100.00%, 2.000 equiv., 0.105 mmol) and (diacetoxyiodo)benzene (100.00%, 2.500 equiv., 0.131 mmol) were dissolved in methanol (10 mL) at room temperature, and then the resulting solution was stirred overnight at the same temperature. Solvent was removed from the reaction mixture under reduced pressure, and then a saturated aqueous sodium hydrogen carbonate solution was poured into the resulting concentrate and extracted with dichloromethane. An organic layer was washed with saturated aqueous sodium chloride solution, dehydrated with anhydrous magnesium sulfate, filtered, and concentrated under reduced pressure. The resulting concentrate was purified via column chromatography (SiO₂, 12 g cartridge; dichloromethane/methanol=0 to 10%), and concentrated to obtain a title compound (0.005 g, 18.78%) in a colorless oil form.

¹H NMR (400 MHz, CDCl₃) δ 7.87 (dd, J=8.0, 1.5 Hz, 1H), 7.75 (dd, J=10.1, 1.5 Hz, 1H), 7.70 (t, J=7.6 Hz, 1H), 7.36-7.32 (m, 2H), 7.19-7.17 (m, 3H), 7.05 (s, 0.25H), 6.92 (s, 0.5H), 6.79 (s, 0.25H), 4.96 (s, 2H), 4.10-4.07 (m, 2H), 2.94-2.88 (m, 2H), 2.84 (s, 3H), 2.68-2.60 (m, 2H), 2.45 (s, 1H), 2.01-1.97 (m, 2H), 1.51-1.44 (m, 2H); LRMS (ES) m/z 508.9 (M⁺+1).

### Example 76: Synthesis of Compound 76, N-[[5-[5-(difluoromethyl)-1,3,4-oxadiazol-2-yl]-2-pyridyl]methyl]-N-(4-fluorophenyl)-1-imino-1-oxo-1,4-thiazinan-4-sulfonamide

### [Step 1] Synthesis of N-(4-fluorophenyl)thiomorpholin-4-sulfonamide

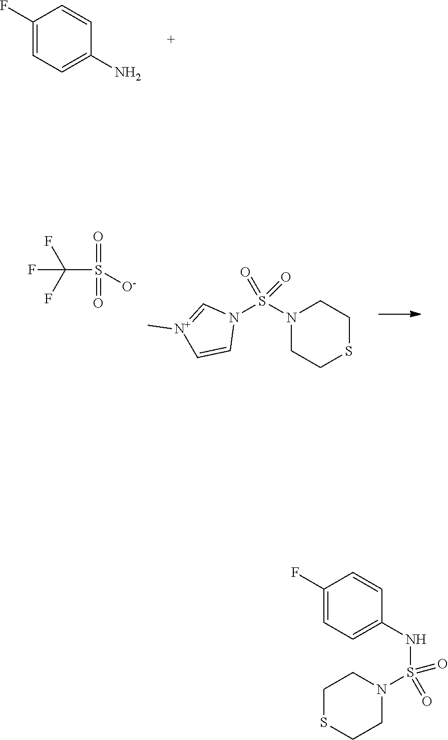 [SMILES: Nc1ccc(F)cc1 ; O=S(=O)([O-])C(F)(F)F ; C[n+]1ccn(S(=O)(=O)N2CCSCC2)c1 ; O=S(=O)(Nc1ccc(F)cc1)N1CCSCC1]

4-(3-methylimidazol-3-ium-1-yl)sulfonylthiomorpholine; trifluoromethanesulfonate (100.00%, 1.000 g, 2.516 mmol) and 4-fluoroaniline (100.00% solution, 0.238 mL, 2.512 mmol) were dissolved in acetonitrile (10 mL) at room temperature, and then the resulting solution was heated under reflux for 18 hours, and a temperature was lowered to room temperature to terminate the reaction. Solvent was removed from the reaction mixture under reduced pressure, and then saturated aqueous sodium hydrogen carbonate solution was poured into the resulting concentrate, extracted with dichloromethane, filtered via a plastic filter to remove a solid residue and an aqueous solution layer therefrom, and concentrated under reduced pressure. The resulting concentrate was purified via column chromatography (SiO₂, 12 g cartridge; ethyl acetate/hexane=5 to 20%) and concentrated to obtain a title compound (0.305 g, 43.85%) in a brown solid form.

### [Step 2] Synthesis of N-[[5-[5-(difluoromethyl)-1,3,4-oxadiazol-2-yl]-2-pyridyl]methyl]-N-(4-fluorophenyl)thiomorpholin-4-sulfonamide

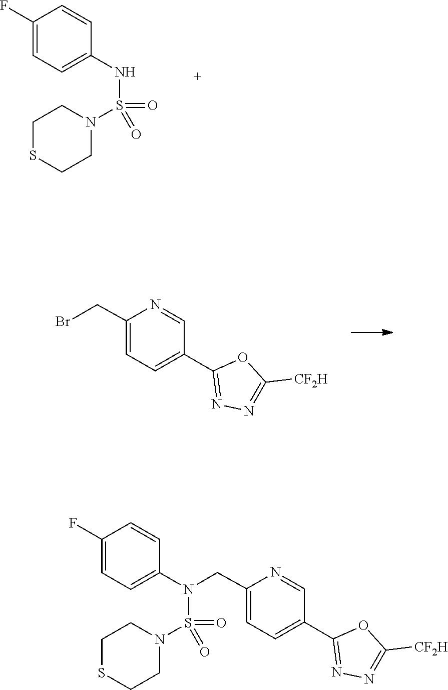 [SMILES: O=S(=O)(Nc1ccc(F)cc1)N1CCSCC1 ; FC(F)c1nnc(-c2ccc(CBr)nc2)o1 ; O=S(=O)(N1CCSCC1)N(Cc1ccc(-c2nnc(C(F)F)o2)cn1)c1ccc(F)cc1]

N-(4-fluorophenyl)thiomorpholin-4-sulfonamide (100.00%, 0.100 g, 0.362 mmol) and sodium hydride (60.00%, 0.016 g, 0.400 mmol) were dissolved in N,N-dimethylformamide (3 mL) at 0° C., and then 2-[6-(bromomethyl)-3-pyridyl]-5-(difluoromethyl)-1,3,4-oxadiazole (100.00%, 0.105 g, 0.362 mmol) was added into the resulting solution and stirred at room temperature for 18 hours. Solvent was removed from the reaction mixture under reduced pressure, and then N-dichloromethane aqueous solution was poured into the resulting concentrate, extracted with dichloromethane, filtered via a plastic filter to remove a solid residue and an aqueous solution layer therefrom, and concentrated under reduced pressure. The resulting concentrate was purified via column chromatography (SiO₂, 4 g cartridge; ethyl acetate/hexane=10 to 50%), and concentrated to obtain a title compound (0.080 g, 45.54%) in a light yellow oil form.

### [Step 3] Synthesis of Compound 76

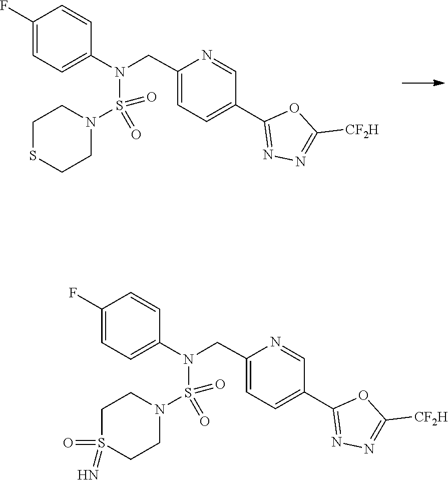 [SMILES: O=S(=O)(N1CCSCC1)N(Cc1ccc(-c2nnc(C(F)F)o2)cn1)c1ccc(F)cc1 ; N=S1(=O)CCN(S(=O)(=O)N(Cc2ccc(-c3nnc(C(F)F)o3)cn2)c2ccc(F)cc2)CC1]

N-[[5-[5-(difluoromethyl)-1,3,4-oxadiazol-2-yl]-2-pyridyl]methyl]-N-(4-fluorophenyl)thiomorpholin-4-sulfonamide (100.00%, 0.080 g, 0.165 mmol), iodobenzene diacetate (100.00%, 0.143 g, 0.444 mmol) and ammonium carbamate (100.00%, 0.028 g, 0.359 mmol) were dissolved in methanol (3 mL) at room temperature, and then the resulting solution was stirred at 50° C. for six hours, after which a temperature was lowered to room temperature to terminate the reaction. Solvent was removed from the resulting mixture under reduced pressure, and then the resulting concentrate was purified via column chromatography (SiO₂, 4 g cartridge; methanol/dichloromethane=0 to 5%) and concentrated to obtain a title compound (0.068 g, 79.90%) in a light orange-colored solid form.

¹H NMR (400 MHz, CDCl₃) δ 9.27 (d, J=1.52 Hz, 1H), 8.37 (dd, J=8.2, 2.2 Hz, 1H), 7.54 (d, J=8.20 Hz, 1H), 7.40-7.37 (m, 2H), 7.08-6.83 (m, 3H), 5.01 (s, 2H), 3.86-3.80 (m, 2H), 3.76-3.70 (m, 2H), 3.19-3.09 (m, 4H); LRMS (ES) m/z 517.8 (M⁺+1).

### Example 77: Synthesis of Compound 77, N-[[5-[5-(difluoromethyl)-1,3,4-oxadiazol-2-yl]-3-fluoro-2-pyridyl]methyl]-N-(4-fluorophenyl)-1-imino-1-oxo-1,4-thiazinan-4-sulfonamide

### [Step 1] Synthesis of N-[[5-[5-(difluoromethyl)-1,3,4-oxadiazol-2-yl]-3-fluoro-2-pyridyl]methyl]-N-(4-fluorophenyl)thiomorpholin-4-sulfonamide

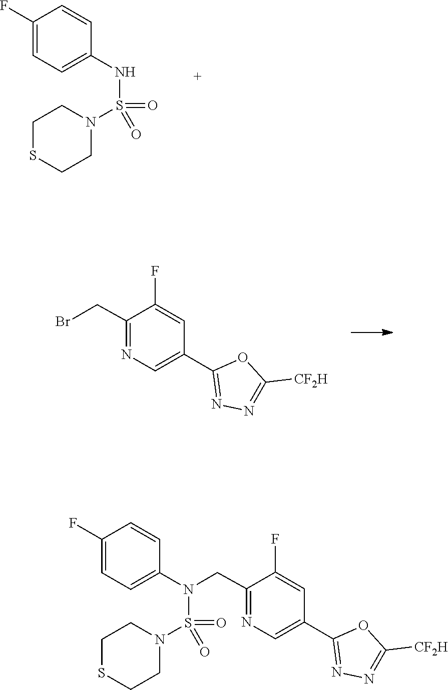 [SMILES: O=S(=O)(Nc1ccc(F)cc1)N1CCSCC1 ; Fc1cc(-c2nnc(C(F)F)o2)cnc1CBr ; O=S(=O)(N1CCSCC1)N(Cc1ncc(-c2nnc(C(F)F)o2)cc1F)c1ccc(F)cc1]

N-(4-fluorophenyl)thiomorpholin-4-sulfonamide (100.00%, 0.100 g, 0.362 mmol) and sodium hydride (60.00%, 0.016 g, 0.400 mmol) were dissolved in N,N-dimethylformamide (3 mL) at 0° C., and then 2-[6-(bromomethyl)-5-fluoro-3-pyridyl]-5-(difluoromethyl)-1,3,4-oxadiazole (100.00%, 0.111 g, 0.360 mmol) was added into the resulting solution and stirred at room temperature for 18 hours. Solvent was removed from the reaction mixture under reduced pressure, and then N-dichloromethane aqueous solution was poured into the resulting concentrate, extracted with dichloromethane, filtered via a plastic filter to remove a solid residue and an aqueous solution layer therefrom, and concentrated under reduced pressure. The resulting concentrate was purified via column chromatography (SiO₂, 4 g cartridge; ethyl acetate/hexane=10 to 50%), and concentrated to obtain a title compound (0.126 g, 69.17%) in a light yellow oil form.

### [Step 2] Synthesis of Compound 77

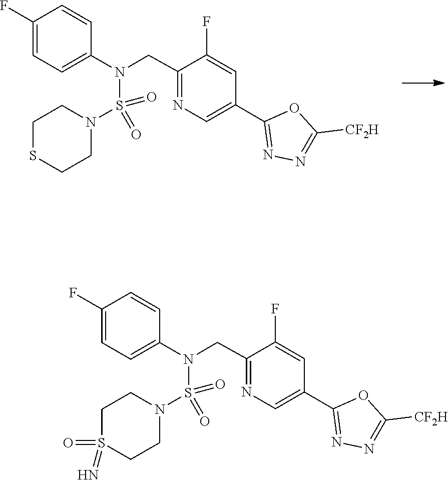 [SMILES: O=S(=O)(N1CCSCC1)N(Cc1ncc(-c2nnc(C(F)F)o2)cc1F)c1ccc(F)cc1 ; N=S1(=O)CCN(S(=O)(=O)N(Cc2ncc(-c3nnc(C(F)F)o3)cc2F)c2ccc(F)cc2)CC1]

N-[[5-[5-(difluoromethyl)-1,3,4-oxadiazol-2-yl]-3-fluoro-2-pyridyl]methyl]-N-(4-fluorophenyl)thiomorpholin-4-sulfonamide (100.00%, 0.126 g, 0.250 mmol), iodobenzene diacetate (100.00%, 0.217 g, 0.674 mmol) and ammonium carbamate (100.00%, 0.042 g, 0.538 mmol) were dissolved in methanol (3 mL) at room temperature, and then the resulting solution was stirred at 50° C. for six hours, after which a temperature was lowered to room temperature to terminate the reaction. Solvent was removed from the resulting mixture under reduced pressure, and then the resulting concentrate was purified via column chromatography (SiO₂, 4 g cartridge; methanol/dichloromethane=0 to 5%) and concentrated to obtain a title compound (0.027 g, 20.19%) in a light yellow solid form.

¹H NMR (400 MHz, CDCl₃) δ 9.14 (d, J=0.72 Hz, 1H), 8.07 (dd, J=9.2, 1.7 Hz, 1H), 7.43-7.39 (m, 2H), 7.09-6.83 (m, 3H), 5.06 (s, 2H), 3.88-3.83 (m, 2H), 3.79-3.73 (m, 2H), 3.16-3.11 (m, 4H); LRMS (ES) m/z 535.8 (M⁺+1).

### Example 78: Synthesis of Compound 78, N-[[4-[5-(difluoromethyl)-1,3,4-oxadiazol-2-yl]-2-fluoro-phenyl]methyl]-N-(4-fluorophenyl)-1-imino-1-oxo-1,4-thiazinan-4-sulfonamide

### [Step 1] Synthesis of N-[[4-[5-(difluoromethyl)-1,3,4-oxadiazol-2-yl]-2-fluoro-phenyl]methyl]-N-(4-fluorophenyl)thiomorpholin-4-sulfonamide

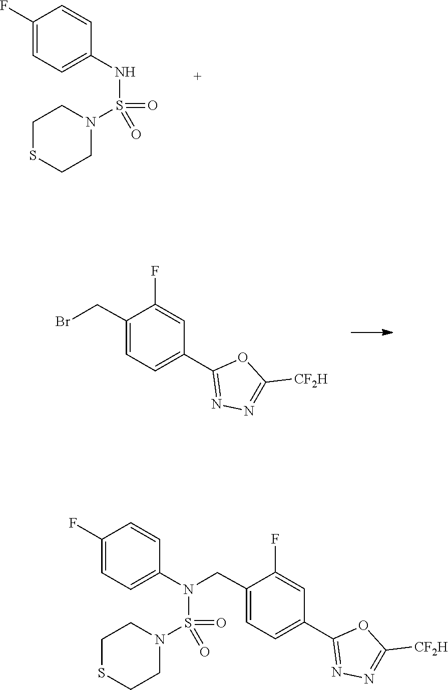 [SMILES: O=S(=O)(Nc1ccc(F)cc1)N1CCSCC1 ; Fc1cc(-c2nnc(C(F)F)o2)ccc1CBr ; O=S(=O)(N1CCSCC1)N(Cc1ccc(-c2nnc(C(F)F)o2)cc1F)c1ccc(F)cc1]

N-(4-fluorophenyl)thiomorpholin-4-sulfonamide (100.00%, 0.100 g, 0.362 mmol) and sodium hydride (60.00%, 0.016 g, 0.400 mmol) were dissolved in N,N-dimethylformamide (3 mL) at 0° C., and then 2-[4-(bromomethyl)-3-fluoro-phenyl]-5-(difluoromethyl)-1,3,4-oxadiazole (100.00%, 0.111 g, 0.361 mmol) was added into the resulting solution and stirred at room temperature for 18 hours. Solvent was removed from the reaction mixture under reduced pressure, and then N-dichloromethane aqueous solution was poured into the resulting concentrate, extracted with dichloromethane, filtered via a plastic filter to remove a solid residue and an aqueous solution layer therefrom, and concentrated under reduced pressure. The resulting concentrate was purified via column chromatography (SiO₂, 4 g cartridge; ethyl acetate/hexane=10 to 50%), and concentrated to obtain a title compound (0.090 g, 49.50%) in a light yellow oil form.

### [Step 2] Synthesis of Compound 78

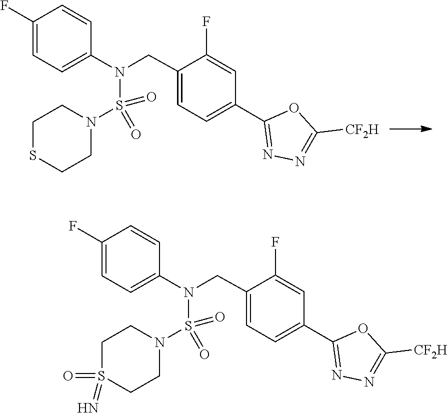 [SMILES: O=S(=O)(N1CCSCC1)N(Cc1ccc(-c2nnc(C(F)F)o2)cc1F)c1ccc(F)cc1 ; N=S1(=O)CCN(S(=O)(=O)N(Cc2ccc(-c3nnc(C(F)F)o3)cc2F)c2ccc(F)cc2)CC1]

N-[[4-[5-(difluoromethyl)-1,3,4-oxadiazol-2-yl]-2-fluoro-phenyl]methyl]-N-(4-fluorophenyl)thiomorpholin-4-sulfonamide (100.00%, 0.090 g, 0.179 mmol), iodobenzene diacetate (100.00%, 0.155 g, 0.481 mmol) and ammonium carbamate (100.00%, 0.030 g, 0.384 mmol) were dissolved in methanol (3 mL) at room temperature, and then the resulting solution was stirred at 50° C. for six hours, after which a temperature was lowered to room temperature to terminate the reaction. Solvent was removed from the resulting mixture under reduced pressure, and then the resulting concentrate was purified via column chromatography (SiO₂, 4 g cartridge; methanol/dichloromethane=0 to 5%) and concentrated to obtain a title compound (0.080 g, 83.72%) in a white solid form.

¹H NMR (400 MHz, CDCl₃) δ 7.85 (dd, J=8.0, 1.5 Hz, 1H), 7.77 (dd, J=9.9, 1.5 Hz, 1H), 7.50 (t, J=7.60 Hz, 1H), 7.27-7.23 (m, 2H), 7.07-6.80 (m, 3H), 4.89 (s, 2H), 3.79-3.73 (m, 2H), 3.70-3.64 (m, 2H), 3.18-3.10 (m, 4H); LRMS (ES) m/z 534.8 (M⁺+1).

### Example 79: Synthesis of Compound 79, Benzyl N-[4-[[6-[5-(difluoromethyl)-1,3,4-oxadiazol-2-yl]pyridazin-3-yl]methyl-(3-fluorophenyl)carbamoyl]-1-oxo-1,4-thiazinan-1-ylidene]carbamate

### [Step 1] Synthesis of benzyl N-[4-[(3-fluorophenyl)carbamoyl]-1-oxo-1,4-thiazinan-1-ylidene]carbamate

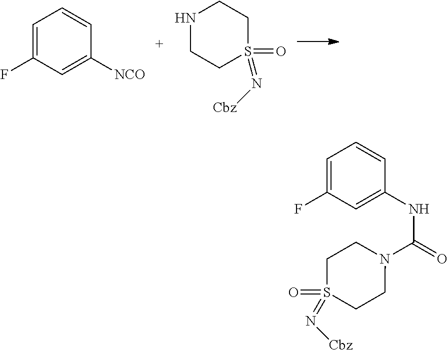

1-fluoro-3-isocyanato-benzene (100.00% solution, 0.41 mL, 3.591 mmol) and benzyl N-(1-oxo-1,4-thiazinan-1-ylidene)carbamate (100.00%, 0.978 g, 3.645 mmol) were dissolved in diethyl ether (40 mL) at room temperature, and then the resulting solution was stirred at the same temperature for two hours. The resulting precipitated solid was filtered, washed with diethyl ether, and dried to obtain a title compound (1.390 g, 95.47%) in a white solid form.

### [Step 2] Synthesis of Compound 79

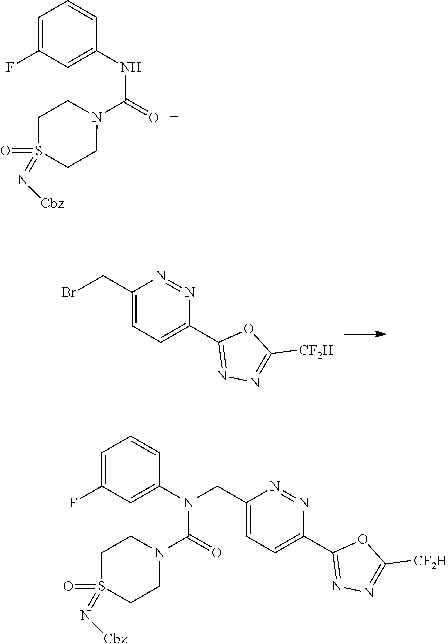

Benzyl N-[4-[(3-fluorophenyl)carbamoyl]-1-oxo-1,4-thiazinan-1-ylidene]carbamate (100.00%, 0.270 g, 0.666 mmol), 2-[6-(bromomethyl)pyridazin-3-yl]-5-(difluoromethyl)-1,3,4-oxadiazole (100.00%, 0.194 g, 0.667 mmol) and cesium carbonate (100.00%, 0.434 g, 1.332 mmol) were dissolved in acetonitrile (5 mL), and then the resulting solution was stirred at room temperature for 10 minutes and further stirred at the same temperature for three hours. Solvent was removed from the reaction mixture under reduced pressure, and then water was poured into the resulting concentrate, extracted with dichloromethane, filtered via a plastic filter to remove a solid residue and an aqueous solution layer therefrom, and concentrated under reduced pressure. The resulting concentrate was purified via column chromatography (SiO₂, 4 g cartridge; ethyl acetate/hexane=20 to 80%), and concentrated to obtain a title compound (0.105 g, 25.61%) in a yellow solid form.

¹H NMR (400 MHz, CDCl₃) δ 8.44 (d, J=8.7 Hz, 1H), 7.83 (d, J=8.7 Hz, 1H), 7.39-7.31 (m, 6H), 5.29 (s, 2H), 3.93-3.89 (m, 2H), 3.58-3.53 (m, 4H), 3.27-3.21 (m, 2H); LRMS (ES) m/z 616.9 (M⁺+1).

### Example 80: Synthesis of Compound 80, N-[[6-[5-(difluoromethyl)-1,3,4-oxadiazol-2-yl]pyridazin-3-yl]methyl]-N-(3-fluorophenyl)-1-imino-1-oxo-1,4-thiazinan-4-carboxamide

### [Step 1] Synthesis of Compound 80

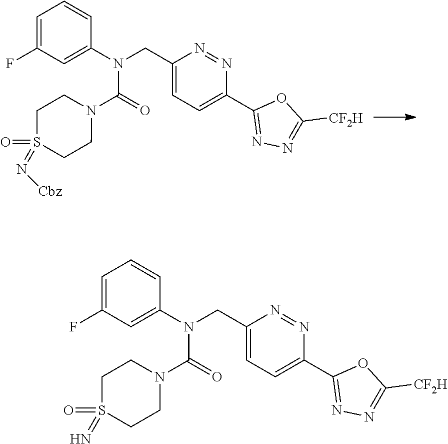 [SMILES: O=C(N=S1(=O)CCN(C(=O)N(Cc2ccc(-c3nnc(C(F)F)o3)nn2)c2cccc(F)c2)CC1)OCc1ccccc1 ; N=S1(=O)CCN(C(=O)N(Cc2ccc(-c3nnc(C(F)F)o3)nn2)c2cccc(F)c2)CC1]

Benzyl N-[4-[[6-[5-(difluoromethyl)-1,3,4-oxadiazol-2-yl]pyridazin-3-yl]methyl-(3-fluorophenyl)carbamoyl]-1-oxo-1,4-thiazinan-1-ylidene]carbamate (100.00%, 0.100 g, 0.162 mmol) was dissolved in methanol (2 mL)/tetrahydrofuran (2 mL) and stirred at room temperature, and then 10%-Pd/C (10 mg) was slowly added thereinto at the same temperature, and stirred for three hours in the presence of a hydrogen balloon attached thereto at the same temperature. The reaction mixture was filtered via a celite pad to remove a solid therefrom, and then solvent was removed from the resulting filtrate without the solid under reduced pressure. Then, the resulting concentrate was purified via column chromatography (SiO₂, 12 g cartridge; ethyl acetate=100%, methanol/dichloromethane=0 to 5%), and concentrated to obtain a title compound (0.054 g, 69.05%) in a light yellow solid form.

¹H NMR (400 MHz, CDCl₃) δ 8.42 (d, J=8.7 Hz, 1H), 7.87 (d, J=8.7 Hz, 1H), 7.38-7.32 (m, 1H), 7.12-6.87 (m, 4H), 5.29 (s, 2H), 3.82-3.76 (m, 2H), 3.71-3.65 (m, 2H), 2.98-2.97 (m, 4H); LRMS (ES) m/z 482.5 (M⁺+1).

### Example 81: Synthesis of Compound 81, N-[[5-[5-(difluoromethyl)-1,3,4-oxadiazol-2-yl]pyrazin-2-yl]methyl]-N-(3-fluorophenyl)-1-imino-1-oxo-1,4-thiazinan-4-carboxamide

### [Step 1] Synthesis of benzyl N-[4-[[5-[5-(difluoromethyl)-1,3,4-oxadiazol-2-yl]pyrazin-2-yl]methyl-(3-fluorophenyl)carbamoyl]-1-oxo-1,4-thiazinan-1-ylidene]carbamate

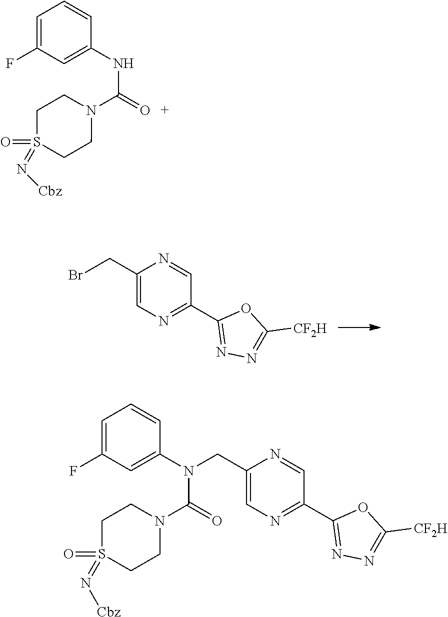

Benzyl N-[4-[(3-fluorophenyl)carbamoyl]-1-oxo-1,4-thiazinan-1-ylidene]carbamate (100.00%, 0.300 g, 0.740 mmol), 2-[5-(bromomethyl)pyrazin-2-yl]-5-(difluoromethyl)-1,3,4-oxadiazole (100.00%, 0.214 g, 0.735 mmol) and cesium carbonate (100.00%, 0.482 g, 1.479 mmol) were dissolved in acetonitrile (5 mL) at room temperature, and then the resulting solution was stirred at the same temperature for 18 hours. Solvent was removed from the reaction mixture under reduced pressure, and then water was poured into the resulting concentrate, extracted with dichloromethane, filtered via a plastic filter to remove a solid residue and an aqueous solution layer therefrom, and concentrated under reduced pressure. The resulting concentrate was purified via column chromatography (SiO₂, 4 g cartridge; ethyl acetate/hexane=20 to 80%), and concentrated to obtain a product, after which the resulting product was purified again via chromatography (SiO₂ plate, 20×20×1 mm; ethyl acetate/hexane=50%), and concentrated to obtain a title compound (0.040 g, 8.78%) in a yellow solid form.

### [Step 2] Synthesis of Compound 81

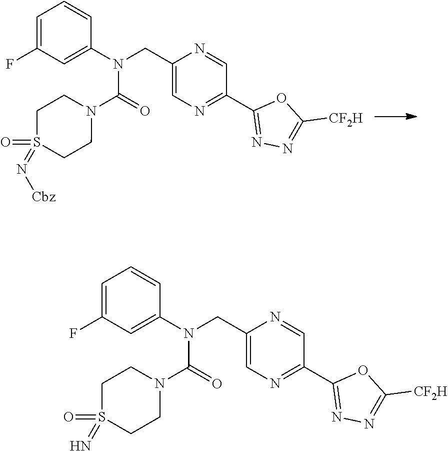 [SMILES: O=C(N=S1(=O)CCN(C(=O)N(Cc2cnc(-c3nnc(C(F)F)o3)cn2)c2cccc(F)c2)CC1)OCc1ccccc1 ; N=S1(=O)CCN(C(=O)N(Cc2cnc(-c3nnc(C(F)F)o3)cn2)c2cccc(F)c2)CC1]

Benzyl N-[4-[[5-[5-(difluoromethyl)-1,3,4-oxadiazol-2-yl]pyrazin-2-yl]methyl-(3-fluorophenyl)carbamoyl]-1-oxo-1,4-thiazinan-1-ylidene]carbamate (100.00%, 0.040 g, 0.065 mmol) was dissolved in methanol (2 mL)/tetrahydrofuran (1 mL) and stirred at room temperature, and then 10%-Pd/C (5 mg) was slowly added thereinto at the same temperature, and stirred for 20 minutes in the presence of a hydrogen balloon attached thereto at the same temperature. The reaction mixture was filtered via a celite pad to remove a solid therefrom, and then solvent was removed from the resulting filtrate without the solid under reduced pressure. Then, the resulting concentrate was purified via chromatography (SiO₂ plate, 20×20×1 mm; ethyl acetate/=100%), and concentrated to obtain a title compound (0.005 g, 15.98%) in a light yellow solid form.

¹H NMR (400 MHz, CDCl₃) δ 9.43 (d, J=1.2 Hz, 1H), 8.86 (d, J=1.0 Hz, 1H), 7.40-7.34 (m, 1H), 7.11-6.86 (m, 4H), 5.15 (s, 2H), 3.81-3.76 (m, 2H), 3.71-3.65 (m, 2H), 2.99-2.98 (m, 4H); LRMS (ES) m/z 482.8 (M⁺+1).

### Example 82: Synthesis of Compound 82, N-[[4-[5-(difluoromethyl)-1,3,4-oxadiazol-2-yl]-2-fluoro-phenyl]methyl]-1-imino-2-methyl-1-oxo-N-phenyl-1,4-thiazinan-4-carboxamide

### [Step 1] Synthesis of 2-methyl-N-phenyl-thiomorpholin-4-carboxamide

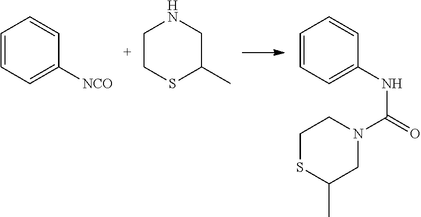

2-methylthiomorpholine (100.00%, 0.500 g, 4.266 mmol) and isocyanatobenzene (100.00%, 1.000 equiv., 4.266 mmol) were dissolved in dichloromethane (10 mL) at room temperature, and then the resulting solution was stirred overnight at the same temperature. Solvent was removed from the reaction mixture under reduced pressure, and then dichloromethane (5 mL) and hexane (30 mL) were added into the resulting concentrate and stirred to filter out a precipitated solid, which was then washed with hexane and dried to obtain a title compound (0.600 g, 59.52%) in a white solid form.

### [Step 2] Synthesis of N-[[4-[5-(difluoromethyl)-1,3,4-oxadiazol-2-yl]-2-fluoro-phenyl]methyl]-2-methyl-N-phenyl-thiomorpholin-4-carboxamide

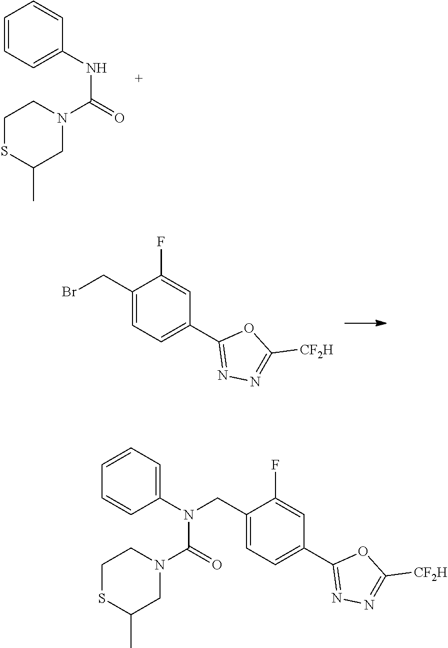

2-methyl-N-phenyl-thiomorpholin-4-carboxamide (100.00%, 0.200 g, 0.846 mmol) prepared in step 1 was dissolved in N,N-dimethylformamide (10 mL) at 0° C., and then sodium hydride (100.00%, 1.500 equiv., 1.270 mmol) was added to the resulting solution and stirred at the same temperature for 30 minutes. 2-[4-(bromomethyl)-3-fluoro-phenyl]-5-(difluoromethyl)-1,3,4-oxadiazole (100.00%, 1.100 equiv., 0.931 mmol) was added into the reaction mixture and further stirred at room temperature for two hours. A saturated aqueous sodium hydrogen carbonate solution was poured into the reaction mixture, and extracted with ethyl acetate. An organic layer was washed with saturated aqueous sodium chloride solution, dehydrated with anhydrous magnesium sulfate, filtered, and concentrated under reduced pressure. The resulting concentrate was purified via column chromatography (SiO₂, 12 g cartridge; hexane/ethyl acetate=0 to 50%) and concentrated to obtain a title compound (0.161 g, 41.13%) as a colorless oil form.

### [Step 3] Synthesis of Compound 82

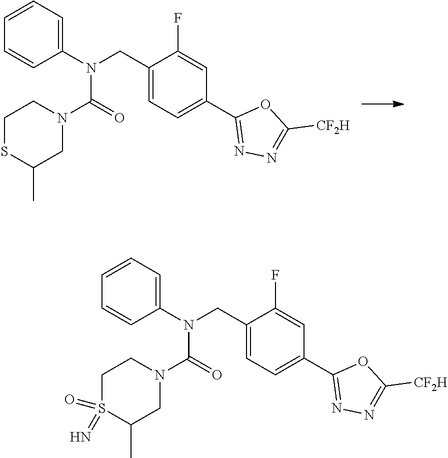 [SMILES: CC1CN(C(=O)N(Cc2ccc(-c3nnc(C(F)F)o3)cc2F)c2ccccc2)CCS1 ; CC1CN(C(=O)N(Cc2ccc(-c3nnc(C(F)F)o3)cc2F)c2ccccc2)CCS1(=N)=O]

N-[[4-[5-(difluoromethyl)-1,3,4-oxadiazol-2-yl]-2-fluoro-phenyl]methyl]-2-methyl-N-phenyl-thiomorpholin-4-carboxamide (100.00%, 0.160 g, 0.346 mmol) prepared in step 2, (diacetoxyiodo)benzene (100.00%, 2.500 equiv., 0.865 mmol) and ammonium carbamate (100.00%, 3.000 equiv., 1.038 mmol) were dissolved in methanol (10 mL) at room temperature, and then the resulting solution was stirred overnight at the same temperature. Solvent was removed from the reaction mixture under reduced pressure, and then saturated aqueous sodium hydrogen carbonate solution was poured into the resulting concentrate and extracted with ethyl acetate. An organic layer was washed with saturated aqueous sodium chloride solution, dehydrated with anhydrous magnesium sulfate, filtered, and concentrated under reduced pressure. The resulting concentrate was purified via column chromatography (SiO₂, 12 g cartridge; dichloromethane/methanol=0 to 10%), and concentrated to obtain a title compound (0.110 g, 64.43%) in a colorless oil form.

¹H NMR (400 MHz, CDCl₃) δ 7.89 (dd, J=8.0, 1.6 Hz, 1H), 7.76 (dd, J=10.2, 1.4 Hz, 1H), 7.70 (t, J=7.6 Hz, 1H), 7.40-7.37 (m, 2H), 7.26-7.22 (m, 1H), 7.15-7.11 (m, 2H), 7.02 (s, 0.25H), 6.93 (s, 0.5H), 6.80 (s, 0.25H), 5.00-4.89 (m, 2H), 4.08-3.92 (m, 2H), 3.45-3.34 (m, 1H), 3.10-2.73 (m, 4H), 1.70 (brs, 1H), 1.19-1.14 (m, 3H); LRMS (ES) m/z 494.9 (M⁺+1).

### Example 83: Synthesis of Compound 83, N-[[4-[5-(difluoromethyl)-1,3,4-oxadiazol-2-yl]-2-fluoro-phenyl]methyl]-6-(1-imino-1-oxo-thiethan-3-yl)-N-phenyl-2,6-diazaspiro[3.3]heptan-2-carboxamide

### [Step 1] Synthesis of N-[[4-[5-(difluoromethyl)-1,3,4-oxadiazol-2-yl]-2-fluoro-phenyl]methyl]-N-phenyl-6-(thiethan-3-yl)-2,6-diazaspiro[3.3]heptan-2-carboxamide

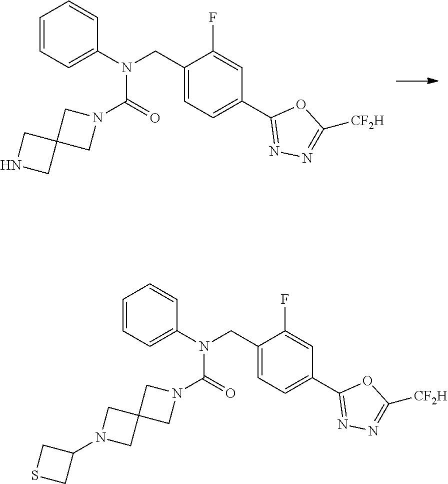 [SMILES: O=C(N1CC2(CNC2)C1)N(Cc1ccc(-c2nnc(C(F)F)o2)cc1F)c1ccccc1 ; O=C(N1CC2(C1)CN(C1CSC1)C2)N(Cc1ccc(-c2nnc(C(F)F)o2)cc1F)c1ccccc1]

N-[[4-[5-(difluoromethyl)-1,3,4-oxadiazol-2-yl]-2-fluoro-phenyl]methyl]-N-phenyl-2,6-diazaspiro[3.3]heptan-2-carboxamide (100.00%, 0.200 g, 0.451 mmol) and thietan-3-one (100.00%, 0.060 g, 0.681 mmol) were dissolved in dichloromethane (4 mL) at room temperature, and then sodium triacetoxyborohydride (100.00%, 0.190 g, 0.901 mmol) was added into the resulting solution and stirred at the same temperature for 18 hours. A saturated aqueous sodium hydrogen carbonate solution was poured into the reaction mixture and extracted with dichloromethane. An organic layer was washed with saturated aqueous sodium chloride solution, dehydrated with anhydrous magnesium sulfate, filtered, and concentrated under reduced pressure. The resulting concentrate was purified via column chromatography (SiO₂, 4 g cartridge; methanol/dichloromethane=0 to 2.5%) and concentrated to obtain a title compound (0.155 g, 66.65%) as a colorless oil form.

### [Step 2] Synthesis of Compound 83

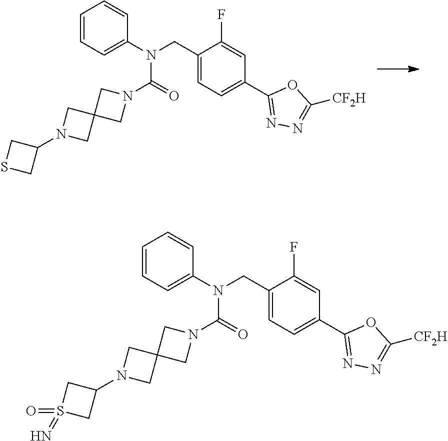 [SMILES: O=C(N1CC2(C1)CN(C1CSC1)C2)N(Cc1ccc(-c2nnc(C(F)F)o2)cc1F)c1ccccc1 ; N=S1(=O)CC(N2CC3(CN(C(=O)N(Cc4ccc(-c5nnc(C(F)F)o5)cc4F)c4ccccc4)C3)C2)C1]

N-[[4-[5-(difluoromethyl)-1,3,4-oxadiazol-2-yl]-2-fluoro-phenyl]methyl]-N-phenyl-6-(thietan-3-yl)-2,6-diazaspiro[3.3]heptan-2-carboxamide (100.00%, 0.155 g, 0.301 mmol), iodobenzene diacetate (100.00%, 0.242 g, 0.751 mmol) and ammonium carbamate (100.00%, 0.047 g, 0.602 mmol) were dissolved in methanol (3 mL) at room temperature, and then the resulting solution was stirred at 50° C. for six hours, after which a temperature was lowered to room temperature to terminate the reaction. Solvent was removed from the resulting mixture under reduced pressure, and then the resulting concentrate was purified via column chromatography (SiO₂, 4 g cartridge; methanol/dichloromethane=0 to 5%) and concentrated to obtain a title compound (0.023 g, 14.00%) in a white solid form.

¹H NMR (400 MHz, CDCl₃) δ 7.87 (dd, J=8.0, 1.5 Hz, 1H), 7.73-7.68 (m, 2H), 7.35-7.32 (m, 2H), 7.27-7.24 (m, 1H), 7.13 (dd, J=5.3, 3.2 Hz, 2H), 7.05-6.79 (m, 1H), 5.00 (s, 2H), 4.09-4.04 (m, 2H), 3.75-3.71 (m, 2H), 3.64 (s, 4H), 3.30-3.26 (m, 1H), 3.23-3.16 (m, 4H); LRMS (ES) m/z 547.8 (M⁺+1).

### Example 84: Synthesis of Compound 84, (1S,4S)—N-[[5-[5-(difluoromethyl)-1,3,4-oxadiazol-2-yl]-2-pyridyl]methyl]-5-(1-imino-1-oxo-thiethan-3-yl)-N-phenyl-2,5-diazabicyclo[2.2.1]heptan-2-carboxamide

### [Step 1] Synthesis of (1S,4S)—N-[[5-[5-(difluoromethyl)-1,3,4-oxadiazol-2-yl]-2-pyridyl]methyl]-N-phenyl-5-(thiethan-3-yl)-2,5-diazabicyclo[2.2.1]heptan-2-carboxamide

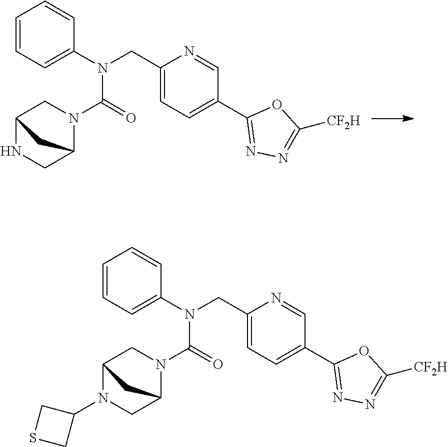 [SMILES: O=C(N(Cc1ccc(-c2nnc(C(F)F)o2)cn1)c1ccccc1)N1C[C@@H]2C[C@H]1CN2 ; O=C(N(Cc1ccc(-c2nnc(C(F)F)o2)cn1)c1ccccc1)N1C[C@@H]2C[C@H]1CN2C1CSC1]

(1S,4S)—N-[[5-[5-(difluoromethyl)-1,3,4-oxadiazol-2-yl]-2-pyridyl]methyl]-N-phenyl-2,5-diazabicyclo[2.2.1]heptan-2-carboxamide (100.00%, 0.110 g, 0.258 mmol) and thietan-3-one (100.00%, 0.034 g, 0.386 mmol) were dissolved in dichloromethane (4 mL) at room temperature, and then sodium triacetoxyborohydride (100.00%, 0.109 g, 0.517 mmol) was added into the resulting solution and stirred at the same temperature for 18 hours. A saturated aqueous sodium hydrogen carbonate solution was poured into the reaction mixture and extracted with dichloromethane. An organic layer was washed with saturated aqueous sodium chloride solution, dehydrated with anhydrous magnesium sulfate, filtered, and concentrated under reduced pressure. The resulting concentrate was purified via column chromatography (SiO₂, 4 g cartridge; methanol/dichloromethane=0 to 2.5%) and concentrated to obtain a title compound (0.066 g, 51.31%) as a colorless oil form.

### [Step 2] Synthesis of Compound 84

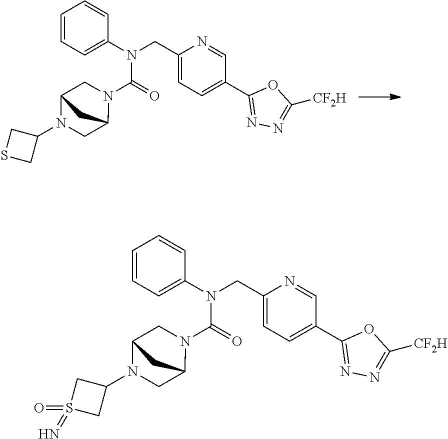 [SMILES: O=C(N(Cc1ccc(-c2nnc(C(F)F)o2)cn1)c1ccccc1)N1C[C@@H]2C[C@H]1CN2C1CSC1 ; N=S1(=O)CC(N2C[C@@H]3C[C@H]2CN3C(=O)N(Cc2ccc(-c3nnc(C(F)F)o3)cn2)c2ccccc2)C1]

(1S,4S)—N-[[5-[5-(difluoromethyl)-1,3,4-oxadiazol-2-yl]-2-pyridyl]methyl]-N-phenyl-5-(thietan-3-yl)-2,5-diazabicyclo[2.2.1]heptan-2-carboxamide (100.00%, 0.066 g, 0.132 mmol), iodobenzene diacetate (100.00%, 0.107 g, 0.332 mmol) and ammonium carbamate (100.00%, 0.021 g, 0.269 mmol) were dissolved in methanol (3 mL) at room temperature, and then the resulting solution was stirred at 50° C. for six hours, after which a temperature was lowered to room temperature to terminate the reaction. Solvent was removed from the resulting mixture under reduced pressure, and then the resulting concentrate was purified via column chromatography (SiO₂, 4 g cartridge; methanol/dichloromethane=0 to 5%) and concentrated to obtain a title compound (0.007 g, 9.99%) in a white solid form.

¹H NMR (400 MHz, CDCl₃) δ 9.25 (d, J=1.9 Hz, 1H), 8.37 (dd, J=8.2, 2.2 Hz, 1H), 7.64 (d, J=8.2 Hz, 1H), 7.34-7.31 (m, 2H), 7.23-7.22 (m, 2H), 7.18-7.14 (m, 1H), 7.08-6.82 (m, 1H), 5.20 (d, J=16.3 Hz, 1H), 5.05 (d, J=16.3 Hz, 1H), 4.40 (s, 1H), 4.10-4.01 (m, 2H), 3.92-3.87 (m, 2H), 3.56-3.50 (m, 1H), 3.26 (s, 1H), 3.04 (d, J=10.1 Hz, 1H), 2.79 (q, J=6.5 Hz, 2H), 2.57 (d, J=8.6 Hz, 1H), 1.77 (d, J=9.8 Hz, 1H), 1.62 (d, J=9.9 Hz, 1H); LRMS (ES) m/z 530.9 (M⁺+1).

### Example 85: Synthesis of Compound 85, N-[[5-[5-(difluoromethyl)-1,3,4-oxadiazol-2-yl]-2-pyridyl]methyl]-1-imino-2-methyl-1-oxo-N-phenyl-1,4-thiazinan-4-carboxamide

### [Step 1] Synthesis of N-[[5-[5-(difluoromethyl)-1,3,4-oxadiazol-2-yl]-2-pyridyl]methyl]-2-methyl-N-phenyl-thiomorpholin-4-carboxamide

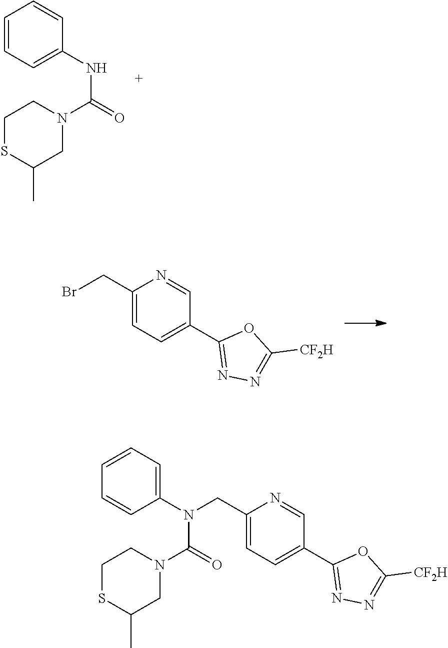

2-methyl-N-phenyl-thiomorpholin-4-carboxamide (100.00%, 0.150 g, 0.635 mmol) was dissolved in N,N-dimethylformamide (10 mL) at 0° C., and then sodium hydride (100.00%, 1.500 equiv., 0.952 mmol) was added to the resulting solution and stirred at the same temperature for 30 minutes. 2-[6-(bromomethyl)-3-pyridyl]-5-(difluoromethyl)-1,3,4-oxadiazole (100.00%, 1.100 equiv., 0.698 mmol) was added into the reaction mixture and further stirred at room temperature for two hours. A saturated aqueous sodium hydrogen carbonate solution was poured into the reaction mixture, and extracted with ethyl acetate. An organic layer was washed with saturated aqueous sodium chloride solution, dehydrated with anhydrous magnesium sulfate, filtered, and concentrated under reduced pressure. The resulting concentrate was purified via column chromatography (SiO₂, 12 g cartridge; hexane/ethyl acetate=0 to 50%) and concentrated to obtain a title compound (0.100 g, 35.36%) as a colorless oil form.

### [Step 2] Synthesis of Compound 85

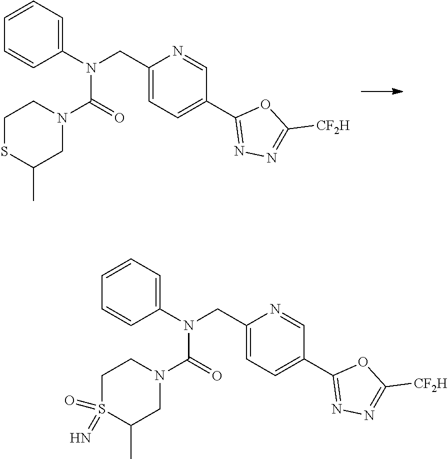 [SMILES: CC1CN(C(=O)N(Cc2ccc(-c3nnc(C(F)F)o3)cn2)c2ccccc2)CCS1 ; CC1CN(C(=O)N(Cc2ccc(-c3nnc(C(F)F)o3)cn2)c2ccccc2)CCS1(=N)=O]

N-[[5-[5-(difluoromethyl)-1,3,4-oxadiazol-2-yl]-2-pyridyl]methyl]-2-methyl-N-phenyl-thiomorpholin-4-carboxamide (100.00%, 0.030 g, 0.067 mmol) prepared in step 2, (diacetoxyiodo)benzene (100.00%, 2.500 equiv., 0.168 mmol) and ammonium carbamate (100.00%, 2.000 equiv., 0.135 mmol) were dissolved in methanol (10 mL) at room temperature, and then the resulting solution was stirred overnight at the same temperature. Solvent was removed from the reaction mixture under reduced pressure, and then saturated aqueous sodium hydrogen carbonate solution was poured into the resulting concentrate and extracted with ethyl acetate. An organic layer was washed with saturated aqueous sodium chloride solution, dehydrated with anhydrous magnesium sulfate, filtered, and concentrated under reduced pressure. The resulting concentrate was purified via column chromatography (SiO₂, 12 g cartridge; dichloromethane/methanol=0 to 10%), and concentrated to obtain a title compound (0.015 g, 46.75%) in a colorless oil form.

¹H NMR (400 MHz, CDCl₃) δ 9.25 (d, J=2.0 Hz, 1H), 8.38 (dt, J=8.4, 2.1 Hz, 1H), 7.58 (d, J=8.0 Hz, 1H), 7.37 (td, J=6.8, 1.7 Hz, 2H), 7.22-7.18 (m, 3H), 7.08 (s, 0.25H), 6.95 (s, 0.5H), 6.82 (s, 0.25H), 5.17-5.03 (m, 2H), 4.09-4.03 (m, 1H), 3.97-3.93 (m, 1H), 3.42-3.32 (m, 1H), 3.11-2.89 (m, 4H), 1.70 (brs, 1H), 1.20-1.17 (m, 3H); LRMS (ES) m/z 477.9 (M⁺+1).

### Example 86: Synthesis of Compound 86, N-[[5-[5-(difluoromethyl)-1,3,4-oxadiazol-2-yl]thiazol-2-yl]methyl]-1-imino-1-oxo-N-phenyl-1,4-thiazinan-4-sulfonamide

### [Step 1] Synthesis of N-[[5-[5-(difluoromethyl)-1,3,4-oxadiazol-2-yl]thiazol-2-yl]methyl]-N-phenyl-thiomorpholin-4-sulfonamide

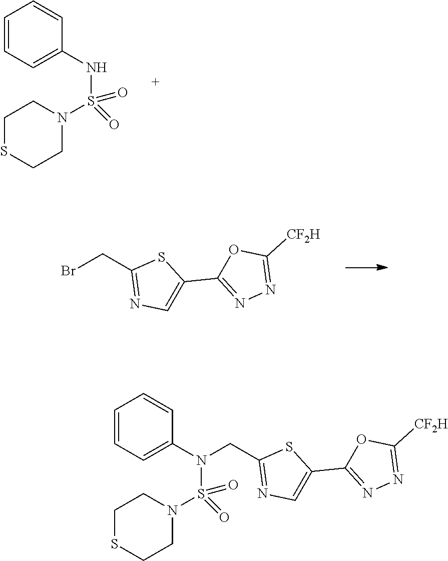 [SMILES: O=S(=O)(Nc1ccccc1)N1CCSCC1 ; FC(F)c1nnc(-c2cnc(CBr)s2)o1 ; O=S(=O)(N1CCSCC1)N(Cc1ncc(-c2nnc(C(F)F)o2)s1)c1ccccc1]

N-phenylthiomorpholin-4-sulfonamide (100.00%, 0.200 g, 0.774 mmol), 2-[2-(bromomethyl)thiazol-5-yl]-5-(difluoromethyl)-1,3,4-oxadiazole (100.00%, 1.100 equiv., 0.851 mmol), potassium carbonate (100.00%, 1.500 equiv., 1.161 mmol), and potassium iodide (100.00%, 0.100 equiv., 0.077 mmol) were dissolved in N,N-dimethylformamide (10 mL) at room temperature, and then the resulting solution was stirred overnight at the same temperature. Solvent was removed from the reaction mixture under reduced pressure, and then saturated aqueous sodium hydrogen carbonate solution was poured into the resulting concentrate and extracted with ethyl acetate. An organic layer was washed with saturated aqueous sodium chloride solution, dehydrated with anhydrous magnesium sulfate, filtered, and concentrated under reduced pressure. The resulting concentrate was purified via column chromatography (SiO₂, 12 g cartridge; dichloromethane/methanol=0 to 10%), and concentrated to obtain a title compound (0.033 g, 9.00%) in a colorless oil form.

### [Step 2] Synthesis of Compound 86

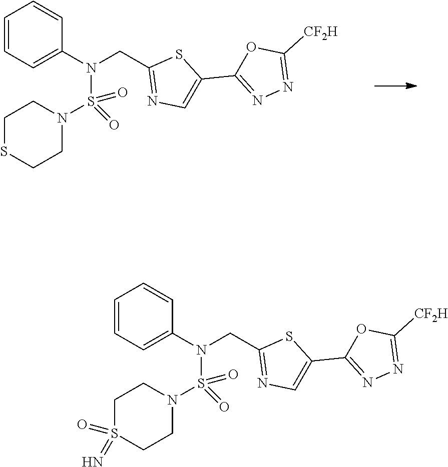 [SMILES: O=S(=O)(N1CCSCC1)N(Cc1ncc(-c2nnc(C(F)F)o2)s1)c1ccccc1 ; N=S1(=O)CCN(S(=O)(=O)N(Cc2ncc(-c3nnc(C(F)F)o3)s2)c2ccccc2)CC1]

N-[[5-[5-(difluoromethyl)-1,3,4-oxadiazol-2-yl]thiazol-2-yl]methyl]-N-phenyl-thiomorpholin-4-sulfonamide (100.00%, 0.033 g, 0.070 mmol) prepared in step 1, (diacetoxyiodo)benzene (100.00%, 2.500 equiv., 0.174 mmol) and ammonium carbamate (100.00%, 2.000 equiv., 0.139 mmol) were dissolved in methanol (10 mL) at room temperature, and then the resulting solution was stirred overnight at the same temperature. Solvent was removed from the reaction mixture under reduced pressure, and then a saturated aqueous sodium hydrogen carbonate solution was poured into the resulting concentrate and extracted with dichloromethane. An organic layer was washed with saturated aqueous sodium chloride solution, dehydrated with anhydrous magnesium sulfate, filtered, and concentrated under reduced pressure. The resulting concentrate was purified via column chromatography (SiO₂, 12 g cartridge; dichloromethane/methanol=0 to 10%), and concentrated to obtain a title compound (0.005 g, 14.22%) in a yellow solid form.

¹H NMR (400 MHz, CDCl₃) δ 8.39 (s, 1H), 7.49-7.36 (m, 5H), 7.05 (s, 0.25H), 6.93 (s, 0.5H), 6.80 (s, 0.25H), 5.20 (s, 2H), 3.80-3.63 (m, 4H), 3.15-3.03 (m, 4H), 2.57 (brs, 1H); LRMS (ES) m/z 505.8 (M⁺+1).

### Example 87: Synthesis of Compound 87, 3-[N-[[5-[5-(difluoromethyl)-1,3,4-oxadiazol-2-yl]-2-pyridyl]methyl]anilino]-4-[(1S,4S)-5-(1-imino-1-oxo-thiethane-3-yl)-2,5-diazabicyclo[2.2.1]heptan-2-yl]cyclobut-3-en-1,2-dione

### [Step 1] Synthesis of 3-[N-[[5-[5-(difluoromethyl)-1,3,4-oxadiazol-2-yl]-2-pyridyl]methyl]anilino]-4-[(1S,4S)-5-(thiethan-3-yl)-2,5-diazabicyclo[2.2.1]heptan-2-yl]cyclobut-3-en-1,2-dione

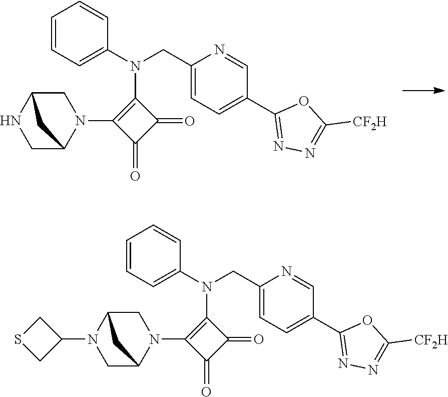 [SMILES: O=c1c(N(Cc2ccc(-c3nnc(C(F)F)o3)cn2)c2ccccc2)c(N2C[C@@H]3C[C@H]2CN3)c1=O ; O=c1c(N(Cc2ccc(-c3nnc(C(F)F)o3)cn2)c2ccccc2)c(N2C[C@@H]3C[C@H]2CN3C2CSC2)c1=O]

3-[(1S,4S)-2,5-diazabicyclo[2.2.1]heptan-2-yl]-4-[N-[[5-[5-(difluoromethyl)-1,3,4-oxadiazol-2-yl]-2-pyridyl]methyl]anilino]cyclobut-3-en-1,2-dione (100.00%, 0.200 g, 0.418 mmol) and thietan-3-one (100.00%, 0.055 g, 0.624 mmol) were dissolved in dichloromethane (4 mL) at room temperature, and then sodium triacetoxyborohydride (100.00%, 0.176 g, 0.834 mmol) was added into the resulting solution and stirred at the same temperature for 18 hours. A saturated aqueous sodium hydrogen carbonate solution was poured into the reaction mixture, extracted with dichloromethane, filtered via a plastic filter to remove a solid residue and an aqueous solution layer therefrom, and concentrated under reduced pressure. The resulting concentrate was purified via column chromatography (SiO₂, 4 g cartridge; ethyl acetate/hexane=60 to 90%), and concentrated to obtain a title compound (0.156 g, 67.77%) in a yellow solid form.

### [Step 2] Synthesis of Compound 87

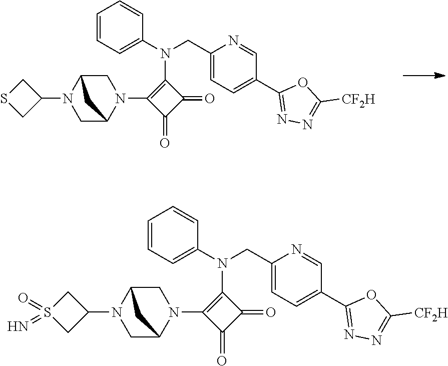 [SMILES: O=c1c(N(Cc2ccc(-c3nnc(C(F)F)o3)cn2)c2ccccc2)c(N2C[C@@H]3C[C@H]2CN3C2CSC2)c1=O ; N=S1(=O)CC(N2C[C@@H]3C[C@H]2CN3c2c(N(Cc3ccc(-c4nnc(C(F)F)o4)cn3)c3ccccc3)c(=O)c2=O)C1]

3-[N-[[5-[5-(difluoromethyl)-1,3,4-oxadiazol-2-yl]-2-pyridyl]methyl]anilino]-4-[(1S,4S)-5-(thietan-3-yl)-2,5-diazabicyclo[2.2.1]heptan-2-yl]cyclobut-3-en-1,2-dione (100.00%, 0.156 g, 0.283 mmol), iodobenzene diacetate (100.00%, 0.228 g, 0.708 mmol) and ammonium carbamate (100.00%, 0.044 g, 0.564 mmol) were dissolved in methanol (3 mL) at room temperature, and then the resulting solution was stirred at 50° C. for 18 hours, after which a temperature was lowered to room temperature to terminate the reaction. Solvent was removed from the reaction mixture under reduced pressure, and then a saturated aqueous sodium hydrogen carbonate solution was poured into the resulting concentrate and extracted with dichloromethane. An organic layer was washed with saturated aqueous sodium chloride solution, dehydrated with anhydrous magnesium sulfate, filtered, and concentrated under reduced pressure. The resulting concentrate was purified via column chromatography (SiO₂, 4 g cartridge; methanol/dichloromethane=0 to 5%), and concentrated to obtain a product, and then the resulting product was purified again via chromatography (SiO₂ plate, 20×20×1 mm; methanol/dichloromethane=5%), and concentrated to obtain a title compound (0.030 g, 18.21%) in a yellow solid form.

¹H NMR (400 MHz, CDCl₃) δ 9.27 (d, J=1.8 Hz, 1H), 8.39 (dd, J=8.2, 2.2 Hz, 1H), 7.63 (d, J=8.2 Hz, 1H), 7.40-7.36 (m, 2H), 7.27-7.25 (m, 1H), 7.22-7.20 (m, 2H), 7.08-6.82 (m, 1H), 5.64-5.52 (m, 2H), 4.13-4.07 (m, 1H), 4.01-3.96 (m, 1H), 3.89-3.80 (m, 2H), 3.45-3.41 (m, 1H), 3.26 (s, 1H), 3.20 (s, 1H), 2.73-2.70 (m, 3H), 1.88-1.83 (m, 2H), 1.63-1.60 (m, 1H); LRMS (ES) m/z 582.4 (M⁺+1).

### Example 88: Synthesis of Compound 88, 3-[N-[[5-[5-(difluoromethyl)-1.3.4-oxadiazol-2-yl]-3-fluoro-2-pyridyl]methyl]anilino]-4-[(1S,4S)-5-(1-imino-1-oxo-thiethan-3-yl)-2,5-diazabicyclo[2.2.1]heptan-2-yl]cyclobut-3-en-1,2-dione

### [Step 1] Synthesis of 3-[N-[[5-[5-(difluoromethyl)-1,3,4-oxadiazol-2-yl]-3-fluoro-2-pyridyl]methyl]anilino]-4-[(1S,4S)-5-(thiethan-3-yl)-2,5-diazabicyclo[2.2.1]heptan-2-yl]cyclobut-3-en-1,2-dione

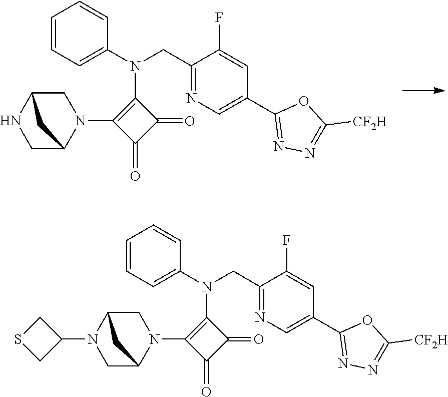 [SMILES: O=c1c(N(Cc2ncc(-c3nnc(C(F)F)o3)cc2F)c2ccccc2)c(N2C[C@@H]3C[C@H]2CN3)c1=O ; O=c1c(N(Cc2ncc(-c3nnc(C(F)F)o3)cc2F)c2ccccc2)c(N2C[C@@H]3C[C@H]2CN3C2CSC2)c1=O]

3-[(1S,4S)-2,5-diazabicyclo[2.2.1]heptan-2-yl]-4-[N-[[5-[5-(difluoromethyl)-1,3,4-oxadiazol-2-yl]-3-fluoro-2-pyridyl]methyl]anilino]cyclobut-3-en-1,2-dione (100.00%, 0.200 g, 0.403 mmol) and thietan-3-one (100.00%, 0.053 g, 0.601 mmol) were dissolved in dichloromethane (4 mL) at room temperature, and then sodium triacetoxyborohydride (100.00%, 0.170 g, 0.806 mmol) was added into the resulting solution and stirred at the same temperature for 18 hours. A saturated aqueous sodium hydrogen carbonate solution was poured into the reaction mixture, extracted with dichloromethane, filtered via a plastic filter to remove a solid residue and an aqueous solution layer therefrom, and concentrated under reduced pressure. The resulting concentrate was purified via column chromatography (SiO₂, 4 g cartridge; ethyl acetate/hexane=60 to 80%), and concentrated to obtain a title compound (0.120 g, 52.38%) in a yellow solid form.

### [Step 2] Synthesis of Compound 88

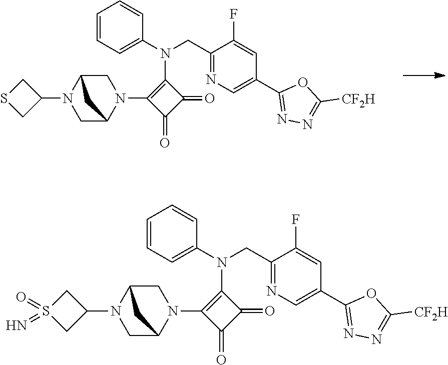 [SMILES: O=c1c(N(Cc2ncc(-c3nnc(C(F)F)o3)cc2F)c2ccccc2)c(N2C[C@@H]3C[C@H]2CN3C2CSC2)c1=O ; N=S1(=O)CC(N2C[C@@H]3C[C@H]2CN3c2c(N(Cc3ncc(-c4nnc(C(F)F)o4)cc3F)c3ccccc3)c(=O)c2=O)C1]

3-[N-[[5-[5-(difluoromethyl)-1,3,4-oxadiazol-2-yl]-3-fluoro-2-pyridyl]methyl]anilino]-4-[(1S,4S)-5-(thietan-3-yl)-2,5-diazabicyclo[2.2.1]heptan-2-yl]cyclobut-3-en-1,2-dione (100.00%, 0.120 g, 0.211 mmol), iodobenzene diacetate (100.00%, 0.170 g, 0.528 mmol) and ammonium carbamate (100.00%, 0.033 g, 0.423 mmol) were dissolved in methanol (3 mL) at room temperature, and then the resulting solution was stirred at 50° C. for 18 hours, after which a temperature was lowered to room temperature to terminate the reaction. Solvent was removed from the reaction mixture under reduced pressure, and then a saturated aqueous sodium hydrogen carbonate solution was poured into the resulting concentrate and extracted with dichloromethane. An organic layer was washed with saturated aqueous sodium chloride solution, dehydrated with anhydrous magnesium sulfate, filtered, and concentrated under reduced pressure. The resulting concentrate was purified via column chromatography (SiO₂, 4 g cartridge; methanol/dichloromethane=0 to 5%), and concentrated to obtain a product, and then the resulting product was purified again via chromatography (SiO₂ plate, 20×20×1 mm; methanol/dichloromethane=5%), and concentrated to obtain a title compound (0.041 g, 32.40%) in a yellow solid form.

¹H NMR (400 MHz, CDCl₃) δ 79.10 (s, 1H), 8.08 (dd, J=9.3, 1.7 Hz, 1H), 7.39-7.35 (m, 2H), 7.28-7.25 (m, 1H), 7.19-7.16 (m, 2H), 7.09-6.83 (m, 1H), 5.73-5.64 (m, 2H), 4.16-4.15 (m, 2H), 4.13-4.09 (m, 1H), 4.02-3.97 (m, 2H), 3.48-3.44 (m, 1H), 3.21-3.20 (m, 1H), 2.76-2.72 (m, 3H), 1.87-1.84 (m, 1H), 1.65-1.63 (m, 1H); LRMS (ES) m/z 600.8 (M⁺+1).

### Example 89: Synthesis of Compound 89, 3-[N-[[4-[5-(difluoromethyl)-1,3,4-oxadiazol-2-yl]-2-fluoro-phenyl]methyl]anilino]-4-[(1S,4S)-5-(1-imino-1-oxo-thiethan-3-yl)-2,5-diazabicyclo[2.2.1]heptan-2-yl]cyclobut-3-en-1,2-dione

### [Step 1] Synthesis of 3-[N-[[4-[5-(difluoromethyl)-1,3,4-oxadiazol-2-yl]-2-fluoro-phenyl]methyl]anilino]-4-[(1S,4S)-5-(thiethane-3-yl)-2,5-diazabicyclo[2.2.1]heptan-2-yl]cyclobut-3-en-1,2-dione

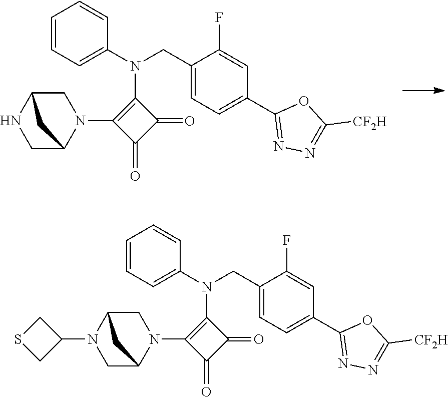 [SMILES: O=c1c(N(Cc2ccc(-c3nnc(C(F)F)o3)cc2F)c2ccccc2)c(N2C[C@@H]3C[C@H]2CN3)c1=O ; O=c1c(N(Cc2ccc(-c3nnc(C(F)F)o3)cc2F)c2ccccc2)c(N2C[C@@H]3C[C@H]2CN3C2CSC2)c1=O]

3-[(1S,4S)-2,5-diazabicyclo[2.2.1]heptan-2-yl]-4-[N-[[4-[5-(difluoromethyl)-1,3,4-oxadiazol-2-yl]-2-fluoro-phenyl]methyl]anilino]cyclobut-3-en-1,2-dione (100.00%, 0.150 g, 0.303 mmol) and thietan-3-one (100.00%, 0.040 g, 0.454 mmol) were dissolved in dichloromethane (4 mL) at room temperature, and then sodium triacetoxyborohydride (100.00%, 0.128 g, 0.607 mmol) was added into the resulting solution and stirred at the same temperature for 18 hours. A saturated aqueous sodium hydrogen carbonate solution was poured into the reaction mixture, extracted with dichloromethane, filtered via a plastic filter to remove a solid residue and an aqueous solution layer therefrom, and concentrated under reduced pressure. The resulting concentrate was purified via column chromatography (SiO₂, 4 g cartridge; ethyl acetate/hexane=5 to 70%), and concentrated to obtain a title compound (0.053 g, 30.84%) in a white solid form.

### [Step 1] Synthesis of Compound 89

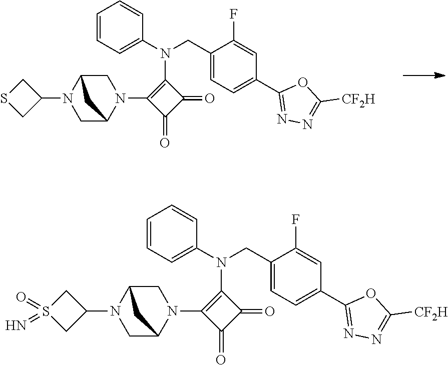 [SMILES: O=c1c(N(Cc2ccc(-c3nnc(C(F)F)o3)cc2F)c2ccccc2)c(N2C[C@@H]3C[C@H]2CN3C2CSC2)c1=O ; N=S1(=O)CC(N2C[C@@H]3C[C@H]2CN3c2c(N(Cc3ccc(-c4nnc(C(F)F)o4)cc3F)c3ccccc3)c(=O)c2=O)C1]

3-[N-[[4-[5-(difluoromethyl)-1,3,4-oxadiazol-2-yl]-2-fluoro-phenyl]methyl]anilino]-4-[(1S,4S)-5-(thietan-3-yl)-2,5-diazabicyclo[2.2.1]heptan-2-yl]cyclobut-3-en-1,2-dione (100.00%, 0.053 g, 0.093 mmol), iodobenzene diacetate (100.00%, 0.075 g, 0.233 mmol) and ammonium carbamate (100.00%, 0.015 g, 0.192 mmol) were dissolved in methanol (3 mL) at room temperature, and then the resulting solution was stirred at 50° C. for 18 hours, after which a temperature was lowered to room temperature to terminate the reaction. Solvent was removed from the reaction mixture under reduced pressure, and then a saturated aqueous sodium hydrogen carbonate solution was poured into the resulting concentrate and extracted with dichloromethane. An organic layer was washed with saturated aqueous sodium chloride solution, dehydrated with anhydrous magnesium sulfate, filtered, and concentrated under reduced pressure. The resulting concentrate was purified via column chromatography (SiO₂, 4 g cartridge; methanol/dichloromethane=0 to 5%), and concentrated to obtain a product, and then the resulting product was purified again via chromatography (SiO₂ plate, 20×20×1 mm; methanol/dichloromethane=5%), and concentrated to obtain a title compound (0.008 g, 14.31%) in a white solid form.

¹H NMR (400 MHz, CDCl₃) δ 7.89 (dd, J=8.0, 1.4 Hz, 1H), 7.74-7.67 (m, 2H), 7.39-7.35 (m, 2H), 7.31-7.28 (m, 1H), 7.09-7.07 (m, 2H), 7.05-6.79 (m, 1H), 5.60 (d, J=15.4 Hz, 1H), 5.47 (d, J=15.4 Hz, 1H), 4.13-4.07 (m, 1H), 4.01-3.95 (m, 1H), 3.86-3.80 (m, 2H), 3.43-3.41 (m, 1H), 3.28 (s, 1H), 3.17 (s, 1H), 2.71 (brs, 3H), 1.83 (d, J=9.9 Hz, 1H), 1.59 (d, J=10.1 Hz, 1H); LRMS (ES) m/z 599.8 (M⁺+1).

### Example 90: Synthesis of Compound 90, N-[[5-[5-(difluoromethyl)-1,3,4-oxadiazol-2-yl]-2-pyridyl]methyl]-N-(3-fluorophenyl)-3-(methylsulfonimidoyl)azetidin-1-carboxamide

### [Step 1] Synthesis of N-(3-fluorophenyl)-3-methylsulfyl-azetidin-1-carboxamide

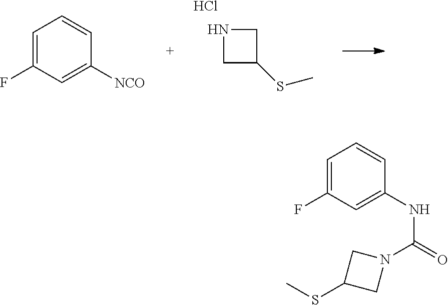

3-methylsulfylazetidine; hydrochloride (100.00%, 0.452 g, 3.237 mmol) and N,N-diisopropylethylamine (100.00% solution, 1.15 mL, 6.585 mmol) were dissolved in dichloromethane (20 mL) at room temperature, and then 1-fluoro-3-isocyanato-benzene (100.00% solution, 0.5 mL, 4.380 mmol) was added into the resulting solution and stirred at the same temperature for two hours. A saturated aqueous sodium hydrogen carbonate solution was poured into the reaction mixture, extracted with dichloromethane, filtered via a plastic filter to remove a solid residue and an aqueous solution layer therefrom, and concentrated under reduced pressure. The resulting concentrate was purified via column chromatography (SiO2, 24 g cartridge; ethyl acetate/hexane=10 to 40%), and concentrated to obtain a title compound (0.815 g, 77.44%) in a white solid form.

### [Step 2] Synthesis of N-[[5-[5-(difluoromethyl)-1,3,4-oxadiazol-2-yl]-2-pyridyl]methyl]-N-(3-fluorophenyl)-3-methylsulfyl-azetidin-1-carboxamide

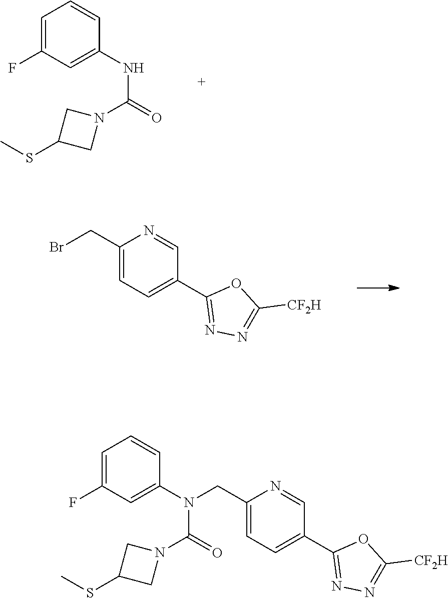 [SMILES: CSC1CN(C(=O)Nc2cccc(F)c2)C1 ; FC(F)c1nnc(-c2ccc(CBr)nc2)o1 ; CSC1CN(C(=O)N(Cc2ccc(-c3nnc(C(F)F)o3)cn2)c2cccc(F)c2)C1]

N-(3-fluorophenyl)-3-methylsulfyl-azetidin-1-carboxamide (100.00%, 0.200 g, 0.832 mmol) and sodium hydride (60.00%, 0.037 g, 0.925 mmol) were dissolved in N,N-dimethylformamide (4 mL) at 0° C., and then 2-[6-(bromomethyl)-3-pyridyl]-5-(difluoromethyl)-1,3,4-oxadiazole (100.00%, 0.266 g, 0.917 mmol) was added into the resulting solution and stirred at room temperature for 18 hours. Solvent was removed from the reaction mixture under reduced pressure, and then saturated aqueous sodium hydrogen carbonate solution was poured into the resulting concentrate, extracted with dichloromethane, filtered via a plastic filter to remove a solid residue and an aqueous solution layer therefrom, and concentrated under reduced pressure. The resulting concentrate was purified via column chromatography (SiO₂, 4 g cartridge; ethyl acetate/hexane=10 to 40%), and concentrated to obtain a title compound (0.207 g, 55.34%) in a light yellow oil form.

### [Step 3] Synthesis of Compound 90

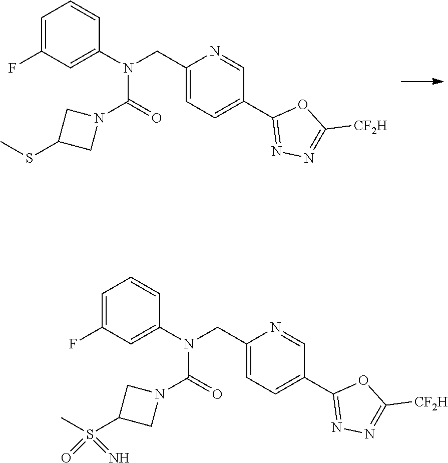 [SMILES: CSC1CN(C(=O)N(Cc2ccc(-c3nnc(C(F)F)o3)cn2)c2cccc(F)c2)C1 ; CS(=N)(=O)C1CN(C(=O)N(Cc2ccc(-c3nnc(C(F)F)o3)cn2)c2cccc(F)c2)C1]

N-[[5-[5-(difluoromethyl)-1,3,4-oxadiazol-2-yl]-2-pyridyl]methyl]-N-(3-fluorophenyl)-3-methylsulfyl-azetidin-1-carboxamide (100.00%, 0.207 g, 0.461 mmol), iodobenzene diacetate (100.00%, 0.371 g, 1.152 mmol) and ammonium carbamate (100.00%, 0.072 g, 0.922 mmol) were dissolved in methanol (5 mL) at room temperature, and then the resulting solution was stirred at 50° C. for 18 hours, after which a temperature was lowered to room temperature to terminate the reaction. Solvent was removed from the reaction mixture under reduced pressure, and then saturated aqueous sodium hydrogen carbonate solution was poured into the resulting concentrate, extracted with dichloromethane, filtered via a plastic filter to remove a solid residue and an aqueous solution layer therefrom, and concentrated under reduced pressure. The resulting concentrate was purified via column chromatography (SiO₂, 4 g cartridge; methanol/dichloromethane=0 to 5%), and concentrated to obtain a product, and then the resulting product was purified again via chromatography (SiO₂ plate, 20×20×1 mm; methanol/dichloromethane=5%), and concentrated to obtain a title compound (0.042 g, 18.98%) in a white solid form.

¹H NMR (400 MHz, CDCl₃) δ 9.26 (d, J=2.0 Hz, 1H), 8.39 (dd, J=8.2, 2.2 Hz, 1H), 7.61 (d, J=8.2 Hz, 1H), 7.36-7.31 (m, 1H), 7.11-6.83 (m, 4H), 5.13-5.04 (m, 2H), 4.00-3.93 (m, 4H), 3.88-3.83 (m, 1H), 2.88 (s, 3H), 2.66 (s, 1H); LRMS (ES) m/z 481.7 (M⁺+1).

### Example 91: Synthesis of Compound 91, N-[[4-[5-(difluoromethyl)-1,3,4-oxadiazol-2-yl]-2-fluoro-phenyl]methyl]-N-(3-fluorophenyl)-3-(methylsulfonimidoyl)azetidin-1-carboxamide

### [Step 1] Synthesis of N-[[4-[5-(difluoromethyl)-1,3,4-oxadiazol-2-yl]-2-fluoro-phenyl]methyl]-N-(3-fluorophenyl)-3-methylsulfyl-azetidin-1-carboxamide

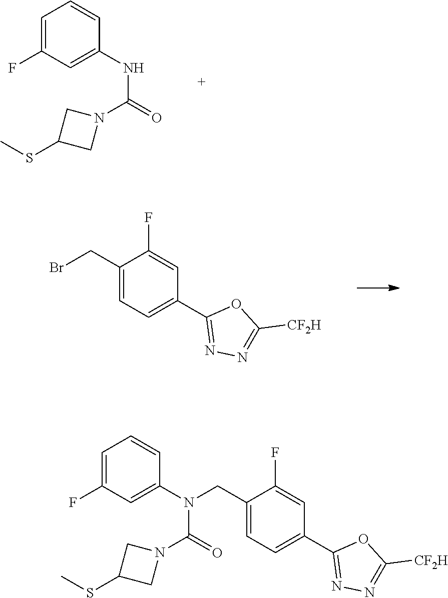 [SMILES: CSC1CN(C(=O)Nc2cccc(F)c2)C1 ; Fc1cc(-c2nnc(C(F)F)o2)ccc1CBr ; CSC1CN(C(=O)N(Cc2ccc(-c3nnc(C(F)F)o3)cc2F)c2cccc(F)c2)C1]

N-(3-fluorophenyl)-3-methylsulfyl-azetidin-1-carboxamide (100.00%, 0.200 g, 0.832 mmol) and sodium hydride (60.00%, 0.037 g, 0.925 mmol) were dissolved in N,N-dimethylformamide (4 mL) at 0° C., and then 2-[4-(bromomethyl)-3-fluoro-phenyl]-5-(difluoromethyl)-1,3,4-oxadiazole (100.00%, 0.281 g, 0.915 mmol) was added into the resulting solution and stirred at room temperature for 18 hours. Solvent was removed from the reaction mixture under reduced pressure, and then saturated aqueous sodium hydrogen carbonate solution was poured into the resulting concentrate, extracted with dichloromethane, filtered via a plastic filter to remove a solid residue and an aqueous solution layer therefrom, and concentrated under reduced pressure. The resulting concentrate was purified via column chromatography (SiO₂, 4 g cartridge; ethyl acetate/hexane=5 to 40%), and concentrated to obtain a title compound (0.194 g, 49.98%) in a white solid form.

### [Step 2] Synthesis of Compound 91

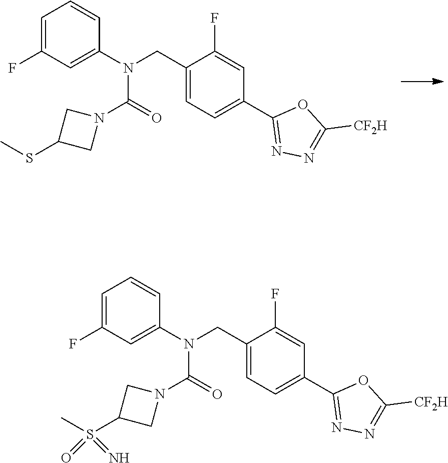 [SMILES: CSC1CN(C(=O)N(Cc2ccc(-c3nnc(C(F)F)o3)cc2F)c2cccc(F)c2)C1 ; CS(=N)(=O)C1CN(C(=O)N(Cc2ccc(-c3nnc(C(F)F)o3)cc2F)c2cccc(F)c2)C1]

N-[[4-[5-(difluoromethyl)-1,3,4-oxadiazol-2-yl]-2-fluoro-phenyl]methyl]-N-(3-fluorophenyl)-3-methylsulfyl-azetidin-1-carboxamide (100.00%, 0.197 g, 0.422 mmol), iodobenzene diacetate (100.00%, 0.340 g, 1.056 mmol) and ammonium carbamate (100.00%, 0.066 g, 0.845 mmol) were dissolved in methanol (5 mL) at room temperature, and then the resulting solution was stirred at 50° C. for 18 hours, after which a temperature was lowered to room temperature to terminate the reaction. Solvent was removed from the reaction mixture under reduced pressure, and then saturated aqueous sodium hydrogen carbonate solution was poured into the resulting concentrate, extracted with dichloromethane, filtered via a plastic filter to remove a solid residue and an aqueous solution layer therefrom, and concentrated under reduced pressure. The resulting concentrate was purified via column chromatography (SiO₂, 4 g cartridge; methanol/dichloromethane=0 to 5%), and concentrated to obtain a title compound (0.064 g, 30.46%) in a white solid form.

¹H NMR (400 MHz, CDCl₃) δ 7.90 (dd, J=8.0, 1.5 Hz, 1H), 7.75-7.69 (m, 2H), 7.36-7.30 (m, 1H), 7.06-6.80 (m, 4H), 5.01 (s, 2H), 3.96-3.91 (m, 4H), 3.87-3.84 (m, 1H), 2.87 (s, 3H); LRMS (ES) m/z 498.7 (M⁺+1).

### Example 92: Synthesis of Compound 92, N-[[4-[5-(difluoromethyl)-1,3,4-oxadiazol-2-yl]-2-fluoro-phenyl]methyl]-2-imino-2-oxo-N-phenyl-2lambda6-thia-6-azaspiro[3.3]heptan-6-carboxamide

### [Step 1] Synthesis of N-[[5-[5-(difluoromethyl)-1,3,4-oxadiazol-2-yl]-2-pyridyl]methyl]-N-(4-fluorophenyl)thiomorpholin-4-carboxamide

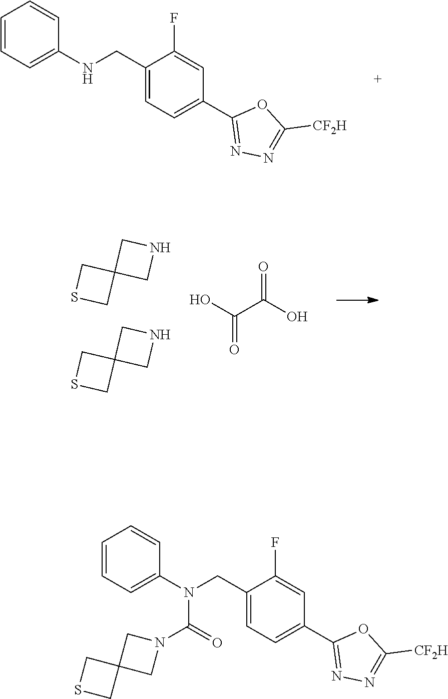

N-[[4-[5-(difluoromethyl)-1,3,4-oxadiazol-2-yl]-2-fluoro-phenyl]methyl]aniline (100.00%, 0.200 g, 0.626 mmol) and N,N-diisopropylethylamine (100.00% solution, 0.55 mL, 3.149 mmol) were dissolved in dichloromethane (5 mL) at 0° C., and then triphosgene (100.00%, 0.074 g, 0.249 mmol) was added into the resulting solution and stirred at the same temperature. Oxalic acid; 2-thia-6-azaspiro[3.3]heptane (100.00%, 0.120 g, 0.375 mmol) was added into the reaction mixture, and further stirred at room temperature for 18 hours. A saturated aqueous sodium hydrogen carbonate solution was poured into the reaction mixture, extracted with dichloromethane, filtered via a plastic filter to remove a solid residue and an aqueous solution layer therefrom, and concentrated under reduced pressure. The resulting concentrate was purified via column chromatography (SiO₂, 4 g cartridge; ethyl acetate/hexane=10 to 50%), and concentrated to obtain a title compound (0.119 g, 41.26%) in a white solid form.

### [Step 2] Synthesis of Compound 92

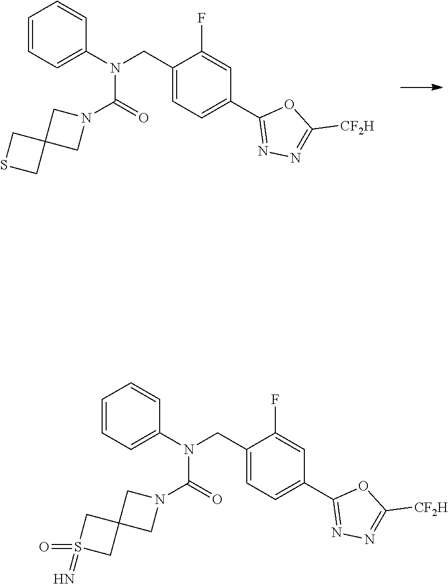 [SMILES: O=C(N1CC2(CSC2)C1)N(Cc1ccc(-c2nnc(C(F)F)o2)cc1F)c1ccccc1 ; N=S1(=O)CC2(CN(C(=O)N(Cc3ccc(-c4nnc(C(F)F)o4)cc3F)c3ccccc3)C2)C1]

N-[[5-[5-(difluoromethyl)-1,3,4-oxadiazol-2-yl]-2-pyridyl]methyl]-N-(4-fluorophenyl)thiomorpholin-4-carboxamide (100.00%, 0.040 g, 0.089 mmol), iodobenzene diacetate (100.00%, 0.072 g, 0.224 mmol) and ammonium carbamate (100.00%, 0.014 g, 0.179 mmol) were dissolved in methanol (3 mL) at room temperature, and then the resulting solution was stirred at 50° C. for 18 hours, after which a temperature was lowered to room temperature to terminate the reaction. Solvent was removed from the resulting mixture under reduced pressure, and then the resulting concentrate was purified via column chromatography (SiO₂, 4 g cartridge; methanol/dichloromethane=0 to 5%) and concentrated to obtain a title compound (0.010 g, 23.38%) in a light yellow solid form.

¹H NMR (400 MHz, CDCl₃) δ 7.86 (dd, J=8.0, 1.4 Hz, 1H), 7.70-7.66 (m, 2H), 7.35-7.32 (m, 2H), 7.28-7.24 (m, 1H), 7.13-7.12 (m, 2H), 7.05-6.79 (m, 1H), 4.99 (s, 2H), 4.09 (q, J=11.8 Hz, 4H), 3.77 (d, J=7.0 Hz, 4H), 3.08 (brs, 1H); LRMS (ES) m/z 492.5 (M⁺+1).

### <Experimental Example> Protocol for Measuring and Analyzing Activity of Compound of Present Invention

### Experimental Example 1. Identification of HDAC Enzyme Activity Inhibition (In Vitro)

A selective HDAC6 inhibitor is important for selectivity of HDAC1 inhibition, which is a cause of side effects, and thus an experiment was performed to identify HDAC1/6 enzyme selectivity and cell selectivity (HDAC1: Histone acetylation/HDAC6: Tubulin acetylation) with respect to the compound of the present invention.

### 1. Experimental Method

A HDAC enzyme inhibitory capacity of a test material was measured by using HDAC1 Fluorimetric Drug Discovery Assay Kit (Enzolifesciences: BML-AK511) and HDAC6 human recombinant (Calbiochem: 382180). For a HDAC1 assay, samples were treated at a concentration of 100, 1000 and 10000 nM. For a HDAC6 assay, samples were treated at a concentration of 0.1, 1, 10, 100 and 1000 nM.

For the test on the HDAC1 enzyme activity, human recombinant HDAC1 (BML-SE456) was used as a zymogen and Fluor de Lys®-SIRT1 (BNL-KI177) was used as a substrate. A 5-fold dilution of the compound was divided into a 96-well plate, after which 0.3 μg of the enzyme and 10 M of the substrate were inserted into each well and subjected to reaction at 37° C. for 60 minutes, such that Fluor de Lys® Developer II (BML-K1176) was inserted thereinto and subjected to reaction for 30 minutes and finished. After that, a fluorescence value (Ex 360, Em 460) was measured with a multi-plate reader (FlexStation 3, Molecular Device). An experiment on HDAC6 enzyme was conducted in accordance with the same protocol as in the HDAC1 enzyme activity test method by using human recombinant HDAC6 (382180) of Calbiochem Inc. For final result values, each IC₅₀ value was calculated with GraphPad Prism 4.0 program.

### 2. Experimental Results

The results of searching HDAC enzyme activity inhibition obtained according to the above experimental method are shown in table 18 below.

TABLE 18

| Compound | HDAC6 IC₅₀ | HDAC1 IC₅₀ | Compound | HDAC6 IC₅₀ | HDAC1 IC₅₀ |
|---|---|---|---|---|---|
| No. | (nM) | (nM) | No. | (nM) | (nM) |
| 1 | 49.4 | >10,000 | 2 | 168.2 | >10,000 |
| 3 | 151.2 | >10,000 | 4 | 390.1 | >10,000 |
| 5 | 91.4 | >10,000 | 6 | 50.0 | >10,000 |
| 7 | 40.9 | >10,000 | 8 | 194.1 | >10,000 |
| 9 | 279.8 | >10,000 | 10 | 108.4 | >10,000 |
| 11 | 1419 | >10,000 | 12 | 62.6 | >10,000 |
| 13 | 44 | >10,000 | 14 | 94.4 | >10,000 |
| 15 | 1346 | >10,000 | 16 | 42.6 | >10,000 |
| 17 | 27.2 | >10,000 | 18 | 32.6 | >10,000 |
| 19 | 19.7 | >10,000 | 20 | 76.7 | >10,000 |
| 21 | 294.4 | >10,000 | 22 | 75.2 | >10,000 |
| 23 | 64.4 | >10,000 | 24 | 1009 | >10,000 |
| 25 | 141.1 | >10,000 | 26 | 98.6 | >10,000 |
| 27 | 86.6 | >10,000 | 28 | 92.2 | >10,000 |
| 29 | 78.8 | >10,000 | 30 | 54.6 | >10,000 |
| 31 | 91.4 | >10,000 | 32 | 80.6 | >10,000 |
| 33 | 51.3 | >10,000 | 34 | 91.5 | >10,000 |
| 35 | 59.7 | >10,000 | 36 | 30.8 | >10,000 |
| 37 | 61.3 | >10,000 | 38 | 67.2 | >10,000 |
| 39 | 80.8 | >10,000 | 40 | 84.8 | >10,000 |
| 41 | 28.9 | >10,000 | 42 | 37.7 | >10,000 |
| 43 | 88.9 | >10,000 | 44 | 129.7 | >10,000 |
| 45 | 63.6 | >10,000 | 46 | 65.9 | >10,000 |
| 47 | 185.3 | >10,000 | 48 | 113.3 | >10,000 |
| 49 | 77.7 | >10,000 | 50 | 66.4 | >10,000 |
| 51 | 69.9 | >10,000 | 52 | 46.9 | >10,000 |
| 53 | 40.6 | >10,000 | 54 | 200.3 | >10,000 |
| 55 | 140.3 | >10,000 | 56 | 72.6 | >10,000 |
| 57 | 130.1 | >10,000 | 58 | 256.0 | >10,000 |
| 59 | 115.7 | >10,000 | 60 | 26.7 | >10,000 |
| 61 | 29.8 | >10,000 | 62 | 88.4 | >10,000 |
| 63 | 99.6 | >10,000 | 64 | 123.3 | >10,000 |
| 65 | 105.3 | >10,000 | 66 | 90.2 | >10,000 |
| 67 | 346.7 | >10,000 | 68 | 39.0 | >10,000 |
| 69 | 124.2 | >10,000 | 70 | 52.2 | >10,000 |
| 71 | 31.7 | >10,000 | 72 | 78.8 | >10,000 |
| 73 | 72.5 | >10,000 | 74 | 63.6 | >10,000 |
| 75 | 96.3 | >10,000 | 76 | 39.9 | >10,000 |
| 77 | 51.6 | >10,000 | 78 | 78.7 | >10,000 |
| 79 | 87.1 | >10,000 | 80 | 145.5 | >10,000 |
| 81 | 241.0 | >10,000 | 82 | 93.1 | >10,000 |
| 83 | 71.3 | >10,000 | 84 | 56.0 | >10,000 |
| 85 | 74.6 | >10,000 | 86 | 50.2 | >10,000 |
| 87 | 37.4 | >10,000 | 88 | 57.0 | >10,000 |
| 89 | 34.1 | >10,000 | 90 | 47.3 | >10,000 |
| 91 | 46.0 | >10,000 | 92 | 93.9 | >10,000 |

As can be seen in above table 18, it was confirmed from the results of testing activity inhibition against HDAC1 and HDAC6 that the sulfoximine compound of the present invention has a IC₅₀ value at HDAC6, which is about 7 times or more and up to 500 times or more lower than that of HDAC1, thus showing excellent selective HDAC6 inhibitory activity.

The present invention has been described with reference to one exemplary embodiment of the present invention, but it will be understood by those skilled in the art that the present invention may be variously changed and modified without departing from the spirit and field of the present invention, as described in the following scope of patent claims.

1. A sulfoximine compound, stereoisomers thereof or pharmaceutically acceptable salts thereof, wherein the sulfoximine compound is represented by formula I below: 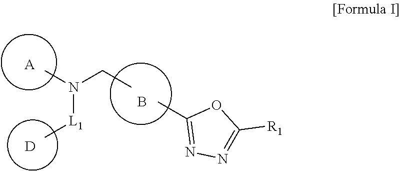
in above formula I,
R₁ is CF₂H or CF₃;
L₁ is —(C₁₋₃ alkylene)-, —C(═O)—, —S(═O)₂—, —S(═O)₂—(C₁₋₃ alkylene)-, —C(═O)N(Rₐ)—, 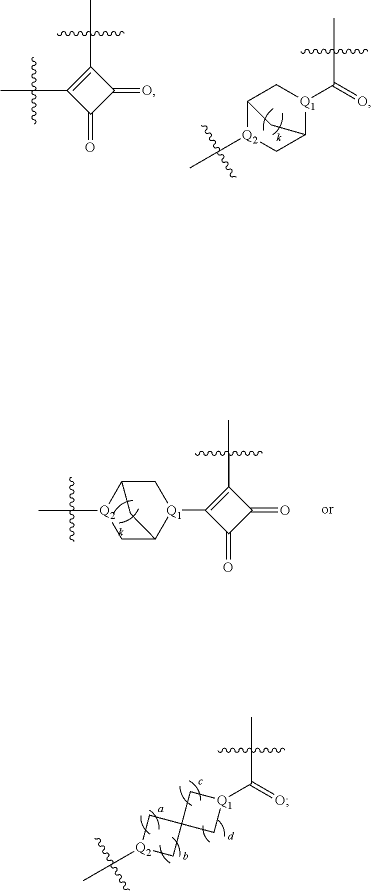
Rₐ is H or C₁₋₆ alkyl;
Q₁ and Q₂ are each independently CH or N;
a, b, c, d and k are each independently 1 or 2;
\textcircled{A} is C₃₋₁₂ cycloalkyl, 3- to 12-membered heterocycloalkyl including in a ring 1 to 3 heteroatoms independently selected from the group consisting of N, O and S, C₆-C₁₂ aryl, —(C₁₋₃ alkylene)C₆-C₁₂ aryl, 5- to 12-membered heteroaryl including in a ring 1 to 3 heteroatoms independently selected from the group consisting of N, O and S, or a 9- to 12-membered fused ring group including a structure in which an aromatic ring group and a non-aromatic ring group are fused;
at least one H of above \textcircled{A} may be each independently substituted with C₁₋₆ alkyl, C₂₋₆ alkenyl, C₂₋₆ alkynyl, C₃₋₁₀ cycloalkyl, C₃₋₁₂ cycloalkenyl, C₁₋₆ alkoxy, 3- to 12-membered heterocycloalkyl including in a ring 1 to 3 heteroatoms independently selected from the group consisting of N, O and S (in which at least one H of heterocycloalkyl may be substituted with —C(═O)(C₁₋₆ alkyl), 5- to 12-membered heteroaryl including in a ring 1 to 3 heteroatoms independently selected from the group consisting of N, O and S, —CF₃, —S(CF₃), halogen or —S(═NH)(═O)R_{c};
R_{c} is H or C₁₋₆ alkyl;
\textcircled{B} is C₆-C₁₂ aryl or 5- to 12-membered heteroaryl including in a ring 1 to 3 heteroatoms independently selected from the group consisting of N, O and S;
at least one H of above \textcircled{B} may be each independently substituted with C₁₋₆ alkyl or halogen;
\textcircled{D} is 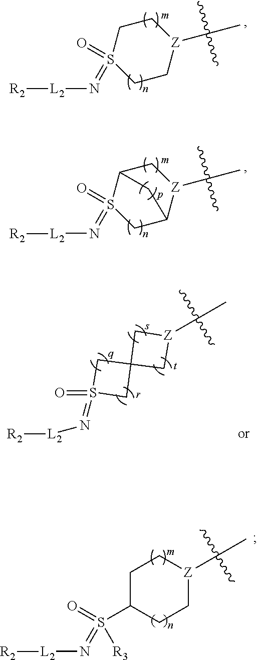
at least one H of above \textcircled{D} may be each independently substituted with C₁₋₆ alkyl or halogen;
Z is CH or N;
m and n are each independently 0, 1 or 2;
p, q, r, s and t are each independently 1 or 2;
L₂ is a single bond, —(C═O)—, —C(═O)O—, —C(═O)O—(C₁₋₃ alkylene)- or —S(═O)₂—;
R₂ is H, C₁₋₆ alkyl, C₆-C₁₂ aryl, CF₃ or —(P═O)(OR_{b})₂;
R_{b} is H or C₁₋₆ alkyl; and
R₃ is C₁₋₆ alkyl.

2. The sulfoximine compound, stereoisomers thereof or pharmaceutically acceptable salts thereof according to claim 1, wherein:
L₁ is —C(═O)—, —S(═O)₂—, —S(═O)₂—(C₁₋₃ alkylene)-, —C(═O)N(Rₐ)—, 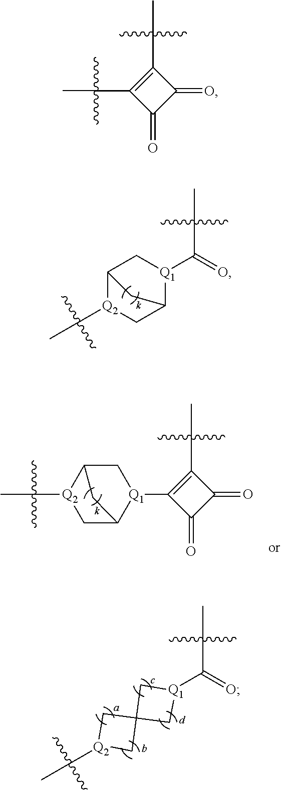
\textcircled{A} is C₆-C₁₂ aryl, —(C₁₋₃ alkylene)C₆-C₁₂ aryl, 5- to 12-membered heteroaryl including in a ring 1 to 3 heteroatoms independently selected from the group consisting of N, O and S, or 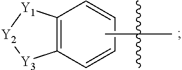
Y₁, Y₂ and Y₃ are each independently —CH₂— or —O—;
at least one H of above \textcircled{A} may be each independently substituted with C₁₋₆ alkyl, C₃₋₁₀ cycloalkyl, C₁₋₆ alkoxy, 3- to 12-membered heterocycloalkyl including in a ring 1 to 3 heteroatoms independently selected from the group consisting of N, O and S {in which at least one H of heterocycloalkyl may be substituted with —C(═O)(C₁₋₆ alkyl)}, 5- to 12-membered heteroaryl including in a ring 1 to 3 heteroatoms independently selected from the group consisting of N, O and S, —CF₃, —S(CF₃), halogen or —S(═NH)(═O)R_{c};
\textcircled{B} is C₆-C₁₂ aryl or 5- to 12-membered heteroaryl including in a ring 1 to 3 heteroatoms independently selected from the group consisting of N, O and S;
at least one H of above \textcircled{B} may be each independently substituted with halogen;
\textcircled{D} is 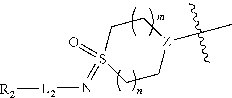
{in which, in 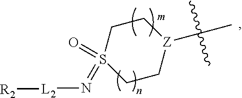
at least one H of 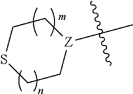
may be each independently substituted with C₁₋₆ alkyl or halogen}, 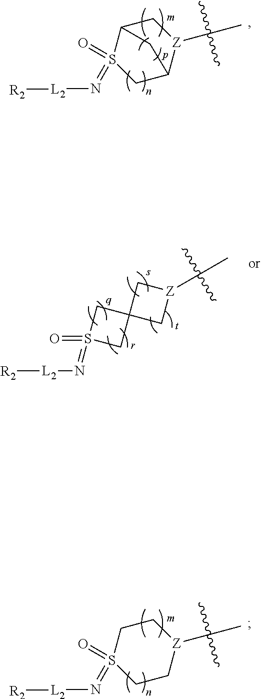
and
R₁, R₂, R₃, Rₐ, R_{b}, R_{c}, Q₁, Q₂, L₂, Z, a, b, c, d, k, m, n, p, q, r, s and t are same as defined in claim 1, respectively.

3. The sulfoximine compound, stereoisomers thereof or pharmaceutically acceptable salts thereof according to claim 1, wherein the sulfoximine compound is represented by formula Ia below: 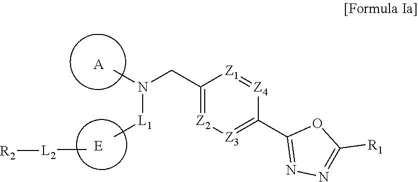
in above formula Ia,
L₁ is —C(═O)—, —S(═O)₂—, —S(═O)₂—(C₁₋₃ alkylene)-, —C(═O)N(Rₐ)—, 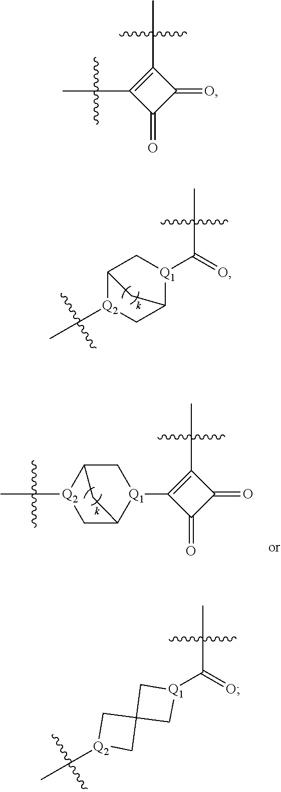
\textcircled{A} is C₆-C₁₂ aryl, —(C₁₋₃ alkylene)C₆-C₁₂ aryl, 5- to 12-membered heteroaryl including in a ring 1 to 3 heteroatoms independently selected from the group consisting of N, O and S, or a 9- to 12-membered fused ring group including a structure in which an aromatic ring group and a non-aromatic ring group are fused;
at least one H of above \textcircled{A} may be each independently substituted with C₁₋₆ alkyl, C₁₋₆ alkoxy, 3- to 12-membered heterocycloalkyl including in a ring 1 to 3 heteroatoms independently selected from the group consisting of N, O and S {in which at least one H of heterocycloalkyl may be substituted with —C(═O)(C₁₋₆ alkyl)}, 5- to 12-membered heteroaryl including in a ring 1 to 3 heteroatoms independently selected from the group consisting of N, O and S, —CF₃, —S(CF₃), halogen or —S(═NH)(═O)R_{c}; and
Z₁, Z₂, Z₃ and Z₄ are each independently CH, CX or N, in which at least one of Z₁ to Z₄ is CX or N;
X is halogen;
\textcircled{E} is 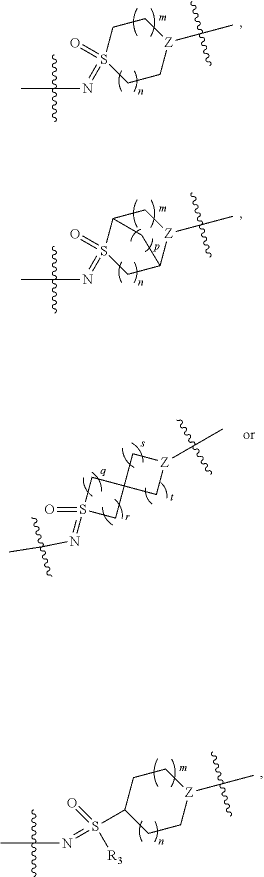
in above 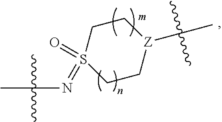
at least one H of 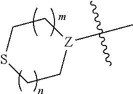
may be substituted with C₁₋₆ alkyl; and
R₁, R₂, R₃, Rₐ, R_{c}, Q₁, Q₂, L₂, Z, k, m, n, p, q, r, s and t are same as defined in claim 1, respectively.

4. The sulfoximine compound, stereoisomers thereof or pharmaceutically acceptable salts thereof according to claim 3, wherein in above formula Ia:
Z₁ is CX or N;
Z₂ is CH, CX or N;
Z₃ and Z₄ are each independently CH or N;
X is halogen;
L₁ is —C(═O)—, —S(═O)₂—, —S(═O)₂—(C₁₋₃ alkylene)-, —C(═O)NH—, 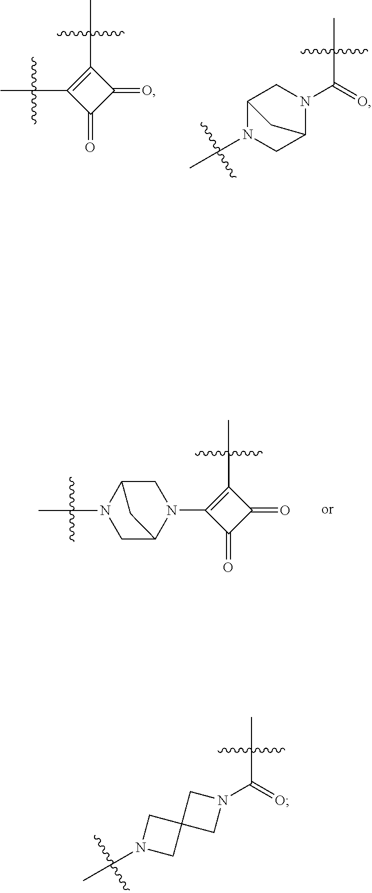 [SMILES: *c1c(*)c(=O)c1=O ; *C(=O)N1CC2CC1CN2* ; *c1c(N2CC3CC2CN3*)c(=O)c1=O ; *C(=O)N1CC2(CN(*)C2)C1]
\textcircled{A} is C₆-C₁₂ aryl, benzyl, 5- to 12-membered heteroaryl including in a ring 1 to 3 heteroatoms independently selected from the group consisting of N, O and S, or 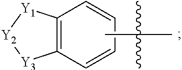
Y₁, Y₂ and Y₃ are each independently —CH₂— or —O—;
At least one H of above \textcircled{A} may be each independently substituted with C₁₋₆ alkyl, C₁₋₆ alkoxy, 3- to 12-membered heterocycloalkyl including in a ring 1 to 3 heteroatoms independently selected from the group consisting of N, O and S {in which at least one H of heterocycloalkyl may be substituted with —C(═O)(C₁₋₆ alkyl)}, 5- to 12-membered heteroaryl including in a ring 1 to 3 heteroatoms independently selected from the group consisting of N, O and S, —CF₃, —S(CF₃), halogen or —S(═NH)(═O)R_{c}; and
R_{c} is same as defined in claim 3.

5. The sulfoximine compound, stereoisomers thereof or pharmaceutically acceptable salts thereof according to claim 3, wherein in above formula Ia:
Z₁ is CF or N;
Z₂ is CH, CF or N;
Z₃ and Z₄ are each independently CH or N;
L₁ is —C(═O)—, —S(═O)₂—, —S(═O)₂—(C₁₋₃ alkylene)-, —C(═O)NH—, 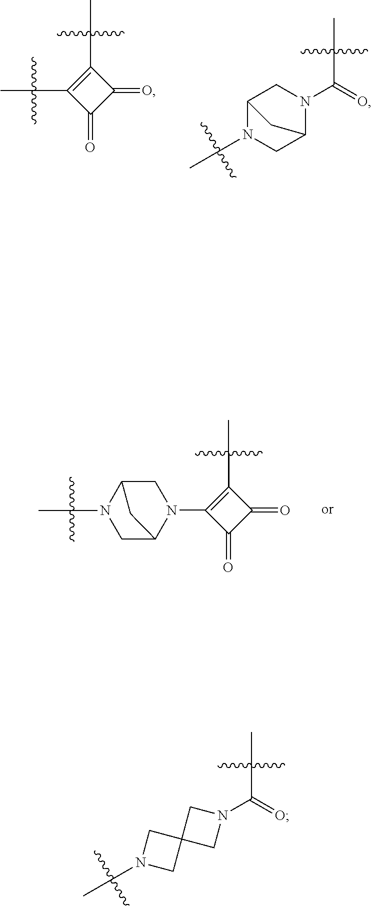 [SMILES: *c1c(*)c(=O)c1=O ; *C(=O)N1CC2CC1CN2* ; *c1c(N2CC3CC2CN3*)c(=O)c1=O ; *C(=O)N1CC2(CN(*)C2)C1]
R₂ is H, C₁₋₆ alkyl, C₆-C₁₂ aryl, CF₃ or —(P═O)(OR_{b})₂;
R_{b} is C₁₋₆ alkyl;
\textcircled{A} is C₆-C₁₂ aryl, benzyl, 5- to 12-membered heteroaryl including in a ring 1 to 3 heteroatoms independently selected from the group consisting of N, O and S, 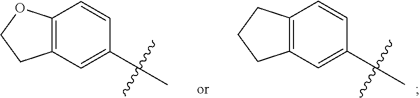 [SMILES: *c1ccc2c(c1)CCO2 ; *c1ccc2c(c1)CCC2]
in above \textcircled{A}, at least one H of C₆-C₁₂ aryl or 5- to 12-membered heteroaryl including in a ring 1 to 3 heteroatoms independently selected from the group consisting of N, O and S may be each independently substituted with C₁₋₆ alkyl, C₁₋₆ alkoxy, 5- to 12-membered heteroaryl including in a ring 1 to 3 heteroatoms independently selected from the group consisting of N, O and S, —CF₃, —S(CF₃), halogen or —S(═NH)(═O)R_{c}; and
R_{c} is C₁₋₆ alkyl.

6. The sulfoximine compound, stereoisomers thereof or pharmaceutically acceptable salts thereof according to claim 1, wherein the sulfoximine compound is represented by formula Ib below: 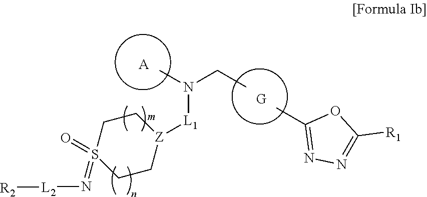
in above formula Ib
\textcircled{G} is 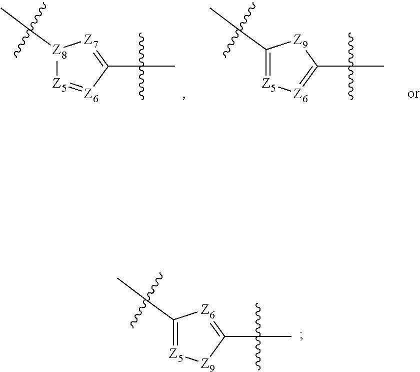
Z₅, Z₆, Z₇ and Z₈ are each independently CH, CX or N, in which at least one of Z₅ to Z₆ is CX or N;
Z₉ is CH₂, CHX, CX₂, NR₇, O or S;
R₇ is H or C₁₋₆ alkyl;
X is halogen;
\textcircled{A}, R₁, R₂, L₂, Z, m and n are same as defined in claim 1, respectively.

7. A sulfoximine compound, stereoisomers thereof or pharmaceutically acceptable salts thereof, wherein the sulfoximine compound is any one selected from the group consisting of compounds shown in table below:

| Com- | |
|---|---|
| pound | |
| No. | Structure |
| 1 | |
| 2 | |
| 3 | |
| 4 | |
| 5 | 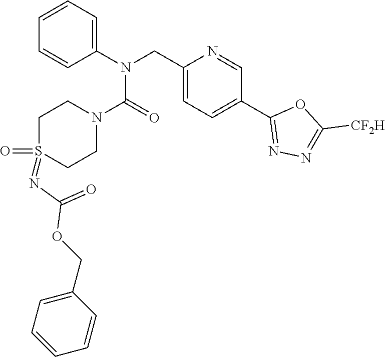 [SMILES: O=C(N=S1(=O)CCN(C(=O)N(Cc2ccc(-c3nnc(C(F)F)o3)cn2)c2ccccc2)CC1)OCc1ccccc1] |
| 6 | 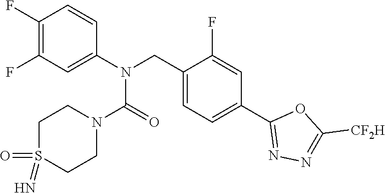 [SMILES: N=S1(=O)CCN(C(=O)N(Cc2ccc(-c3nnc(C(F)F)o3)cc2F)c2ccc(F)c(F)c2)CC1] |
| 7 | 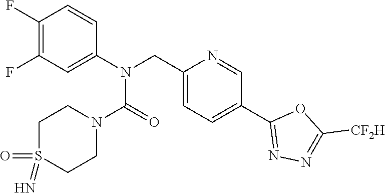 [SMILES: N=S1(=O)CCN(C(=O)N(Cc2ccc(-c3nnc(C(F)F)o3)cn2)c2ccc(F)c(F)c2)CC1] |
| 8 | 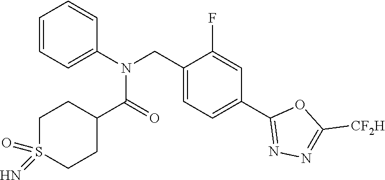 [SMILES: N=S1(=O)CCC(C(=O)N(Cc2ccc(-c3nnc(C(F)F)o3)cc2F)c2ccccc2)CC1] |
| 9 | 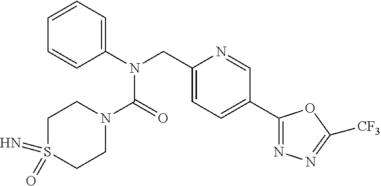 [SMILES: N=S1(=O)CCN(C(=O)N(Cc2ccc(-c3nnc(C(F)(F)F)o3)cn2)c2ccccc2)CC1] |
| 10 | 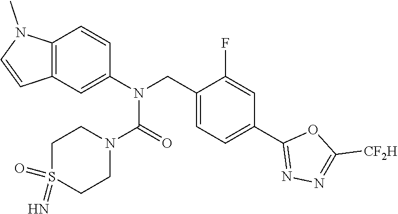 [SMILES: Cn1ccc2cc(N(Cc3ccc(-c4nnc(C(F)F)o4)cc3F)C(=O)N3CCS(=N)(=O)CC3)ccc21] |
| 11 | 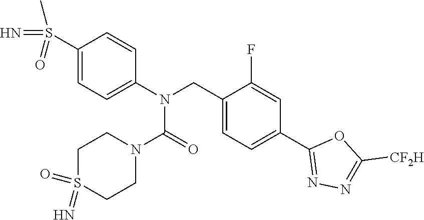 [SMILES: CS(=N)(=O)c1ccc(N(Cc2ccc(-c3nnc(C(F)F)o3)cc2F)C(=O)N2CCS(=N)(=O)CC2)cc1] |
| 12 | 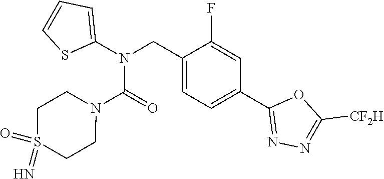 [SMILES: N=S1(=O)CCN(C(=O)N(Cc2ccc(-c3nnc(C(F)F)o3)cc2F)c2cccs2)CC1] |
| 13 | 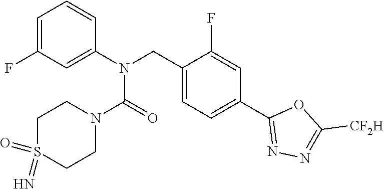 [SMILES: N=S1(=O)CCN(C(=O)N(Cc2ccc(-c3nnc(C(F)F)o3)cc2F)c2cccc(F)c2)CC1] |
| 14 | 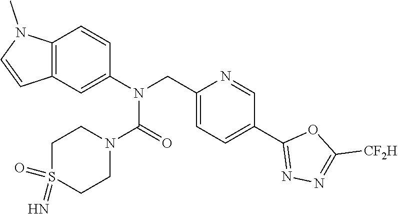 [SMILES: Cn1ccc2cc(N(Cc3ccc(-c4nnc(C(F)F)o4)cn3)C(=O)N3CCS(=N)(=O)CC3)ccc21] |
| 15 | 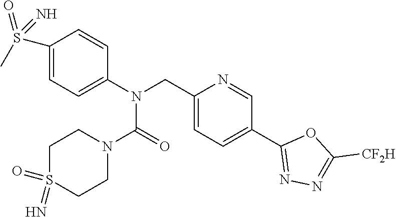 [SMILES: CS(=N)(=O)c1ccc(N(Cc2ccc(-c3nnc(C(F)F)o3)cn2)C(=O)N2CCS(=N)(=O)CC2)cc1] |
| 16 | 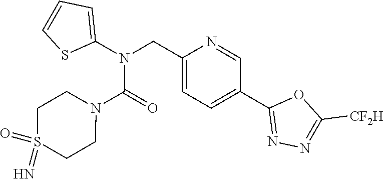 [SMILES: N=S1(=O)CCN(C(=O)N(Cc2ccc(-c3nnc(C(F)F)o3)cn2)c2cccs2)CC1] |
| 17 | 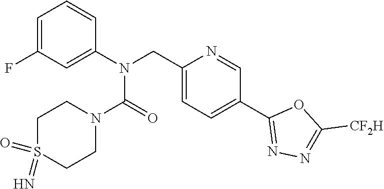 [SMILES: N=S1(=O)CCN(C(=O)N(Cc2ccc(-c3nnc(C(F)F)o3)cn2)c2cccc(F)c2)CC1] |
| 18 | 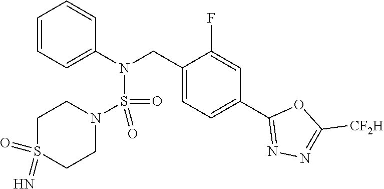 [SMILES: N=S1(=O)CCN(S(=O)(=O)N(Cc2ccc(-c3nnc(C(F)F)o3)cc2F)c2ccccc2)CC1] |
| 19 | 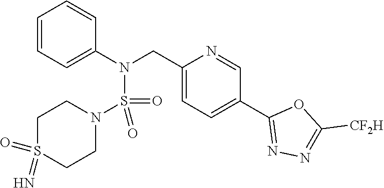 [SMILES: N=S1(=O)CCN(S(=O)(=O)N(Cc2ccc(-c3nnc(C(F)F)o3)cn2)c2ccccc2)CC1] |
| 20 | 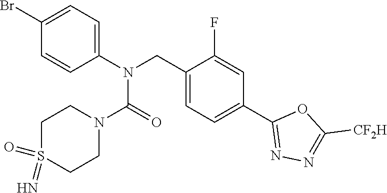 [SMILES: N=S1(=O)CCN(C(=O)N(Cc2ccc(-c3nnc(C(F)F)o3)cc2F)c2ccc(Br)cc2)CC1] |
| 21 | 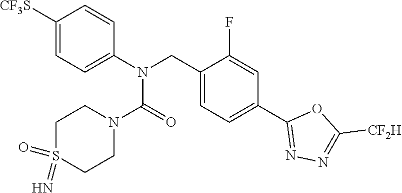 |
| 22 | 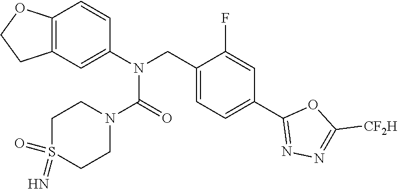 [SMILES: N=S1(=O)CCN(C(=O)N(Cc2ccc(-c3nnc(C(F)F)o3)cc2F)c2ccc3c(c2)CCO3)CC1] |
| 23 | 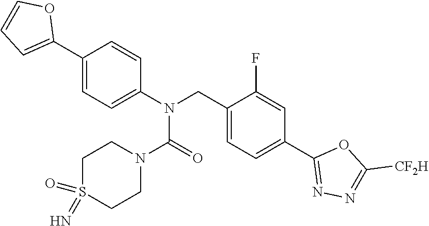 [SMILES: N=S1(=O)CCN(C(=O)N(Cc2ccc(-c3nnc(C(F)F)o3)cc2F)c2ccc(-c3ccco3)cc2)CC1] |
| 24 | 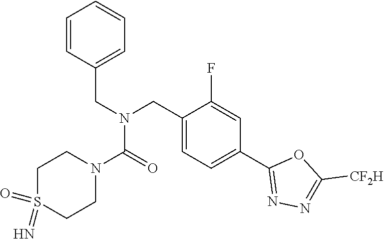 [SMILES: N=S1(=O)CCN(C(=O)N(Cc2ccccc2)Cc2ccc(-c3nnc(C(F)F)o3)cc2F)CC1] |
| 25 | 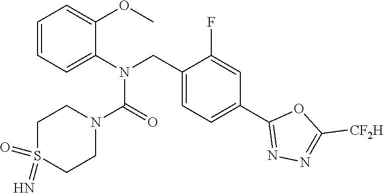 [SMILES: COc1ccccc1N(Cc1ccc(-c2nnc(C(F)F)o2)cc1F)C(=O)N1CCS(=N)(=O)CC1] |
| 26 | 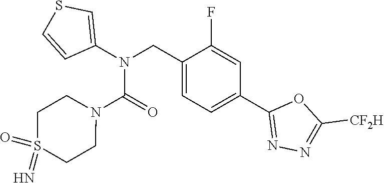 [SMILES: N=S1(=O)CCN(C(=O)N(Cc2ccc(-c3nnc(C(F)F)o3)cc2F)c2ccsc2)CC1] |
| 27 | 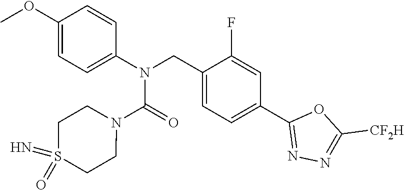 [SMILES: COc1ccc(N(Cc2ccc(-c3nnc(C(F)F)o3)cc2F)C(=O)N2CCS(=N)(=O)CC2)cc1] |
| 28 | 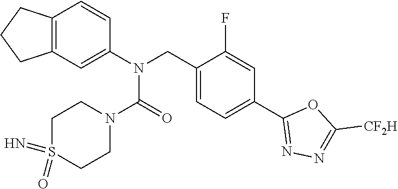 [SMILES: N=S1(=O)CCN(C(=O)N(Cc2ccc(-c3nnc(C(F)F)o3)cc2F)c2ccc3c(c2)CCC3)CC1] |
| 29 | 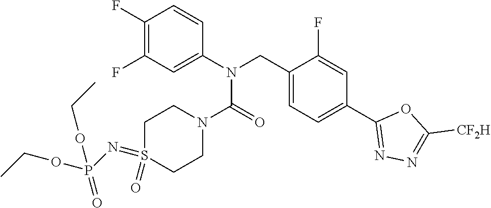 [SMILES: CCOP(=O)(N=S1(=O)CCN(C(=O)N(Cc2ccc(-c3nnc(C(F)F)o3)cc2F)c2ccc(F)c(F)c2)CC1)OCC] |
| 30 | 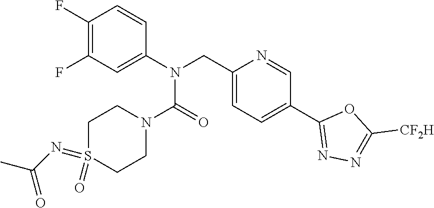 [SMILES: CC(=O)N=S1(=O)CCN(C(=O)N(Cc2ccc(-c3nnc(C(F)F)o3)cn2)c2ccc(F)c(F)c2)CC1] |
| 31 | 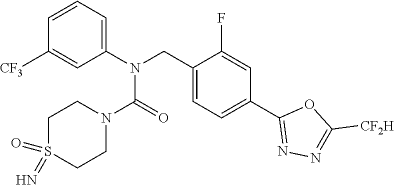 [SMILES: N=S1(=O)CCN(C(=O)N(Cc2ccc(-c3nnc(C(F)F)o3)cc2F)c2cccc(C(F)(F)F)c2)CC1] |
| 32 | 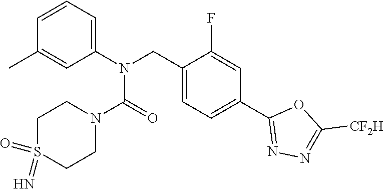 [SMILES: Cc1cccc(N(Cc2ccc(-c3nnc(C(F)F)o3)cc2F)C(=O)N2CCS(=N)(=O)CC2)c1] |
| 33 | 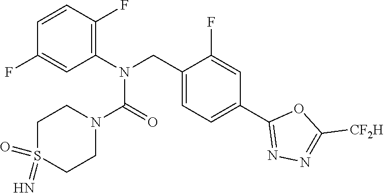 [SMILES: N=S1(=O)CCN(C(=O)N(Cc2ccc(-c3nnc(C(F)F)o3)cc2F)c2cc(F)ccc2F)CC1] |
| 34 | 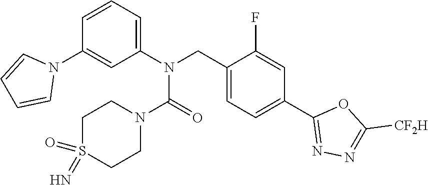 [SMILES: N=S1(=O)CCN(C(=O)N(Cc2ccc(-c3nnc(C(F)F)o3)cc2F)c2cccc(-n3cccc3)c2)CC1] |
| 35 | 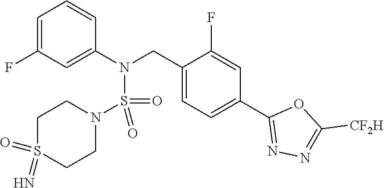 [SMILES: N=S1(=O)CCN(S(=O)(=O)N(Cc2ccc(-c3nnc(C(F)F)o3)cc2F)c2cccc(F)c2)CC1] |
| 36 | 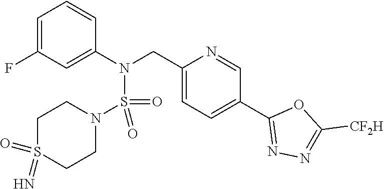 [SMILES: N=S1(=O)CCN(S(=O)(=O)N(Cc2ccc(-c3nnc(C(F)F)o3)cn2)c2cccc(F)c2)CC1] |
| 37 | 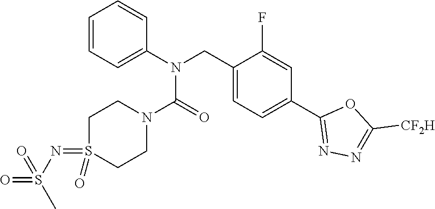 [SMILES: CS(=O)(=O)N=S1(=O)CCN(C(=O)N(Cc2ccc(-c3nnc(C(F)F)o3)cc2F)c2ccccc2)CC1] |
| 38 | 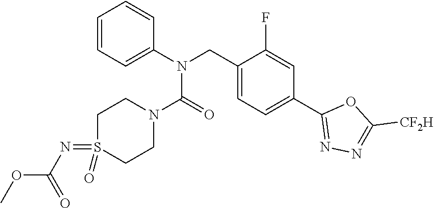 [SMILES: COC(=O)N=S1(=O)CCN(C(=O)N(Cc2ccc(-c3nnc(C(F)F)o3)cc2F)c2ccccc2)CC1] |
| 39 | 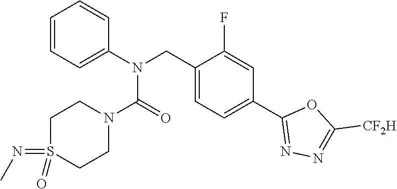 [SMILES: CN=S1(=O)CCN(C(=O)N(Cc2ccc(-c3nnc(C(F)F)o3)cc2F)c2ccccc2)CC1] |
| 40 | 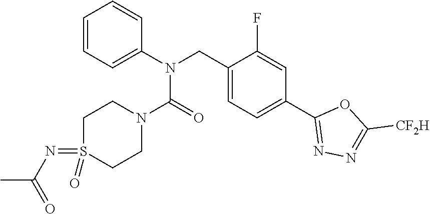 [SMILES: CC(=O)N=S1(=O)CCN(C(=O)N(Cc2ccc(-c3nnc(C(F)F)o3)cc2F)c2ccccc2)CC1] |
| 41 | 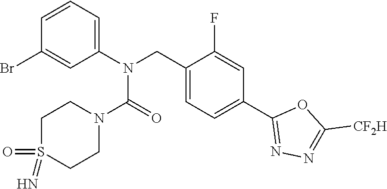 [SMILES: N=S1(=O)CCN(C(=O)N(Cc2ccc(-c3nnc(C(F)F)o3)cc2F)c2cccc(Br)c2)CC1] |
| 42 | 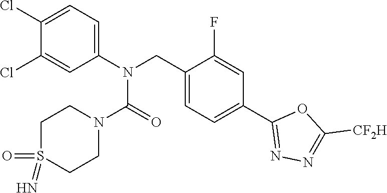 [SMILES: N=S1(=O)CCN(C(=O)N(Cc2ccc(-c3nnc(C(F)F)o3)cc2F)c2ccc(Cl)c(Cl)c2)CC1] |
| 43 | 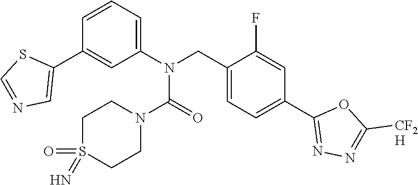 [SMILES: N=S1(=O)CCN(C(=O)N(Cc2ccc(-c3nnc(C(F)F)o3)cc2F)c2cccc(-c3cncs3)c2)CC1] |
| 44 | 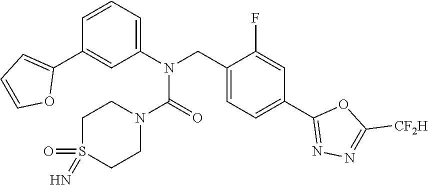 [SMILES: N=S1(=O)CCN(C(=O)N(Cc2ccc(-c3nnc(C(F)F)o3)cc2F)c2cccc(-c3ccco3)c2)CC1] |
| 45 | 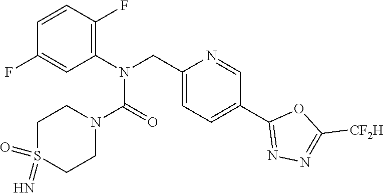 [SMILES: N=S1(=O)CCN(C(=O)N(Cc2ccc(-c3nnc(C(F)F)o3)cn2)c2cc(F)ccc2F)CC1] |
| 46 | 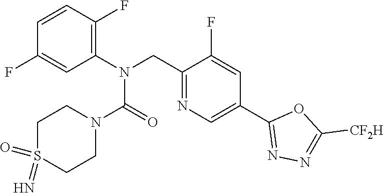 [SMILES: N=S1(=O)CCN(C(=O)N(Cc2ncc(-c3nnc(C(F)F)o3)cc2F)c2cc(F)ccc2F)CC1] |
| 47 | 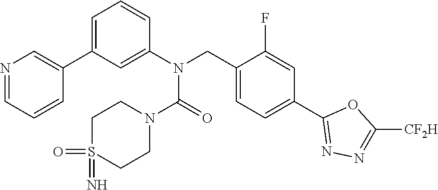 [SMILES: N=S1(=O)CCN(C(=O)N(Cc2ccc(-c3nnc(C(F)F)o3)cc2F)c2cccc(-c3cccnc3)c2)CC1] |
| 48 | 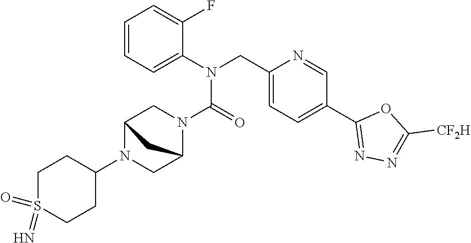 [SMILES: N=S1(=O)CCC(N2C[C@@H]3C[C@H]2CN3C(=O)N(Cc2ccc(-c3nnc(C(F)F)o3)cn2)c2ccccc2F)CC1] |
| 49 | 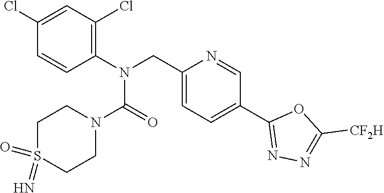 [SMILES: N=S1(=O)CCN(C(=O)N(Cc2ccc(-c3nnc(C(F)F)o3)cn2)c2ccc(Cl)cc2Cl)CC1] |
| 50 | 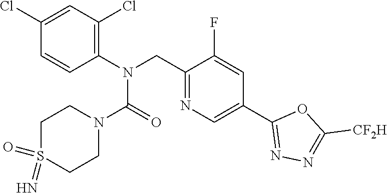 [SMILES: N=S1(=O)CCN(C(=O)N(Cc2ncc(-c3nnc(C(F)F)o3)cc2F)c2ccc(Cl)cc2Cl)CC1] |
| 51 | 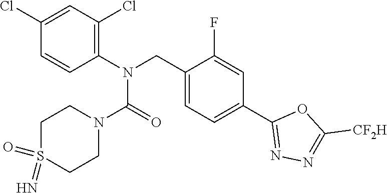 [SMILES: N=S1(=O)CCN(C(=O)N(Cc2ccc(-c3nnc(C(F)F)o3)cc2F)c2ccc(Cl)cc2Cl)CC1] |
| 52 | 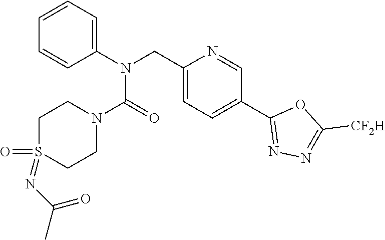 [SMILES: CC(=O)N=S1(=O)CCN(C(=O)N(Cc2ccc(-c3nnc(C(F)F)o3)cn2)c2ccccc2)CC1] |
| 53 | 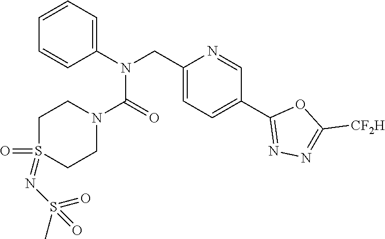 [SMILES: CS(=O)(=O)N=S1(=O)CCN(C(=O)N(Cc2ccc(-c3nnc(C(F)F)o3)cn2)c2ccccc2)CC1] |
| 54 | 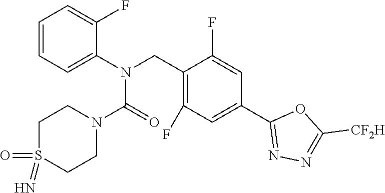 [SMILES: N=S1(=O)CCN(C(=O)N(Cc2c(F)cc(-c3nnc(C(F)F)o3)cc2F)c2ccccc2F)CC1] |
| 55 | 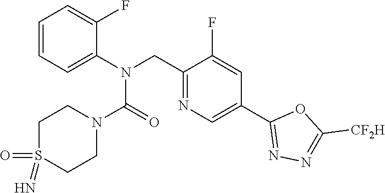 [SMILES: N=S1(=O)CCN(C(=O)N(Cc2ncc(-c3nnc(C(F)F)o3)cc2F)c2ccccc2F)CC1] |
| 56 | 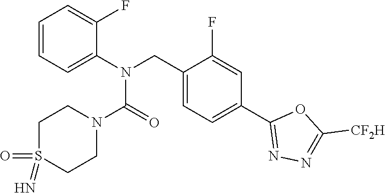 [SMILES: N=S1(=O)CCN(C(=O)N(Cc2ccc(-c3nnc(C(F)F)o3)cc2F)c2ccccc2F)CC1] |
| 57 | 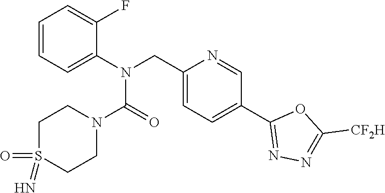 [SMILES: N=S1(=O)CCN(C(=O)N(Cc2ccc(-c3nnc(C(F)F)o3)cn2)c2ccccc2F)CC1] |
| 58 | 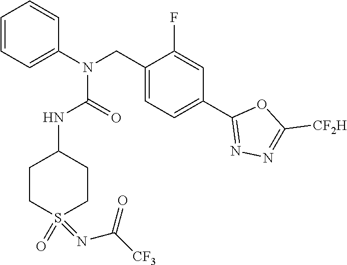 [SMILES: O=C(NC1CCS(=O)(=NC(=O)C(F)(F)F)CC1)N(Cc1ccc(-c2nnc(C(F)F)o2)cc1F)c1ccccc1] |
| 59 | 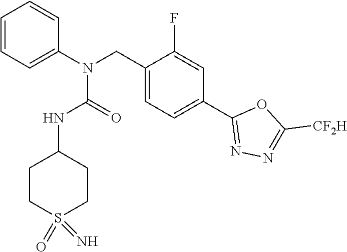 [SMILES: N=S1(=O)CCC(NC(=O)N(Cc2ccc(-c3nnc(C(F)F)o3)cc2F)c2ccccc2)CC1] |
| 60 | 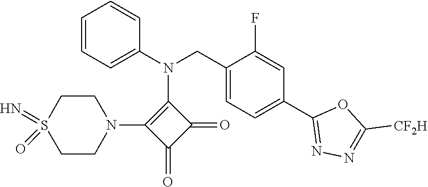 [SMILES: N=S1(=O)CCN(c2c(N(Cc3ccc(-c4nnc(C(F)F)o4)cc3F)c3ccccc3)c(=O)c2=O)CC1] |
| 61 | 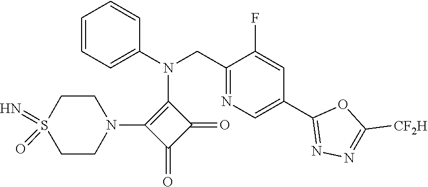 [SMILES: N=S1(=O)CCN(c2c(N(Cc3ncc(-c4nnc(C(F)F)o4)cc3F)c3ccccc3)c(=O)c2=O)CC1] |
| 62 | 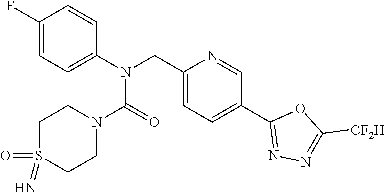 [SMILES: N=S1(=O)CCN(C(=O)N(Cc2ccc(-c3nnc(C(F)F)o3)cn2)c2ccc(F)cc2)CC1] |
| 63 | 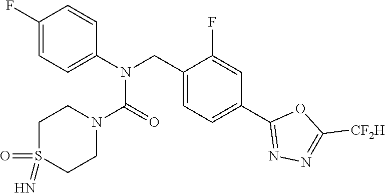 [SMILES: N=S1(=O)CCN(C(=O)N(Cc2ccc(-c3nnc(C(F)F)o3)cc2F)c2ccc(F)cc2)CC1] |
| 64 | 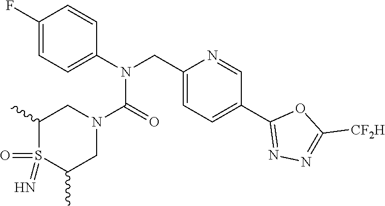 |
| 65 | 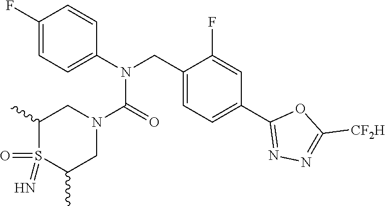 |
| 66 | 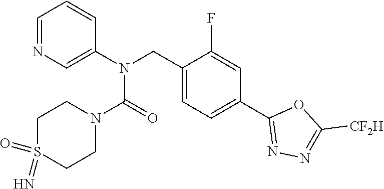 [SMILES: N=S1(=O)CCN(C(=O)N(Cc2ccc(-c3nnc(C(F)F)o3)cc2F)c2cccnc2)CC1] |
| 67 | 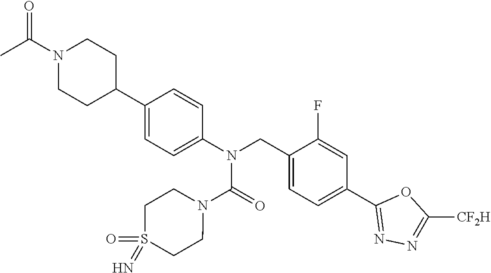 [SMILES: CC(=O)N1CCC(c2ccc(N(Cc3ccc(-c4nnc(C(F)F)o4)cc3F)C(=O)N3CCS(=N)(=O)CC3)cc2)CC1] |
| 68 | 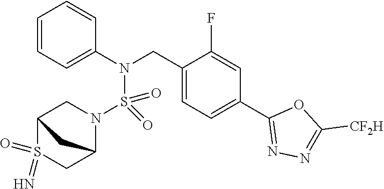 [SMILES: N=S1(=O)C[C@@H]2C[C@H]1CN2S(=O)(=O)N(Cc1ccc(-c2nnc(C(F)F)o2)cc1F)c1ccccc1] |
| 69 | 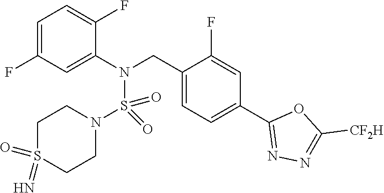 [SMILES: N=S1(=O)CCN(S(=O)(=O)N(Cc2ccc(-c3nnc(C(F)F)o3)cc2F)c2cc(F)ccc2F)CC1] |
| 70 | 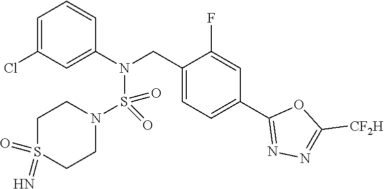 [SMILES: N=S1(=O)CCN(S(=O)(=O)N(Cc2ccc(-c3nnc(C(F)F)o3)cc2F)c2cccc(Cl)c2)CC1] |
| 71 | 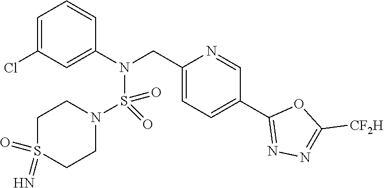 [SMILES: N=S1(=O)CCN(S(=O)(=O)N(Cc2ccc(-c3nnc(C(F)F)o3)cn2)c2cccc(Cl)c2)CC1] |
| 72 | 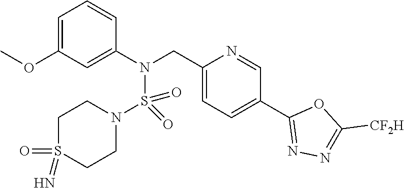 [SMILES: COc1cccc(N(Cc2ccc(-c3nnc(C(F)F)o3)cn2)S(=O)(=O)N2CCS(=N)(=O)CC2)c1] |
| 73 | 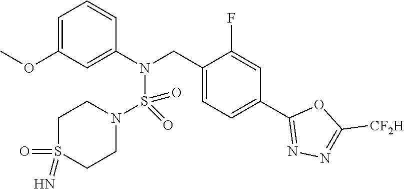 [SMILES: COc1cccc(N(Cc2ccc(-c3nnc(C(F)F)o3)cc2F)S(=O)(=O)N2CCS(=N)(=O)CC2)c1] |
| 74 | 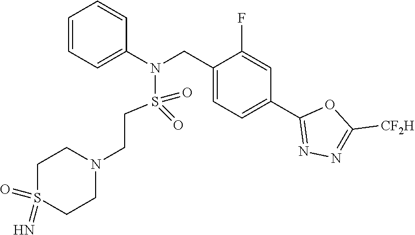 [SMILES: N=S1(=O)CCN(CCS(=O)(=O)N(Cc2ccc(-c3nnc(C(F)F)o3)cc2F)c2ccccc2)CC1] |
| 75 | 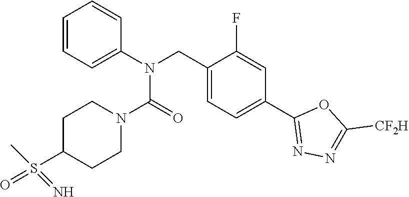 [SMILES: CS(=N)(=O)C1CCN(C(=O)N(Cc2ccc(-c3nnc(C(F)F)o3)cc2F)c2ccccc2)CC1] |
| 76 | 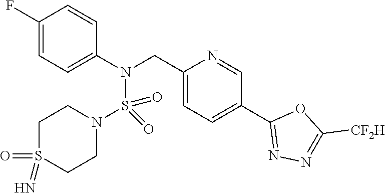 [SMILES: N=S1(=O)CCN(S(=O)(=O)N(Cc2ccc(-c3nnc(C(F)F)o3)cn2)c2ccc(F)cc2)CC1] |
| 77 | 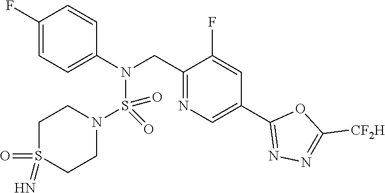 [SMILES: N=S1(=O)CCN(S(=O)(=O)N(Cc2ncc(-c3nnc(C(F)F)o3)cc2F)c2ccc(F)cc2)CC1] |
| 78 | 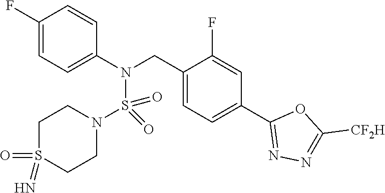 [SMILES: N=S1(=O)CCN(S(=O)(=O)N(Cc2ccc(-c3nnc(C(F)F)o3)cc2F)c2ccc(F)cc2)CC1] |
| 79 | 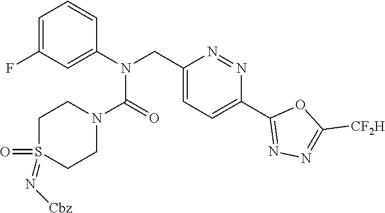 [SMILES: O=C(N=S1(=O)CCN(C(=O)N(Cc2ccc(-c3nnc(C(F)F)o3)nn2)c2cccc(F)c2)CC1)OCc1ccccc1] |
| 80 | 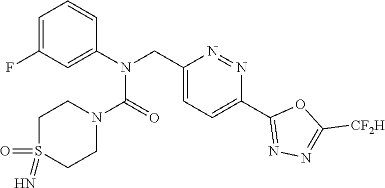 [SMILES: N=S1(=O)CCN(C(=O)N(Cc2ccc(-c3nnc(C(F)F)o3)nn2)c2cccc(F)c2)CC1] |
| 81 | 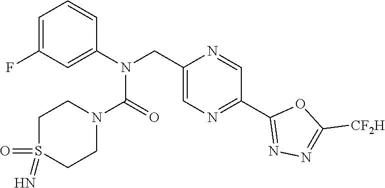 [SMILES: N=S1(=O)CCN(C(=O)N(Cc2cnc(-c3nnc(C(F)F)o3)cn2)c2cccc(F)c2)CC1] |
| 82 | 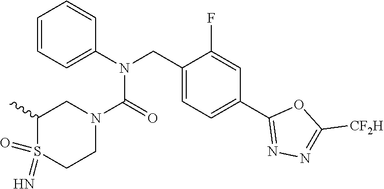 [SMILES: CC1CN(C(=O)N(Cc2ccc(-c3nnc(C(F)F)o3)cc2F)c2ccccc2)CCS1(=N)=O] |
| 83 | 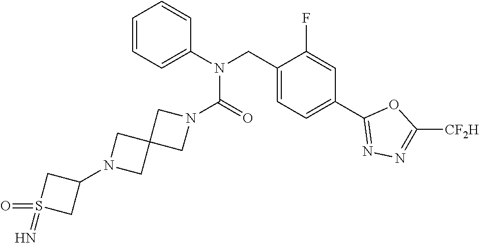 [SMILES: N=S1(=O)CC(N2CC3(CN(C(=O)N(Cc4ccc(-c5nnc(C(F)F)o5)cc4F)c4ccccc4)C3)C2)C1] |
| 84 | 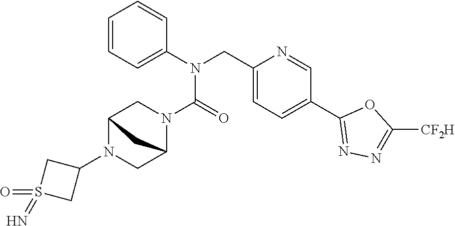 [SMILES: N=S1(=O)CC(N2C[C@@H]3C[C@H]2CN3C(=O)N(Cc2ccc(-c3nnc(C(F)F)o3)cn2)c2ccccc2)C1] |
| 85 | 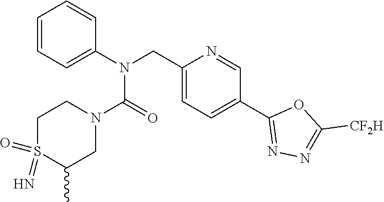 [SMILES: CC1CN(C(=O)N(Cc2ccc(-c3nnc(C(F)F)o3)cn2)c2ccccc2)CCS1(=N)=O] |
| 86 | 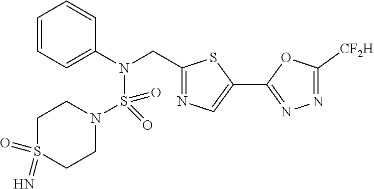 [SMILES: N=S1(=O)CCN(S(=O)(=O)N(Cc2ncc(-c3nnc(C(F)F)o3)s2)c2ccccc2)CC1] |
| 87 | 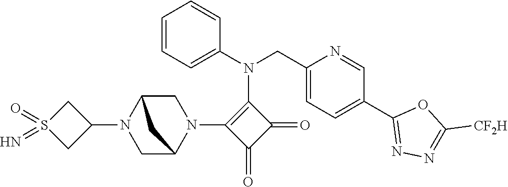 [SMILES: N=S1(=O)CC(N2C[C@@H]3C[C@H]2CN3c2c(N(Cc3ccc(-c4nnc(C(F)F)o4)cn3)c3ccccc3)c(=O)c2=O)C1] |
| 88 | 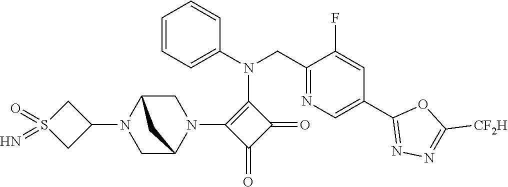 [SMILES: N=S1(=O)CC(N2C[C@@H]3C[C@H]2CN3c2c(N(Cc3ncc(-c4nnc(C(F)F)o4)cc3F)c3ccccc3)c(=O)c2=O)C1] |
| 89 | 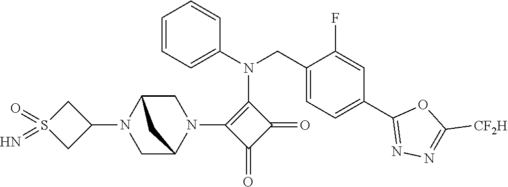 [SMILES: N=S1(=O)CC(N2C[C@@H]3C[C@H]2CN3c2c(N(Cc3ccc(-c4nnc(C(F)F)o4)cc3F)c3ccccc3)c(=O)c2=O)C1] |
| 90 | 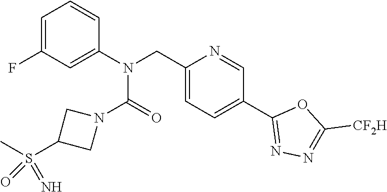 [SMILES: CS(=N)(=O)C1CN(C(=O)N(Cc2ccc(-c3nnc(C(F)F)o3)cn2)c2cccc(F)c2)C1] |
| 91 | 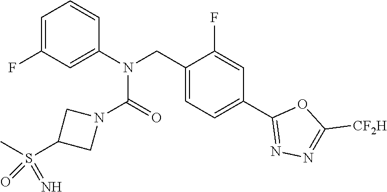 [SMILES: CS(=N)(=O)C1CN(C(=O)N(Cc2ccc(-c3nnc(C(F)F)o3)cc2F)c2cccc(F)c2)C1] |
| 92 | 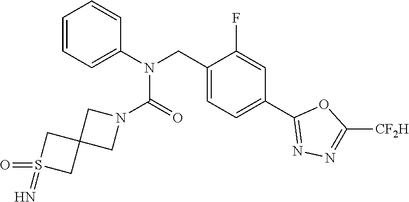 [SMILES: N=S1(=O)CC2(CN(C(=O)N(Cc3ccc(-c4nnc(C(F)F)o4)cc3F)c3ccccc3)C2)C1] |

8. A pharmaceutical composition comprising the sulfoximine compound according to claim 1, stereoisomers thereof or pharmaceutically acceptable salts thereof as an active ingredient.

9. The pharmaceutical composition according to claim 8, wherein the composition is for preventing or treating histone deacetylase-mediated diseases.

10. The pharmaceutical composition according to claim 9, wherein the histone deacetylase-mediated diseases comprise infectious diseases; neoplasm; endocrinopathy, nutritional and metabolic diseases; mental and behavioral disorders; neurological diseases; eye and ocular adnexal diseases; circulatory diseases; respiratory diseases; digestive troubles; renal failure; skin and subcutaneous tissue diseases; musculoskeletal system and connective tissue diseases; or teratosis, deformities and chromosomal aberration.

11. The pharmaceutical composition according to claim 10, wherein the endocrinopathy, nutritional and metabolic diseases comprise Wilson's disease, amyloidosis or diabetes;
the mental and behavioral disorders comprise depression or Rett syndrome;
the neurological diseases comprise central nervous system atrophy, neurodegenerative disease, motor disorder, neuropathy, motor neuron disease, central nervous system demyelinating disease, or Charcot-Marie-Tooth (CMT) disease;
the eye and ocular adnexal diseases comprise uveitis;
the skin and subcutaneous tissue diseases comprise psoriasis;
the musculoskeletal system and connective tissue diseases comprise rheumatoid arthritis, osteoarthritis or systemic lupus erythematosis;
the teratosis, deformities and chromosomal aberration comprise autosomal dominant polycystic kidney disease;
the infectious disease comprises prion disease;
the neoplasm comprises benign tumor or malignant tumor;
the circulatory disease comprises atrial fibrillation, stroke, heart failure, or pulmonary hypertension;
the respiratory disease comprises asthma or idiopathic pulmonary fibrosis; and
the digestive troubles comprise alcoholic liver disease, inflammatory bowel disease, Crohn's disease or ulcerative bowel disease; and
the renal failure comprises acute renal failure and chronic renal failure.

12. A method for preventing or treating HDAC-mediated diseases including administering the sulfoximine compound, stereoisomers thereof or pharmaceutically acceptable salts thereof according to claim 1 into an individual.

13. Use of the sulfoximine compound, stereoisomers thereof or pharmaceutically acceptable salts thereof according to claim 1 for inhibiting histone deacetylase.

14. Use of the sulfoximine compound, stereoisomers thereof or pharmaceutically acceptable salts thereof according to claim 1 in preparing a medicament for preventing or treating HDAC-mediated diseases.

15. The use according to claim 13, wherein the histone deacetylase is a histone deacetylase 6 (HDAC6).
